(19) United States
(12) Patent Application Publication
(10) Pub. No.: US 2004/0204450 A1
(43) Pub. Date: Oct. 14, 2004

(54) Quinoline and quinoxaline compounds

(75) Inventors: Bruce M. Bechle, Oakdale, CT (US); George C. Chang, Old Saybrook, CT (US); Mary Didiuk, Groton, CT (US); Jari I. Finneman, Salem, CT (US); Ravi S. Garigipati, South Glastonbury, CT (US); Ryan M. Kelley, Groton, CT (US); David A. Perry, Mystic, CT (US); Roger B. Ruggeri, Waterford, CT (US)

Correspondence Address: PFIZER INC., PATENT DEPARTMENT, MS8260-1611, EASTERN POINT ROAD, GROTON, CT, 06340, US

(73) Assignee: Pfizer Inc

(21) Appl. No.: 10/807,838

(22) Filed: Mar. 23, 2004

Related U.S. Application Data
(60) Provisional application No. 60/536,217, filed on Jan. 14, 2004.
(60) Provisional application No. 60/458,274, filed on Mar. 28, 2003.

(51) Int. Cl. A61K 31/4709; C 07D 4 1/02

(52) U.S. Cl. 514/314; 546/167

(57) ABSTRACT

Quinoline and quinoxaline compounds, pharmaceutical compositions containing such compounds and the use of such compounds to elevate certain plasma lipid levels, including high density lipoprotein-cholesterol and to lower certain other plasma lipid levels, such as LDL-cholesterol and triglycerides and accordingly to treat diseases which are exacerbated by low levels of HDL cholesterol and/or high levels of LDL-cholesterol and triglycerides, such as atherosclerosis and cardiovascular diseases in some mammals, including humans.

### CROSS-REFERENCE TO RELATED APPLICATION

This application claims the benefit of U.S. Provisional application Nos. 60/458,274 and 60/536,217, filed Mar. 28, 2003 and Jan. 24, 2004, respectively, both of which are incorporated herein by reference for all purposes.

### BACKGROUND OF INVENTION

This invention relates to quinoline and quinoxaline compounds, pharmaceutical compositions containing such inhibitors and the use of such inhibitors to elevate certain plasma lipid levels, including high density lipoprotein (HDL)-cholesterol and to lower certain other plasma lipid levels, such as low density lipoprotein (LDL)-cholesterol and triglycerides and accordingly to treat diseases which are affected by low levels of HDL cholesterol and/or high levels of LDL-cholesterol and triglycerides, such as atherosclerosis and cardiovascular diseases in certain mammals (i.e., those which have CETP in their plasma), including humans.

Atherosclerosis and its associated coronary artery disease (CAD) is the leading cause of mortality in the industrialized world. Despite attempts to modify secondary risk factors (smoking, obesity, lack of exercise) and treatment of dyslipidemia with dietary modification and drug therapy, coronary heart disease (CHD) remains the most common cause of death in the U.S., where cardiovascular disease accounts for 44% of all deaths, with 53% of these associated with atherosclerotic coronary heart disease.

Risk for development of this condition has been shown to be strongly correlated with certain plasma lipid levels. While elevated LDL-C may be the most recognized form of dyslipidemia, it is by no means the only significant lipid associated contributor to CHD. Low HDL-C is also a known risk factor for CHD (Gordon, D. J., et al.,: “High-density Lipoprotein Cholesterol and Cardiovascular Disease”, Circulation, (1989), 79: 8-15).

High LDL-cholesterol and triglyceride levels are positively correlated, while high levels of HDL-cholesterol are negatively correlated with the risk for developing cardiovascular diseases. Thus, dyslipidemia is not a unitary risk profile for CHD but may be comprised of one or more lipid aberrations.

Among the many factors controlling plasma levels of these disease dependent principles, cholesteryl ester transfer protein (CETP) activity affects all three. The role of this 70,000 dalton plasma glycoprotein found in a number of animal species, including humans, is to transfer cholesteryl ester and triglyceride between lipoprotein particles, including high density lipoproteins (HDL), low density lipoproteins (LDL), very low density lipoproteins (VLDL), and chylomicrons. The net result of CETP activity is a lowering of HDL cholesterol and an increase in LDL cholesterol. This effect on lipoprotein profile is believed to be pro-atherogenic, especially in subjects whose lipid profile constitutes an increased risk for CHD.

No wholly satisfactory HDL-elevating therapies are on the market today. Niacin can significantly increase HDL, but has serious toleration issues which reduce compliance. Fibrates and the HMG CoA reductase inhibitors raise HDL-C, but in some patients, the result is an increase of modest proportions (˜10-12%). As a result, there is an unmet medical need for an approved therapeutic agent that elevates plasma HDL levels, thereby reversing or slowing the progression of atherosclerosis.

Thus, although there are a variety of anti-atherosclerosis therapies, there is a continuing need and a continuing search in this field of art for alternative therapies.

### SUMMARY OF THE INVENTION

This invention is directed to compounds according to Formula I

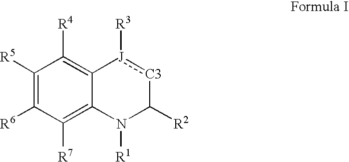

Wherein

C3 is carbon;

J is nitrogen or carbon, wherein if J is carbon, then the bond between C3 and J is a single or double bond and if J is nitrogen, then the bond between C3 and J is a single bond;

R¹ is Y, W—X or W—Y¹; wherein W is a carbonyl, thiocarbonyl, sulfinyl or sulfonyl; X is —O—Y, —S—Y, —N(H)—Y or —N—(Y)₂; Y for each occurrence is independently Z or a fully saturated, partially unsaturated or fully unsaturated one to ten membered straight or branched carbon chain wherein each carbon, other than the connecting carbon, may optionally be replaced with one or two heteroatoms selected independently from oxygen, sulfur and nitrogen and said carbon is optionally mono-, di- or tri-substituted independently with halo, said carbon is optionally mono-substituted with hydroxy, said carbon is optionally mono-substituted with oxo, said sulfur is optionally mono- or di-substituted with oxo, said nitrogen is optionally mono-, or di-substituted with oxo, and said carbon chain is optionally mono-substituted with Z; and Y¹ for each occurrence is independently Z or a fully saturated, partially unsaturated or fully unsaturated one to ten membered straight or branched carbon chain wherein each carbon, other than the connecting carbon, may optionally be replaced with one or two heteroatoms selected independently from oxygen, sulfur and nitrogen and said carbon is optionally mono-, di- or tri-substituted independently with halo, said carbon is optionally mono-substituted with hydroxy, said carbon is optionally mono-substituted with oxo, said sulfur is optionally mono- or di-substituted with oxo, said nitrogen is optionally mono-, or di-substituted with oxo, and said carbon chain is optionally mono-substituted with Z; wherein Z is a partially saturated, fully saturated or fully unsaturated three to eight membered ring optionally having one to four heteroatoms selected independently from oxygen, sulfur and nitrogen, or a bicyclic ring consisting of two fused partially saturated, fully saturated or fully unsaturated three to six membered rings, taken independently, optionally having one to four heteroatoms selected independently from nitrogen, sulfur and oxygen; and said Z substituent is optionally mono-, di- or tri-substituted independently with halo, (C₂-C₆)alkenyl, (C₁-C₆) alkyl, hydroxy, (C₁-C₆)alkoxy, (C₁-C₄)alkylthio, amino, nitro, cyano, oxo, carboxy, (C₁-C₆)alkyloxycarbonyl, mono-N— or di-N,N—(C₁-C₆)alkylamino wherein said (C₁-C₆)alkyl substituent is optionally mono-, di- or tri-substituted independently with halo, hydroxy, (C₁-C₆)alkoxy, (C₁-C₄)alkylthio, amino, nitro, cyano, oxo, carboxy, (C₁-C₆)alkyloxycarbonyl, mono-N— or di-N,N—(C₁-C₆)alkylamino, said (C₁-C₆)alkyl or (C₁-C₆)alkoxy substituent is also optionally substituted with from one to nine fluorines;

R² is a partially saturated, fully saturated or fully unsaturated one to six membered straight or branched carbon chain wherein each carbon, other than the connecting carbon, may optionally be replaced with one or two heteroatoms selected independently from oxygen and sulfur, and said carbon is optionally mono-, di- or tri-substituted independently with halo, said carbon chain is optionally mono-substituted with oxo, said carbon is optionally mono-substituted with hydroxy, said sulfur is optionally mono- or di-substituted with oxo,; or said R² is a partially saturated, fully saturated or fully unsaturated three to seven membered ring optionally having one to two heteroatoms selected independently from oxygen and sulfur, wherein said R² ring is optionally attached through (C₁-C₄)alkyl; wherein said R² ring is optionally mono-, di- or tri-substituted independently with halo, (C₂-C₆)alkenyl, (C₁-C₆) alkyl, hydroxy, (C₁-C₆)alkoxy, (C₁-C₄)alkylthio, amino, nitro, cyano, oxo, carboxy, (C₁-C₆)alkyloxycarbonyl, mono-N— or di-N,N—(C₁-C₆)alkylamino wherein said (C₁-C₆)alkyl substituent is optionally mono-, di- or tri-substituted independently with halo, hydroxy, (C₁-C₆)alkoxy, (C₁-C₄)alkylthio, oxo or (C₁-C₆)alkyloxycarbonyl;

R³ is a fully saturated, partially unsaturated or fully unsaturated one to six membered straight or branched carbon chain containing C4a, wherein C4a is a carbon atom that connects to J, wherein each carbon in the carbon chain may optionally be replaced with one heteroatom selected from oxygen, sulfur and nitrogen, and said carbon is optionally mono-, di- or tri-substituted with halo, said carbon is optionally mono-substituted with hydroxy, said carbon is optionally mono-substituted with oxo or nitrogen, said sulfur is optionally mono- or di-substituted with oxo, said nitrogen is optionally mono- or di-substituted with hydrogen or oxo, and said carbon chain is mono, di-, or tri-substituted with V at C4a or the R³ carbon adjacent to C4a; provided that in R³, when J is carbon, it is other than C4a that is optionally replaced with one heteroatom; and provided that in R³, when J is nitrogen, it is other than C4a that is optionally replaced with a heteroatom and it is other than C4a that is optionally mono-substituted with hydroxy or nitrogen; wherein V is a partially saturated, fully saturated or fully unsaturated three to eight membered ring optionally having one to four heteroatoms selected independently from oxygen, sulfur and nitrogen such that V is not imidazolyl or a fully saturated heterocyclic nitrogen-containing ring wherein nitrogen of the ring is connected to the R³ group; a bicyclic ring consisting of two fused partially saturated, fully saturated or fully unsaturated three to six membered rings, taken independently, optionally having one to four heteroatoms selected independently from nitrogen, sulfur and oxygen; or a tricyclic ring consisting of three fused partially saturated, fully saturated or fully unsaturated three to six membered rings, taken independently, optionally having one to four heteroatoms selected independently from nitrogen, sulfur and oxygen; and said V substituent is optionally mono-, di-, tri-, tetra- or penta-substituted independently with V¹, (C₁-C₆)alkyl-V¹, C(O)—V¹, O—(C₀-C₆)alkyl-V¹, (C₁-C₆)alkyl-O—V¹, C(O)-mono-N— or di-N,N—(C₁-C₆)alkyl-V¹, halo, (C₁-C₆)alkyl, (C₂-C₆)alkenyl, hydroxy, (C₁-C₆)alkoxy, (C₁-C₄)alkylthio, (C₁-C₄)alkylsulfinyl, (C₁-C₄)alkylsulfonyl, mono-N— or di-N,N—(C₁-C₆)alkylsulfonyl, amino, nitro, cyano, oxo, carboxamoyl, mono-N— or di-N,N—(C₁-C₆) alkylcarboxamoyl, carboxy, (C₁-C₆)alkyloxycarbonyl, mono-N— or di-N,N—(C₁-C₆)alkylamino, wherein said (C₁-C₆)alkyl or (C₂-C₆)alkenyl substituent is optionally mono-, di- or tri-substituted independently with hydroxy, (C₁-C₆)alkoxy, (C₁-C₄)alkylthio, amino, nitro, cyano, oxo, carboxy, (C₁-C₆)alkyloxycarbonyl, mono-N— or di-N,N—(C₁-C₆)alkylamino, wherein each (C₁-C₆)alkyl, (C₁-C₆)alkoxy, (C₁-C₄)alkylthio, (C₁-C₄)alkylsulfonyl or (C₂-C₆)alkenyl substituents are also optionally substituted with from one to nine fluorines; wherein V¹ is a partially saturated, fully saturated or fully unsaturated three to six membered ring optionally having one to two heteroatoms selected independently from oxygen, sulfur and nitrogen, or a bicyclic ring consisting of two fused partially saturated, fully saturated or fully unsaturated three to six membered rings, taken independently, optionally having one to four heteroatoms selected independently from nitrogen, sulfur and oxygen; and said V¹ substituent is optionally mono-, di-, tri-, tetra- or penta-substituted independently with halo, (C₁-C₆)alkyl, (C₁-C₆)alkoxy, hydroxy, oxo, amino, nitro, cyano, (C₁-C₆)alkyloxycarbonyl, mono-N— or di-N,N—(C₁-C₆)alkylamino wherein said (C₁-C₆)alkyl substituent is optionally mono-substituted with oxo, said (C₁-C₆)alkyl or (C₁-C₆)alkoxy substituent is also optionally substituted with from one to nine fluorines; and

each of R⁴, R⁵, R⁶ and R⁷are independently hydrogen, a bond, nitro or halo wherein said bond is substituted with T or a partially saturated, fully saturated or fully unsaturated (C₁-C₂) straight or branched carbon chain wherein each carbon may optionally be replaced with one or two heteroatoms per carbon chain wherein the heteroatoms are selected independently from oxygen, sulfur and nitrogen, wherein said carbon is optionally mono-, di- or tri-substituted independently with halo, said carbon is optionally mono-substituted with hydroxy, said carbon is optionally mono-substituted with oxo or nitrogen, said sulfur is optionally mono- or di-substituted with oxo, said nitrogen is optionally mono- or di-substituted with hydrogen or oxo, and said carbon chain is optionally mono-substituted with T; wherein T is a partially saturated, fully saturated or fully unsaturated three to twelve membered ring optionally having one to four heteroatoms selected independently from oxygen, sulfur and nitrogen, or a bicyclic ring consisting of two fused partially saturated, fully saturated or fully unsaturated three to six membered rings, taken independently, optionally having one to four heteroatoms selected independently from nitrogen, sulfur and oxygen; and said T substituent is optionally mono-, di- or tri-substituted independently with halo, (C₁-C₆)alkyl, (C₂-C₆)alkenyl, hydroxy, (C₁-C₆)alkoxy, (C₁-C₄)alkylthio, amino, nitro, cyano, oxo, carboxy, (C₁-C₆)alkyloxycarbonyl, mono-N— or di-N,N—(C₁-C₆)alkylamino wherein said (C₁-C₆)alkyl substituent is optionally mono-, di- or tri-substituted independently with hydroxy, (C₁-C₆)alkoxy, (C₁-C₄)alkylthio, amino, nitro, cyano, oxo, carboxy, (C₁-C₆)alkyloxycarbonyl, mono-N— or di-N,N—(C₁-C₆)alkylamino, said (C₁-C₆)alkyl or (C₁-C₆)alkoxy substituent also optionally has from one to nine fluorines;

R⁴ and R⁵, R⁵ and R⁶, and/or R⁶ and R⁷ may optionally be taken together and can form at least one ring that is a partially saturated or fully unsaturated four to eight membered ring optionally having one to three heteroatoms independently selected from nitrogen, sulfur and oxygen; wherein each ring formed by R⁴ and R⁵, or R⁵ and R⁶, and/or R⁶ and R⁷ is optionally mono-, di- or tri-substituted independently with halo, (C₁-C₆)alkyl, (C₁-C₄)alkylsulfonyl, (C₂-C₆)alkenyl, hydroxy, (C₁-C₆)alkoxy, (C₁-C₄)alkylthio, amino, nitro, cyano, oxo, carboxy, (C₁-C₆)alkyloxycarbonyl, mono-N— or di-N,N—(C₁-C₆)alkylamino wherein said (C₁-C₆)alkyl substituent is optionally mono-, di- or tri-substituted independently with hydroxy, (C₁-C₆)alkoxy, (C₁-C₄)alkylthio, amino, nitro, cyano, oxo, carboxy, (C₁-C₆)alkyloxycarbonyl, mono-N— or di-N,N—(C₁-C₆)alkylamino, said (C₁-C₆)alkyl substituent also optionally has from one to nine fluorines;

or a pharmaceutically acceptable salt or prodrug thereof;

with the following provisos:

a) when there is a single bond between C3 and J, and R³ is a fully saturated one to six membered straight or branched carbon chain substituted on C4a with V then R¹ is other than C(O)—(C₁-C₄)alkyl optionally mono-, di- or tri-substituted with halo and R¹ is other than C(O)-monocyclicaromatic ring; or

b) when there is a single bond between C3 and J, and R³ is —C(O)—O—V, and R² is phenyl then R¹ is other than (C₁-C₄)alkyl; and

c) when there is a double bond between C3 and J, and R² is methyl then R³ is other than —CH₂—O—V, —CH₂—V or —CH₂—CH₂—V.

Furthermore, the present invention is directed to compounds according to Formula II

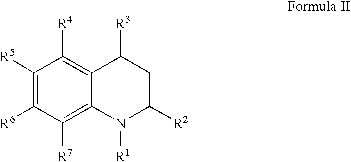

wherein

R¹ is W—X;

W is carbonyl;

X is —O—Y;

Y for each occurrence is independently (C₁-C₆)alkyl, said (C₁-C₆)alkyl optionally having one to nine fluorines or said (C₁-C₆)alkyl optionally mono-substituted with Z;

wherein Z is a partially saturated, fully saturated or fully unsaturated three to six membered ring optionally having one to two heteroatoms selected independently from oxygen, sulfur and nitrogen;

wherein said Z substituent is optionally mono-, di- or tri-substituted independently with halo, (C₁-C₄)alkyl, (C₁-C₄)alkoxy, (C₁-C₄)alkylthio, nitro, cyano, oxo, or (C₁-C₄)alkyloxycarbonyl, said (C₁-C₄)alkyl optionally substituted with from one to nine fluorines;

R² is a partially saturated, fully saturated or fully unsaturated (C₁-C₄) straight or branched carbon chain wherein each carbon, other than the connecting carbon, may optionally be replaced with one heteroatom selected independently from oxygen and sulfur wherein said carbon is optionally mono-, di- or tri-substituted independently with halo, said carbon chain is optionally mono-substituted with oxo, said carbon is optionally mono-substituted with hydroxy, said sulfur is optionally mono- or di-substituted with oxo,; or said R² is a partially saturated, fully saturated or fully unsaturated three to five membered ring optionally having one heteroatom selected independently from oxygen and sulfur;

wherein said R² ring is optionally mono-, di- or tri-substituted independently with halo or (C₁-C₆)alkoxy;

wherein R³is —CH₂NR⁸R⁹ or —C(O)NR⁸R⁹;

wherein R⁸ and R⁹ are independently hydrogen or a (C₁-C₂) straight carbon chain wherein at least one of R⁸ and R⁹ are not hydrogen and wherein said carbon is optionally mono-, di- or tri-substituted independently with halo, said carbon, other than the connecting carbon, is optionally mono-substituted with hydroxy, said carbon is optionally mono-substituted with oxo, and said carbon chain is optionally mono, di-, or tri-substituted with V³, wherein either R⁸ or R⁹ is substituted with V³, or both R⁸ and R⁹ is substituted with V³;

wherein V³ is a partially saturated, fully saturated or fully unsaturated three to six membered ring optionally having one to two heteroatoms selected independently from oxygen, sulfur and nitrogen, or a bicyclic ring consisting of two fused partially saturated, fully saturated or fully unsaturated three to six membered rings, taken independently, optionally having one to four heteroatoms selected independently from nitrogen, sulfur and oxygen;

wherein said V³ substituent is optionally mono-, di-, tri-, tetra- or penta-substituted independently with V⁴, (C₁-C₆)alkyl-V⁴, C(O)—V⁴, O—(C₀-C₆)alkyl-V⁴, (C₁-C₆)alkyl-O—V⁴, halo, (C₁-C₆)alkyl, (C₁-C₆)alkoxy, hydroxy, oxo, amino, nitro, cyano, (C₁-C₄)alkylthio, (C₁-C₄)alkylsulfinyl, (C₁-C₄)alkylsulfonyl, mono-N— or di-N,N—(C₁-C₆)alkylsulfonyl, (C₁-C₆)alkyloxycarbonyl, mono-N— or di-N,N—(C₁-C₆)alkylamino, wherein said (C₁-C₆)alkyl substituent is optionally mono-substituted with oxo, wherein said (C₁-C₆)alkyl substituent is optionally mono-substituted with hydroxy, said (C₁-C₆)alkyl or (C₁-C₆)alkoxy substituents are also optionally substituted with from one to nine fluorines;

wherein V⁴ is a partially saturated, fully saturated or fully unsaturated three to six membered ring optionally having one to two heteroatoms selected independently from oxygen, sulfur and nitrogen, or a bicyclic ring consisting of two fused partially saturated, fully saturated or fully unsaturated three to six membered rings, taken independently, optionally having one to four heteroatoms selected independently from nitrogen, sulfur and oxygen;

wherein said V⁴ substituent is optionally mono-, di-, tri-, or tetra-substituted independently with halo, (C₁-C₆)alkyl, (C₁-C₆)alkoxy, hydroxy, oxo, amino, nitro, cyano, (C₁-C₆)alkyloxycarbonyl, mono-N— or di-N,N—(C₁-C₆)alkylamino wherein said (C₁-C₆)alkyl substituent is optionally mono-substituted with oxo, said (C₁-C₆)alkyl or (C₁-C₆)alkoxy substituent is also optionally substituted with from one to nine fluorines;

or wherein R⁸and R⁹ are taken together to form a partially saturated, fully saturated or fully unsaturated three to eight membered ring optionally having one to four heteroatoms selected independently from oxygen, sulfur, and nitrogen or a bicyclic ring consisting of two fused partially saturated, fully saturated or fully unsaturated three to six membered rings, selected independently from nitrogen, sulfur and oxygen;

R⁴ is hydrogen;

R⁵ and R⁶ are each independently hydrogen, halo, T, (C₁-C₆)alkoxy or (C₁-C₆)alkyl, said (C₁-C₆)alkoxy or (C₁-C₆)alkyl substituent optionally having from one to nine fluorines or said (C₁-C₆)alkoxy or (C₁-C₆)alkyl substituent optionally mono-substituted with T;

wherein T is a partially saturated, fully saturated or fully unsaturated five to six membered ring optionally having one to two heteroatoms selected independently from oxygen, sulfur and nitrogen;

wherein said T substituent is optionally mono-, di- or tri-substituted independently with halo, (C₁-C₆)alkyl, hydroxy, (C₁-C₆)alkoxy, (C₁-C₄)alkylthio, amino, oxo, carboxy, (C₁-C₆)alkyloxycarbonyl, mono-N— or di-N,N—(C₁-C₆)alkylamino wherein said (C₁-C₆)alkyl substituent optionally has from one to nine fluorines;

R⁷ is hydrogen;

or a pharmaceutically acceptable salt or prodrug thereof.

In addition, the present invention provides compounds of Formula III

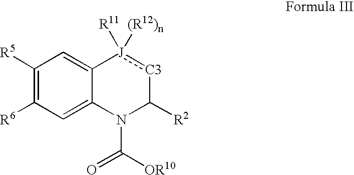

wherein

C3 is carbon;

J is carbon, wherein the bond between C3 and J is a single or double bond;

n is zero if the bond between C3 and J is a double bond or one if the bond between C3 and J is a single bond;

R² is (C₁-C₄)alkyl, cyclopropyl or cyclobutyl;

R⁵ is CF₃;

R⁶ is hydrogen;

R¹⁰ is a fully saturated (C₁-C₄) straight or branched carbon chain;

R¹¹ is halo, hydroxy, —C(O)(O(C₁-C₄)alkyl), —C(O)C(O)(O(C₁-C₄)alkyl), —C(O)NH(O(C₁-C₄)alkyl), or —C(O)N((C₁-C₄)alkyl)(O(C₁-C₄)alkyl);

R¹² is hydrogen or halo, wherein R¹¹ is not halo when R¹² is halo;

or R¹¹ and R¹² are taken together to form oxo or N₂;

or a pharmaceutically acceptable salt or prodrug thereof.

In addition, the present invention provides methods for treating atherosclerosis, coronary artery disease, coronary heart disease, coronary vascular disease, peripheral vascular disease, dyslipidemia, hyperbetalipoproteinemia, hypoalphalipoproteinemia, hypercholesterolemia, hypertriglyceridemia, familial-hypercholesterolemia or myocardial infarction in a mammal by administering to a mammal in need of such treatment an atherosclerosis, coronary artery disease, coronary heart disease, coronary vascular disease, peripheral vascular disease, dyslipidemia, hyperbetalipoproteinemia, hypoalphalipoproteinemia, hypercholesterolemia, hypertriglyceridemia, familial-hypercholesterolemia or myocardial infarction treating amount of a compound of the present invention, or a pharmaceutically acceptable form of said compound.

In addition, the present invention provides pharmaceutical compositions which comprise a therapeutically effective amount of a compound of the present invention, or a pharmaceutically acceptable form of said compound and a pharmaceutically acceptable vehicle, diluent or carrier.

In addition, the present invention provides pharmaceutical compositions for the treatment of atherosclerosis, coronary artery disease, coronary heart disease, coronary vascular disease, peripheral vascular disease, dyslipidemia, hyperbetalipoproteinemia, hypoalphalipoproteinemia, hypercholesterolemia, hypertriglyceridemia, familial-hypercholesterolemia or myocardial infarction in a mammal which comprise a therapeutically effective amount of a compound of the present invention, or a pharmaceutically acceptable form of said compound and a pharmaceutically acceptable vehicle, diluent or carrier.

Moreover, the present invention provides pharmaceutical combination compositions comprising: a therapeutically effective amount of a composition comprising a first compound, said first compound being a compound of the present invention, or a pharmaceutically acceptable form of said compound;

a second compound, said second compound being an HMG CoA reductase inhibitor, an MTP/Apo B secretion inhibitor, a PPAR modulator, a bile acid reuptake inhibitor, a cholesterol absorption inhibitor, a cholesterol synthesis inhibitor, a fibrate, niacin, slow-release niacin, a combination of niacin and lovastatin, an ion-exchange resin, an antioxidant, an ACAT inhibitor or a bile acid sequestrant (preferably an HMG-CoA reductase inhibitor, a PPAR modulator, lovastatin, simvastatin, pravastatin, fluvastatin, atorvastatin, rivastatin, rosuvastatin or pitavastatin); and

a pharmaceutical vehicle, diluent or carrier. This composition may be used to treat the aforementioned diseases, including atherosclerosis.

Also, the present invention provides a kit for achieving a therapeutic effect in a mammal comprising packaged in association a first therapeutic agent comprising a therapeutically effective amount of a compound of claim 1, 8, 12, or 13, a prodrug thereof, or a pharmaceutically acceptable salt of said compound or of said prodrug and a pharmaceutically acceptable carrier, a second therapeutic agent comprising a therapeutically effective amount of an HMG CoA reductase inhibitor, a PPAR modulator, a cholesterol absorption inhibitor, a cholesterol synthesis inhibitor, a fibrate, niacin, slow-release niacin, a combination of niacin and lovastatin, an ion-exchange resin, an antioxidant, an ACAT inhibitor or a bile acid sequestrant and a pharmaceutically acceptable carrier and directions for administration of said first and second agents to achieve the therapeutic effect.

It is to be understood that both the foregoing general description and the following detailed description are exemplary and explanatory only and are not restrictive of the invention, as claimed.

### DETAILED DESCRIPTION OF THE INVENTION

The present invention may be understood more readily by reference to the following detailed description of exemplary embodiments of the invention and the examples included therein.

Before the present compounds, compositions and methods are disclosed and described, it is to be understood that this invention is not limited to specific synthetic methods of making that may of course vary. It is also to be understood that the terminology used herein is for the purpose of describing particular embodiments only and is not intended to be limiting.

The present invention also relates to the pharmaceutically acceptable acid addition salts of compounds of the present invention. The acids which are used to prepare the pharmaceutically acceptable acid addition salts of the aforementioned base compounds of this invention are those which form non-toxic acid addition salts, i.e., salts containing pharmacologically acceptable anions, such as the hydrochloride, hydrobromide, hydroiodide, nitrate, sulfate, bisulfate, phosphate, acid phosphate, acetate, lactate, citrate, acid citrate, tartrate, bitartrate, succinate, maleate, fumarate, gluconate, saccharate, benzoate, methanesulfonate, ethanesulfonate, benzenesulfonate, p-toluenesulfonate and pamoate (i.e., 1,1′-methylene-bis-(2-hydroxy-3-naphthoate)) salts.

The invention also relates to base addition salts of the compounds of the present invention. The chemical bases that may be used as reagents to prepare pharmaceutically acceptable base salts of those compounds of the present invention that are acidic in nature are those that form non-toxic base salts with such compounds. Such non-toxic base salts include, but are not limited to those derived from such pharmacologically acceptable cations such as alkali metal cations (e.g., potassium and sodium) and alkaline earth metal cations (e.g., calcium and magnesium), ammonium or water-soluble amine addition salts such as N-methylglucamine-(meglumine), and the lower alkanolammonium and other base salts of pharmaceutically acceptable organic amines.

The chemist of ordinary skill will recognize that certain compounds of this invention will contain one or more atoms which may be in a particular stereochemical or geometric configuration, giving rise to stereoisomers and configurational isomers. All such isomers and mixtures thereof are included in this invention. Hydrates and solvates of the compounds of this invention are also included.

Where the compounds of the present invention possess two or more stereogenic centers and the absolute or relative stereochemistry is given in the name, the designations R and S refer respectively to each stereogenic center in ascending numerical order (1, 2, 3, etc.) according to the conventional IUPAC number schemes for each molecule. Where the compounds of the present invention possess one or more stereogenic centers and no stereochemistry is given in the name or structure, it is understood that the name or structure is intended to encompass all forms of the compound, including the racemic form.

The compounds of this invention may contain olefin-like double bonds. When such bonds are present, the compounds of the invention exist as cis and trans configurations and as mixtures thereof. The term “cis” refers to the orientation of two substituents with reference to each other and the plane of the ring (either both “up” or both “down”). Analogously, the term “trans” refers to the orientation of two substituents with reference to each other and the plane of the ring (the substituents being on opposite sides of the ring).

Alpha and Beta refer to the orientation of a substituent with reference to the plane of the ring. Beta is above the plane of the ring and Alpha is below the plane of the ring.

This invention also includes isotopically-labeled compounds, which are identical to those described by formulas I and II, except for the fact that one or more atoms are replaced by one or more atoms having specific atomic mass or mass numbers. Examples of isotopes that can be incorporated into compounds of the invention include isotopes of hydrogen, carbon, nitrogen, oxygen, sulfur, fluorine, and chlorine such as ²H, ³H, ¹³C, ¹⁴C, ¹⁵N, ¹⁸O, ¹⁷O, ¹⁸F, and 36Cl respectively. Compounds of the present invention, prodrugs thereof, and pharmaceutically acceptable salts of the compounds or of the prodrugs which contain the aforementioned isotopes and/or other isotopes of other atoms are within the scope of this invention. Certain isotopically-labeled compounds of the present invention, for example those into which radioactive isotopes such as ³H and ¹⁴C are incorporated, are useful in drug and/or substrate tissue distribution assays. Tritiated (i.e., ³H), and carbon-14 (i.e., ¹⁴C), isotopes are particularly preferred for their ease of preparation and detectability. Further, substitution with heavier isotopes such as deuterium (i.e., 2H), can afford certain therapeutic advantages resulting from greater metabolic stability, for example increased in vivo half-life or reduced dosage requirements and, hence, may be preferred in some circumstances. Isotopically labeled compounds of this invention and prodrugs thereof can generally be prepared by carrying out the procedures disclosed in the schemes and/or in the Examples below, by substituting a readily available isotopically labeled reagent for a non-isotopically labeled reagent.

In this specification and in the claims that follow, reference will be made to a number of terms that shall be defined to have the following meanings:

As used herein, the term mammals is meant to refer to all mammals which contain CETP in their plasma, for example, rabbits and primates such as monkeys and humans, including males and females. Certain other mammals e.g., dogs, cats, cattle, goats, sheep and horses do not contain CETP in their plasma and so are not included herein.

The term “treating”, “treat” or “treatment” as used herein includes preventative (e.g., prophylactic) and palliative treatment.

By “pharmaceutically acceptable” is meant the carrier, diluent, excipients, and/or salt must be compatible with the other ingredients of the formulation, and not deleterious to the recipient thereof.

“Compounds” when used herein includes any pharmaceutically acceptable derivative or variation, including conformational isomers (e.g., cis and trans isomers) and all optical isomers (e.g., enantiomers and diastereomers), racemic, diastereomeric and other mixtures of such isomers, as well as solvates, hydrates, isomorphs, polymorphs, tautomers, esters, salt forms, and prodrugs. By “tautomers” is meant chemical compounds that may exist in two or more forms of different structure (isomers) in equilibrium, the forms differing, usually, in the position of a hydrogen atom. Various types of tautomerism can occur, including keto-enol, ring-chain and ring-ring tautomerism. The expression “prodrug” refers to compounds that are drug precursors which following administration, release the drug in vivo via some chemical or physiological process (e.g., a prodrug on being brought to the physiological pH or through enzyme action is converted to the desired drug form). Exemplary prodrugs upon cleavage release the corresponding free acid, and such hydrolyzable ester-forming residues of the compounds of the present invention include but are not limited to those having a carboxyl moiety wherein the free hydrogen is replaced by (C₁-C₄)alkyl, (C₂-C₇)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as β-dimethylaminoethyl), carbamoyl-(C₁-C₂)alkyl, N,N-di(C₁-C₂)alkylcarbamoyl-(C₁-C₂)alkyl and piperidino-, pyrrolidino- or morpholino(C₂-C₃)alkyl.

The following paragraphs describe exemplary ring(s) for the generic ring descriptions contained herein.

Exemplary five to six membered aromatic rings optionally having one or two heteroatoms selected independently from oxygen, nitrogen and sulfur include phenyl, furyl, thienyl, pyrrolyl, oxazolyl, thiazolyl, imidazolyl, pyrazolyl, isoxazolyl, isothiazolyl, pyridinyl, pyridiazinyl, pyrimidinyl and pyrazinyl.

Exemplary partially saturated, fully saturated or fully unsaturated five to eight membered rings optionally having one to four heteroatoms selected independently from oxygen, sulfur and nitrogen include cyclopentyl, cyclohexyl, cycloheptyl, cyclooctyl and phenyl. Further exemplary five membered rings include 2H-pyrrolyl, 3H-pyrrolyl, 2-pyrrolinyl, 3-pyrrolinyl, pyrrolidinyl, 1,3-dioxolanyl, oxazolyl, thiazolyl, imidazolyl, 2H-imidazolyl, 2-imidazolinyl, imidazolidinyl, pyrazolyl, 2-pyrazolinyl, pyrazolidinyl, isoxazolyl, isothiazolyl, 1,2-dithiolyl, 1,3-dithiolyl, 3H-1,2-oxathiolyl, 1,2,3-oxadiazolyl, 1,2,4-oxadiazolyl, 1,2,5-oxadiazolyl, 1,3,4-oxadiazolyl, 1,2,3-triazolyl, 1,2,4-triazolyl, 1,3,4-thiadiazolyl, 1,2,3,4-oxatriazolyl, 1,2,3,5-oxatriazolyl, 3H-1,2,3-dioxazolyl, 1,2,4-dioxazolyl, 1,3,2-dioxazolyl, 1,3,4-dioxazolyl, 5H-1,2,5-oxathiazolyl and 1,3-oxathiolyl.

Further exemplary six membered rings include 2H-pyranyl, 4H-pyranyl, pyridinyl, piperidinyl, 1,2-dioxinyl, 1,3-dioxinyl, 1,4-dioxanyl, morpholinyl, 1,4-dithianyl, thiomorpholinyl, pyridazinyl, pyrimidinyl, pyrazinyl, piperazinyl, 1,3,5-triazinyl, 1,2,4-triazinyl, 1,2,3-triazinyl, 1,3,5-trithianyl, 4H-1,2-oxazinyl, 2H-1,3-oxazinyl, 6H-1,3-oxazinyl, 6H-1,2-oxazinyl, 1,4-oxazinyl, 2H-1,2-oxazinyl, 4H-1,4-oxazinyl, 1,2,5-oxathiazinyl, 1,4-oxazinyl, o-isoxazinyl, p-isoxazinyl, 1,2,5-oxathiazinyl, 1,2,6-oxathiazinyl, 1,4,2-oxadiazinyl and 1,3,5,2-oxadiazinyl.

Further exemplary seven membered rings include azepinyl, oxepinyl, and thiepinyl.

Further exemplary eight membered rings include cyclooctyl, cyclooctenyl and cyclooctadienyl.

Exemplary bicyclic rings consisting of two fused partially saturated, fully saturated or fully unsaturated five or six membered rings, taken independently, optionally having one to four heteroatoms selected independently from nitrogen, sulfur and oxygen include indolizinyl, indolyl, isoindolyl, 3H-indolyl, 1H-isoindolyl, indolinyl, cyclopenta(b)pyridinyl, pyrano(3,4-b)pyrrolyl, benzofuryl, isobenzofuryl, benzo(b)thienyl, benzo(c)thienyl, 1H-indazolyl, indoxazinyl, benzoxazolyl, benzimidazolyl, benzthiazolyl, purinyl, 4H-quinolizinyl, quinolinyl, isoquinolinyl, cinnolinyl, phthalazinyl, quinazolinyl, quinoxalinyl, 1,8-naphthyridinyl, pteridinyl, indenyl, isoindenyl, naphthyl, tetralinyl, decalinyl, 2H-1-benzopyranyl, pyrido(3,4-b)-pyridinyl, pyrido(3,2-b)-pyridinyl, pyrido(4,3-b)-pyridinyl, 2H-1,3-benzoxazinyl, 2H-1,4-benzoxazinyl, 1H-2,3-benzoxazinyl, 4H-3,1-benzoxazinyl, 2H-1,2-benzoxazinyl and 4H-1,4-benzoxazinyl.

By “alkylene” is meant saturated hydrocarbon (straight chain or branched) wherein a hydrogen atom is removed from each of two adjacent carbons. Exemplary of such groups (assuming the designated length encompasses the particular example) are methylene, ethylene, propylene, butylene, pentylene, hexylene, heptylene).

By “halo” or “halogen” is meant chloro, bromo, iodo, or fluoro.

By “alkyl” is meant straight chain saturated hydrocarbon or branched chain saturated hydrocarbon. Exemplary of such alkyl groups (assuming the designated length encompasses the particular example) are methyl, ethyl, propyl, isopropyl, butyl, sec-butyl, tertiary butyl, pentyl, isopentyl, neopentyl, tertiary pentyl, 1-methylbutyl, 2-methylbutyl, 3-methylbutyl, hexyl, isohexyl, heptyl and octyl.

“Alkenyl” referred to herein may be linear or branched, and they may also be cyclic (e.g. cyclobutenyl, cyclopentenyl, cyclohexenyl) or bicyclic or contain cyclic groups. They contain 1-3 carbon-carbon double bonds, which can be cis or trans.

By “alkoxy” is meant straight chain saturated alkyl or branched chain saturated alkyl bonded through an oxy. Exemplary of such alkoxy groups (assuming the designated length encompasses the particular example) are methoxy, ethoxy, propoxy, isopropoxy, butoxy, isobutoxy, tertiary butoxy, pentoxy, isopentoxy, neopentoxy, tertiary pentoxy, hexoxy, isohexoxy, heptoxy and octoxy.

As used herein the term “mono-N—” or “di-N,N—(C₁-Cₓ)alkyl” refers to the (C₁-Cₓ)alkyl moiety taken independently when it is di-N,N—(C₁-Cₓ)alkyl (x refers to integers).

References (e.g., claim 1) to “said carbon” in the phrase “said carbon is optionally mono-, di- or tri-substituted independently with halo, said carbon is optionally mono-substituted with hydroxy, said carbon is optionally mono-substituted with oxo” refers to each of the carbons in the carbon chain including the connecting carbon.

References to a “nitrogen . . . di-substituted with oxo” herein (e.g., claim 1) refer to a terminal nitrogen which constitutes a nitro functionality.

It is to be understood that if a carbocyclic or heterocyclic moiety may be bonded or otherwise attached to a designated substrate through differing ring atoms without denoting a specific point of attachment, then all possible points are intended, whether through a carbon atom or, for example, a trivalent nitrogen atom. For example, the term “pyridyl” means 2-, 3- or 4-pyridyl, the term “thienyl” means 2- or 3-thienyl, and so forth.

DTT means dithiothreitol. DMSO means dimethyl sulfoxide. EDTA means ethylenediamine tetraacetic acid.

As used herein, the expressions “reaction-inert solvent” and “inert solvent” refer to a solvent or a mixture thereof which does not interact with starting materials, reagents, intermediates or products in a manner which adversely affects the yield of the desired product.

In one embodiment of formula I compounds of the present invention,

J is carbon;

R¹ is W—X;

W is carbonyl;

X is —O—Y;

Y for each occurrence is independently (C₁-C₆)alkyl, said (C₁-C₆)alkyl optionally having one to nine fluorines or said (C₁-C₆)alkyl optionally mono-substituted with Z;

wherein Z is a partially saturated, fully saturated or fully unsaturated three to six membered ring optionally having one to two heteroatoms selected independently from oxygen, sulfur and nitrogen;

wherein said Z substituent is optionally mono-, di- or tri-substituted independently with halo, (C₁-C₄)alkyl, (C₁-C₄)alkoxy, (C₁-C₄)alkylthio, nitro, cyano, oxo, or (C₁-C₄)alkyloxycarbonyl, said (C₁-C₄)alkyl or (C₁-C₄)alkoxy is optionally substituted with from one to nine fluorines;

R² is beta and is a partially saturated, fully saturated or fully unsaturated (C₁-C₄) straight or branched carbon chain wherein each carbon, other than the connecting carbon, may optionally be replaced with one heteroatom selected independently from oxygen and sulfur wherein said carbon is optionally mono-, di- or tri-substituted independently with halo, said carbon chain is optionally mono-substituted with oxo or hydroxy, said sulfur is optionally mono- or di-substituted with oxo,; or said R² is a partially saturated, fully saturated or fully unsaturated three to five membered ring optionally having one heteroatom selected independently from oxygen and sulfur;

wherein said R² ring is optionally mono-, di- or tri-substituted independently with halo, hydroxy, (C₁-C₆)alkoxy, amino, nitro, (C₁-C₄)alkyloxycarbonyl or carboxy;

wherein R³ is a fully saturated, partially unsaturated or fully unsaturated one to six membered straight or branched carbon chain wherein each carbon, other than C4a, may optionally be replaced with one heteroatom selected from oxygen, sulfur and nitrogen, and said carbon is optionally mono-, di- or tri-substituted independently with halo, said carbon is optionally mono-substituted with hydroxy, said carbon is optionally mono-substituted with cyano, said carbon is optionally mono-substituted with oxo or nitrogen, said sulfur is optionally mono- or di-substituted with oxo, said nitrogen is optionally mono- or di-substituted with hydrogen or oxo, and said carbon chain is optionally mono, di-, or tri-substituted with V at C4a or at the R³ carbon adjacent to C4a; V is a three, four, five or six membered partially saturated, fully saturated or fully unsaturated ring optionally having one to three heteroatoms selected independently from oxygen, sulfur and nitrogen such that V is not imidazolyl or a fully saturated heterocyclic nitrogen-containing ring wherein nitrogen of the ring is connected to the R³ group;

wherein said V ring is optionally mono-, di-, tri-, tetra- or penta-substituted independently with halo, (C₁-C₆)alkyl, hydroxy, (C₁-C₆)alkoxy, (C₁-C₆)alkoxycarbonyl, nitro, cyano or oxo, wherein said (C₁-C₆)alkyl or (C₁-C₆)alkoxy substituent optionally has from one to nine fluorines;

R⁴ is hydrogen;

R⁵ and R⁶ are each independently hydrogen, halo, T, (C₁-C₆)alkoxy or (C₁-C₆)alkyl, said (C₁-C₆)alkoxy or (C₁-C₆)alkyl substituent optionally having from one to nine fluorines or said (C₁-C₆)alkoxy or (C₁-C₆)alkyl substituent optionally mono-substituted with T;

wherein T is a partially saturated, fully saturated or fully unsaturated five to six membered ring optionally having one to two heteroatoms selected independently from oxygen, sulfur and nitrogen;

wherein said T substituent is optionally mono-, di- or tri-substituted independently with halo, (C₁-C₆)alkyl, hydroxy, (C₁-C₆)alkoxy, (C₁-C₄)alkylthio, amino, oxo, carboxy, (C₁-C₆)alkyloxycarbonyl, mono-N— or di-N,N—(C₁-C₆)alkylamino wherein said (C₁-C₆)alkyl or (C₁-C₆)alkoxy substituent optionally has from one to nine fluorines; and

R⁷ is hydrogen.

In another embodiment of formula I compounds of the present invention,

Y is (C₁-C₄)alkyl, wherein said (C₁-C₄)alkyl substituent optionally has one to nine fluorines;

R² is (C₁-C₄)alkyl, cyclopropyl or cyclobutyl;

R³ is —((C₁-C₄)alkyl)(NH₂)(V), —((C₁-C₃)alkyl)(NH(C₁-C₂)alkyl))(V), —((C₁-C₄)alkyl)(OH)(V), —((C₁-C₄)alkyl)(F)(V), —((C₁-C₂)alkyl)(—O—C(O)(C₁-C₂)alkyl)(V), —C(O)—V, —C(OH)(C(O)O(C₁-C₃)alkyl)(V), —CF₂(V), —((C₁-C₂)alkyl)(NHC(O)(C₁-C₂)alkyl)(V), —CH₂(V), —((C₁-C₂)alkyl)(C(O)O(C₁-C₂)alkyl)(V), —((C₁-C₄)alkyl)(C(O)NH₂)(V), —((C₁-C₄)alkyl)(CN)(V), or —((C₁-C₃)alkyl)((C₁-C₃)alkoxy)(V),

V is phenyl optionally mono-, di- or tri-substituted independently with halo, (C₁-C₆)alkyl, hydroxy, (C₁-C₆)alkoxy, nitro, cyano or oxo wherein said (C₁-C₆)alkyl or (C₁-C₆)alkoxy substituent optionally has from one to nine fluorines;

R⁵ and R⁶ are each independently hydrogen, halo, (C₁-C₃)alkoxy or (C₁-C₆)alkyl, said (C₁-C₃)alkoxy optionally having from one to seven halo, said (C₁-C₆)alkyl optionally having from one to nine halo.

In a further embodiment of formula I compounds of the present invention,

Y is methyl, ethyl, 1-propyl, 2-propyl or tert-butyl;

R² is methyl, ethyl, 2-propyl, cyclopropyl or cyclobutyl;

R³ is —C(O)—V, —C(OH)(C(O)OCH₃)(V), —CH(F)(V), —CF₂(V), —CH(OCH₃)(V), —CH(C(O)OCH₃)(V), —CH(CN)(V), —CH(OH)(V), —CH₂(V), —CH(NH₂)(V), —CH(NH(CH₃))(V), —CH(C(O)NH₂)(V), —CH(CH₂OH)V, —CH(CH₂OCH₃)V, —CH(CH₂OC(O)CH₃)V, —CH(CH₂F)V, or —CH(CH₂NH₂)V; and

V is phenyl optionally mono-, di- or tri-substituted independently with halo, nitro, or (C₁-C₂)alkyl, wherein said (C₁-C₂)alkyl optionally has from one to five fluorines;

R⁵ and R⁶ are each independently hydrogen, methyl, methoxy or chloro; said methoxy optionally having from one to three fluorines, said methyl optionally having from one to three fluorines.

In another embodiment of the present invention, formula I compounds include substituents wherein

Y is ethyl;

R² is ethyl or methyl;

R³ is (3,5-bis-(trifluoromethyl)-phenyl)-hydroxy-methoxycarbonyl-methyl; (3,5-bis-trifluoromethyl-phenyl)-methoxycarbonyl-methyl; (3,5-bis-trifluoromethyl-phenyl)-cyano-methyl, 3,5-bis-trifluoromethyl-benzoyl; (3,5-bis-trifluoromethyl-phenyl)-hydroxy-methyl; (3,5-bis-trifluoromethyl-phenyl)-fluoro-methyl; (3,5-bis-trifluoromethyl-phenyl)-difluoro-methyl; (3,5-bis-(trifluoromethyl)-benzyl); (3,5-bis-trifluoromethyl-phenylcarbamoyl)-methyl; amino-(3,5-bis-(trifluoromethyl)-phenyl)-methyl; (3,5-bis-(trifluoromethyl)-phenyl)-methylamine-methyl; 1-(3,5-bis-(trifluoromethyl)-phenyl)-2-amino-ethyl; 1-(3,5-bis-(trifluoromethyl)-phenyl)-2-fluoro-ethyl; 1-(3,5-bis-(trifluoromethyl)-phenyl)-2-methoxy-ethyl; 1-(3,5-bis-(trifluoromethyl)-phenyl)-2-hydroxy-ethyl; or 2-acetoxy-1-(3,5-bis-(trifluoromethyl)-phenyl)-ethyl;

R⁵ is methoxy or trifluoromethyl; and

R⁶ is hydrogen or methoxy.

In another embodiment of compounds of formula I, the bond between C3 and J is a single bond.

In yet another embodiment of compounds of formula I, the bond between C3 and J is a double bond.

In another embodiment, the compounds of formula I are selected from the group consisting of:

(R, R, S)-4-[Amino-(3,5-bis-trifluoromethyl-phenyl)-methyl]-2-ethyl-6-trifluoromethyl-3,4-dihydro-2H-quinoline-1-carboxylic acid ethyl ester;

(R, S, S)-4-[Amino-(3,5-bis-trifluoromethyl-phenyl)-methyl]-2-ethyl-6-trifluoromethyl-3,4-dihydro-2H-quinoline-1-carboxylic acid ethyl ester;

(R, R)-4-(3,5-bis-trifluoromethyl-benzyl)-2-ethyl-6-trifluoromethyl-3,4-dihydro-2H-quinoline-1-carboxylic acid ethyl ester;

(R, R, S)-4-[(3,5-bis-trifluoromethyl-phenyl)-methylaminomethyl]-2-ethyl-6-trifluoromethyl-3,4-dihydro-2H-quinoline-1-carboxylic acid ethyl ester;

(R, S, S)-4-[(3,5-bis-trifluoromethyl-phenyl)-methylaminomethyl]-2-ethyl-6-trifluoromethyl-3,4-dihydro-2H-quinoline-1-carboxylic acid ethyl ester;

(R, R)-4-[(3,5-bis-trifluoromethyl-phenyl)-hydroxyl-methoxycarbonyl-methyl]-6,7-dimethoxy-2-methyl-2H-quinoline-1-carboxylic acid ethyl ester;

(R, S)-4-[(3,5-bis-trifluoromethyl-phenyl)-hydroxyl-methoxycarbonyl-methyl]-6,7-dimethoxy-2-methyl-2H-quinoline-1-carboxylic acid ethyl ester;

(R, S, R)-4-[(3,5-bis-trifluoromethyl-phenyl)-hydroxyl-methoxycarbonyl-methyl]-6,7-dimethoxy-2-methyl-3,4-dihydro-2H-quinoline-1-carboxylic acid ethyl ester;

(R, S, S)-4-[(3,5-bis-trifluoromethyl-phenyl)-hydroxyl-methoxycarbonyl-methyl]-6,7-dimethoxy-2-methyl-3,4-dihydro-2H-quinoline-1-carboxylic acid ethyl ester;

(R, R, S)-4-[(3,5-bis-trifluoromethyl-phenyl)-hydroxyl-methoxycarbonyl-methyl]-6,7-dimethoxy-2-methyl-3,4-dihydro-2H-quinoline-1-carboxylic acid ethyl ester;

(R, R, R)-4-[(3,5-bis-trifluoromethyl-phenyl)-hydroxyl-methoxycarbonyl-methyl]-6,7-dimethoxy-2-methyl-3,4-dihydro-2H-quinoline-1-carboxylic acid ethyl ester;

(R, S, R)-4-[(3,5-Bis-trifluoromethyl-phenyl)-hydroxy-methoxycarbonyl-methyl]-2-ethyl-6-trifluoromethyl-3,4-dihydro-2H-quinoline-1-carboxylic acid ethyl ester;

(R, S, S)-4-[(3,5-Bis-trifluoromethyl-phenyl)-hydroxy-methoxycarbonyl-methyl]-2-ethyl-6-trifluoromethyl-3,4-dihydro-2H-quinoline-1-carboxylic acid ethyl ester;

(R, R, S)-4-[(3,5-Bis-trifluoromethyl-phenyl)-hydroxy-methoxycarbonyl-methyl]-2-ethyl-6-trifluoromethyl-3,4-dihydro-2H-quinoline-1-carboxylic acid ethyl ester;

(R, R, R)-4-[(3,5-Bis-trifluoromethyl-phenyl)-hydroxy-methoxycarbonyl-methyl]-2-ethyl-6-trifluoromethyl-3,4-dihydro-2H-quinoline-1-carboxylic acid ethyl ester;

(R, R, S)-4-[(3,5-Bis-trifluoromethyl-phenyl)-methoxycarbonyl-methyl]-6,7-dimethoxy-2-methyl-3,4-dihydro-2H-quinoline-1-carboxylic acid ethyl ester;

(R, R, S)-4-[(3,5-Bis-trifluoromethyl-phenyl)-methoxy-methyl]-2-ethyl-6-trifluoromethyl-3,4-dihydro-2H-quinoline-1-carboxylic acid ethyl ester;

(R, R)-4-[(3,5-Bis-trifluoromethyl-phenyl)-hydroxyl-methoxycarbonyl-methyl]-2-ethyl-6-trifluoromethyl-2H-quinoline-1-carboxylic acid ethyl ester;

(R, S)-4-[(3,5-Bis-trifluoromethyl-phenyl)-hydroxyl-methoxycarbonyl-methyl]-2-ethyl-6-trifluoromethyl-2H-quinoline-1-carboxylic acid ethyl ester;

(R, R, S)-4-[1-(3,5-Bis-trifluoromethyl-phenyl)-2-hydroxy-ethyl]-2-ethyl-6-trifluoromethyl-3,4-dihydro-2H-quinoline-1-carboxylic acid ethyl ester;

(R, S, S)-4-[1-(3,5-Bis-trifluoromethyl-phenyl)-2-hydroxy-ethyl]-2-ethyl-6-trifluoromethyl-3,4-dihydro-2H-quinoline-1-carboxylic acid ethyl ester;

(R, R, S)-4-[2-Acetoxy-1-(3,5-bis-trifluoromethyl-phenyl)-ethyl]-2-ethyl-6-trifluoromethyl-3,4-dihydro-2H-quinoline-1-carboxylic acid ethyl ester;

(R, S, S)-4-[2-Acetoxy-1-(3,5-bis-trifluoromethyl-phenyl)-ethyl]-2-ethyl-6-trifluoromethyl-3,4-dihydro-2H-quinoline-1-carboxylic acid ethyl ester;

(R, R, S)-4-[1-(3,5-Bis-trifluoromethyl-phenyl)-2-methoxy-ethyl]-2-ethyl-6-trifluoromethyl-3,4-dihydro-2H-quinoline-1-carboxylic acid ethyl ester;

(R, S, S)-4-[1-(3,5-Bis-trifluoromethyl-phenyl)-2-methoxy-ethyl]-2-ethyl-6-trifluoromethyl-3,4-dihydro-2H-quinoline-1-carboxylic acid ethyl ester;

(R, R, S)-4-[1-(3,5-Bis-trifluoromethyl-phenyl)-2-fluoro-ethyl]-2-ethyl-6-trifluoromethyl-3,4-dihydro-2H-quinoline-1-carboxylic acid ethyl ester;

(R, S, S)-4-[1-(3,5-Bis-trifluoromethyl-phenyl)-2-fluoro-ethyl]-2-ethyl-6-trifluoromethyl-3,4-dihydro-2H-quinoline-1-carboxylic acid ethyl ester;

(R, R, S)-4-[1-(3,5-Bis-trifluoromethyl-phenyl)-2-amino-ethyl]-2-ethyl-6-trifluoromethyl-3,4-dihydro-2H-quinoline-1-carboxylic acid ethyl ester;

(R, S, S)-4-[1-(3,5-Bis-trifluoromethyl-phenyl)-2-amino-ethyl]-2-ethyl-6-trifluoromethyl-3,4-dihydro-2H-quinoline-1-carboxylic acid ethyl ester;

(R,R)-4-[(3,5-Bis-trifluoromethyl-phenyl)-methoxycarbonyl-methyl]-2-ethyl-6-trifluoromethyl-2H-quinoline-1-carboxylic acid ethyl ester;

(R,S)-4-[(3,5-Bis-trifluoromethyl-phenyl)-methoxycarbonyl-methyl]-2-ethyl-6-trifluoromethyl-2H-quinoline-1-carboxylic acid ethyl ester;

(R,S,S)-4-[(3,5-Bis-trifluoromethyl-phenyl)-methoxycarbonyl-methyl]-2-ethyl-6-trifluoromethyl-3,4-dihydro-2H-quinoline-1-carboxylic acid ethyl ester;

(R,R,S)-4-[(3,5-Bis-trifluoromethyl-phenyl)-methoxycarbonyl-methyl]-2-ethyl-6-trifluoromethyl-3,4-dihydro-2H-quinoline-1-carboxylic acid ethyl ester;

(R,S,S)-4-[(3,5-Bis-trifluoromethyl-phenyl)-cyano-methyl]-2-ethyl-6-trifluoromethyl-3,4-dihydro-2H-quinoline-1-carboxylic acid ethyl ester;

(R,S,R)-4-[(3,5-Bis-trifluoromethyl-phenyl)-cyano-methyl]-2-ethyl-6-trifluoromethyl-3,4-dihydro-2H-quinoline-1-carboxylic acid ethyl ester;

(R,R,S)-4-[(3,5-Bis-trifluoromethyl-phenyl)-cyano-methyl]-2-ethyl-6-trifluoromethyl-3,4-dihydro-2H-quinoline-1-carboxylic acid ethyl ester;

(R,R,R)-4-[(3,5-Bis-trifluoromethyl-phenyl)-cyano-methyl]-2-ethyl-6-trifluoromethyl-3,4-dihydro-2H-quinoline-1-carboxylic acid ethyl ester;

(R,S)-4-[(3,5-Bis-trifluoromethyl-phenyl)-cyano-methyl]-2-ethyl-6-trifluoromethyl-2H-quinoline-1-carboxylic acid ethyl ester;

(R,R)-4-[(3,5-Bis-trifluoromethyl-phenyl)-cyano-methyl]-2-ethyl-6-trifluoromethyl-2H-quinoline-1-carboxylic acid ethyl ester;

(R,S)-4-(3,5-Bis-trifluoromethyl-benzoyl)-6,7-dimethoxy-2-methyl-3,4-dihydro-2H-quinoline-1-carboxylic acid ethyl ester;

(R,R)-4-[(3,5-Bis-trifluoromethyl-benzoyl)-6,7-dimethoxy-2-methyl]-3,4-dihydro-2H-quinoline-1-carboxylic acid ethyl ester;

(R,R,R)-4-[(3,5-Bis-trifluoromethyl-phenyl)-fluoro-methyl]-6,7-dimethoxy-2-methyl-3,4-dihydro-2H-quinoline-1-carboxylic acid ethyl ester;

(R,R,S)-4-[(3,5-Bis-trifluoromethyl-phenyl)-fluoro-methyl]-6,7-dimethoxy-2-methyl-3,4-dihydro-2H-quinoline-1-carboxylic acid ethyl ester;

(R,S,R)-4-[(3,5-Bis-trifluoromethyl-phenyl)-fluoro-methyl]-6,7-dimethoxy-2-methyl-3,4-dihydro-2H-quinoline-1-carboxylic acid ethyl ester;

(R,S,S)-4-[(3,5-Bis-trifluoromethyl-phenyl)-fluoro-methyl]-6,7-dimethoxy-2-methyl-3,4-dihydro-2H-quinoline-1-carboxylic acid ethyl ester;

(R,R)-4-(3,5-bis-trifluoromethyl-benzoyl)-2-ethyl-6-trifluoromethyl-3,4-dihydro-2H-quinoline-1-carboxylic acid ethyl ester;

(R,S)-4-(3,5-bis-trifluoromethyl-benzoyl)-2-ethyl-6-trifluoromethyl-3,4-dihydro-2H-quinoline-1-carboxylic acid ethyl ester;

(R,R,R)-4-[(3,5-Bis-trifluoromethyl-phenyl)-hydroxy-methyl]-6,7-dimethoxy-2-methyl-3,4-dihydro-2H-quinoline-1-carboxylic acid ethyl ester;

(R,R,S)-4-[(3,5-Bis-trifluoromethyl-phenyl)-hydroxy-methyl]-6,7-dimethoxy-2-methyl-3,4-dihydro-2H-quinoline-1-carboxylic acid ethyl ester;

(R,S,R)-4-[(3,5-Bis-trifluoromethyl-phenyl)-hydroxy-methyl]-6,7-dimethoxy-2-methyl-3,4-dihydro-2H-quinoline-1-carboxylic acid ethyl ester;

(R,S,S)-4-[(3,5-Bis-trifluoromethyl-phenyl)-hydroxy-methyl]-6,7-dimethoxy-2-methyl-3,4-dihydro-2H-quinoline-1-carboxylic acid ethyl ester;

(R,S,S)-4-[(3,5-Bis-trifluoromethyl-phenyl)-hydroxy-methyl]-2-ethyl-6-trifluoromethyl-3,4-dihydro-2H-quinoline-1-carboxylic acid ethyl ester;

(R,S,R)-4-[(3,5-Bis-trifluoromethyl-phenyl)-hydroxy-methyl]-2-ethyl-6-trifluoromethyl-3,4-dihydro-2H-quinoline-1-carboxylic acid ethyl ester;

(R,R,S)-4-[(3,5-Bis-trifluoromethyl-phenyl)-hydroxy-methyl]-2-ethyl-6-trifluoromethyl-3,4-dihydro-2H-quinoline-1-carboxylic acid ethyl ester; and

(R,R,R)-4-[(3,5-Bis-trifluoromethyl-phenyl)-hydroxy-methyl]-2-ethyl-6-trifluoromethyl-3,4-dihydro-2H-quinoline-1-carboxylic acid ethyl ester;

or a pharmaceutically acceptable salt or prodrug thereof.

In a further embodiment of compounds of formula I of the present invention,

J is nitrogen;

the bond between C3 and J is a single bond;

R¹ is W—X;

W is carbonyl;

X is —O—Y;

Y for each occurrence is independently (C₁-C₆)alkyl, said (C₁-C₆)alkyl optionally having one to nine fluorines or said (C₁-C₆)alkyl optionally mono-substituted with Z;

wherein Z is a partially saturated, fully saturated or fully unsaturated three to six membered ring optionally having one to two heteroatoms selected independently from oxygen, sulfur and nitrogen;

wherein said Z substituent is optionally mono-, di- or tri-substituted independently with halo, (C₁-C₄)alkyl, (C₁-C₄)alkoxy, (C₁-C₄)alkylthio, nitro, cyano, oxo, or (C₁-C₄)alkyloxycarbonyl, said (C₁-C₄)alkyl or (C₁-C₄)alkoxy optionally substituted with from one to nine fluorines;

R is a partially saturated, fully saturated or fully unsaturated (C₁-C₄) straight or branched carbon chain wherein each carbon, other than the connecting carbon, may optionally be replaced with one heteroatom selected independently from oxygen and sulfur wherein said carbon is optionally mono-, di- or tri-substituted independently with halo, said carbon chain is optionally mono-substituted with oxo, said carbon is optionally monosubstituted with hydroxy, said sulfur is optionally mono- or di-substituted with oxo,; or said R² is a partially saturated, fully saturated or fully unsaturated three to five membered ring optionally having one heteroatom selected independently from oxygen and sulfur; wherein said R² ring is optionally mono-, di- or tri-substituted independently with halo, hydroxy, (C₁-C₆)alkoxy, amino, nitro, (C₁-C₄)alkyloxycarbonyl or carboxy;

wherein R³ is a fully saturated, partially unsaturated or fully unsaturated one to six membered straight or branched carbon chain wherein each carbon, other than C4a or the R³ carbon adjacent to C4a, may optionally be replaced with one heteroatom selected from oxygen, sulfur and nitrogen, and said carbon is optionally mono-, di- or tri-substituted independently with halo, said carbon, other than C4a, is optionally mono-substituted with hydroxy, said carbon is optionally mono-substituted with cyano, said carbon is optionally mono-substituted with oxo or nitrogen, said sulfur is optionally mono- or di-substituted with oxo, said nitrogen is optionally mono- or di-substituted with hydrogen or oxo, and said carbon chain is optionally mono, di-, or tri-substituted with V at C4a or at the the R³ carbon adjacent to C4a;

V is a five or six membered partially saturated, fully saturated or fully unsaturated ring optionally having one to three heteroatoms selected independently from oxygen, sulfur and nitrogen such that V is not imidazolyl or a fully saturated heterocyclic nitrogen-containing ring wherein nitrogen of the ring is connected to the R³ group; wherein said V ring is optionally mono-, di-, tri-, tetra- or penta-substituted independently with halo, (C₁-C₆)alkyl, hydroxy, (C₁-C₆)alkoxy, (C₁-C₆)alkoxycarbonyl, nitro, cyano or oxo, wherein said (C₁-C₆)alkyl or (C₁-C₆)alkoxy substituent optionally has from one to nine fluorines;

R⁴ is hydrogen;

R⁵ and R⁶ are each independently hydrogen, halo, T, (C₁-C₆)alkoxy or (C₁-C₆)alkyl, said (C₁-C₆)alkoxy or (C₁-C₆)alkyl substituent optionally having from one to nine fluorines or said (C₁-C₆)alkoxy or (C₁-C₆)alkyl substituent optionally mono-substituted with T;

wherein T is a partially saturated, fully saturated or fully unsaturated five to six membered ring optionally having one to two heteroatoms selected independently from oxygen, sulfur and nitrogen;

wherein said T substituent is optionally mono-, di- or tri-substituted independently with halo, (C₁-C₆)alkyl, hydroxy, (C₁-C₆)alkoxy, (C₁-C₄)alkylthio, amino, oxo, carboxy, (C₁-C₆)alkyloxycarbonyl, mono-N— or di-N,N—(C₁-C₆)alkylamino wherein said (C₁-C₆)alkyl or (C₁-C₆)alkoxy substituent optionally has from one to nine fluorines;

R⁷ is hydrogen.

In a further embodiment of compounds of formula I of the present invention,

Y is (C₁-C₄)alkyl, wherein said (C₁-C₄)alkyl substituent optionally has one to nine fluorines;

R² is (C₁-C₄)alkyl, cyclopropyl or cyclobutyl;

R³ is —C(O)—V, —CH(C(O)O(C₁-C₃)alkyl)(V), or —CH(CN)(V);

V is phenyl optionally mono-, di- or tri-substituted independently with halo, (C₁-C₆)alkyl, hydroxy, (C₁-C₆)alkoxy, nitro, cyano or oxo wherein said (C₁-C₆)alkyl substituent optionally has from one to nine fluorines;

R⁵ and R⁶ are each independently hydrogen, (C₁-C₃)alkoxy or (C₁-C₆)alkyl, said (C₁-C₃)alkoxy optionally having from one to nine fluorines, said (C₁-C₆)alkyl optionally having from one to seven fluorines;

or a pharmaceutically acceptable salt thereof.

In a further embodiment of compounds of formula I of the present invention,

Y is methyl, ethyl, 1-propyl, 2-propyl or tert-butyl;

R² is methyl, ethyl, 2-propyl, cyclopropyl or cyclobutyl;

R³ is 3,5-bis-trifluoromethyl-benzoyl, (3,5-bis-trifluoromethyl-phenyl)-cyano-methyl, or (3,5-bis-trifluoromethyl-phenyl)-methoxycarbonyl-methyl;

R⁵ is methyl or trifluoromethyl;

R⁶ is hydrogen or methyl.

In yet another embodiment of formula I compounds, the compound is selected from the group consisting of:

(R)-4-(3,5-Bis-trifluoromethyl-benzoyl)-2-ethyl-6,7-dimethyl-3,4-dihydro-2H-quinoxaline-1-carboxylic acid ethyl ester;

(R,R)-4-[(3,5-Bis-trifluoromethyl-phenyl)-cyano-methyl]-2-ethyl-6,7-dimethyl-3,4-dihydro-2H-quinoxaline-1-carboxylic acid ethyl ester;

(R,S)-4-[(3,5-Bis-trifluoromethyl-phenyl)-cyano-methyl]-2-ethyl-6,7-dimethyl-3,4-dihydro-2H-quinoxaline-1-carboxylic acid ethyl ester;

(R,R)-4-[(3,5-Bis-trifluoromethyl-phenyl)-methoxycarbonyl-methyl]-2-ethyl-6,7-dimethyl-3,4-dihydro-2H-quinoxaline-1-carboxylic acid ethyl ester;

(R,S)-4-[(3,5-Bis-trifluoromethyl-phenyl)-methoxycarbonyl-methyl]-2-ethyl-6,7-dimethyl-3,4-dihydro-2H-quinoxaline-1-carboxylic acid ethyl ester;

(R,R)-4-[(3,5-Bis-trifluoromethyl-phenyl)-methoxycarbonyl-methyl]-2-ethyl-6,7-dimethyl-3,4-dihydro-2H-quinoxaline-1-carboxylic acid methyl ester;

(R,S)-4-[(3,5-Bis-trifluoromethyl-phenyl)-methoxycarbonyl-methyl]-2-ethyl-6,7-dimethyl-3,4-dihydro-2H-quinoxaline-1-carboxylic acid methyl ester;

(R,R)-4-[(3,5-Bis-trifluoromethyl-phenyl)-methoxycarbonyl-methyl]-2-ethyl-6,7-dimethyl-3,4-dihydro-2H-quinoxaline-1-carboxylic acid isopropyl ester;

(R,S)-4-[(3,5-Bis-trifluoromethyl-phenyl)-methoxycarbonyl-methyl]-2-ethyl-6,7-dimethyl-3,4-dihydro-2H-quinoxaline-1-carboxylic acid isopropyl ester;

(R,R)-4-[(3,5-Bis-trifluoromethyl-phenyl)-methoxycarbonyl-methyl]-2-ethyl-6-trifluoromethyl-3,4-dihydro-2H-quinoxaline-1-carboxylic acid ethyl ester; and

(R,S)-4-[(3,5-Bis-trifluoromethyl-phenyl)-methoxycarbonyl-methyl]-2-ethyl-6-trifluoromethyl-3,4-dihydro-2H-quinoxaline-1-carboxylic acid ethyl ester;

or a pharmaceutically acceptable salt or prodrug thereof.

Moreover, one embodiment of the present invention includes a method for treating atherosclerosis, coronary artery disease, coronary heart disease, coronary vascular disease, peripheral vascular disease, dyslipidemia, hyperbetalipoproteinemia, hypoalphalipoproteinemia, hypercholesterolemia, hypertriglyceridemia, familial-hypercholesterolemia or myocardial infarction in a mammal by administering to a mammal in need of such treatment an atherosclerosis, coronary artery disease, coronary heart disease, coronary vascular disease, peripheral vascular disease, dyslipidemia, hyperbetalipoproteinemia, hypoalphalipoproteinemia, hypercholesterolemia, hypertriglyceridemia, familial-hypercholesterolemia or myocardial infarction treating amount of a compound of formula I or II, a prodrug thereof, or a pharmaceutically acceptable salt of said compound or of said prodrug.

In another embodiment, atherosclerosis is treated.

In another embodiment, peripheral vascular disease is treated.

In another embodiment, dyslipidemia is treated.

In another embodiment, hyperbetalipoproteinemia is treated.

In another embodiment, hypoalphalipoproteinemia is treated.

In another embodiment, familial-hypercholesterolemia is treated.

In another embodiment, coronary artery disease is treated.

In another embodiment, myocardial infarction is treated.

Furthermore, the present invention includes a pharmaceutical composition which comprises a therapeutically effective amount of a compound of formula I or II, a prodrug thereof, or a pharmaceutically acceptable salt of said compound or of said prodrug and a pharmaceutically acceptable vehicle, diluent or carrier.

In one embodiment, the present invention is a pharmaceutical composition for the treatment of atherosclerosis, coronary artery disease, coronary heart disease, coronary vascular disease, peripheral vascular disease, dyslipidemia, hyperbetalipoproteinemia, hypoalphalipoproteinemia, hypercholesterolemia, hypertriglyceridemia, familial-hypercholesterolemia or myocardial infarction in a mammal which comprises a therapeutically effective amount of a compound of formula I or II, a prodrug thereof, or a pharmaceutically acceptable salt of said compound or of said prodrug and a pharmaceutically acceptable vehicle, diluent or carrier.

In another embodiment, the present invention is a pharmaceutical composition for the treatment of atherosclerosis in a mammal which comprises an atherosclerosis treating amount of a compound of formula I or II, a prodrug thereof, or a pharmaceutically acceptable salt of said compound or of said prodrug and a pharmaceutically acceptable vehicle, diluent or carrier.

In another embodiment, the present invention includes a pharmaceutical combination composition comprising: a therapeutically effective amount of a composition comprising: a first compound, said first compound being a compound of formula I or II, a prodrug thereof, or a pharmaceutically acceptable salt of said compound or of said prodrug; a second compound, said second compound being an HMG CoA reductase inhibitor, an MTP/Apo B secretion inhibitor, a PPAR modulator, a bile acid reuptake inhibitor, a cholesterol absorption inhibitor, a cholesterol synthesis inhibitor, a fibrate, niacin, slow-release niacin, a combination of niacin and lovastatin, an ion-exchange resin, an antioxidant, an ACAT inhibitor or a bile acid sequestrant; and a pharmaceutical vehicle, diluent or carrier.

In another embodiment, the present invention includes a pharmaceutical combination composition wherein the second compound is an HMG-CoA reductase inhibitor or a PPAR modulator.

In another embodiment, the present invention includes a pharmaceutical combination composition wherein the second compound is lovastatin, simvastatin, pravastatin, fluvastatin, atorvastatin, rivastatin, rosuvastatin or pitavastatin.

In another embodiment, the present invention includes a pharmaceutical combination composition further comprising a cholesterol absorption inhibitor. In another embodiment, the cholesterol absorption inhibitor is ezetimibe.

One further embodiment of the present invention includes a method for treating atherosclerosis in a mammal comprising administering to a mammal in need of treatment thereof; a first compound, said first compound being a compound of formula (I), a prodrug thereof, or a pharmaceutically acceptable salt of said compound or of said prodrug; and a second compound, said second compound being an HMG CoA reductase inhibitor, a PPAR modulator, a cholesterol absorption inhibitor, a cholesterol synthesis inhibitor, a fibrate, niacin, slow-release niacin, a combination of niacin and lovastatin, an ion-exchange resin, an antioxidant, an ACAT inhibitor or a bile acid sequestrant; wherein the amounts of first and second compounds result in a therapeutic effect.

In another embodiment, the present invention includes a method for treating atherosclerosis wherein the second compound is an HMG-CoA reductase inhibitor or a PPAR modulator.

In another embodiment, the present invention includes a method for treating atherosclerosis wherein the second compound is lovastatin, simvastatin, pravastatin, fluvastatin, atorvastatin, rivastatin, rosuvastatin or pitavastatin.

In another embodiment, the present invention includes a method for treating atherosclerosis wherein the method further comprises administering a cholesterol absorption inhibitor. In another embodiment, the cholesterol absorption inhibitor is ezetimibe.

A further embodiment of the present invention includes a kit for achieving a therapeutic effect in a mammal comprising packaged in association a first therapeutic agent comprising a therapeutically effective amount of a compound of claim 1, 8, 12, or 13, a prodrug thereof, or a pharmaceutically acceptable salt of said compound or of said prodrug and a pharmaceutically acceptable carrier, a second therapeutic agent comprising a therapeutically effective amount of an HMG CoA reductase inhibitor, a PPAR modulator, a cholesterol absorption inhibitor, a cholesterol synthesis inhibitor, a fibrate, niacin, slow-release niacin, a combination of niacin and lovastatin, an ion-exchange resin, an antioxidant, an ACAT inhibitor or a bile acid sequestrant and a pharmaceutically acceptable carrier and directions for administration of said first and second agents to achieve the therapeutic effect.

In another embodiment, the present invention includes a kit wherein said second compound is an HMG-CoA reductase inhibitor or a PPAR modulator.

In a further embodiment, the present invention includes a kit wherein said second compound is lovastatin, simvastatin, pravastatin, fluvastatin, atorvastatin, rivastatin, rosuvastatin or pitavastatin.

In another embodiment, the present invention includes a kit that further comprises a cholesterol absorption inhibitor. In another embodiment, the cholesterol absorption inhibitor is ezetimibe.

Specific compounds of formula III include:

2-Ethyl-4-iodo-6-trifluoromethyl-2H-quinoline-1-carboxylic acid ethyl ester;

2-Ethyl-4-iodo-6-trifluoromethyl-2H-quinoline-1-carboxylic acid ethyl ester;

4-Chloro-2-ethyl-6-trifluoromethyl-3,4-dihydro-2H-quinoline-1-carboxylic acid ethyl ester;

4-Bromo-2-ethyl-6-trifluoromethyl-3,4-dihydro-2H-quinoline-1-carboxylic acid ethyl ester;

4-Diazo-2-ethyl-6-trifluoromethyl-3,4-dihydro-2H-quinoline-1-carboxylic acid ethyl ester;

4-Chloro-2-ethyl-6-trifluoromethyl-3,4-dihydro-2H-quinoline-1,4-dicarboxylic acid 1-ethyl ester 4-methyl ester;

2-Ethyl-6-trifluoromethyl-3,4-dihydro-2H-quinoline-1,4-dicarboxylic acid-1-ethyl ester-4-methyl ester;

2-Ethyl-4-(methoxy-methyl-carbamoyl)-6-trifluoromethyll-3,4-dihydro-2H-quinoline-1-carboxylic acid ethyl ester;

2-Ethyl-6-trifluoromethyl-2H-quinoline-1,4-dicarboxylic acid-1-ethyl ester-4-methyl ester;

4-Chloro-2-ethyl-4-methoxycarboncarbonyl-6-trifluoromethyl-3,4-dihydro-2H-quinoline-1-carboxylic acid ethyl ester;

2-Ethyl-4-methoxycarboncarbonyl-6-trifluoromethyl-2H-quinoline-1-carboxylic acid-ethyl ester;

4-Diazo-6,7-dimethoxy-2-methyl-3,4-dihydro-2H-quinoline-1-carboxylic acid ethyl ester;

6,7-Dimethoxy-4-methoxycarboncarbonyl-2-methyl-2H-quinoline-1-carboxylic acid-ethyl ester;

6,7-Dimethoxy-2-methyl-3,4-dihydro-2H-quinoline-1,4-dicarboxylic acid-1-ethyl ester-4-methyl ester;

6,7-Dimethoxy-4-(methoxy-methyl-carbamoyl)-2-methyl-3,4-dihydro-2H-quinoline-1-carboxylic acid ethyl ester;

2-Ethyl-4-hydroxy-6-trifluoromethyl-3,4-dihydro-2H-quinoline-1-carboxylic acid ethyl ester;

2-Ethyl-4-oxo-6-trifluoromethyl-3,4-dihydro-2H-quinoline-1-carboxylic acid ethyl ester;

2-Ethyl-4-hydroxy-6-trifluoromethyl-3,4-dihydro-2H-quinoline-1-carboxylic acid methyl ester;

2-Ethyl-4-oxo-6-trifluoromethyl-3,4-dihydro-2H-quinoline-1-carboxylic acid methyl ester;

2-Ethyl-4-hydroxy-6-trifluoromethyl-3,4-dihydro-2H-quinoline-1-carboxylic acid 1-propyl ester;

2-Ethyl-4-oxo-6-trifluoromethyl-3,4-dihydro-2H-quinoline-1-carboxylic acid 1-propyl ester;

2-Ethyl-4-hydroxy-6-trifluoromethyl-3,4-dihydro-2H-quinoline-1-carboxylic acid 2-propyl ester;

2-Ethyl-4-oxo-6-trifluoromethyl-3,4-dihydro-2H-quinoline-1-carboxylic acid 2-propyl ester;

2-Ethyl-4-hydroxy-6-trifluoromethyl-3,4-dihydro-2H-quinoline-1-carboxylic acid tert-butyl ester;

2-Ethyl-4-oxo-6-trifluoromethyl-3,4-dihydro-2H-quinoline-1-carboxylic acid tert-butyl ester; and

2-Ethyl-6-trifluoromethyl-3,4-dihydro-2H-quinoline-1,4-dicarboxylic acid-1-ethyl ester-4-methyl ester.

In general, the compounds of this invention can be made by processes which include processes analogous to those known in the chemical arts, particularly in light of the description contained herein. Certain processes for the manufacture of the compounds of this invention are provided as further features of the invention and are illustrated by the following reaction schemes. Other processes may be described in the experimental section. Analogous processes are disclosed in the following U.S. patents, which are hereby incorporated by reference herein in their entirety: U.S. Pat. No. 6,140,342; U.S. Pat. No. 6,362,198; U.S. Pat. No. 6,147,090; U.S. Pat. No. 6,395,751; U.S. Pat. No. 6,147,089; U.S. Pat. No. 6,310,075; U.S. Pat. No. 6,197,786; U.S. Pat. No. 6,140,343; U.S. Pat. No. 6,489,478; and International Publication No. WO 00/17164.

The Reaction Schemes herein described are intended to provide a general description of the methodology employed in the preparation of many of the Examples given. However, it will be evident from the detailed descriptions given in the Experimental section that the modes of preparation employed extend further than the general procedures described herein. In particular, it is noted that the compounds prepared according to these Schemes may be modified further to provide new Examples within the scope of this invention. For example, an ester functionality may be reacted further using procedures well known to those skilled in the art to give another ester, an amide, a carbinol or a ketone.

### Scheme 1

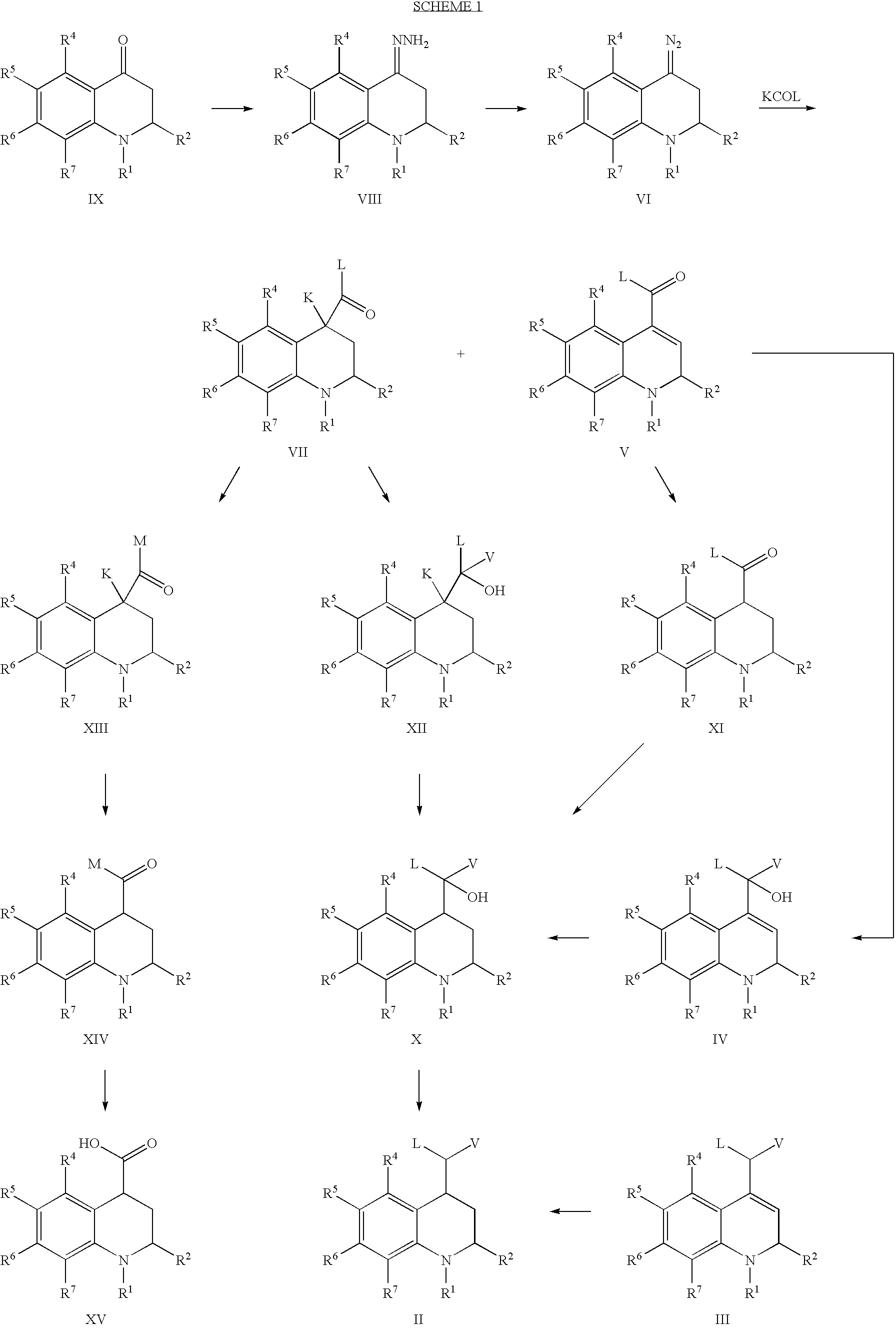

According to reaction Scheme 1, the desired compounds wherein J is carbon, the optional double bond is absent, R³ is a group CH(V)(L) wherein L is a (C₁-C₆) alkoxycarbonyl group and V, R¹, R², R⁴, R⁵, R⁶, and R⁷ are as described above (depicted in Scheme 1 as Formula II compounds) may be prepared as a mixture of diastereoisomers from the corresponding Scheme 1, Formula III compounds by reduction of the double bond or L is R³ as defined herein. This may be achieved by hydrogenation in a reaction inert solvent such as methanol, ethanol or acetic acid with a catalyst such as palladium or rhodium on carbon under a hydrogen pressure equal to 15-50 psi for a period between 2-24 hrs, or by transfer hydrogenation using ammonium formate in refluxing methanol in the presence of a catalyst such as palladium on carbon in a reaction inert solvent such as methanol or ethanol at a temperature between 0° C. to 80° C., typically 25° C. to 60° C. This method of preparing these particular Formula I compounds typically provides a preponderance of those diastereoisomers in which the R² and R³ groups are cis to one another.

The desired Formula III compounds wherein L, V, R¹, R², R⁴, R⁵, R⁶, and R⁷ are as described above may be prepared as a mixture of diastereoisomers from the corresponding Formula IV compounds by removal of the hydroxyl group. This may be achieved by treatment with a chlorinating agent such as phosphorus (III) chloride or thionyl chloride in a reaction inert solvent such as methylene chloride or chloroform optionally containing a base such as pyridine, diisopropylethylamine or 2,6-di-tert-butyl-4-methylpyridine at a temperature between 0° C. to 60° C., typically ambient, for a period between 1 to 24 hr. The chloro-derivative thus formed is then treated with a finely divided metal such as zinc in the presence of an acid, or mixture of acids, such as acetic acid or hydrochloric acid in a suitable solvent, or mixture of solvents such as methanol, water or tetrahydrofuran at a temperature between 25° C. to 60° C., typically ambient, to provide the desired product of Formula III.

The desired Formula IV compounds wherein L is a (C₁-C₆) alkoxycarbonyl group and V, R¹, R², R⁴, R⁵, R⁶, and R⁷ are as described above may be prepared as a mixture of diastereoisomers from the corresponding Formula V compounds by reaction with a suitable organometallic derivative of the V group such as a magnesium or lithium derivative, prepared in turn from a compound V-Hal where Hal represents a chlorine, bromine or iodine atom, using methods well known to those skilled in the art, for example as described in L. A. Paquette (Ed), Encyclopedia of Reagents for Organic Synthesis, John Wiley and Sons, Chichester, England, 1995. This reaction is conducted in a suitable reaction inert solvent such tetrahydrofuran or diethyl ether at a temperature between −78° C. to 25° C., typically −78° C., to provide the desired product of Formula IV.

The desired Formula V compounds wherein L is a (C₁-C₆) alkoxycarbonyl group and R¹, R², R⁴, R⁵, R⁶, and R⁷ are as described above may be prepared from the corresponding Formula VI compounds by reaction with an acyl compound KCOL wherein K is a leaving group such as chlorine or bromine, at a temperature between 0° C. to 25° C., typically ambient, in a reaction inert solvent such as acetonitrile or toluene optionally in the presence of a base such as diisopropylethylamine or triethylamine to remove traces of HK which may be present. Depending on the nature of the substitutents on the Formula VI compounds and the nature of L, the Formula V compound may be obtained as a mixture with the corresponding Formula VII compound.

The desired Formula VI compounds wherein R¹, R², R⁴, R⁵, R⁶, and R⁷ are as described above may be prepared from the corresponding Formula VIII compounds by reaction with a suitable oxidizing agent, typically manganese (IV) oxide, in a suitable reaction inert solvent such as diethyl ether at a temperature between 0° C. to 25° C., typically ambient. Since this reaction produces an equivalent of water, which may be deleterious in the subsequent step, this may optionally be removed by addition of a solvent suitable for the next reaction such as acetonitrile or toluene and evaporation of the solvent mixture to a volume somewhat less than that of the added solvent, but not to dryness.

The desired Formula VIII compounds wherein R¹, R², R⁴, R⁵, R⁶, and R⁷ are as described above may be prepared from the corresponding Formula IX compounds by reaction with hydrazine hydrate in a suitable reaction inert solvent such as ethanol or toluene at a temperature between 25° C. to 180° C., typically 80° C. to 170° C. The hydrazone formation may be assisted by continuous removal of water, such as by the use of a Dean-Stark apparatus, or by heating in a closed vessel to a temperature beyond the boiling point of the solvent such as by a microwave oven.

An alternative preparation of the desired Formula II compounds wherein L is a (C₁-C₆) alkoxycarbonyl group and V, R¹, R², R⁴, R⁴, R⁵, and R⁷ are as described above may be accomplished from the corresponding Formula X compounds by treatment with a chlorinating agent such as phosphorus (III) chloride or thionyl chloride in a reaction inert solvent such as methylene chloride or chloroform optionally containing a base such as pyridine, diisopropylethylamine or 2,6-di-tert-butyl-4-methylpyridine at a temperature between 0° C. to 60° C., typically ambient, for a period between 1 to 24hr. The chloro-derivative thus formed as a mixture of diastereoisomers is then treated with a finely divided metal such as zinc in the presence of an acid, or mixture of acids, such as acetic acid or hydrochloric acid in a suitable solvent, or mixture of solvents such as methanol, water or tetrahydrofuran at a temperature between 25° C. to 60° C., typically ambient, to provide the desired product of Formula II.

The desired Formula X compounds wherein L is a (C₁-C₆) alkoxycarbonyl group and V, R¹, R², R⁴, R⁵, R⁶, and R⁷ are as described above may be prepared as a mixture of diastereoisomers from the corresponding Formula XI compounds by reaction with a suitable organometallic derivative of the V group such as a magnesium or lithium derivative, prepared in turn from a compound V-Hal where Hal represents a chlorine, bromine or iodine atom, using methods well known to those skilled in the art, for example as described in L. A. Paquette (Ed), Encyclopedia of Reagents for Organic Synthesis, John Wiley and Sons, Chichester, England, 1995. This reaction is conducted in a suitable reaction inert solvent such tetrahydrofuran or diethyl ether at a temperature between −78° C. to 25° C., typically −78° C., to provide the desired product of Formula X.

The desired Formula XI compounds wherein L is a (C₁-C₆) alkoxycarbonyl group and R¹, R², R⁴, R⁵, R⁶, and R⁷ are as described above may be prepared as a mixture of diastereoisomers from the corresponding Formula V compounds by reduction of the double bond. This may be achieved by hydrogenation in a reaction inert solvent such as methanol, ethanol or acetic acid with a catalyst such as palladium or rhodium on carbon under a hydrogen pressure equal to 15-50 psi for a period of 2-24 hrs, or by transfer hydrogenation using ammonium formate in refluxing methanol in the presence of a catalyst such as palladium on carbon in a reaction inert solvent such as methanol or ethanol at a temperature between 0° C. to 80° C., typically 25° C. to 60° C.

An alternative preparation of the desired Formula X compounds wherein L is a (C₁-C₆) alkoxycarbonyl group and V, R¹, R², R⁴, R⁵, R⁶, and R⁷ are as described above may be prepared as a mixture of diastereoisomers from the corresponding Formula XII compounds, wherein K is a leaving group such as chlorine or bromine, by hydrogenation in a reaction inert solvent such as methanol, ethanol or acetic acid with a catalyst such as palladium or rhodium on carbon under a hydrogen pressure equal to 15-50 psi for a period between 2-24 hrs, or by transfer hydrogenation using ammonium formate in refluxing methanol in the presence of a catalyst such as palladium on carbon in a reaction inert solvent such as methanol or ethanol at a temperature between 0° C. to 80° C., typically 25° C. to 60° C. to provide the desired product of Formula X.

Another alternative preparation of the desired Formula X compounds wherein L is a (C₁-C₆) alkoxycarbonyl group and V, R¹, R², R⁴, R⁵, R⁶, and R⁷ are as described above may be prepared as a mixture of diastereoisomers from the corresponding Formula IV compounds by reduction of the double bond. This may be accomplished by hydrogenation in a reaction inert solvent such as methanol, ethanol or acetic acid with a catalyst such as palladium or rhodium on carbon under a hydrogen pressure equal to 15-50 psi for a period between 2-24 hrs, or by transfer hydrogenation using ammonium formate in refluxing methanol in the presence of a catalyst such as palladium on carbon in a reaction inert solvent such as methanol or ethanol at a temperature between 0° C. to 80° C., typically 25° C. to 60° C. to provide the desired product of Formula X. An alternative method of reduction involves treatment with diimide which is generated in situ in a reaction-inert solvent in the presence of the Formula IV compounds by a number of methods known to those skilled in the arts, such as those described in L. A. Paquette (Ed), Encyclopedia of Reagents for Organic Synthesis, John Wiley and Sons, Chichester, England, 1995.

The desired Formula XII compounds wherein L is a (C₁-C₆) alkoxycarbonyl group, K is a leaving group such as chlorine or bromine, and V, R¹, R², R⁴, R⁵, R⁶, and R⁷ are as described above may be prepared as a mixture of diastereoisomers from the corresponding Formula VII compounds by reaction with a suitable organometallic derivative of the V group such as a magnesium or lithium derivative, prepared in turn from a compound V-Hal where Hal represents a chlorine, bromine or iodine atom. This reaction is conducted in a suitable reaction inert solvent such tetrahydrofuran or diethyl ether at a temperature between −78° C. to 25° C., typically −78° C., to provide the desired product of Formula XII.

As referred to above, the desired Formula VII compounds wherein L is a (C₁-C₆) alkoxycarbonyl group, K is a leaving group such as chlorine or bromine, and R¹, R², R⁴, R⁵, R⁶, and R⁷ are as described above may be prepared as a mixture of diastereoisomers from the corresponding Formula VI compounds by reaction with an acyl compound KCOL at a temperature between 0° C. to 25° C., typically ambient, in a reaction inert solvent such as acetonitrile or toluene, optionally in the presence of a base such as diisopropylethylamine or triethylamine to remove traces of HK which may be present. Depending on the nature of the substitutents on the Formula VI compounds and the nature of L, the Formula VII compound may be obtained as a mixture with the corresponding Formula V compound.

The desired Formula XIII compounds wherein M is a (C₁-C₆) alkoxy group and R¹, R², R⁴, R⁵, R⁶, and R⁷ are as described above may be prepared as a mixture of diastereoisomers from the corresponding Formula VI compounds by reaction with phosgene at a temperature between 0° C. to 25° C., typically ambient, in a reaction inert solvent such as acetonitrile or toluene in the presence of a base such as diisopropylethylamine or triethylamine. Addition of the desired alcohol MOH to the acid chloride in the presence of excess base then provides the desired Formula XIII compound.

The desired Formula XIV compounds wherein M is a (C₁-C₆) alkoxy group and R¹, R², R⁴, R⁵, R⁶, and R⁷ are as described above may be prepared as a mixture of diastereoisomers from the corresponding Formula XIII compounds by hydrogenolysis. This may be achieved by hydrogenation in a reaction inert solvent such as methanol, ethanol or acetic acid with a catalyst such as palladium or rhodium on carbon under a hydrogen pressure equal to 15-50 psi for a period between 2-24 hrs, or by transfer hydrogenation using ammonium formate in refluxing methanol in the presence of a catalyst such as palladium on carbon in a reaction inert solvent such as methanol or ethanol at a temperature between 0° C. to 80° C., typically 25° C. to 60° C. Alternatively, the Formula XIII compounds may be treated with a finely divided metal such as zinc in the presence of an acid, or mixture of acids, such as acetic acid or hydrochloric acid in a suitable solvent, or mixture of solvents such as methanol, water or tetrahydrofuran at a temperature between 25° C. to 60° C., typically ambient, to provide the desired Formula XIV compounds.

The desired Formula XV compounds wherein R¹, R², R⁴, R⁵, R⁶, and R⁷ are as described above may be prepared by hydrolysis of the ester group of the Formula XIV compounds, using methods well known to those skilled in the arts, such as can be found in L. A. Paquette (Ed), Encyclopedia of Reagents for Organic Synthesis, John Wiley and Sons, Chichester, England, 1995, for example, by treatment with an aqueous base, preferably, lithium, sodium, or potassium hydroxide, in a polar solvent, preferably dioxane, at a temperature between 0° C. and 100° C. (preferably room temperature) for between 1 to 20 hours to provide the desired Formula XV compounds.

### Scheme 2

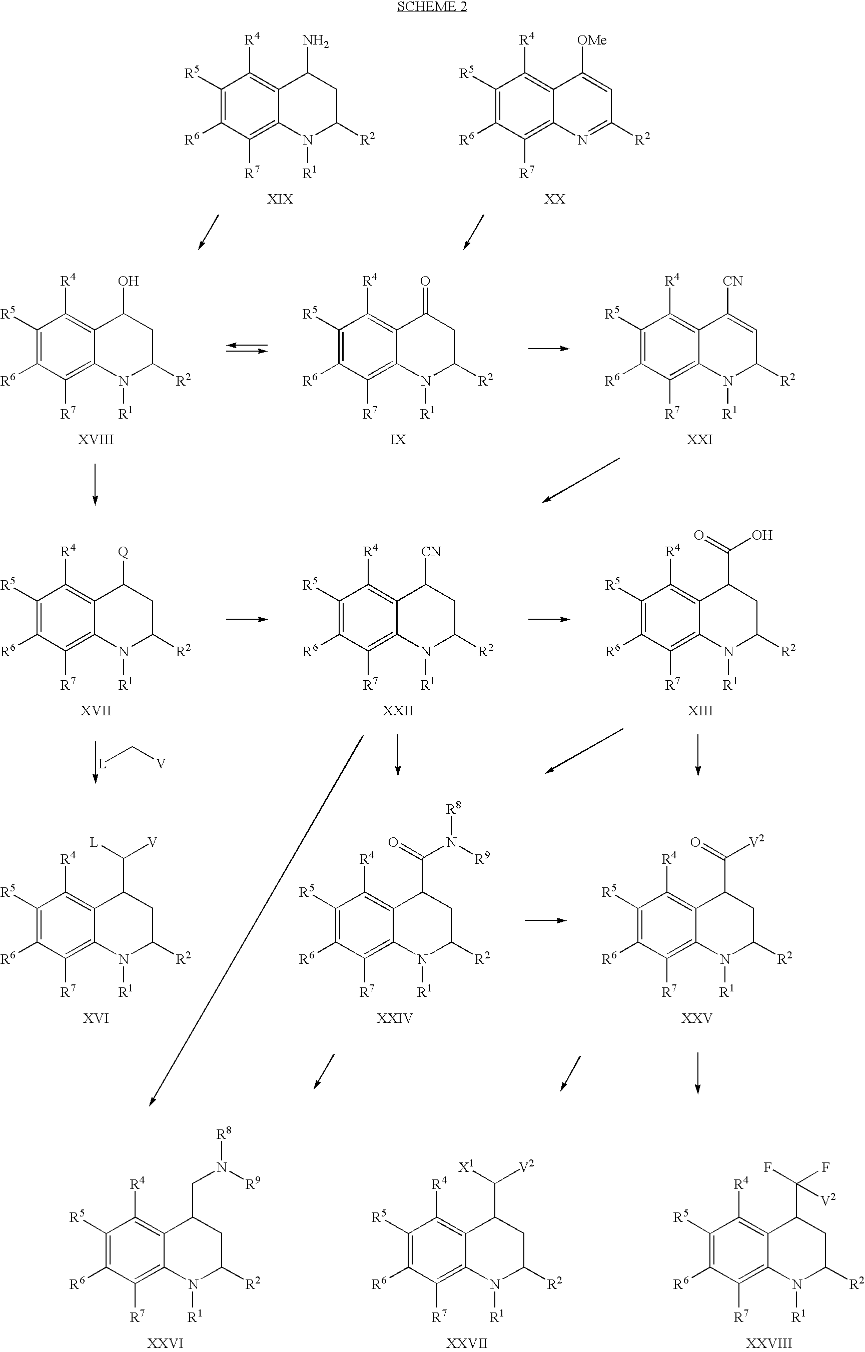

According to reaction Scheme 2, the desired compounds wherein J is carbon, the optional double bond is absent, R³ is a group CH(V)(L) wherein L is a (C₁-C₆) alkoxycarbonyl group or a cyano group and V, R¹, R², R⁴, R⁵, R⁶, and R⁷ are as described above (depicted as Formula XVI compounds) may be prepared as a mixture of diastereoisomers from the corresponding Formula XVII compounds by reaction with a compound VCH₂L in the presence of a suitable base such as 1,8-diazabicyclo[5.4.0]undec-7-ene, diisopropylethylamine, triethylamine or sodium hydride in a reaction inert solvent such as N,N-dimethylformamide, dimethylsulfoxide, acetonitrile or toluene at a temperature between 0° C. to 60° C., typically ambient.

The desired Formula XVII compounds wherein Q is a leaving group such as chlorine, bromine, methanesulfonyloxy or p-toluenesulfonyloxy and R¹, R², R⁴, R⁵, R⁶, and R⁷ are as described above may be prepared as a mixture of diastereoisomers from the corresponding Formula XVIII compounds by reaction with the appropriate reagent such as methanesulfonyl chloride or toluenesulfonyl chloride in the presence of a suitable base such as diisopropylethylamine or triethylamine in a reaction inert solvent such as N,N-dimethylformamide, dimethylsulfoxide, chloroform, methylene chloride or toluene at a temperature between 0° C. to 60° C., typically ambient. Other suitable reagents for formation of the Formula XVII compounds include phosphorus (III) chloride, phosphorus (III) bromide and thionyl chloride optionally in a reaction inert solvent such as chloroform, methylene chloride, pyridine or toluene at a temperature between 0° C. to 60° C., typically ambient.

The desired Formula XVIII compounds wherein R¹, R², R⁴, R⁵, R⁶, and R⁷ are as described above may be prepared as a mixture of diastereoisomers from the corresponding Formula IX compounds by reduction of the carbonyl group using methods and reagents well known to those skilled in the arts, such as can be found in L. A. Paquette (Ed), Encyclopedia of Reagents for Organic Synthesis, John Wiley and Sons, Chichester, England, 1995, for example using sodium borohydride in an alcohol solvent such as methanol of ethanol at a temperature between 0° C. to 60° C., typically ambient or using potassium tri-sec-butylborohydride (K-Selectride®) in a reaction inert solvent such as tetrahydrofuran or diethyl ether at a temperature between −78° C. to 25° C., typically 0° C.

In an alternative procedure, the desired Formula XVIII compounds wherein R¹, R², R⁴, R⁵, R⁶, and R⁷ are as described above may be obtained by treatment of the corresponding Formula XIX compounds with sodium nitrite in the presence of an acid, preferably acetic acid, followed by hydrolysis with a suitable base such as lithium, sodium, or potassium hydroxide, preferably sodium hydroxide in a suitable hydroxylic solvent such as ethanol to give the desired Formula XIX compounds. Methods for the preparation of Formula XIX compounds are described in U.S. Pat. No. 6,197,786 and International Application WO 0140190.

The desired Formula IX compounds wherein R¹ is an alkoxycarbonyl group and R², R⁴, R⁵, R⁶, and R⁷ are as described above may be prepared from the corresponding 4-methoxyquinoline compounds of Formula XX by treatment with an organomagnesium derivative of the R² group together with an acylating agent such as ethyl chloroformate at a temperature between −100° C. to 70° C., typically −78° C. in a reaction inert solvent such as tetrahydrofuran followed by warming to a temperature between 0° C. and about 70° C. (preferably ambient) for between 0.1 and 24 hr, preferably 1 hr, followed by hydrolysis in aqueous acid, preferably 1N hydrochloric acid to give the desired Formula IX compounds, as described in U.S. Pat. No. 6,197,786.

In an alternative procedure, the desired Formula IX compounds wherein R¹, R², R⁴, R⁵, R⁶, and R⁷ are as described above may be obtained by oxidation of the corresponding Formula XVIII compounds using a variety of methods and reagents well known to those skilled in the arts, such as can be found in L. A. Paquette (Ed), Encyclopedia of Reagents for Organic Synthesis, John Wiley and Sons, Chichester, England, 1995, for example pyridinium chlorochromate, aqueous sodium hypochlorite in the presence of a catalytic amount of 2,2,6,6-tetramethyl-1-piperidinyloxy (TEMPO) free radical and catalytic potassium bromide in a suitable reaction inert solvent such as methylene chloride, or alternatively with acetic anhydride and dimethylsulfoxide.

The desired Formula XXI compounds wherein R¹, R², R⁴, R⁵, R⁶, R⁷ are as described above may be prepared from the corresponding Formula IX compounds by treatment with trimethylsilylcyanide in an inert solvent such as an aromatic hydrocarbon (e.g., benzene, toluene, xylene) in the presence of a catalytic amount of Lewis acid, preferably zinc iodide, at a temperature of about 25° C. to about 140° C., preferably about 80° C. to about 100° C., for 1-12 hours, preferably 5 hours. The resulting solution is concentrated to dryness and added directly without further purification to a polar solvent (e.g., methanol, ethanol). A solution of acid (preferably hydrochloric) in a polar aprotic solvent (preferably dioxane) is added to the solution and the mixture is stirred at a temperature from 0° C. to about 100° C., preferably room temperature, for 1 to 24 hours, preferably 12 hours, to yield the Formula XXI compounds.

The desired Formula XXII compounds wherein R¹, R², R⁴, R⁵, R⁶, R⁷ are as described above may be prepared from the corresponding Formula XXI compounds by treatment with a reducing agent such as sodium borohydride or sodium cyanoborohydride in a reaction inert solvent such as methanol or ethanol, preferably ethanol, at a temperature of about 0° C. to about 100° C. (preferably reflux temperature) for 0.1 to 5 hours (preferably 0.75 hour) to provide the desired Formula XXII compounds.

Alternatively, the desired Formula XXII compounds wherein R¹, R², R⁴, R⁵, R⁶, R⁷ are as described above may be prepared from the corresponding Formula XVII compounds, wherein Q is a leaving group as described above, by treatment with a cyanide salt such as lithium, sodium, potassium or a tetraalkylammonium cyanide in a reaction inert solvent such as dimethylformamide at a temperature between 0° C. to 100° C. for 1 to 12 hours, to provide the Formula XXII compounds.

The desired Formula XXIII compounds wherein R¹, R², R⁴, R⁵, R⁶, R⁷ are as described above may be prepared from the corresponding Formula XXII compounds by dissolving in concentrated sulfuric acid containing five equivalents of water at a temperature from 0° C. to 100° C. (preferably room temperature) for 1 to 20 hours. The resulting amide is then dissolved in a polar solvent (preferably methylene chloride) and treated with trimethyloxonium tetrafluoroborate at a temperature from 0° C. to 100° C. (preferably room temperature) for 1-20 hours (preferably 12 hours). The resulting imino ester is then treated with an aqueous base, preferably, lithium, sodium, or potassium hydroxide, in a polar solvent, preferably dioxane, at a temperature between 0° C. and 100° C. (preferably room temperature) for between 1 to 20 hours to provide the Formula XXIII Compounds.

The desired Formula XXIV compounds wherein R¹, R², R⁴, R⁵, R⁶, R⁷, R⁸ and R⁹ are as described above may be prepared from the corresponding Formula XXIII compounds by treating the acid in a reaction inert solvent (preferably dichloromethane) with the corresponding amine (NHR⁸R⁹) in the presence of 1-hydroxybenzotriazole hydrate (HOBT) and 1-(3-dimethylaminopropyl)-3-ethylcarbodiimide hydrochloride (EDCl) at a temperature between 0° C. to 100° C. (preferably ambient temperature) for 1 to 24 hours (preferably 12 hours).

The desired Formula XXV compounds wherein R¹, R², R⁴, R⁵, R⁶, R⁷are as described above and V² is a group V or CH₂V where V is as described above may be prepared from the corresponding Formula XXIV compounds when R⁸ is methyl and R⁹ is methoxy (the ‘Weinreb’ amide) by treatment with a variety of V²Met compounds where Met is a metal, preferably magnesium or lithium, to produce the desired Formula XXV compounds.

The desired Formula XXVI compounds wherein R¹, R², R⁴, R⁵, R⁶, R⁷, R⁸ and R⁹ (which are within the scope of the present invention) are as described above may be prepared from the corresponding Formula XXIV compounds by reduction with a hydride source, preferably sodium borohydride, in the presence of an acid such as trifluoroacetic acid in a reaction inert solvent (preferably tetrahydrofuran) at a temperature of between 0° C. and 100° C. for 1 to 20 hours (preferably 12 hour). When R⁸ and/or R⁹ is H, the amine may be acylated using standard amide coupling conditions known to those skilled in the art such as those described in L. A. Paquette (Ed), Encyclopedia of Reagents for Organic Synthesis, John Wiley and Sons, Chichester, England, 1995, to produce further Formula XXVI compounds.

An alternative preparation of the desired Formula XXVI compounds wherein R¹, R², R⁴, R⁵, R⁶ and R⁷ are as described above and R⁸ and R⁹ are H involves reduction of the corresponding Formula XXII compounds using procedures known to those skilled in the art, such as those described in L. A. Paquette (Ed), Encyclopedia of Reagents for Organic Synthesis, John Wiley and Sons, Chichester, England, 1995, for example using borane-tetrahydrofuran complex in a reaction inert solvent. These primary amines may subsequently be converted to amides as described above to produce further compounds within the scope of the present invention. The desired Formula XXVII compounds wherein R¹, R², R⁴, R⁵, R⁶, R⁷and V² are as described above and X¹ is OH, F, or H, may be prepared from the corresponding Formula XXV compounds by treatment with a reducing agent such as sodium borohydride in a polar solvent such as methanol or ethanol at a temperature of about 0° C. to about 100° C. for 1 to 10 hours (preferably 1 hour) to produce Formula XXVII compounds wherein X¹ is OH.

Formula XXVII compounds when X¹=N may be prepared from the corresponding alcohol (X¹=OH) which is converted to the mesylate and displaced with sodium azide. The azide is hydrogenated to NH₂. One skilled in the art can convert NH₂ to NR¹H by standard reductive amination conditions using the corresponding aldehyde and reducing agent, such as sodium borohydride or sodium cyano borohydrite. NR¹H can be converted to NR¹R² using the same conditions of reductive amination to produce NR¹R².

Moreover, when X¹ is NH₂, the corresponding alcohol (X¹=OH) may be converted to the mesylate, displaced with an azide and reduced with hydrogen to produce one single diastereomer. When X¹ is NHR¹ (R¹=Me), the corresponding primary amine NH₂ may be converted to NHR¹ by treatment with ethyl formate at a temperature between 0° C. to 100° C. between 1-24 hours (preferably 12 hours). The resulting formamide is added directly without further purification to a nonpolar solvent (e.g., benzene, toluene, preferably toluene) and a reducing agent such as borane methyl sulfide complex at a temperature between 0° C. to 100° C. between 1-24 hours (preferably 12 hours) to provide the desired mono methyl amine product. When X¹ is NHR¹, the corresponding primary amine NH₂ may be converted to NHR¹ by standard reductive amination conditions by treatment with an aldehyde R¹ in a polar solvent such as methanol or ethanol in the presence of sodium borohydride or sodium cyanoborohydride at a temperature between 0° C. to 100° C. between 1-24 hours (preferably 12 hours). When X¹ is NR¹R², the corresponding secondary amine NHR¹ may be converted to NR¹R²by standard reductive as described above. Compound XXV may also be converted to NH₂, NHR¹, and NR¹R² by standard reductive amination conditions with the corresponding aldehyde or ketone as described above. This method produces a mixture of amine diastereomers that may be separated by silica gel chromatography.

These may be converted to the corresponding Formula XXVII compounds where X¹ is F by treatment with a fluorinating agent, such as diethylaminosulfur trifluoride (DAST) or [bis(2-methoxyethyl)amino]sulfur trifluoride (Deoxyfluor) in a reaction inert solvent such as dichloromethane or 1,2 dichloroethane at a temperature between −78° C. to about 100° C. (preferably ambient temperature) for 0.1 to 10 hours (preferably 1 hour). These may be converted to the corresponding Formula XXVII compounds wherein X¹ is H by reduction with a suitable hydride source such as diisobutyl aluminum hydride in a reaction inert solvent such as tetrahydrofuran at a temperature between −78° C. to about 100° C., preferably about 0° C., for 0.1 to 10 hours.

The desired Formula XXVIII compounds wherein R¹, R², R⁴, R⁵, R⁶, R⁷ are as described above and V² is a group V or CH₂V where V is as described above may be prepared from the corresponding Formula XXV compounds by treatment with a fluorinating agent, such as diethylaminosulfur trifluoride (DAST) or [bis(2-methoxyethyl)amino]sulfur trifluoride (Deoxyfluor) in a reaction inert solvent such as dichloromethane or 1,2-dichloroethane at a temperature between −78° C. to about 100° C. (preferably ambient temperature) for 0.1 to 24 hours (preferably 12 hours).

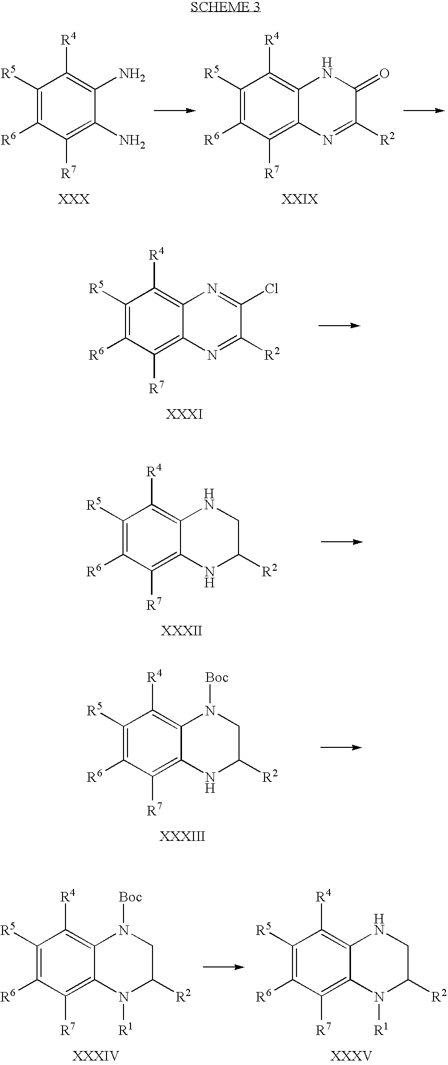

According to Scheme 3, the desired Formula XXIX compounds wherein J is nitrogen, the optional double bond is absent, and R², R⁴, R⁵, R⁶, and R⁷ are as described above, may be prepared from the corresponding Formula XXX compounds by reaction with an alpha-ketocarboxylic acid R²COCO₂H in a protic solvent, such as ethanol or methanol at high temperature. These temperatures can be conveniently and safely achieved using a microwave apparatus familiar to one skilled in the art, such as a Emrys Optimizer (Personal Chemistry, Uppsala, Sweden) or Milestone microwave (Milestone Laboratories, Sorisole, Italy). The resulting reaction mixture is concentrated to dryness and the Formula XXIX compounds can be usually crystallized or alternatively can be purified by flash chromatography on silica gel.

The desired Formula XXXI compounds wherein R², R⁴, R⁵, R⁶, and R⁷ are as described above may be prepared from the corresponding Formula XXIX compounds by treatment with a chlorinating agent such as phosphorus (III) chloride, phosphorus (V) oxychloride, thionyl chloride or triphenylphosphine/carbon tetrachloride. Typically Formula XXIX compounds are dissolved in excess phosphorus oxychloride and the mixture is heated to about 100° C., for 12-18 h. After cooling excess phosphorus (V) oxychloride is distilled off and the residue is carefully quenched with saturated NaHCO₃. The resulting aqueous suspension is extracted with an appropriate organic solvent, preferably methylene chloride.

The desired Formula XXXII compounds wherein R², R⁴, R⁵, R⁶, and R⁷ are as described above may be prepared from the corresponding Formula XXXI compounds by catalytic hydrogenation in the presence of standard catalysts well known to those skilled in the art, for example as described in L. A. Paquette (Ed), Encyclopedia of Reagents for Organic Synthesis, John Wiley and Sons, Chichester, England, 1995. Typically, the compound is dissolved in an organic solvent, preferably a polar solvent such as acetic acid. Additives such as sodium acetate are usually added to improve the reaction rate. An appropriate catalyst is chosen, such as palladium on carbon. Hydrogenation is carried out at elevated pressures in an appropriate apparatus, preferably for about 6 h. The catalyst is filtered off to yield the Formula XXXII compound, which is typically isolated after chromatography on silica gel.

The desired Formula XXXIII compounds wherein R², R⁴, R⁵, R⁶, and R⁷ are as described above may be prepared from the corresponding Formula XXXII compounds by reaction with boc-anhydride as described in the T. W. Greene and G. M. Wuts, Protective Groups in Organic Synthesis, Wiley Interscience, 1991. Typically, the reaction is carried out in a solvent such as methylene chloride at −78° C. to 0° C., typically −40° C. In general, in this procedure, there is a selective functionalization of the less hindered nitrogen in Formula XXXII compounds. The Formula XXXIII compounds can be purified by standard silica gel chromatography, if needed.

The desired Formula XXXIV compounds wherein R¹, R², R⁴, R⁵, R⁶, and R⁷ are as described above may be prepared from the corresponding Formula XXXIII compounds by introducing the acyl, carbamoyl, sulfinyl or sulfonyl group R¹. This may be achieved by treatment with an appropriate reagent, for example, ethyl chloroformate or isopropyl chloroformate, in a suitable reaction inert solvent such as methylene chloride or chloroform optionally containing a base such as pyridine, diisopropylethylamine, 4-dimethylaminopyridine or 2,6-di-tert-butyl-4-methylpyridine at a temperature between 0° C. to 60° C., typically ambient, for a period of 1 to 24 hr. At times, the reaction is carried out in pyridine as a solvent. The product is usually isolated by standard extractive workup and flash chromatography on silica gel.

The desired Formula XXXV compounds wherein R¹, R², R⁴, R⁵, R⁶, and R⁷ are as described above may be prepared from the corresponding Formula XXXIV compound by treatment with an acid as described in the T. W. Greene and G. M. Wuts, Protective Groups in Organic Synthesis, Wiley Interscience, 1991. In a typical procedure, the bis-carbamate is treated with trifluoroacetic acid at ambient temperature for 1-24 hr, typically 3 hr. Upon completion, the acid is removed by evaporation and the residue is partitioned between an organic solvent, preferably methylene chloride and aqueous sodium hydrogen carbonate. Evaporation of the organic solvent affords the desired Formula XXXV compounds.

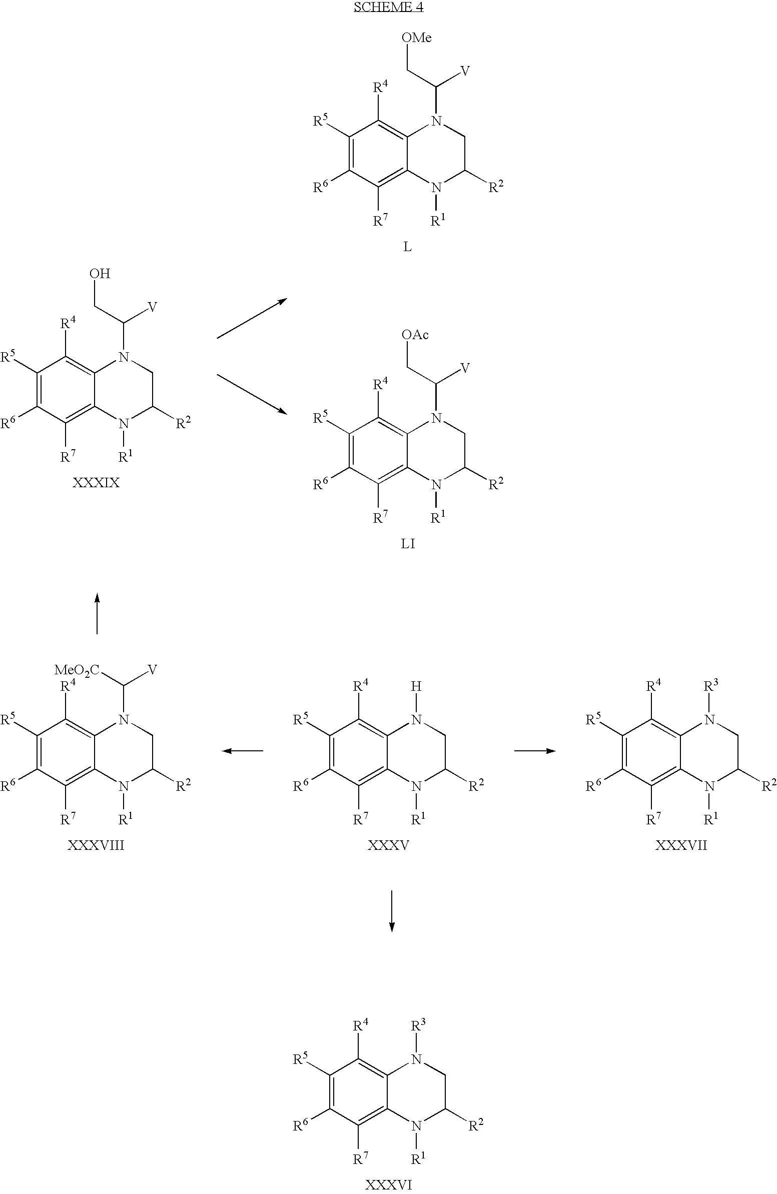

According to Scheme 4, the desired compounds wherein J is nitrogen, the optional double bond is absent, R¹, R², R⁴, R⁵, R⁶ and R⁷ are as described above and R³ is as described above wherein the connecting carbon is oxo substituted (depicted as Formula XXXVI compounds) may be prepared from the corresponding Formula XXXV compounds by reaction with an acid chloride, in a solvent such as methylene chloride or chloroform, optionally containing a base such as pyridine, diisopropylethylamine, 4-dimethylaminopyridine or 2,6-di-tert-butyl-4-methylpyridine, at a temperature between 0° C. to 60° C., typically ambient, for a period of 1 to 24 hr. Upon completion of the reaction, the mixture is washed with aqueous acid and brine to afford the Formula XXXVI compounds after silica gel chromatography. If desired, the acid chloride can be generated in situ, from the corresponding carboxylic acid and triphenylphosphine in conjunction with agents such as carbon tetrachloride, hexachloroethane or trichloroacetonitrile. This latter procedure can be carried out with resin bound triphenylphosphine which makes it amenable to automated chemistry. Filtration of the resin followed by evaporation and purification on silica gel yields the required compound of Formula XXXVI.

The desired Formula XXXVII compounds wherein R¹, R², R⁴, R⁵, R⁶, and R⁷ are as described above and R³ is as described above, wherein the group V is attached to the connecting carbon, may be prepared from the corresponding Formula XXXV compounds by alkylation with the appropriate alkyl bromides. These alkylations are typically carried out in a polar solvent such as dimethylformamide, dimethyl sulfoxide, N-methylpyrrolidone etc. in the presence of a base (e.g., potassium carbonate, triethylamine, pyridine, 4-dimethylaminopyridine, lutidine) at a temperature between 25° C. to 200° C., typically 150° C. Due to the unreactivity of quinoxalines, heating in the microwave at the appropriate temperature is particularly suitable for this procedure. Alkylations can be performed with a variety of alkyl bromides, such as arylmethyl bromides or alpha-substituted arylmethyl bromides. Use of suitable microwave equipment such as the Emrys Optimizer (Personal Chemistry, Uppsala, Sweden) or Milestone microwave (Milestone Laboratories, Sorisole, Italy) facilitates these reactions.

The desired Formula XXXVIII compounds, wherein R¹, R², R⁴, R⁵, R⁶, R⁷ and V are as described above may be prepared from the corresponding alpha-bromoester VCHBrCO₂Me compounds. These alkylations are typically carried out in a polar solvent such as dimethylformamide, dimethyl sulfoxide, N-methylpyrrolidone etc. in the presence of a base (e.g., potassium carbonate, triethylamine, pyridine, 4-dimethylaminopyridine, lutidine) at a temperature between 25° C. to 200° C., typically 150° C. Due to the unreactivity of quinoxalines, heating in the microwave at the appropriate temperature is particularly suitable for this procedure. Use of suitable microwave equipment such as the Emrys Optimizer (Personal Chemistry, Uppsala, Sweden) or Milestone microwave (Milestone Laboratories, Sorisole, Italy) facilitates these reactions.

The desired Formula XXXIX compounds, wherein R¹, R², R⁴, R⁵, R⁶, R⁷ and V are as described above may be prepared from the corresponding Formula XXXVIII compounds by the reduction of the ester functionality using reagents and conditions well known to those skilled in the art. For example, Formula XXXVIII compounds can be treated with lithium aluminum hydride in anhydrous ethereal solvents such as tetrahydrofuran and ether at temperatures ranging between −78° C. and 0° C. Typically treating the reaction mixture with sodium sulfate hexahydrate or silica gel/chloroform and removal of the solids by filtration gives a crude product from which Formula XXXIX compounds are typically isolated by silica gel chromatography.

The desired Formula L compounds, wherein R¹, R², R⁴, R⁵, R⁶, R⁷ and V are as described above may be prepared from the corresponding Formula XXXIX compounds by using an appropriate alkylating agent, such as an alkyl iodide or bromide and a base, such as sodium hydride, in a reaction inert solvent such as tetrahydrofuran or dimethylformamide to obtain the desired Formula L compounds.

The desired Formula LI compounds, wherein R¹, R², R⁴, R⁵, R⁶, R⁷ and V are as described above and Ac is an acyl group (which are within the scope of the present invention) may be prepared from the corresponding Formula XXXIX compounds by using a suitable acyl chloride and a base such as pyridine, triethylamine or 4-dimethylaminopyridine in an anhydrous solvent such as dichloromethane. Depending on the reactivity of the acyl chloride, one can use the base, such as pyridine, as the solvent. The products can usually be isolated by concentrating the reaction mixture and purifying the product by silica gel chromatography.

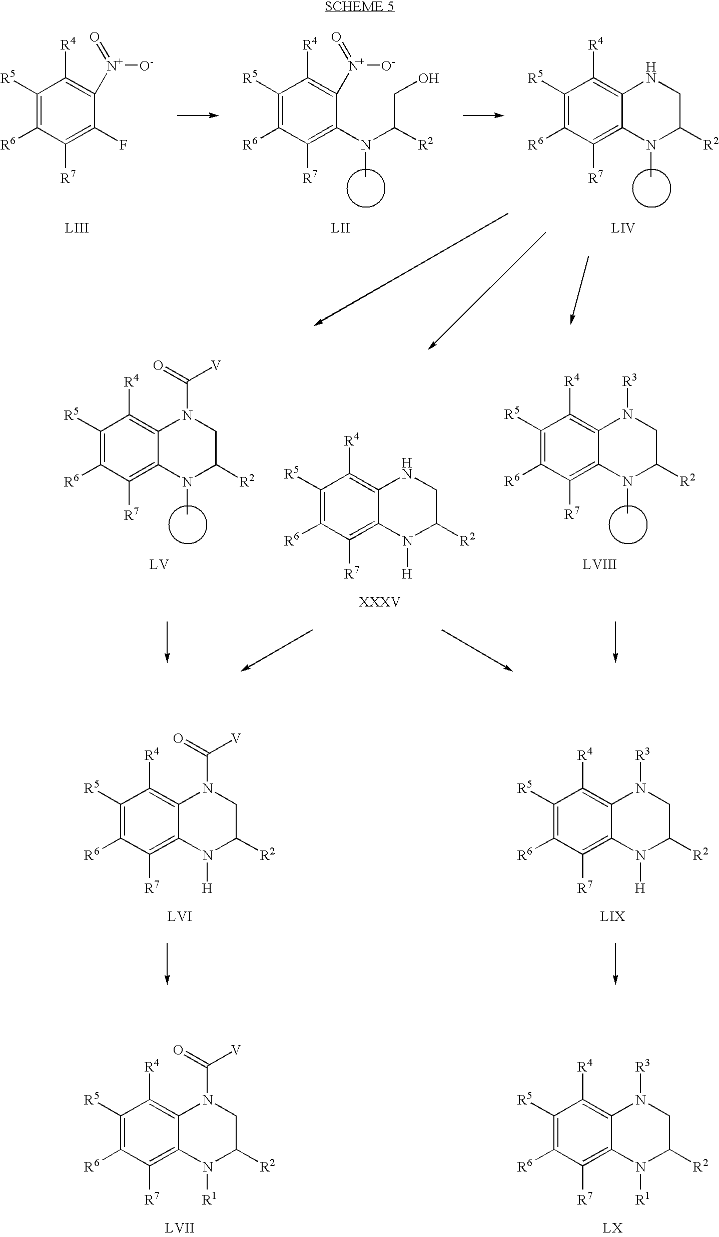

Scheme 5 describes the preparation of further compounds, in which J is nitrogen and the optional double bond is absent (i.e. quinoxalines), using solid phase chemistry similar to that described by V. Krchnak et. al., Tetrahedron Lett. 42, 2443-2446 (2001), and which offers several advantages. This chemistry can also be carried out in the solution phase by substituting the resin with an electron rich arylmethyl group, such as 4-methoxybenzyl or 2,4-dimethoxybenzyl and using it like a protecting group and cleaving under appropriate conditions as described in T. W. Greene and G. M. Wuts, Protective Groups in Organic Synthesis, Wiley Interscience, 1991. In particular, this scheme provides a method for preparation of chiral quinoxalines from chiral aminoalcohols, which are readily prepared from widely available chiral aminoacids using methods and reagents well known to those skilled in the art.

The resin bound Formula LII compounds wherein R², R⁴, R⁵, R⁶ and R⁷ are as described above can be prepared from the corresponding aminoalcohol R²CH(CH₂OH)NH₂ which is first bound to a resin by the method described by V. Krchnak et al., Tetrahedron Lett. 42, 2443-2446 (2001). This is then treated with the corresponding Formula LIII compounds typically in a polar solvent such as dimethyl sulfoxide, dimethylformamide, N-methylpyrrolidone or dimethylacetamide which is capable of allowing polystyrene based resin to swell as known to one skilled in the art. The reaction is carried out for 12-36 h, and the resin is then washed to isolate the desired resin bound Formula LII compounds.

The desired resin bound Formula LIV compounds, wherein R², R⁴, R⁵, R⁶, and R⁷ are as described above may be prepared from the corresponding resin bound alcohol of Formula LII compounds by activating the alcohol functionality as familiar to one skilled in the art. This involves the conversion of the alcohol to a sulfonate (such as a methanesulfonate or toluenesulfonate), halide (such as chloride or bromide) or an acetate. Preferably the reaction is carried out by treatment of the resin bound Formula LII compounds with methanesulfonyl chloride in the presence of a base such as pyridine, 4-dimethylaminopyridine or proton sponge in solvents such as dichloromethane or dichloroethane in which case the reaction is typically carried out for 1-5 h. The resulting resin bound sulfonate is washed of all the reagents and the nitro group can be reduced by a variety of reducing agents well known to those skilled in the art, for example as described in L. A. Paquette (Ed), Encyclopedia of Reagents for Organic Synthesis, John Wiley and Sons, Chichester, England, 1995. For example, one can use tin (II) chloride in a polar solvent such as N-methylpyrrolidone or dimethylformamide, which are capable of swelling a polystyrene based resin. The reaction is typically carried out for 1-5 h, the reagents are washed away and the resulting primary amine undergoes an intramolecular ring closure to afford the resin bound Formula LIV compounds. If one uses an activating group other than a methanesulfonate as described above, the reaction times for the ring closure might be longer as known to one skilled in the art.

The resin bound Formula LV compounds wherein R², R⁴, R⁵, R⁶, R⁷ and V are as described above may be prepared from the corresponding Formula LIV compounds, by reaction with an acid chloride VOCl, in a solvent such as methylene chloride or chloroform optionally containing a base such as pyridine, diisopropylethylamine, 4-dimethylaminopyridine or 2,6-di-tert-butyl-4-methylpyridine at a temperature between 0° C. to 60° C., typically ambient, for a period of 1 to 24 hr. The resin bound Formula LV compound is subsequently filtered and repeatedly washed with solvents such as dichloromethane, methanol and water to remove the excess reagents.

The desired Formula LVI compounds wherein R², R⁴, R⁵, R⁶, R⁷ and V are as described above may be prepared from the corresponding resin bound Formula LV compounds, by treatment with a strong acid well known to those skilled in the art, such as trifluoroacetic acid or hydrofluoric acid, optionally in a reaction-inert solvent such as dichloromethane or dichloroethane. Typically the desired Formula LVI compounds can be isolated by filtration and washing of the resin with an appropriate organic solvent such as dichloromethane, dichloroethane or tetrahydrofuran. If necessary, the Formula LVI compounds can be further purified by silica gel chromatography under standard conditions.

The desired Formula LVII compounds wherein R¹, R², R⁴, R⁵, R⁶, R⁷ and V are as described above may be prepared from the corresponding Formula LVI compounds by treatment with an appropriate reagent, for example, ethyl chloroformate or isopropyl chloroformate, in a reaction inert solvent such as methylene chloride or chloroform, optionally containing a base such as pyridine, diisopropylethylamine, 4-dimethylaminopyridine or 2,6-di-tert-butyl-4-methylpyridine at a temperature between 0° C. to 60° C., typically ambient, for a period of 1 to 24 hr. At times, the reaction is carried out in pyridine as a solvent. In the particular case where R¹ is a carbamate or urea, the product can be obtained by treating Formula LVI compounds with phosgene in toluene, optionally containing a base such as pyridine, diisopropylethylamine, 4-dimethylaminopyridine or 2,6-di-tert-butyl-4-methylpyridine at a temperature between 0° C. to 60° C., followed by treatment with, respectively, an alcohol or an amine to afford the desired carbamate or urea of Formula LVII. The product is usually isolated by standard extractive workup and flash chromatography on silica gel.

The desired resin bound Formula LVIII compounds, wherein R¹, R², R⁴, R⁵, R⁶, and R⁷ are as described above and R³ is as described above, wherein the group V is attached to the connecting carbon, may be prepared from the corresponding resin bound Formula LIV compounds by alkylation with the appropriate alkyl bromides or iodides. These alkylations are typically carried out in a polar solvent such as dimethylformamide, dimethyl sulfoxide or N-methylpyrrolidone in the presence of a base (triethylamine, pyridine, 4-dimethylaminopyridine, lutidine). The solvents used in this reaction are capable of allowing the polystyrene resin to swell as familiar to one skilled in the art. These reactions are usually carried out from ambient temperatures to about 150° C. Due to the unreactivity of quinoxalines, heating in a microwave oven at an appropriate temperature is preferred. Alkylations can be performed with a variety of alkyl bromides such as arylmethyl bromides or alpha-substituted arylmethyl bromides. The alkylation reactions typically proceed in better yield if the bromides are substituted with an alpha electron-withdrawing group (such as in the preparation of the Formula XXXVIII compounds in Scheme 4).

The quinoxaline compounds of Formula LIX wherein R¹, R², R⁴, R⁵, R⁶, and R⁷ are as described above may be prepared from the corresponding resin bound Formula LVIII compounds, by treatment with strong acids well known to those skilled in the art, such as trifluoroacetic acid or hydrofluoric acid, with or without additional solvents (such as dichloromethane or dichloroethane). Typically, the quinoxaline compounds of Formula LIX, can be isolated by filtration and washing of the resin with an appropriate organic solvent such as dichloromethane, dichloroethane or tetrahydrofuran. Evaporation of the solvent usually affords clean desired Formula LIX compounds. If necessary, the isolated products can be further purified by silica gel chromatography under standard conditions.

The desired Formula LX compounds wherein R¹, R², R³, R⁴, R⁵, R⁶, and R⁷ are as described above may be prepared from the corresponding Formula LIX compound by treatment with an acylating/sulfonating agent solvent, for example ethyl or isopropyl chloroformate in a reaction-inert solvent such as methylene chloride or chloroform optionally containing a base such as pyridine, diisopropylethylamine, 4-dimethylaminopyridine or 2,6-di-tert-butyl-4-methylpyridine at a temperature between 0° C. to 60° C., typically ambient, for a period of 1 to 24 hr. At times, the reaction is carried out in pyridine as a solvent. In the particular case when R¹ is a carbamate or urea, the product can be obtained by treating Formula LX compound with phosgene in toluene optionally containing a base such as pyridine, diisopropylethylamine, 4-dimethylaminopyridine 2,6-di-tert-butyl-4-methylpyridine at a temperature between 0° C. to 60° C., followed by treatment with, respectively, an alcohol or an amine to afford the desired carbamate or urea Formula LX compound. The product is usually isolated by standard extractive workup and flash chromatography on silica gel.

An alternative preparation of the desired Formula LVI compounds wherein R², R⁴, R⁵, R⁶, R⁷ and V are as described above may be achieved from the corresponding Formula XXXV compounds by reaction with an acylating agent such as acid chloride VOCl, in a solvent such as methylene chloride or chloroform optionally containing a base such as pyridine, diisopropylethylamine, 4-dimethylaminopyridine or 2,6-di-tert-butyl-4-methylpyridine at a temperature between 0° C. to 60° C., typically ambient, for a period of 1 to 24 hr. Typically under these conditions, when R² is other than hydrogen, the less hindered nitrogen atom of the quinoxaline preferentially undergoes acylation to give the desired Formula LVI compounds. The desired compounds of Formula LVII wherein R¹, R², R⁴, R⁵, R⁶, R⁷ are as described above and R³ is as described above, wherein the group V is attached to the connecting carbon, may be achieved from the corresponding Formula LVI compounds by alkylation or acylation, using procedures as described above.

An alternative preparation of the desired Formula LIX compounds wherein R², R⁴, R⁵, R⁶, R⁷ are as described above and R³ is as described above, wherein the group V is attached to the connecting carbon, may be achieved from the corresponding Formula XXXV compounds, via the compounds of Formula LIX, by alkylation with the appropriate alkyl bromides or iodides. These alkylations are typically carried out in a polar solvent such as dimethylformamide, dimethyl sulfoxide, and N-methylpyrrolidone in the presence of a base (e.g., triethylamine, pyridine, 4-dimethylaminopyridine, lutidine). These reactions are usually carried out from ambient temperatures to about 150° C. Due to the unreactivity of quinoxalines, heating in a microwave oven at an appropriate temperature is preferred. Alkylations can be performed with a variety of alkyl bromides such as arylmethyl bromides, alpha-substituted arylmethyl bromides. Typically under these conditions, when R² is other than hydrogen, the less hindered nitrogen atom of the quinoxaline preferentially undergoes alkylation to give the desired Formula LIX compounds.

The desired Formula XXXV compounds wherein R², R⁴, R⁵, R⁶, and R⁷ are as described above may be prepared from the corresponding resin bound Formula LIV compounds by treatment with strong acids well known to those skilled in the art, such as trifluoroacetic acid or hydrofluoric acid, optionally in a reaction inert solvent such as dichloromethane or dichloroethane.

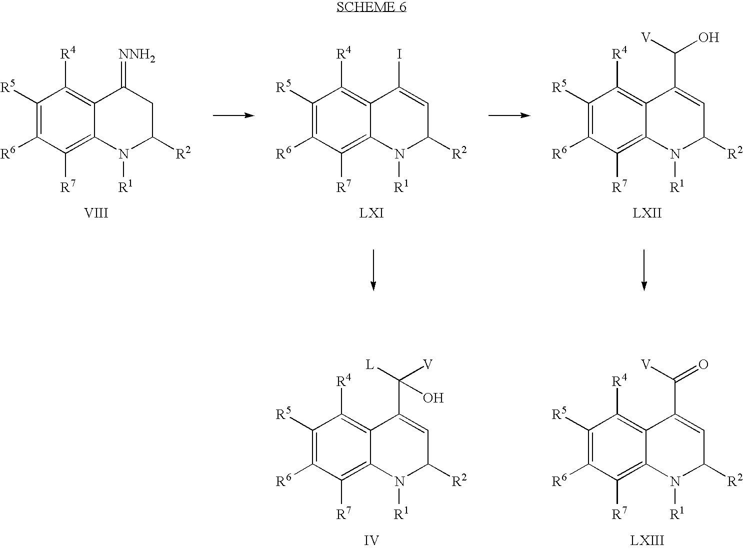

According to reaction Scheme 6, the desired compounds wherein J is carbon, the optional double bond is present, R³ is a group COV and V, R¹, R², R⁴, R⁵, R⁶, and R⁷ are as described above (depicted as Formula LXIII compounds) can be prepared from the corresponding Formula LXII compounds by oxidation of the alcohol. This may be achieved by a wide variety of methods well known to those skilled in the art, for example as described in L. A. Paquette (Ed), Encyclopedia of Reagents for Organic Synthesis, John Wiley and Sons, Chichester, England, 1995. For example the Formula LXII compounds can be treated with activated manganese (IV) oxide in a suitable reaction inert solvent such as tetrahydrofuran, diethyl ether or dichloromethane at a temperature between 0° C. to 25° C., typically ambient, to provide the desired product of Formula LXIII.

The desired Formula LXII compounds wherein V, R¹, R², R⁴, R⁵, R⁶, and R⁷ are as described above can be prepared as a mixture of diastereoisomers from the corresponding Formula LXI compounds by treatment with a suitable transmetallating agent such as an alkyllithium compound, such as n-butyllthium or s-butyllithium, or an alkylmagnesium halide such as isopropylmagnesium chloride in a suitable reaction inert solvent such as tetrahydrofuran or diethyl ether at a temperature between −120° C. to 0° C., typically −78° C., to provide a vinylmetal species which is then reacted with the appropriate aldehyde of formula VCHO at a temperature between −120° C. to 0° C., typically −78° C. to −23° C., to provide the desired product of Formula LXII. In some cases, it is convenient to add the transmetallation agent to a mixture of the aldehyde and the iodide.

The desired Formula LXI compounds wherein R¹, R², R⁴, R⁵, R⁶, and R⁷ are as described above can be prepared from the corresponding Formula VIII compounds (prepared as described in Scheme I) using the general procedure described by D. H. R. Barton et al. (Tetrahedron Letters 1983 24, 1605) in which the hydrazone is reacted with iodine in the presence of a suitable hindered base such as 1,1,3,3-tetramethylguanidine in a suitable reaction inert solvent such as tetrahydrofuran at a temperature between 25° C. to 100° C., typically 25° C. to 85° C., removing the solvent in the course of the reaction, to provide the desired product of Formula LXI.

An alternative preparation of the desired Formula IV compounds wherein L is a (C₁-C₆)alkoxycarbonyl group and V, R¹, R², R⁴, R⁵, R⁶, and R⁷ are as described above can be accomplished in a manner similar to that described for the preparation of the Formula LXII compounds from the corresponding Formula LXI compounds by treatment with a suitable transmetallating agent such as an alkyllithium compound such as n-butyllthium or s-butyllithium, or an alkylmagnesium halide such as isopropylmagnesium chloride in a suitable reaction inert solvent such as tetrahydrofuran or diethyl ether at a temperature between −120° C. to 0° C., typically −78° C., to provide a vinylmetal species, which is then reacted with the appropriate ketone of formula VCOL at a temperature between −120° C. to 0° C., typically −78° C. to −23° C., to provide the desired product of Formula IV. In some cases, it is convenient to add the transmetallation agent to a mixture of the ketone and the iodide.

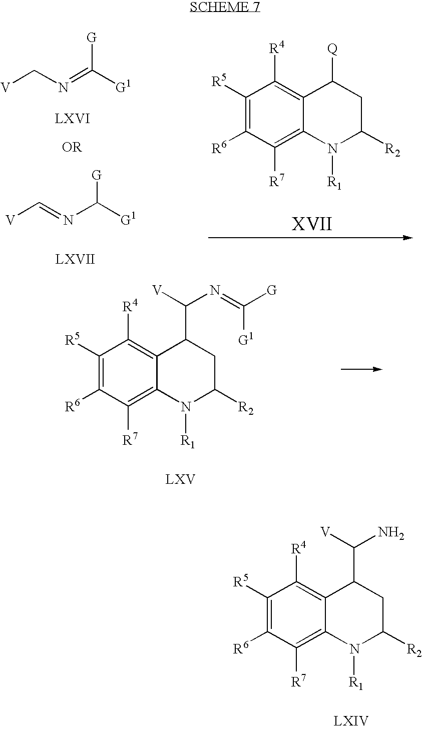

According to reaction Scheme 7, the desired Formula I compounds wherein J is carbon, the optional double bond is absent, R³ is a group CH(V)(NH₂) and V, R¹, R², R⁴, R⁵, R⁶, and R⁷ are as described above (depicted as Formula LXIV compounds) may be prepared as a mixture of diastereoisomers from the corresponding Formula LXV compounds by hydrolysis with an acid such as hydrochloric acid or methanesulfonic acid in the presence of water in a reaction inert solvent such as tetrahydrofuran, dioxane, isopropanol or diisopropyl ether at a temperature between 0° C. to 120° C., typically at reflux. The desired Formula LXIV compounds may be isolated as the salt by crystallization or converted to the free base by treatment with a base such as aqueous sodium hydroxide.

Alternatively, the imine LXV may be treated with a reagent such as hydroxylamine or hydrazine in the presence of water in a reaction inert solvent such as tetrahydrofuran, dioxane, isopropanol or diisopropyl ether at a temperature between 0° C. to 120° C., typically at reflux. This method typically produces a mixture of amine diastereomers LXIV that may be separated by silica gel chromatography.

The desired imine Formula LXV compounds wherein G is V or an aryl group such as phenyl and G¹ is either an aryl group such as phenyl or hydrogen in the case where G is V may be prepared as a mixture of diastereoisomers from the corresponding Formula XVII compounds where Q is a leaving group such as chlorine, bromine, methanesulfonyloxy or p-toluenesulfonyloxy, preferably chlorine or bromine and R¹, R², R⁴, R⁵, R⁶, and R⁷ are as described above by reaction with an imine of Formula LXVI or LXVII in the presence of a suitable base such as sodium hydride, sodium hexamethyldisilazide or potassium hexamethyldisilazide in a reaction inert solvent or mixture of solvents such as tetrahydrofuran, N,N-dimethylformamide or N-methylpyrrolidone at a temperature between −78° C. to 60° C., typically ambient.

The desired Formula LXVI compounds wherein V, G and G¹ are as described above may be prepared from the corresponding benzylamine VCH₂NH₂ by treatment with an aldehyde VCHO (when G is V and G¹ is H) or the ketone GC(O)G¹ or the imine GC(═NH)G¹ using methods and reagents well known to those skilled in the arts, such as can be found in L. A. Paquette (Ed), Encyclopedia of Reagents for Organic Synthesis, John Wiley and Sons, Chichester, England, 1995, for example using benzophenone imine in a reaction inert solvent such as diisopropyl ether or toluene at a temperature between 20° C. to 120° C., typically at reflux.

The desired Formula LXVII compounds wherein V, G and G¹ are as described above may be prepared from the corresponding aldehyde VCHO by treatment with an amine VCH₂NH₂ (when G is V and G¹ is H) or GCH(NH₂)G¹ under conditions where the water produced by the reaction can be continuously removed either by azeotropic distillation, for example in a Dean-Stark apparatus or by use of a drying agent such as anhydrous magnesium sulfate, in a reaction inert solvent such as toluene or diisopropyl ether at a temperature between 20° C. to 120° C., typically at reflux. Examples of alternative conditions are well known to those skilled in the arts and can be found in L. A. Paquette (Ed), Encyclopedia of Reagents for Organic Synthesis, John Wiley and Sons, Chichester, England, 1995.

As an initial note, in the preparation of compounds, it is noted that some of the preparation methods useful for the preparation of the compounds described herein may require protection of remote functionality (e.g., primary amine, secondary amine, carboxyl in intermediates). The need for such protection will vary depending on the nature of the remote functionality and the conditions of the preparation methods. The need for such protection is readily determined by one skilled in the art. The use of such protection/deprotection methods is also within the skill in the art. For a general description of protecting groups and their use, see T. W. Greene, Protective Groups in Organic Synthesis, John Wiley & Sons, New York, 1991.

For example, in the reaction schemes, certain compounds contain primary amines or carboxylic acid functionalities which may interfere with reactions at other sites of the molecule if left unprotected. Accordingly, such functionalities may be protected by an appropriate protecting group which may be removed in a subsequent step. Suitable protecting groups for amine and carboxylic acid protection include those protecting groups commonly used in peptide synthesis (such as N-t-butoxycarbonyl, benzyloxycarbonyl, and 9-fluorenylmethylenoxycarbonyl for amines and lower alkyl or benzyl esters for carboxylic acids) which are generally not chemically reactive under the reaction conditions described and can typically be removed without chemically altering other functionality in the compound.

Prodrugs of the compounds of the present invention may be prepared according to methods known to those skilled in the art. Exemplary processes are described below.

Prodrugs of this invention where a carboxyl group in a carboxylic acid of the compounds is replaced by an ester may be prepared by combining the carboxylic acid with the appropriate alkyl halide in the presence of a base such as potassium carbonate in an inert solvent such as dimethylformamide at a temperature of about 0 to 100° C. for about 1 to about 24 hours. Alternatively the acid is combined with appropriate alcohol as solvent in the presence of a catalytic amount of acid such as concentrated sulfuric acid at a temperature of about 20 to 100° C., preferably at a reflux, for about 1 hour to about 24 hours. Another method is the reaction of the acid with a stoichiometric amount of the alcohol in the presence of a catalytic amount of acid in an inert solvent such as toluene or tetrahydrofuran, with concomitant removal of the water being produced by physical (e.g., Dean-Stark trap) or chemical (e.g., molecular sieves) means.

Prodrugs of this invention where an alcohol function has been derivatized as an ether may be prepared by combining the alcohol with the appropriate alkyl bromide or iodide in the presence of a base such as potassium carbonate in an inert solvent such as dimethylformamide at a temperature of about 0 to 100° C. for about 1 to about 24 hours. Alkanoylaminomethyl ethers may be obtained by reaction of the alcohol with a bis-(alkanoylamino)methane in the presence of a catalytic amount of acid in an inert solvent such as tetrahydrofuran, according to a method described in U.S. Pat. No. 4,997,984. Alternatively, these compounds may be prepared by the methods described by Hoffman et al. in J. Org. Chem. 1994, 59, 3530.

Glycosides are prepared by reaction of the alcohol and a carbohydrate in an inert solvent such as toluene in the presence of acid. Typically the water formed in the reaction is removed as it is being formed as described above. An alternate procedure is the reaction of the alcohol with a suitably protected glycosyl halide in the presence of base followed by deprotection.

N-(1-hydroxyalkyl) amides, N-(1-hydroxy-1-(alkoxycarbonyl)methyl) amides may be prepared by the reaction of the parent amide with the appropriate aldehyde under neutral or basic conditions (e.g., sodium ethoxide in ethanol) at temperatures between 25 and 70° C. N-alkoxymethyl or N-1-(alkoxy)alkyl derivatives can be obtained by reaction of the N-unsubstituted compound with the necessary alkyl halide in the presence of a base in an inert solvent.

The compounds of this invention may also be used in conjunction with other pharmaceutical agents (e.g., LDL-cholesterol lowering agents, triglyceride lowering agents) for the treatment of the disease/conditions described herein. For example, they may be used in combination with a HMG-CoA reductase inhibitor, a cholesterol synthesis inhibitor, a cholesterol absorption inhibitor, a MTP/Apo B secretion inhibitor, a PPAR modulator and other cholesterol lowering agents such as a fibrate, niacin, an ion-exchange resin, an antioxidant, an ACAT inhibitor, and a bile acid sequestrant. Other pharmaceutical agents would also include the following: a bile acid reuptake inhibitor, an ileal bile acid transporter inhibitor, an ACC inhibitor, an antihypertensive (such as NORVASC®), a selective estrogen receptor modulator, a selective androgen receptor modulator, an antibiotic, an antidiabetic (such as metformin, a PPARγ activator, a sulfonylurea, insulin, an aldose reductase inhibitor (ARI) and a sorbitol dehydrogenase inhibitor (SDI)), and aspirin (acetylsalicylic acid). A slow-release form of niacin is available and is known as Niaspan. Niacin may also be combined with other therapeutic agents such as statins, i.e. lovastatin, which is an HMG-CoA reductase inhibitor and described further below. This combination therapy is known as ADVICOR® (Kos Pharmaceuticals Inc.) In combination therapy treatment, both the compounds of this invention and the other drug therapies are administered to mammals (e.g., humans, male or female) by conventional methods.

Any HMG-CoA reductase inhibitor may be used in the combination aspect of this invention. The term HMG-CoA reductase inhibitor refers to compounds which inhibit the bioconversion of hydroxymethylglutaryl-coenzyme A to mevalonic acid catalyzed by the enzyme HMG-CoA reductase. Such inhibition is readily determined by those skilled in the art according to standard assays (e.g., Meth. Enzymol. 1981; 71:455-509 and references cited therein). A variety of these compounds are described and referenced below however other HMG-CoA reductase inhibitors will be known to those skilled in the art. U.S. Pat. No. 4,231,938 (the disclosure of which is hereby incorporated by reference) discloses certain compounds isolated after cultivation of a microorganism belonging to the genus Aspergillus, such as lovastatin. Also, U.S. Pat. No. 4,444,784 (the disclosure of which is hereby incorporated by reference) discloses synthetic derivatives of the aforementioned compounds, such as simvastatin. Also, U.S. Pat. No. 4,739,073 (the disclosure of which is incorporated by reference) discloses certain substituted indoles, such as fluvastatin. Also, U.S. Pat. No. 4,346,227 (the disclosure of which is incorporated by reference) discloses ML-236B derivatives, such as pravastatin. Also, EP-491226A (the disclosure of which is incorporated by reference) discloses certain pyridyldihydroxyheptenoic acids, such as cerivastatin. In addition, U.S. Pat. No. 5,273,995 (the disclosure of which is incorporated by reference) discloses certain 6-[2-(substituted-pyrrol-1-yl)alkyl]pyran-2-ones such as atorvastatin and any pharmaceutically acceptable form thereof (i.e. LIPITOR®). Additional HMG-CoA reductase inhibitors include rosuvastatin and pitavastatin.

Any PPAR modulator may be used in the combination aspect of this invention. The term PPAR modulator refers to compounds which modulate peroxisome proliferator activator receptor (PPAR) activity in mammals, particularly humans. Such modulation is readily determined by those skilled in the art according to standard assays known in the literature. It is believed that such compounds, by modulating the PPAR receptor, regulate transcription of key genes involved in lipid and glucose metabolism such as those in fatty acid oxidation and also those involved in high density lipoprotein (HDL) assembly (for example, apolipoprotein AI gene transcription), accordingly reducing whole body fat and increasing HDL cholesterol. By virtue of their activity, these compounds also reduce plasma levels of triglycerides, VLDL cholesterol, LDL cholesterol and their associated components such as apolipoprotein B in mammals, particularly humans, as well as increasing HDL cholesterol and apolipoprotein AI. Hence, these compounds are useful for the treatment and correction of the various dyslipidemias observed to be associated with the development and incidence of atherosclerosis and cardiovascular disease, including hypoalphalipoproteinemia and hypertriglyceridemia. A variety of these compounds are described and referenced below, however, others will be known to those skilled in the art. International Publication Nos. WO 02/064549 and 02/064130 and U.S. patent application Ser. No. 10/720942, filed Nov. 24, 2003 (the disclosures of which are hereby incorporated by reference) disclose certain compounds which are PPARα activators.

Any MTP/Apo B (microsomal triglyceride transfer protein and or apolipoprotein B) secretion inhibitor may be used in the combination aspect of this invention. The term MTP/Apo B secretion inhibitor refers to compounds which inhibit the secretion of triglycerides, cholesteryl ester, and phospholipids. Such inhibition is readily determined by those skilled in the art according to standard assays (e.g., Welterau, J. R. 1992; Science 258:999). A variety of these compounds are described and referenced below however other MTP/Apo B secretion inhibitors will be known to those skilled in the art, including imputapride (Bayer) and additional compounds such as those disclosed in WO 96/40640 and WO 98/23593, (two exemplary publications).

For example, the following MTP/Apo B secretion inhibitors are particularly useful:

4′-trifluoromethyl-biphenyl-2-carboxylic acid [2-(1H-[1,2,4,]triazol-3-ylmethyl)-1,2,3,4-tetrahydro-isoquinolin-6-yl]-amide;

4′-trifluoromethyl-biphenyl-2-carboxylic acid [2-(2-acetylamino-ethyl)-1,2,3,4-tetrahydro-isoquinolin-6-yl]-amide;

(2-{6-[(4′-trifluoromethyl-biphenyl-2-carbonyl)-amino]-3,4-dihydro-1H-isoquinolin-2-yl}-ethyl)-carbamic acid methyl ester;

4′-trifluoromethyl-biphenyl-2-carboxylic acid [2-(1H-imidazol-2-ylmethyl)-1,2,3,4-tetrahydro-isoquinolin-6-yl]-amide;

4′-trifluoromethyl-biphenyl-2-carboxylic acid [2-(2,2-diphenyl-ethyl)-1,2,3,4-tetrahydro-isoquinolin-6-yl]-amide; and

4′-trifluoromethyl-biphenyl-2-carboxylic acid [2-(2-ethoxy-ethyl)-1,2,3,4-tetrahydro-isoquinolin-6-yl]-amide.

Any HMG-CoA synthase inhibitor may be used in the combination aspect of this invention. The term HMG-CoA synthase inhibitor refers to compounds which inhibit the biosynthesis of hydroxymethylglutaryl-coenzyme A from acetyl-coenzyme A and acetoacetyl-coenzyme A, catalyzed by the enzyme HMG-CoA synthase. Such inhibition is readily determined by those skilled in the art according to standard assays (Meth Enzymol. 1975; 35:155-160: Meth. Enzymol. 1985; 110:19-26 and references cited therein). A variety of these compounds are described and referenced below, however other HMG-CoA synthase inhibitors will be known to those skilled in the art. U.S. Pat. No. 5,120,729 (the disclosure of which is hereby incorporated by reference) discloses certain beta-lactam derivatives. U.S. Pat. No. 5,064,856 (the disclosure of which is hereby incorporated by reference) discloses certain spiro-lactone derivatives prepared by culturing a microorganism (MF5253). U.S. Pat. No. 4,847,271 (the disclosure of which is hereby incorporated by reference) discloses certain oxetane compounds such as 11-(3-hydroxymethyl-4-oxo-2-oxetayl)-3,5,7-trimethyl-2,4-undeca-dienoic acid derivatives.

Any compound that decreases HMG-CoA reductase gene expression may be used in the combination aspect of this invention. These agents may be HMG-CoA reductase transcription inhibitors that block the transcription of DNA or translation inhibitors that prevent or decrease translation of mRNA coding for HMG-CoA reductase into protein. Such compounds may either affect transcription or translation directly, or may be biotransformed to compounds that have the aforementioned activities by one or more enzymes in the cholesterol biosynthetic cascade or may lead to the accumulation of an isoprene metabolite that has the aforementioned activities. Such compounds may cause this effect by decreasing levels of SREBP (sterol receptor binding protein) by inhibiting the activity of site-1 protease (S1P) or agonizing the oxzgenal receptor or SCAP. Such regulation is readily determined by those skilled in the art according to standard assays (Meth. Enzymol. 1985; 110:9-19). Several compounds are described and referenced below, however other inhibitors of HMG-CoA reductase gene expression will be known to those skilled in the art. U.S. Pat. No. 5,041,432 (the disclosure of which is incorporated by reference) discloses certain 15-substituted lanosterol derivatives. Other oxygenated sterols that suppress synthesis of HMG-CoA reductase are discussed by E. I. Mercer (Prog.Lip. Res. 1993;32:357-416).

Any squalene synthetase inhibitor may be used in the combination aspect of this invention. The term squalene synthetase inhibitor refers to compounds which inhibit the condensation of 2 molecules of farnesylpyrophosphate to form squalene, catalyzed by the enzyme squalene synthetase. Such inhibition is readily determined by those skilled in the art according to standard assays (Meth. Enzymol. 1969; 15: 393-454 and Meth. Enzymol. 1985; 110:359-373 and references contained therein). A variety of these compounds are described in and referenced below however other squalene synthetase inhibitors will be known to those skilled in the art. U.S. Pat. No. 5,026,554 (the disclosure of which is incorporated by reference) discloses fermentation products of the microorganism MF5465 (ATCC 74011) including zaragozic acid. A summary of other patented squalene synthetase inhibitors has been compiled (Curr. Op. Ther. Patents (1993) 861-4).

Any squalene epoxidase inhibitor may be used in the combination aspect of this invention. The term squalene epoxidase inhibitor refers to compounds which inhibit the bioconversion of squalene and molecular oxygen into squalene-2,3-epoxide, catalyzed by the enzyme squalene epoxidase. Such inhibition is readily determined by those skilled in the art according to standard assays (Biochim. Biophys. Acta 1984; 794:466-471). A variety of these compounds are described and referenced below, however other squalene epoxidase inhibitors will be known to those skilled in the art. U.S. Pat. Nos. 5,011,859 and 5,064,864 (the disclosures of which are incorporated by reference) disclose certain fluoro analogs of squalene. EP publication 395,768 A (the disclosure of which is incorporated by reference) discloses certain substituted allylamine derivatives. PCT publication WO 9312069 A (the disclosure of which is hereby incorporated by reference) discloses certain amino alcohol derivatives. U.S. Pat. No. 5,051,534 (the disclosure of which is hereby incorporated by reference) discloses certain cyclopropyloxy-squalene derivatives.

Any squalene cyclase inhibitor may be used as the second component in the combination aspect of this invention. The term squalene cyclase inhibitor refers to compounds which inhibit the bioconversion of squalene-2,3-epoxide to lanosterol, catalyzed by the enzyme squalene cyclase. Such inhibition is readily determined by those skilled in the art according to standard assays (FEBS Lett. 1989;244:347-350.). In addition, the compounds described and referenced below are squalene cyclase inhibitors, however other squalene cyclase inhibitors will also be known to those skilled in the art. PCT publication WO9410150 (the disclosure of which is hereby incorporated by reference) discloses certain 1,2,3,5,6,7,8,8a-octahydro-5,5,8(beta)-trimethyl-6-isoquinolineamine derivatives, such as N-trifluoroacetyl-1,2,3,5,6,7,8,8a-octahydro-2-allyl-5,5,8(beta)-trimethyl-6(beta)-isoquinolineamine. French patent publication 2697250 (the disclosure of which is hereby incorporated by reference) discloses certain beta, beta-dimethyl-4-piperidine ethanol derivatives such as 1-(1,5,9-trimethyldecyl)-beta,beta-dimethyl-4-piperidineethanol

Any combined squalene epoxidase/squalene cyclase inhibitor may be used as the second component in the combination aspect of this invention. The term combined squalene epoxidase/squalene cyclase inhibitor refers to compounds that inhibit the bioconversion of squalene to lanosterol via a squalene-2,3-epoxide intermediate. In some assays it is not possible to distinguish between squalene epoxidase inhibitors and squalene cyclase inhibitors, however, these assays are recognized by those skilled in the art. Thus, inhibition by combined squalene epoxidase/squalene cyclase inhibitors is readily determined by those skilled in art according to the aforementioned standard assays for squalene cyclase or squalene epoxidase inhibitors. A variety of these compounds are described and referenced below, however other squalene epoxidase/squalene cyclase inhibitors will be known to those skilled in the art. U.S. Pat. Nos. 5,084,461 and 5,278,171 (the disclosures of which are incorporated by reference) disclose certain azadecalin derivatives. EP publication 468,434 (the disclosure of which is incorporated by reference) discloses certain piperidyl ether and thio-ether derivatives such as 2-(1-piperidyl)pentyl isopentyl sulfoxide and 2-(1-piperidyl)ethyl ethyl sulfide. PCT publication WO 9401404 (the disclosure of which is hereby incorporated by reference) discloses certain acyl-piperidines such as 1-(1-oxopentyl-5-phenylthio)-4-(2-hydroxy-1-methyl)-ethyl)piperidine. U.S. Pat. No. 5,102,915 (the disclosure of which is hereby incorporated by reference) discloses certain cyclopropyloxy-squalene derivatives.

The compounds of the present invention can also be administered in combination with naturally occurring compounds that act to lower plasma cholesterol levels. These naturally occurring compounds are commonly called nutraceuticals and include, for example, garlic extract and niacin. A slow-release form of niacin is available and is known as Niaspan. Niacin may also be combined with other therapeutic agents such as lovastatin, or another is an HMG-CoA reductase inhibitor. This combination therapy with lovastatin is known as ADVICOR™ (Kos Pharmaceuticals Inc.).

Any cholesterol absorption inhibitor can be used as an additional in the combination aspect of the present invention. The term cholesterol absorption inhibition refers to the ability of a compound to prevent cholesterol contained within the lumen of the intestine from entering into the intestinal cells and/or passing from within the intestinal cells into the lymph system and/or into the blood stream. Such cholesterol absorption inhibition activity is readily determined by those skilled in the art according to standard assays (e.g., J. Lipid Res. (1993) 34: 377-395). Cholesterol absorption inhibitors are known to those skilled in the art and are described, for example, in PCT WO 94/00480. An example of a recently approved cholesterol absorption inhibitor is ZETIA™ (ezetimibe) (Schering-Plough/Merck).

Any ACAT inhibitor may be used in the combination therapy aspect of the present invention. The term ACAT inhibitor refers to compounds that inhibit the intracellular esterification of dietary cholesterol by the enzyme acyl CoA: cholesterol acyltransferase. Such inhibition may be determined readily by one of skill in the art according to standard assays, such as the method of Heider et al. described in Journal of Lipid Research., 24:1127 (1983). A variety of these compounds are known to those skilled in the art, for example, U.S. Pat. No. 5,510,379 discloses certain carboxysulfonates, while WO 96/26948 and WO 96/10559 both disclose urea derivatives having ACAT inhibitory activity. Examples of ACAT inhibitors include compounds such as Avasimibe (Pfizer), CS-505 (Sankyo) and Eflucimibe (Eli Lilly and Pierre Fabre).

A lipase inhibitor may be used in the combination therapy aspect of the present invention. A lipase inhibitor is a compound that inhibits the metabolic cleavage of dietary triglycerides or plasma phospholipids into free fatty acids and the corresponding glycerides (e.g. EL, HL, etc.). Under normal physiological conditions, lipolysis occurs via a two-step process that involves acylation of an activated serine moiety of the lipase enzyme. This leads to the production of a fatty acid-lipase hemiacetal intermediate, which is then cleaved to release a diglyceride. Following further deacylation, the lipase-fatty acid intermediate is cleaved, resulting in free lipase, a glyceride and fatty acid. In the intestine, the resultant free fatty acids and monoglycerides are incorporated into bile acid-phospholipid micelles, which are subsequently absorbed at the level of the brush border of the small intestine. The micelles eventually enter the peripheral circulation as chylomicrons. Such lipase inhibition activity is readily determined by those skilled in the art according to standard assays (e.g., Methods Enzymol. 286: 190-231).

Pancreatic lipase mediates the metabolic cleavage of fatty acids from triglycerides at the 1- and 3-carbon positions. The primary site of the metabolism of ingested fats is in the duodenum and proximal jejunum by pancreatic lipase, which is usually secreted in vast excess of the amounts necessary for the breakdown of fats in the upper small intestine. Because pancreatic lipase is the primary enzyme required for the absorption of dietary triglycerides, inhibitors have utility in the treatment of obesity and the other related conditions. Such pancreatic lipase inhibition activity is readily determined by those skilled in the art according to standard assays (e.g., Methods Enzymol. 286: 190-231).

Gastric lipase is an immunologically distinct lipase that is responsible for approximately 10 to 40% of the digestion of dietary fats. Gastric lipase is secreted in response to mechanical stimulation, ingestion of food, the presence of a fatty meal or by sympathetic agents. Gastric lipolysis of ingested fats is of physiological importance in the provision of fatty acids needed to trigger pancreatic lipase activity in the intestine and is also of importance for fat absorption in a variety of physiological and pathological conditions associated with pancreatic insufficiency. See, for example, C. K. Abrams, et al., Gastroenterology, 92,125 (1987). Such gastric lipase inhibition activity is readily determined by those skilled in the art according to standard assays (e.g., Methods Enzymol. 286: 190-231).

A variety of gastric and/or pancreatic lipase inhibitors are known to one of ordinary skill in the art. Preferred lipase inhibitors are those inhibitors that are selected from the group consisting of lipstatin, tetrahydrolipstatin (orlistat), valilactone, esterastin, ebelactone A, and ebelactone B. The compound tetrahydrolipstatin is especially preferred. The lipase inhibitor, N-3-trifluoromethylphenyl-N′-3-chloro-4′-trifluoromethylphenylurea, and the various urea derivatives related thereto, are disclosed in U.S. Pat. No. 4,405,644. The lipase inhibitor, esteracin, is disclosed in U.S. Pat. Nos. 4,189,438 and 4,242,453. The lipase inhibitor, cyclo-O,O′-[(1,6-hexanediyl)-bis-(iminocarbonyl)]dioxime, and the various bis(iminocarbonyl)dioximes related thereto may be prepared as described in Petersen et al., Liebig's Annalen, 562, 205-229 (1949).

A variety of pancreatic lipase inhibitors are described herein below. The pancreatic lipase inhibitors lipstatin, (2S,3S,5S,7Z,10Z)-5-[(S)-2-formamido-4-methyl-valeryloxy]-2-hexyl-3-hydroxy-7,10-hexadecanoic acid lactone, and tetrahydrolipstatin (orlistat), (2S,3S,5S)-5-[(S)-2-formamido-4-methyl-valeryloxy]-2-hexyl-3-hydroxy-hexadecanoic 1,3 acid lactone, and the variously substituted N-formylleucine derivatives and stereoisomers thereof, are disclosed in U.S. Pat. No. 4,598,089. For example, tetrahydrolipstatin is prepared as described in, e.g., U.S. Pat. Nos. 5,274,143; 5,420,305; 5,540,917; and 5,643,874. The pancreatic lipase inhibitor, FL-386, 1-[4-(2-methylpropyl)cyclohexyl]-2-[(phenylsulfonyl)oxy]-ethanone, and the variously substituted sulfonate derivatives related thereto, are disclosed in U.S. Pat. No. 4,452,813. The pancreatic lipase inhibitor, WAY-121898, 4-phenoxyphenyl-4-methylpiperidin-1-yl-carboxylate, and the various carbamate esters and pharmaceutically acceptable salts related thereto, are disclosed in U.S. Pat. Nos. 5,512,565; 5,391,571 and 5,602,151. The pancreatic lipase inhibitor, valilactone, and a process for the preparation thereof by the microbial cultivation of Actinomycetes strain MG147-CF2, are disclosed in Kitahara, et al., J. Antibiotics, 40 (11), 1647-1650 (1987). The pancreatic lipase inhibitors, ebelactone A and ebelactone B, and a process for the preparation thereof by the microbial cultivation of Actinomycetes strain MG7-G1, are disclosed in Umezawa, et al., J. Antibiotics, 33, 1594-1596 (1980). The use of ebelactones A and B in the suppression of monoglyceride formation is disclosed in Japanese Kokai 08-143457, published Jun. 4, 1996.

Other compounds that are marketed for hyperlipidemia, including hypercholesterolemia and which are intended to help prevent or treat atherosclerosis include bile acid sequestrants, such as Welchol®, Colestid®, LoCholest® and Questran®; and fibric acid derivatives, such as Atromid®, Lopid®and Tricor®.

Diabetes can be treated by administering to a patient having diabetes (especially Type II), insulin resistance, impaired glucose tolerance, metabolic syndrome, or the like, or any of the diabetic complications such as neuropathy, nephropathy, retinopathy or cataracts, a therapeutically effective amount of a compound of the present invention in combination with other agents (e.g., insulin) that can be used to treat diabetes. This includes the classes of anti-diabetic agents (and specific agents) described herein.

Any glycogen phosphorylase inhibitor can be used as the second agent in combination with a compound of the present invention. The term glycogen phosphorylase inhibitor refers to compounds that inhibit the bioconversion of glycogen to glucose-1-phosphate which is catalyzed by the enzyme glycogen phosphorylase. Such glycogen phosphorylase inhibition activity is readily determined by those skilled in the art according to standard assays (e.g., J. Med. Chem. 41 (1998) 2934-2938). A variety of glycogen phosphorylase inhibitors are known to those skilled in the art including those described in WO 96/39384 and WO 96/39385.

Any aldose reductase inhibitor can be used in combination with a compound of the present invention. The term aldose reductase inhibitor refers to compounds that inhibit the bioconversion of glucose to sorbitol, which is catalyzed by the enzyme aldose reductase. Aldose reductase inhibition is readily determined by those skilled in the art according to standard assays (e.g., J. Malone, Diabetes, 29:861-864 (1980). “Red Cell Sorbitol, an Indicator of Diabetic Control”). A variety of aldose reductase inhibitors are known to those skilled in the art.

Any sorbitol dehydrogenase inhibitor can be used in combination with a compound of the present invention. The term sorbitol dehydrogenase inhibitor refers to compounds that inhibit the bioconversion of sorbitol to fructose which is catalyzed by the enzyme sorbitol dehydrogenase. Such sorbitol dehydrogenase inhibitor activity is readily determined by those skilled in the art according to standard assays (e.g., Analyt. Biochem (2000) 280: 329-331). A variety of sorbitol dehydrogenase inhibitors are known, for example, U.S. Pat. Nos. 5,728,704 and 5,866,578 disclose compounds and a method for treating or preventing diabetic complications by inhibiting the enzyme sorbitol dehydrogenase.

Any glucosidase inhibitor can be used in combination with a compound of the present invention. A glucosidase inhibitor inhibits the enzymatic hydrolysis of complex carbohydrates by glycoside hydrolases, for example amylase or maltase, into bioavailable simple sugars, for example, glucose. The rapid metabolic action of glucosidases, particularly following the intake of high levels of carbohydrates, results in a state of alimentary hyperglycemia which, in adipose or diabetic subjects, leads to enhanced secretion of insulin, increased fat synthesis and a reduction in fat degradation. Following such hyperglycemias, hypoglycemia frequently occurs, due to the augmented levels of insulin present. Additionally, it is known chyme remaining in the stomach promotes the production of gastric juice, which initiates or favors the development of gastritis or duodenal ulcers. Accordingly, glucosidase inhibitors are known to have utility in accelerating the passage of carbohydrates through the stomach and inhibiting the absorption of glucose from the intestine. Furthermore, the conversion of carbohydrates into lipids of the fatty tissue and the subsequent incorporation of alimentary fat into fatty tissue deposits is accordingly reduced or delayed, with the concomitant benefit of reducing or preventing the deleterious abnormalities resulting therefrom. Such glucosidase inhibition activity is readily determined by those skilled in the art according to standard assays (e.g., Biochemistry (1969) 8: 4214).

A generally preferred glucosidase inhibitor includes an amylase inhibitor. An amylase inhibitor is a glucosidase inhibitor that inhibits the enzymatic degradation of starch or glycogen into maltose. Such amylase inhibition activity is readily determined by those skilled in the art according to standard assays (e.g., Methods Enzymol. (1955) 1: 149). The inhibition of such enzymatic degradation is beneficial in reducing amounts of bioavailable sugars, including glucose and maltose, and the concomitant deleterious conditions resulting therefrom.

A variety of glucosidase inhibitors are known to one of ordinary skill in the art and examples are provided below. Preferred glucosidase inhibitors are those inhibitors that are selected from the group consisting of acarbose, adiposine, voglibose, miglitol, emiglitate, camiglibose, tendamistate, trestatin, pradimicin-Q and salbostatin. The glucosidase inhibitor, acarbose, and the various amino sugar derivatives related thereto are disclosed in U.S. Pat. Nos. 4,062,950 and 4,174,439 respectively. The glucosidase inhibitor, adiposine, is disclosed in U.S. Pat. No. 4,254,256. The glucosidase inhibitor, voglibose, 3,4-dideoxy-4-[[2-hydroxy-1-(hydroxymethyl)ethyl]amino]-2-C-(hydroxymethyl)-D-epi-inositol, and the various N-substituted pseudo-aminosugars related thereto, are disclosed in U.S. Pat. No. 4,701,559. The glucosidase inhibitor, miglitol, (2R,3R,4R,5S)-1-(2-hydroxyethyl)-2-(hydroxymethyl)-3,4,5-piperidinetriol, and the various 3,4,5-trihydroxypiperidines related thereto, are disclosed in U.S. Pat. No. 4,639,436. The glucosidase inhibitor, emiglitate, ethyl p-[2-[(2R,3R,4R,5S)-3,4,5-trihydroxy-2-(hydroxymethyl)piperidino]ethoxy]-benzoate, the various derivatives related thereto and pharmaceutically acceptable acid addition salts thereof, are disclosed in U.S. Pat. No. 5,192,772. The glucosidase inhibitor, MDL-25637, 2,6-dideoxy-7-O-β-D-glucopyrano-syl-2,6-imino-D-glycero-L-gluco-heptitol, the various homodisaccharides related thereto and the pharmaceutically acceptable acid addition salts thereof, are disclosed in U.S. Pat. No. 4,634,765. The glucosidase inhibitor, camiglibose, methyl 6-deoxy-6-[(2R,3R,4R,5S)-3,4,5-trihydroxy-2-(hydroxymethyl)piperidino]-α-D-glucopyranoside sesquihydrate, the deoxynojirimycin derivatives related thereto, the various pharmaceutically acceptable salts thereof and synthetic methods for the preparation thereof, are disclosed in U.S. Pat. Nos. 5,157,116 and 5,504,078. The glycosidase inhibitor, salbostatin and the various pseudosaccharides related thereto, are disclosed in U.S. Pat. No. 5,091,524.

A variety of amylase inhibitors are known to one of ordinary skill in the art. The amylase inhibitor, tendamistat and the various cyclic peptides related thereto, are disclosed in U.S. Pat. No. 4,451,455. The amylase inhibitor AI-3688 and the various cyclic polypeptides related thereto are disclosed in U.S. Pat. No. 4,623,714. The amylase inhibitor, trestatin, consisting of a mixture of trestatin A, trestatin B and trestatin C and the various trehalose-containing aminosugars related thereto are disclosed in U.S. Pat. No. 4,273,765.

Additional anti-diabetic compounds, which can be used as the second agent in combination with a compound of the present invention, includes, for example, the following: biguanides (e.g., metformin), insulin secretagogues (e.g., sulfonylureas and glinides), glitazones, non-glitazone PPARγ agonists, PPARβ agonists, inhibitors of DPP-IV, inhibitors of PDE5, inhibitors of GSK-3, glucagon antagonists, inhibitors of f-1,6-BPase (Metabasis/Sankyo), GLP-1/analogs (AC 2993, also known as exendin-4), insulin and insulin mimetics (Merck natural products). Other examples would include PKC-β inhibitors and AGE breakers.

The compounds of the present invention can be used in combination with other anti-obesity agents. Any anti-obesity agent can be used as the second agent in such combinations and examples are provided herein. Such anti-obesity activity is readily determined by those skilled in the art according to standard assays known in the art.

Suitable anti-obesity agents include phenylpropanolamine, ephedrine, pseudoephedrine, phentermine, β₃ adrenergic receptor agonists, apolipoprotein-B secretion/microsomal triglyceride transfer protein (apo-B/MTP) inhibitors, MCR-4 agonists, cholecystokinin-A (CCK-A) agonists, monoamine reuptake inhibitors (e.g., sibutramine), sympathomimetic agents, serotoninergic agents, cannabinoid receptor antagonists (e.g., rimonabant (SR-141,716A)), dopamine agonists (e.g., bromocriptine), melanocyte-stimulating hormone receptor analogs, 5HT2c agonists, melanin concentrating hormone antagonists, leptin (the OB protein), leptin analogs, leptin receptor agonists, galanin antagonists, lipase inhibitors (e.g., tetrahydrolipstatin, i.e. orlistat), bombesin agonists, anorectic agents (e.g., a bombesin agonist), Neuropeptide-Y antagonists, thyroxine, thyromimetic agents, dehydroepiandrosterones or analogs thereof, glucocorticoid receptor agonists or antagonists, orexin receptor antagonists, urocortin binding protein antagonists, glucagon-like peptide-1 receptor agonists, ciliary neurotrophic factors (e.g., Axokine™), human agouti-related proteins (AGRP), ghrelin receptor antagonists, histamine 3 receptor antagonists or inverse agonists, neuromedin U receptor agonists, and the like.

Any thyromimetic can be used as the second agent in combination with a compound of the present invention. Such thyromimetic activity is readily determined by those skilled in the art according to standard assays (e.g., Atherosclerosis (1996) 126: 53-63). A variety of thyromimetic agents are known to those skilled in the art, for example those disclosed in U.S. Pat. Nos. 4,766,121; 4,826,876; 4,910,305; 5,061,798; 5,284,971; 5,401,772; 5,654,468; and 5,569,674. Other antiobesity agents include sibutramine which can be prepared as described in U.S. Pat. No. 4,929,629. and bromocriptine which can be prepared as described in U.S. Pat. Nos. 3,752,814 and 3,752,888.

The compounds of the present invention can also be used in combination with other antihypertensive agents. Any anti-hypertensive agent can be used as the second agent in such combinations and examples are provided herein. Such antihypertensive activity is readily determined by those skilled in the art according to standard assays (e.g., blood pressure measurements).

Examples of presently marketed products containing antihypertensive agents include calcium channel blockers, such as Cardizem®, Adalat®, Calan®, Cardene®, Covera®, Dilacor®, DynaCirc®, Procardia XL®, Sular®, Tiazac®, Vascor®, Verelan®, Isoptin®, Nimotop®, Norvasc®, and Plendil®; angiotensin converting enzyme (ACE) inhibitors, such as Accupril®, Altace®, Captopril®, Lotensin®, Mavik®, Monopril®, Prinivil®, Univasc®, Vasotec® and Zestril®.

Osteoporosis is a systemic skeletal disease, characterized by low bone mass and deterioration of bone tissue, with a consequent increase in bone fragility and susceptibility to fracture. In the U.S., the condition affects more than 25 million people and causes more than 1.3 million fractures each year, including 500,000 spine, 250,000 hip and 240,000 wrist fractures annually. Hip fractures are the most serious consequence of osteoporosis, with 5-20% of patients dying within one year, and over 50% of survivors being incapacitated.

The elderly are at greatest risk of osteoporosis, and the problem is therefore predicted to increase significantly with the aging of the population. Worldwide fracture incidence is forecasted to increase three-fold over the next 60 years, and one study has estimated that there will be 4.5 million hip fractures worldwide in 2050.

Women are at greater risk of osteoporosis than men. Women experience a sharp acceleration of bone loss during the five years following menopause. Other factors that increase the risk include smoking, alcohol abuse, a sedentary lifestyle and low calcium intake.

Those skilled in the art will recognize that anti-resorptive agents (for example progestins, polyphosphonates, bisphosphonate(s), estrogen agonists/antagonists, estrogen, estrogen/progestin combinations, Premarin®, estrone, estriol or 17α- or 17β-ethynyl estradiol) may be used in conjunction with the compounds of the present invention.

Exemplary progestins are available from commercial sources and include: algestone acetophenide, altrenogest, amadinone acetate, anagestone acetate, chlormadinone acetate, cingestol, clogestone acetate, clomegestone acetate, delmadinone acetate, desogestrel, dimethisterone, dydrogesterone, ethynerone, ethynodiol diacetate, etonogestrel, flurogestone acetate, gestaclone, gestodene, gestonorone caproate, gestrinone, haloprogesterone, hydroxyprogesterone caproate, levonorgestrel, lynestrenol, medrogestone, medroxyprogesterone acetate, melengestrol acetate, methynodiol diacetate, norethindrone, norethindrone acetate, norethynodrel, norgestimate, norgestomet, norgestrel, oxogestone phenpropionate, progesterone, quingestanol acetate, quingestrone, and tigestol.

Preferred progestins are medroxyprogestrone, norethindrone and norethynodrel.

Exemplary bone resorption inhibiting polyphosphonates include polyphosphonates of the type disclosed in U.S. Pat. No. 3,683,080, the disclosure of which is incorporated herein by reference. Preferred polyphosphonates are geminal diphosphonates (also referred to as bis-phosphonates). Tiludronate disodium is an especially preferred polyphosphonate. Ibandronic acid is an especially preferred polyphosphonate. Alendronate and resindronate are especially preferred polyphosphonates. Zoledronic acid is an especially preferred polyphosphonate. Other preferred polyphosphonates are 6-amino-1-hydroxy-hexylidene-bisphosphonic acid and 1-hydroxy-3(methylpentylamino)-propylidene-bisphosphonic acid. The polyphosphonates may be administered in the form of the acid, or of a soluble alkali metal salt or alkaline earth metal salt. Hydrolyzable esters of the polyphosphonates are likewise included. Specific examples include ethane-1-hydroxy 1,1-diphosphonic acid, methane diphosphonic acid, pentane-1-hydroxy-1,1-diphosphonic acid, methane dichloro diphosphonic acid, methane hydroxy diphosphonic acid, ethane-1-amino-1,1-diphosphonic acid, ethane-2-amino-1,1-diphosphonic acid, propane-3-amino-1-hydroxy-1,1-diphosphonic acid, propane-N,N-dimethyl-3-amino-1-hydroxy-1,1-diphosphonic acid, propane-3,3-dimethyl-3-amino-1-hydroxy-1,1-diphosphonic acid, phenyl amino methane diphosphonic acid, N,N-dimethylamino methane diphosphonic acid, N(2-hydroxyethyl) amino methane diphosphonic acid, butane-4-amino-1-hydroxy-1,1-diphosphonic acid, pentane-5-amino-1-hydroxy-1,1-diphosphonic acid, hexane-6-amino-1-hydroxy-1,1-diphosphonic acid and pharmaceutically acceptable esters and salts thereof.

In particular, the compounds of this invention may be combined with a mammalian estrogen agonist/antagonist. Any estrogen agonist/antagonist may be used in the combination aspect of this invention. The term estrogen agonist/antagonist refers to compounds which bind with the estrogen receptor, inhibit bone turnover and/or prevent bone loss. In particular, estrogen agonists are herein defined as chemical compounds capable of binding to the estrogen receptor sites in mammalian tissue, and mimicking the actions of estrogen in one or more tissue. Estrogen antagonists are herein defined as chemical compounds capable of binding to the estrogen receptor sites in mammalian tissue, and blocking the actions of estrogen in one or more tissues. Such activities are readily determined by those skilled in the art of standard assays including estrogen receptor binding assays, standard bone histomorphometric and densitometer methods, and Eriksen E. F. et al., Bone Histomorphometry, Raven Press, New York, 1994, pages 1-74; Grier S. J. et. al., The Use of Dual-Energy X-Ray Absorptiometry In Animals, Inv. Radiol., 1996, 31(1):50-62; Wahner H. W. and Fogelman I., The Evaluation of Osteoporosis: Dual Energy X-Ray Absorptiometry in Clinical Practice., Martin Dunitz Ltd., London 1994, pages 1-296). A variety of these compounds are described and referenced below.

Another preferred estrogen agonist/antagonist is 3-(4-(1,2-diphenyl-but-1-enyl)-phenyl)-acrylic acid, which is disclosed in Willson et al., Endocrinology, 1997, 138, 3901-3911.

Another preferred estrogen agonist/antagonist is tamoxifen: (ethanamine, 2-(-4-(1,2-diphenyl-1-butenyl)phenoxy)-N,N-dimethyl, (Z)-2-, 2-hydroxy-1,2,3-propanetricarboxylate(1:1)) and related compounds which are disclosed in U.S. Pat. No. 4,536,516, the disclosure of which is incorporated herein by reference.

Another related compound is 4-hydroxy tamoxifen, which is disclosed in U.S. Pat. No. 4,623,660, the disclosure of which is incorporated herein by reference.

A preferred estrogen agonist/antagonist is raloxifene: (methanone, (6-hydroxy-2-(4-hydroxyphenyl)benzo[b]thien-3-yl)(4-(2-(1-piperidinyl)ethoxy)phenyl)-hydrochloride) which is disclosed in U.S. Pat. No. 4,418,068, the disclosure of which is incorporated herein by reference.

Another preferred estrogen agonist/antagonist is toremifene: (ethanamine, 2-(4-(4-chloro-1,2-diphenyl-1-butenyl)phenoxy)-N,N-dimethyl-, (Z)-, 2-hydroxy-1,2,3-propanetricarboxylate (1:1) which is disclosed in U.S. Pat. No. 4,996,225, the disclosure of which is incorporated herein by reference.

Another preferred estrogen agonist/antagonist is centchroman: 1-(2-((4-(-methoxy-2,2, dimethyl-3-phenyl-chroman-4-yl)-phenoxy)-ethyl)-pyrrolidine, which is disclosed in U.S. Pat. No. 3,822,287, the disclosure of which is incorporated herein by reference. Also preferred is levormeloxifene.

Another preferred estrogen agonist/antagonist is idoxifene: (E)-1-(2-(4-(1-(4-iodo-phenyl)-2-phenyl-but-1-enyl)-phenoxy)-ethyl)-pyrrolidinone, which is disclosed in U.S. Pat. No. 4,839,155, the disclosure of which is incorporated herein by reference.

Another preferred estrogen agonist/antagonist is 2-(4-methoxy-phenyl)-3-[4-(2-piperidin-1-yl-ethoxy)-phenoxy]-benzo[b]thiophen-6-ol which is disclosed in U.S. Pat. No. 5,488,058, the disclosure of which is incorporated herein by reference.

Another preferred estrogen agonist/antagonist is 6-(4-hydroxy-phenyl)-5-(4-(2-piperidin-1-yl-ethoxy)-benzyl)-naphthalen-2-ol, which is disclosed in U.S. Pat. No. 5,484,795, the disclosure of which is incorporated herein by reference.

Another preferred estrogen agonist/antagonist is (4-(2-(2-aza-bicyclo[2.2.1]hept-2-yl)-ethoxy)-phenyl)-(6-hydroxy-2-(4-hydroxy-phenyl)-benzo[b]thiophen-3-yl)-methanone which is disclosed, along with methods of preparation, in PCT publication no. WO 95/10513 assigned to Pfizer Inc.

Other preferred estrogen agonist/antagonists include the compounds, TSE-424 (Wyeth-Ayerst Laboratories) and arazoxifene.

Other preferred estrogen agonist/antagonists include compounds as described in commonly assigned U.S. Pat. No. 5,552,412, the disclosure of which is incorporated herein by reference. Especially preferred compounds described therein are:

cis-6-(4-fluoro-phenyl)-5-(4-(2-piperidin-1-yl-ethoxy)-phenyl)-5,6,7,8-tetrahydro-naphthalene-2-ol;

(−)-cis-6-phenyl-5-(4-(2-pyrrolidin-1-yl-ethoxy)-phenyl)-5,6,7,8-tetrahydro-naphthalene-2-ol (also known as lasofoxifene);

cis-6-phenyl-5-(4-(2-pyrrolidin-1-yl-ethoxy)-phenyl)-5,6,7,8-tetrahydro-naphthalene-2-ol;

cis-1-(6′-pyrrolodinoethoxy-3′-pyridyl)-2-phenyl-6-hydroxy-1,2,3,4-tetrahydronaphthalene;

1-(4′-pyrrolidinoethoxyphenyl)-2-(4″-fluorophenyl)-6-hydroxy-1,2,3,4-tetrahydroisoquinoline;

cis-6-(4-hydroxyphenyl)-5-(4-(2-piperidin-1-yl-ethoxy)-phenyl)-5,6,7,8-tetrahydro-naphthalene-2-ol; and

1-(4′-pyrrolidinolethoxyphenyl)-2-phenyl-6-hydroxy-1,2,3,4-tetrahydroisoquinoline.

Other estrogen agonist/antagonists are described in U.S. Pat. No. 4,133,814 (the disclosure of which is incorporated herein by reference). U.S. Pat. No. 4,133,814 discloses derivatives of 2-phenyl-3-aroyl-benzothiophene and 2-phenyl-3-aroylbenzothiophene-1-oxide.

Other anti-osteoporosis agents, which can be used as the second agent in combination with a compound of the present invention, include, for example, the following: parathyroid hormone (PTH) (a bone anabolic agent); parathyroid hormone (PTH) secretagogues (see, e.g., U.S. Pat. No. 6,132,774), particularly calcium receptor antagonists; calcitonin; and vitamin D and vitamin D analogs.

Any selective androgen receptor modulator (SARM) can be used in combination with a compound of the present invention. A selective androgen receptor modulator (SARM) is a compound that possesses androgenic activity and which exerts tissue-selective effects. SARM compounds can function as androgen receptor agonists, partial agonists, partial antagonists or antagonists. Examples of suitable SARMs include compounds such as cyproterone acetate, chlormadinone, flutamide, hydroxyflutamide, bicalutamide, nilutamide, spironolactone, 4-(trifluoromethyl)-2(1H)-pyrrolidino[3,2-g] quinoline derivatives, 1,2-dihydropyridino[5,6-g]quinoline derivatives and piperidino[3,2-g]quinolinone derivatives.

Cypterone, also known as (1b,2b)-6-chloro-1,2-dihydro-17-hydroxy-3′H-cyclopropa[1,2]pregna-1,4,6-triene-3,20-dione is disclosed in U.S. Pat. No. 3,234,093. Chlormadinone, also known as 17-(acetyloxy)-6-chloropregna-4,6-diene-3,20-dione, in its acetate form, acts as an anti-androgen and is disclosed in U.S. Pat. No. 3,485,852. Nilutamide, also known as 5,5-dimethyl-3-[4-nito-3-(trifluoromethyl)phenyl]-2,4-imidazolidinedione and by the trade name Nilandron® is disclosed in U.S. Pat. No. 4,097,578. Flutamide, also known as 2-methyl-N-[4-nitro-3-(trifluoromethyl)phenyl]propanamide and the trade name Eulexin® is disclosed in U.S. Pat. No. 3,847,988. Bicalutamide, also known as 4′-cyano-a′,a′,a′-trifluoro-3-(4-fluorophenylsulfonyl)-2-hydroxy-2-methylpropiono-m-toluidide and the trade name Casodex® is disclosed in EP-100172. The enantiomers of biclutamide are discussed by Tucker and Chesterton, J. Med. Chem. 1988, 31, 885-887. Hydroxyflutamide, a known androgen receptor antagonist in most tissues, has been suggested to function as a SARM for effects on IL-6 production by osteoblasts as disclosed in Hofbauer et al. J. Bone Miner. Res. 1999, 14, 1330-1337. Additional SARMs have been disclosed in U.S. Pat. No. 6,017,924; WO 01/16108, WO 01/16133, WO 01/16139, WO 02/00617, WO 02/16310, U.S. Patent Application Publication No. US 2002/0099096, U.S. Patent Application Publication No. US 2003/0022868, WO 03/011302 and WO 03/011824. All of the above references are hereby incorporated by reference herein.

The starting materials and reagents for the above described compounds, are also readily available or can be easily synthesized by those skilled in the art using conventional methods of organic synthesis. For example, many of the compounds used herein, are related to, or are derived from compounds in which there is a large scientific interest and commercial need, and accordingly many such compounds are commercially available or are reported in the literature or are easily prepared from other commonly available substances by methods which are reported in the literature.

Some of the compounds of this invention or intermediates in their synthesis have asymmetric carbon atoms and therefore are enantiomers or diastereomers. Diasteromeric mixtures can be separated into their individual diastereomers on the basis of their physical chemical differences by methods known per se, for example, by chromatography and/or fractional crystallization. Enantiomers can be separated by, for example, chiral HPLC methods or converting the enantiomeric mixture into a diastereomeric mixture by reaction with an appropriate optically active compound (e.g., alcohol), separating the diastereomers and converting (e.g., hydrolyzing) the individual diastereomers to the corresponding pure enantiomers. Also, an enantiomeric mixture of the compounds or an intermediate in their synthesis which contain an acidic or basic moiety may be separated into their corresponding pure enantiomers by forming a diastereomic salt with an optically pure chiral base or acid (e.g., 1-phenyl-ethyl amine or tartaric acid) and separating the diasteromers by fractional crystallization followed by neutralization to break the salt, thus providing the corresponding pure enantiomers. All such isomers, including diastereomers, enantiomers and mixtures thereof are considered as part of this invention for all of the compounds of the present invention, including the compounds of the present invention. Also, some of the compounds of this invention are atropisomers (e.g., substituted biaryls) and are considered as part of this invention.

More specifically, the compounds of this invention may be obtained in enantiomerically enriched form by resolving the racemate of the final compound or an intermediate in its synthesis, employing chromatography (preferably high pressure liquid chromatography [HPLC]) on an asymmetric resin (preferably Chiralcel™ AD or OD (obtained from Chiral Technologies, Exton, Pa.)) with a mobile phase consisting of a hydrocarbon (preferably heptane or hexane) containing between 0 and 50% isopropanol (preferably between 2 and 20%) and between 0 and 5% of an alkyl amine (preferably 0.1% of diethylamine). Concentration of the product containing fractions affords the desired materials.

Some of the compounds of this invention are acidic and they form a salt with a pharmaceutically acceptable cation. Some of the compounds of this invention are basic and they form a salt with a pharmaceutically acceptable anion. All such salts are within the scope of this invention and they can be prepared by conventional methods such as combining the acidic and basic entities, usually in a stoichiometric ratio, in either an aqueous, non-aqueous or partially aqueous medium, as appropriate. The salts are recovered either by filtration, by precipitation with a non-solvent followed by filtration, by evaporation of the solvent, or, in the case of aqueous solutions, by lyophilization, as appropriate. The compounds can be obtained in crystalline form by dissolution in an appropriate solvent(s) such as ethanol, hexanes or water/ethanol mixtures.

In addition, when the compounds of this invention form hydrates or solvates they are also within the scope of the invention.

The compounds of this invention, their prodrugs and the salts of such compounds and prodrugs are all adapted to therapeutic use as agents that inhibit cholesterol ester transfer protein activity in mammals, particularly humans. Thus, the compounds of this invention elevate plasma HDL cholesterol, its associated components, and the functions performed by them in mammals, particularly humans. By virtue of their activity, these agents also reduce plasma levels of triglycerides, VLDL cholesterol, Apo-B, LDL cholesterol and their associated components in mammals, particularly humans. Moreover, these compounds are useful in equalizing LDL cholesterol and HDL cholesterol. Hence, these compounds are useful for the treatment and correction of the various dyslipidemias observed to be associated with the development and incidence of atherosclerosis and cardiovascular disease, including coronary artery disease, coronary heart disease, coronary vascular disease, peripheral vascular disease, hypoalphalipoproteinemia, hyperbetalipoproteinemia, hypertriglyceridemia, hypercholesterolemia, familial-hypercholesterolemia, low HDL and associated components, elevated LDL and associated components, elevated Lp(a), elevated small-dense LDL, elevated VLDL and associated components and post-prandial lipemia.

Further, introduction of a functional CETP gene into an animal lacking CETP (mouse) results in reduced HDL levels (Agellon, L. B., et al: J. Biol. Chem. (1991) 266: 10796-10801.) and increased susceptibility to atherosclerosis.(Marotti, K. R., et al: Nature (1993) 364: 73-75.). Also, inhibition of CETP activity with an inhibitory antibody raises HDL-cholesterol in hamster (Evans, G. F., et al: J. of Lipid Research (1994) 35:1634-1645.) and rabbit (Whitlock, M. E., et al: J. Clin. Invest. (1989) 84: 129-137). Suppression of increased plasma CETP by intravenous injection with antisense oligodeoxynucleotides against CETP mRNA reduced atherosclerosis in cholesterol-fed rabbits (Sugano, M., et al: J. of Biol. Chem. (1998) 273: 5033-5036.) Importantly, human subjects deficient in plasma CETP, due to a genetic mutation possess markedly elevated plasma HDL-cholesterol levels and apolipoprotein A-I, the major apoprotein component of HDL. In addition, most demonstrate markedly decreased plasma LDL cholesterol and apolipoprotein B (the major apolipoprotein component of LDL. (Inazu, A., Brown, M. L., Hesler, C. B., et al.: N. Engl. J. Med. (1990) 323: 1234-1238.)

Given the negative correlation between the levels of HDL cholesterol and HDL associated lipoproteins, and the positive correlation between triglycerides, LDL cholesterol, and their associated apolipoproteins in blood with the development of cardiovascular, cerebral vascular and peripheral vascular diseases, the compounds of this invention, their prodrugs and the salts of such compounds and prodrugs, by virtue of their pharmacologic action, are useful for the prevention, arrestment and/or regression of atherosclerosis and its associated disease states. These include cardiovascular disorders (e.g., angina, ischemia, cardiac ischemia and myocardial infarction), complications due to cardiovascular disease therapies (e.g., reperfusion injury and angioplastic restenosis), hypertension, elevated cardiovascular risk associated with hypertension, stroke, atherosclerosis associated with organ transplantation, cerebrovascular disease, cognitive dysfunction (including, but not limited to, dementia secondary to atherosclerosis, transient cerebral ischemic attacks, neurodegeneration, neuronal deficient, and delayed onset or procession of Alzheimer's disease), elevated levels of oxidative stress, elevated levels of C-Reactive Protein, Metabolic Syndrome and elevated levels of HbA1C.

Because of the beneficial effects widely associated with elevated HDL levels, an agent which inhibits CETP activity in humans, by virtue of its HDL increasing ability, also provides valuable avenues for therapy in a number of other disease areas as well.

Thus, given the ability of the compounds of this invention, their prodrugs and the salts of such compounds and prodrugs to alter lipoprotein composition via inhibition of cholesterol ester transfer, they are of use in the treatment of vascular complications associated with diabetes, lipoprotein abnormalities associated with diabetes and sexual dysfunction associated with diabetes and vascular disease. Hyperlipidemia is present in most subjects with diabetes mellitus (Howard, B. V. 1987. J. Lipid Res. 28, 613). Even in the presence of normal lipid levels, diabetic subjects experience a greater risk of cardiovascular disease (Kannel, W. B. and McGee, D. L. 1979. Diabetes Care 2, 120). CETP-mediated cholesteryl ester transfer is known to be abnormally increased in both insulin-dependent (Bagdade, J. D., Subbaiah, P. V. and Ritter, M. C. 1991. Eur. J. Clin. Invest. 21, 161) and non-insulin dependent diabetes (Bagdade. J. D., Ritter, M. C., Lane, J. and Subbaiah. 1993. Atherosclerosis 104, 69). It has been suggested that the abnormal increase in cholesterol transfer results in changes in lipoprotein composition, particularly for VLDL and LDL, that are more atherogenic (Bagdade, J. D., Wagner, J. D., Rudel, L. L., and Clarkson, T. B. 1995. J. Lipid Res. 36, 759). These changes would not necessarily be observed during routine lipid screening. Thus the present invention will be useful in reducing the risk of vascular complications as a result of the diabetic condition.

The described agents are useful in the treatment of obesity and elevated cardiovascular risk associated with obesity. In both humans (Radeau, T., Lau, P., Robb, M., McDonnell, M., Ailhaud, G. and McPherson, R., 1995. Journal of Lipid Research. 36 (12):2552-61) and nonhuman primates (Quinet, E., Tall, A., Ramakrishnan, R. and Rudel, L., 1991. Journal of Clinical Investigation. 87 (5):1559-66) mRNA for CETP is expressed at high levels in adipose tissue. The adipose message increases with fat feeding (Martin, L. J., Connelly, P. W., Nancoo, D., Wood, N., Zhang, Z. J., Maguire, G., Quinet, E., Tall, A. R., Marcel, Y. L. and McPherson, R., 1993. Journal of Lipid Research. 34 (3):437-46), and is translated into functional transfer protein and through secretion contributes significantly to plasma CETP levels. In human adipocytes the bulk of cholesterol is provided by plasma LDL and HDL (Fong, B. S., and Angel, A., 1989. Biochimica et Biophysica Acta. 1004 (1):53-60). The uptake of HDL cholesteryl ester is dependent in large part on CETP (Benoist, F., Lau, P., McDonnell, M., Doelle, H., Milne, R. and McPherson, R., 1997. Journal of Biological Chemistry. 272 (38):23572-7). This ability of CETP to stimulate HDL cholesteryl uptake, coupled with the enhanced binding of HDL to adipocytes in obese subjects (Jimenez, J. G., Fong, B., Julien, P., Despres, J. P., Rotstein, L., and Angel, A., 1989. International Journal of Obesity. 13 (5):699-709), suggests a role for CETP, not only in generating the low HDL phenotype for these subjects, but in the development of obesity itself by promoting cholesterol accumulation. Inhibitors of CETP activity that block this process therefore serve as useful adjuvants to dietary therapy in causing weight reduction.

CETP inhibitors are useful in the treatment of inflammation due to Gram-negative sepsis and septic shock. For example, the systemic toxicity of Gram-negative sepsis is in large part due to endotoxin, a lipopolysaccharide (LPS) released from the outer surface of the bacteria, which causes an extensive inflammatory response. Lipopolysaccharide can form complexes with lipoproteins (Ulevitch, R. J., Johnston, A. R., and Weinstein, D. B., 1981. J. Clin. Invest. 67, 827-37). In vitro studies have demonstrated that binding of LPS to HDL substantially reduces the production and release of mediators of inflammation (Ulevitch, R. J., Johnston, A. R., 1978. J. Clin. Invest. 62, 1313-24). In vivo studies show that transgenic mice expressing human apo-AI and elevated HDL levels are protected from septic shock (Levine, D. M., Parker, T. S., Donnelly, T. M., Walsh, A. M., and Rubin, A. L. 1993. Proc. Natl. Acad. Sci. 90, 12040-44). Importantly, administration of reconstituted HDL to humans challenged with endotoxin resulted in a decreased inflammatory response (Pajkrt, D., Doran, J. E., Koster, F., Lerch, P. G., Arnet, B., van der Poll, T., ten Cate, J. W., and van Deventer, S. J. H. 1996. J. Exp. Med. 184, 1601-08). The CETP inhibitors, by virtue of the fact that they raise HDL levels, attenuate the development of inflammation and septic shock. These compounds would also be useful in the treatment of endotoxemia, autoimmune diseases and other systemic disease indications, organ or tissue transplant rejection and cancer.

The utility of the compounds of the invention, their prodrugs and the salts of such compounds and prodrugs as medical agents in the treatment of the above described disease/conditions in mammals (e.g. humans, male or female) is demonstrated by the activity of the compounds of this invention in conventional assays and the in vivo assay described below. The in vivo assay (with appropriate modifications within the skill in the art) may be used to determine the activity of other lipid or triglyceride controlling agents as well as the compounds of this invention. The combination protocol described below is useful for demonstrating the utility of the combinations of the lipid and triglyceride agents (e.g., the compounds of this invention) described herein. Such assays also provide a means whereby the activities of the compounds of this invention, their prodrugs and the salts of such compounds and prodrugs (or the other agents described herein) can be compared to each other and with the activities of other known compounds. The results of these comparisons are useful for determining dosage levels in mammals, including humans, for the treatment of such diseases.

The following protocols can of course be varied by those skilled in the art.

The hyperalphacholesterolemic activity of the compounds can be determined by assessing the effect of these compounds on the action of cholesteryl ester transfer protein by measuring the relative transfer ratio of radiolabeled lipids between lipoprotein fractions, essentially as previously described by Morton in J. Biol. Chem. 256, 11992, 1981 and by Dias in Clin. Chem. 34, 2322,1988.

### CETP in Vitro Assay

The following is a brief description of assays of cholesteryl ester transfer in 97% (whole) or diluted human plasma (in vitro) and animal plasma (ex vivo): CETP activity in the presence or absence of drug is assayed by determining the transfer of ³H-labeled cholesteryl oleate (CO) from exogenous tracer HDL or LDL to the nonHDL or HDL lipoprotein fraction in human plasma, respectively, or from ³H-labeled LDL to the HDL fraction in animal plasma. Labeled human lipoprotein substrates are prepared similarly to the method described by Morton in which the endogenous CETP activity in plasma is employed to transfer ³H—CO from phospholipid liposomes to all the lipoprotein fractions in plasma. ³H-labeled LDL and HDL are subsequently isolated by sequential ultracentrifugation at the density cuts of 1.019-1.063 and 1.10-1.21 g/ml, respectively.

For the 97% or whole plasma activity assay, ³H-labeled HDL is added to plasma at 10-25 mmoles CO/ml and the samples incubated at 37° C. for 2.5-3 hrs. Non-HDL lipoproteins are then precipitated by the addition of an equal volume of 20% (wt/vol) polyethylene glycol 8000 (Dias). The samples are centrifuged 750 g×20 minutes and the radioactivity contained in the HDL-containing supernatant determined by liquid scintillation counting. Introducing varying quantities of the compounds of this invention as a solution in dimethylsulfoxide into human plasma, before addition of the radiolabeled cholesteryl oleate, and comparing the amounts of radiolabel transferred compared to incubations containing no inhibitor compounds allows the cholesteryl ester transfer inhibitory activities to be determined.

When a more sensitive assay is desirable, an in vitro assay using diluted human plasma is utilized. For this assay, ³H-labeled LDL is added to plasma at 50 nmoles CO/ml and the samples incubated at 37° C. for 7 hrs. Non-HDL lipoproteins are then precipitated by the addition of potassium phosphate to 100 mM final concentration followed by manganese chloride to 20 mM final concentration. After vortexing, the samples are centrifuged 750 g×20 minutes and the radioactivity contained in the HDL-containing supernatant determined by liquid scintillation counting. Introducing varying quantities of the compounds of this invention as a solution in dimethylsulfoxide into diluted human plasma, before addition of the radiolabeled cholesteryl oleate, and comparing the amounts of radiolabel transferred compared to incubations containing no inhibitor compounds allows the cholesteryl ester transfer inhibitory activities to be determined. This assay has been adapted to run in microtiter plate format with liquid scintillation counting accomplished using a Wallac plate reader.

### CETP in Vivo Assay

Activity of these compounds in vivo can be determined by the amount of agent required to be administered, relative to control, to inhibit cholesteryl ester transfer activity by 50% at various time points ex vivo or to elevate HDL cholesterol by a given percentage in a CETP-containing animal species. Transgenic mice expressing both human CETP and human apolipoprotein AI (Charles River, Boston, Mass.) may be used to assess compounds in vivo. The compounds to be examined are administered by oral gavage in an emulsion vehicle containing 20% (v:v) olive oil and 80% sodium taurocholate (0.5%). Blood is taken from mice retroorbitally before dosing, if a predose blood sample is desirable. At various times after dosing, ranging from 4 h to 24 h, the animals are sacrificed, blood obtained by heart puncture, and lipid parameters measured, including total cholesterol, HDL and LDL cholesterol, and triglycerides. CETP activity is determined by a method similar to that described above except that ³H-cholesteryl oleate-containing LDL is used as the donor source as opposed to HDL. The values obtained for lipids and transfer activity are compared to those obtained prior to dosing and/or to those from mice receiving vehicle alone.

### Plasma Lipids Assay

The activity of these compounds may also be demonstrated by determining the amount of agent required to alter plasma lipid levels, for example HDL cholesterol levels, LDL cholesterol levels, VLDL cholesterol levels or triglycerides, in the plasma of certain mammals, for example marmosets that possess CETP activity and a plasma lipoprotein profile similar to that of humans (Crook et al. Arteriosclerosis 10, 625, 1990). Adult marmosets are assigned to treatment groups so that each group has a similar mean±SD for total, HDL, and/or LDL plasma cholesterol concentrations. After group assignment, marmosets are dosed daily with compound as a dietary admix or by intragastric intubation for from one to eight days. Control marmosets receive only the dosing vehicle. Plasma total, LDL VLDL and HDL cholesterol values can be determined at any point during the study by obtaining blood from an antecubital vein and separating plasma lipoproteins into their individual subclasses by density gradient centrifugation, and by measuring cholesterol concentration as previously described (Crook et al. Arteriosclerosis 10, 625, 1990).

### In Vivo Atherosclerosis Assay

Anti-atherosclerotic effects of the compounds can be determined by the amount of compound required to reduce the lipid deposition in rabbit aorta. Male New Zealand White rabbits are fed a diet containing 0.2% cholesterol and 10% coconut oil for 4 days (meal-fed once per day). Rabbits are bled from the marginal ear vein and total plasma cholesterol values are determined from these samples. The rabbits are then assigned to treatment groups so that each group has a similar mean±SD for total plasma cholesterol concentration, HDL cholesterol concentration, triglyceride concentration and/or cholesteryl ester transfer protein activity. After group assignment, rabbits are dosed daily with compound given as a dietary admix or on a small piece of gelatin based confection. Control rabbits receive only the dosing vehicle, be it the food or the gelatin confection. The cholesterol/coconut oil diet is continued along with the compound administration throughout the study. Plasma cholesterol values and cholesteryl ester transfer protein activity can be determined at any point during the study by obtaining blood from the marginal ear vein. After 3-5 months, the rabbits are sacrificed and the aortae are removed from the thoracic arch to the branch of the iliac arteries. The aortae are cleaned of adventitia, opened longitudinally and then analyzed unstained or stained with Sudan IV as described by Holman et. al. (Lab. Invest. 1958, 7, 42-47). The percent of the lesioned surface area is quantitated by densitometry using an Optimas Image Analyzing System (Image Processing Systems). Reduced lipid deposition is indicated by a reduction in the percent of lesioned surface area in the compound-receiving group in comparison with the control rabbits.

### Antiobesity Protocol

The ability of CETP inhibitors to cause weight loss can be assessed in obese human subjects with body mass index (BMI)≧30 kg/m². Doses of inhibitor are administered sufficient to result in an increase of ≧25% in HDL cholesterol levels. BMI and body fat distribution, defined as waist (W) to hip (H) ratio (WHR), are monitored during the course of the 3-6 month studies, and the results for treatment groups compared to those receiving placebo.

### In Vivo Sepsis Assay

In vivo studies show that transgenic mice expressing human apo-AI and elevated HDL levels are protected from septic shock. Thus the ability of CETP inhibitors to protect from septic shock can be demonstrated in transgenic mice expressing both human apo-AI and human CETP transgenes (Levine, D. M., Parker, T. S., Donnelly, T. M., Walsh, A. M. and Rubin, A. L., 1993. Proc. Natl. Acad. Sci. 90, 12040-44). LPS derived from E. coli is administered at 30 mg/kg by i.p. injection to animals which have been administered a CETP inhibitor at an appropriate dose to result in elevation of HDL. The number of surviving mice is determined at times up to 48 h after LPS injection and compared to those mice administered vehicle (minus CETP inhibitor) only.

Administration of the compounds of this invention can be via any method which delivers a compound of this invention systemically and/or locally. These methods include oral routes, parenteral, intraduodenal routes, etc. Generally, the compounds of this invention are administered orally, but parenteral administration (e.g., intravenous, intramuscular, subcutaneous or intramedullary) may be utilized, for example, where oral administration is inappropriate for the target or where the patient is unable to ingest the drug.

In general an amount of a compound of this invention is used that is sufficient to achieve the therapeutic effect desired (e.g., HDL elevation).

In general an effective dosage for the compounds of this invention is about 0.001 to 100 mg/kg/day of the compound, a prodrug thereof, or a pharmaceutically acceptable salt of said compound or of said prodrug. An especially preferred dosage is about 0.01 to 10 mg/kg/day of the compound, a prodrug thereof, or a pharmaceutically acceptable salt of said compound or of said prodrug.

A dosage of the combination pharmaceutical agents to be used in conjuction with the CETP inhibitors is used that is effective for the indication being treated.

For example, typically an effective dosage for HMG-CoA reductase inhibitors is in the range of 0.01 to 100 mg/kg/day. In general an effect dosage for a PPAR modulator is in the range of 0.01 to 100 mg/kg/day.

The compounds of the present invention are generally administered in the form of a pharmaceutical composition comprising at least one of the compounds of this invention together with a pharmaceutically acceptable vehicle, diluent or carrier as described below. Thus, the compounds of this invention can be administered individually or together in any conventional oral, parenteral, rectal or transdermal dosage form.

For oral administration a pharmaceutical composition can take the form of solutions, suspensions, tablets, pills, capsules, powders, and the like. Tablets containing various excipients such as sodium citrate, calcium carbonate and calcium phosphate are employed along with various disintegrants such as starch and preferably potato or tapioca starch and certain complex silicates, together with binding agents such as polyvinylpyrrolidone, sucrose, gelatin and acacia. Additionally, lubricating agents such as magnesium stearate, sodium lauryl sulfate and talc are often very useful for tabletting purposes. Solid compositions of a similar type are also employed as fillers in soft and hard-filled gelatin capsules; preferred materials in this connection also include lactose or milk sugar as well as high molecular weight polyethylene glycols. A preferred formulation is a solution or suspension in an oil, for example, a vegetable oil, such as olive oil; triglycerides such as those marketed under the name, Miglyol™; or mono- or diglycerides such as those marketed under the name, Capmul™, for example, in a soft gelatin capsule. Antioxidants may be added to prevent long-term degradation as appropriate. When aqueous suspensions and/or elixirs are desired for oral administration, the compounds of this invention can be combined with various sweetening agents, flavoring agents, coloring agents, emulsifying agents and/or suspending agents, as well as such diluents as water, ethanol, propylene glycol, glycerin and various like combinations thereof.

Pharmaceutical compositions comprising a solid amorphous dispersion of a cholesteryl ester transfer protein (CETP) inhibitor and a concentration-enhancing polymer are described in International Publication No. WO 02/11710, which is hereby incorporated by reference herein. Self-emulsifying formulations of cholesteryl ester transfer protein (CETP) inhibitors are described in International Publication No. WO 03/000295, which is hereby incorporated by reference herein. Methods for depositing small drug crystals on excipients are set forth in the literature, such as in J. Pharm. Pharmacol. 1987, 39:769-773, which is hereby incorporated by reference herein.

For purposes of parenteral administration, solutions in sesame or peanut oil or in aqueous propylene glycol can be employed, as well as sterile aqueous solutions of the corresponding water-soluble salts. Such aqueous solutions may be suitably buffered, if necessary, and the liquid diluent first rendered isotonic with sufficient saline or glucose. These aqueous solutions are especially suitable for intravenous, intramuscular, subcutaneous and intraperitoneal injection purposes. In this connection, the sterile aqueous media employed are all readily obtainable by standard techniques well-known to those skilled in the art.

For purposes of transdermal (e.g., topical) administration, dilute sterile, aqueous or partially aqueous solutions (usually in about 0.1% to 5% concentration), otherwise similar to the above parenteral solutions, are prepared.

Methods of preparing various pharmaceutical compositions with a certain amount of active ingredient are known, or will be apparent in light of this disclosure, to those skilled in this art. For examples of methods of preparing pharmaceutical compositions, see Remington's Pharmaceutical Sciences, Mack Publishing Company, Easter, Pa., 15th Edition (1975).

Pharmaceutical compositions according to the invention may contain 0.1%-95% of the compound(s) of this invention, preferably 1%-70%. In any event, the composition or formulation to be administered will contain a quantity of a compound(s) according to the invention in an amount effective to treat the disease/condition of the subject being treated, e.g., atherosclerosis.

Since the present invention has an aspect that relates to the treatment of the disease/conditions described herein with a combination of active ingredients which may be administered separately, the invention also relates to combining separate pharmaceutical compositions in kit form. The kit comprises two separate pharmaceutical compositions: a compound of the present invention, a prodrug thereof or a salt of such compound or prodrug and a second compound as described above. The kit comprises means for containing the separate compositions such as a container, a divided bottle or a divided foil packet. Typically the kit comprises directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician.

An example of such a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process recesses are formed in the plastic foil. The recesses have the size and shape of the tablets or capsules to be packed. Next, the tablets or capsules are placed in the recesses and the sheet of relatively stiff material is sealed against the plastic foil at the face of the foil which is opposite from the direction in which the recesses were formed. As a result, the tablets or capsules are sealed in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It may be desirable to provide a memory aid on the kit, e.g., in the form of numbers next to the tablets or capsules whereby the numbers correspond with the days of the regimen which the tablets or capsules so specified should be ingested. Another example of such a memory aid is a calendar printed on the card, e.g., as follows “First Week, Monday, Tuesday, . . . etc . . . Second Week, Monday, Tuesday, . . . ” etc. Other variations of memory aids will be readily apparent. A “daily dose” can be a single tablet or capsule or several pills or capsules to be taken on a given day. Also, a daily dose of compounds of the present invention can consist of one tablet or capsule while a daily dose of the second compound can consist of several tablets or capsules and vice versa. The memory aid should reflect this.

In another specific embodiment of the invention, a dispenser designed to dispense the daily doses one at a time in the order of their intended use is provided. Preferably, the dispenser is equipped with a memory-aid, so as to further facilitate compliance with the regimen. An example of such a memory-aid is a mechanical counter which indicates the number of daily doses that has been dispensed. Another example of such a memory-aid is a battery-powered micro-chip memory coupled with a liquid crystal readout, or audible reminder signal which, for example, reads out the date that the last daily dose has been taken and/or reminds one when the next dose is to be taken.

The compounds of this invention either alone or in combination with each other or other compounds generally will be administered in a convenient formulation. The following formulation examples only are illustrative and are not intended to limit the scope of the present invention.

In the formulations which follow, “active ingredient” means a compound of this invention.

Formulation 1: Gelatin Capsules

Hard gelatin capsules are prepared using the following:

| Ingredient | Quantity (mg/capsule) |
|---|---|
| Active ingredient | 0.25-100 |
| Starch, NF | 0-650 |
| Starch flowable powder | 0-50 |
| Silicone fluid 350 centistokes | 0-15 |

A tablet formulation is prepared using. the ingredients below:

Formulation 2: Tablets

| Ingredient | Quantity (mg/tablet) |
|---|---|
| Active ingredient | 0.25-100 |
| Cellulose, microcrystalline | 200-650 |
| Silicon dioxide, fumed | 10-650 |
| Stearate acid | 5-15 |

The components are blended and compressed to form tablets.

Alternatively, tablets each containing 0.25-100 mg of active ingredients are made up as follows:

Formulation 3: Tablets

| Ingredient | Quantity (mg/tablet) |
|---|---|
| Active ingredient | 0.25-100 |
| Starch | 45 |
| Cellulose, microcrystalline | 35 |
| Polyvinylpyrrolidone (as 10% solution in water) | 4 |
| Sodium carboxymethyl cellulose | 4.5 |
| Magnesium stearate | 0.5 |
| Talc | 1 |

The active ingredients, starch, and cellulose are passed through a No. 45 mesh U.S. sieve and mixed thoroughly. The solution of polyvinylpyrrolidone is mixed with the resultant powders which are then passed through a No. 14 mesh U.S. sieve. The granules so produced are dried at 50°-60° C. and passed through a No. 18 mesh U.S. sieve. The sodium carboxymethyl starch, magnesium stearate, and talc, previously passed through a No. 60 U.S. sieve, are then added to the granules which, after mixing, are compressed on a tablet machine to yield tablets.

Suspensions each containing 0.25-100 mg of active ingredient per 5 ml dose are made as follows:

Formulation 4: Suspensions

| Ingredient | Quantity (mg/5 ml) | |
|---|---|---|
| Active ingredient | 0.25-100 | mg |
| Sodium carboxymethyl cellulose | 50 | mg |
| Syrup | 1.25 | mg |
| Benzoic acid solution | 0.10 | mL |
| Flavor | q.v. | |
| Color | q.v. | |
| Purified Water to | 5 | mL |

The active ingredient is passed through a No. 45 mesh U.S. sieve and mixed with the sodium carboxymethyl cellulose and syrup to form smooth paste. The benzoic acid solution, flavor, and color are diluted with some of the water and added, with stirring. Sufficient water is then added to produce the required volume.

An aerosol solution is prepared containing the following ingredients:

Formulation 5: Aerosol

| Ingredient | Quantity (% by weight) |
|---|---|
| Active ingredient | 0.25 |
| Ethanol | 25.75 |
| Propellant 22 (Chlorodifluoromethane) | 70.00 |

The active ingredient is mixed with ethanol and the mixture added to a portion of the propellant 22, cooled to 30° C., and transferred to a filling device. The required amount is then fed to a stainless steel container and diluted with the remaining propellant. The valve units are then fitted to the container.

Suppositories are prepared as follows:

Formulation 6: Suppositories

| Ingredient | Quantity (mg/suppository) |
|---|---|
| Active ingredient | 250 |
| Saturated fatty acid glycerides | 2,000 |

The active ingredient is passed through a No. 60 mesh U.S. sieve and suspended in the saturated fatty acid glycerides previously melted using the minimal necessary heat. The mixture is then poured into a suppository mold of nominal 2 g capacity and allowed to cool.

An intravenous formulation is prepared as follows:

Formulation 7: Intravenous Solution

| Ingredient | Quantity | |
|---|---|---|
| Active ingredient dissolved in ethanol 1% | 20 | mg |
| Intralipid ™ emulsion | 1,000 | ml |

The solution of the above ingredients is intravenously administered to a patient at a rate of about 1 mL per minute.

Soft gelatin capsules are prepared using the following:

Formulation 8: Soft Gelatin Capsule with Oil Formulation

| Ingredient | Quantity (mg/capsule) |
|---|---|
| Active ingredient | 10-500 |
| Olive Oil or Miglyol ™ Oil | 500-1000 |

The active ingredient above may also be a combination of agents.

### General Experimental Procedures

The following examples are put forth so as to provide those of ordinary skill in the art with a disclosure and description of how the compounds, compositions, and methods claimed herein are made and evaluated, and are intended to be purely exemplary of the invention and are not intended to limit the scope of what the inventors regard as their invention. Unless indicated otherwise, percent is percent by weight given the component and the total weight of the composition, temperature is in ° C. or is at ambient temperature, and pressure is at or near atmospheric. Commercial reagents were utilized without further purification. Room or ambient temperature refers to 20-25° C. All non-aqueous reactions were run under a nitrogen atmosphere for convenience and to maximize yields. Concentration in vacuo means that a rotary evaporator was used. The names for the compounds of the invention were created by the Autonom 2.0 PC-batch version from Beilstein Informationssysteme GmbH (ISBN 3-89536-9764). The chemical structures depicted may be only exemplary of the general structure or of limited isomers, and not include specific stereochemistry as recited in the chemical name.

NMR spectra were recorded on a Varian Unity 400 (Varian Co., Palo Alto, Calif.) NMR spectrometer at ambient temperature. Chemical shifts are expressed in parts per million (δ) relative to an external standard (tetramethylsilane). The peak shapes are denoted as follows: s, singlet; d, doublet, t, triplet, q, quartet, m, multiplet with the prefix br indicating a broadened signal. The coupling constant (J) data given have a maximum error of ±0.41 Hz due to the digitization of the spectra that are acquired. Mass spectra were obtained by (1) atmospheric pressure chemical ionization (APCI) in alternating positive and negative ion mode using a Fisons Platform II Spectrometer or a Micromass MZD Spectrometer (Micromass, Manchester, UK) or (2) electrospray ionization in alternating positive and negative ion mode using a Micromass MZD Spectrometer (Micromass, Manchester, UK) with a Gilson LC-MS interface (Gilson Instruments, Middleton, Wis.) or (3) a QP-8000 mass spectrometer (Shimadzu Corporation, Kyoto, Japan) operating in positive or negative single ion monitoring mode, utilizing electrospray ionization or atmospheric pressure chemical ionization. Where the intensity of chlorine- or bromine-containing ions are described, the expected intensity ratio was observed (approximately 3:1 for ³⁵Cl/³⁷Cl-containing ions and 1:1 for ⁷⁹Br/⁸¹Br-containing ions) and the position of only the lower mass ion is given.

Column chromatography was performed with either Baker Silica Gel (40 μm) (J.T. Baker, Phillipsburg, N.J.) or Silica Gel 60 (40-63 μm) (EM Sciences, Gibbstown, N.J.). Flash chromatography was performed using a Flash 12 or Flash 40 column (Biotage, Dyar Corp., Charlottesville, Va.). Preparative HPLC purification was performed on a Shimadzu 10A preparative HPLC system (Shimadzu Corporation, Kyoto, Japan) using a model SIL-10A autosampler and model 8A HPLC pumps. Preparative HPLC-MS was performed on an identical system, modified with a QP-8000 mass spectrometer operating in positive or negative single ion monitoring mode, utilizing electrospray ionization or atmospheric pressure chemical ionization. Elution was carried out using water/acetonitrile gradients containing either 0.1% formic acid or ammonium hydroxide as a modifier. In acidic mode, typical columns used include Waters Symmetry C8, 5 μm, 19×50 mm or 30×50 mm, Waters XTerra C18, 5 μm, 50×50 (Waters Corp, Milford, Mass.) or Phenomenex Synergi Max-RP 4 μm, 50×50 mm (Phenomenex Inc., Torrance, Calif.). In basic mode, the Phenomenex Synergi Max-RP 4 μm, 21.2×50 mm or 30×50 mm columns (Phenomenex Inc., Torrance, Calif.) were used.

Optical rotations were determined using a Jasco P-1020 Polarimeter Jasco Inc., Easton, Md.)

Dimethylformamide, tetrahydrofuran, toluene and dichloromethane were the anhydrous grade supplied by Aldrich Chemical Company (Milwaukee, Wis.). Unless otherwise specified, reagents were used as obtained from commercial sources. The terms “concentrated” and “evaporated” refer to removal of solvent at 1-200 mm of mercury pressure on a rotary evaporator with a bath temperature of less than 45° C. The abbreviation “min” stand for “minutes” and “h” or “hr” stand for “hours.” The abbreviation “gm” or “g” stand for grams. The abbreviation “μl” or “μL” stand for microliters.

### Preparation 1

### (R,S)-2-Ethyl-4-hydroxy-6-trifluoromethyl-3,4-dihydro-2H-quinoline-1-carboxylic Acid Ethyl Ester (Method 1)

To a solution of (R,S)-4-amino-2-ethyl-6-trifluoromethyl-3,4-dihydro-2H-quinoline-1-carboxylic acid ethyl ester (1294 gm, 4.09 mol, prepared according to the procedure described in WO 0140190) in glacial acetic acid (3882 ml) was added a solution of sodium nitrite (582 gm, 8.18 mol) in water (1618 ml), maintaining a temperature of 20 to 25° C. The solvent was removed under vacuum, the residue dissolved in methylene chloride (2006 ml), and the solution was washed with saturated sodium hydrogen carbonate solution. The solvent was removed by distillation at atmospheric pressure and the residue was dissolved in anhydrous ethanol (2688 ml) and treated with aqueous sodium hydroxide (62.2 gm, 1.55 mol). The solvent was removed under vacuum, the residue dissolved in methylene chloride (2000 ml), washed with water, dried over anhydrous magnesium sulfate, and evaporated to dryness under vacuum to give the title compound as an oil (1219 gm) which was used without further purification in the following procedure.

### Preparation 2

### (RS)-2-Ethyl-4-oxo-6-trifluoromethyl-3,4-dihydro-2H-quinoline-1-carboxylic Acid Ethyl Ester

To a solution of (R,S)-2-ethyl-4-hydroxy-6-trifluoromethyl-3,4-dihydro-2H-quinoline-1-carboxylic acid ethyl ester (1208 gm, 3.81 mol) in methylene chloride (4663 ml) was added 2,2,6,6-tetramethyl-1-piperidinyloxy (TEMPO), free radical (6.1 gm, 0.038 mol), and a solution of potassium bromide (45.8 gm, 0.381 mol) dissolved in water (191 ml). A 6% aqueous sodium hypochlorite solution (7748 ml), which had been buffered to pH 8.6 to 9.5 with solid sodium hydrogen carbonate (78 gm), was slowly added at 0 to 5° C. The aqueous layer was washed with methylene chloride (1208 ml). The combined organic layers were washed with 1.4N hydrochloric acid (1493 ml) to which potassium iodide (12.8 gm, 0.076 moles) had been added, then aqueous sodium thiosulfate (60.8 gm, 0.381 mol) dissolved in water (1208 ml) and finally water (1691 ml). The organic layer was dried over anhydrous magnesium sulfate and evaporated to dryness under vacuum to give the title compound as a yellow oil (1193 gm).

¹H-NMR (DMSO-d₆) δ 8.03 (m, 2H), 7.91 (dd, J=9.12, 2.49 Hz, 1H), 4.79 (m, 1H), 4.23 (q, J=7.05 Hz, 2H), 3.25 (dd, J=17.42, 5.81 Hz, 1H), 2.61 (dd, J=17.42, 1.66 Hz, 1H), 1.42 (m, 2H), 1.25 (t, J=7.05 Hz, 3H), 0.76 (t, J=7.05 Hz, 3H).

### Preparations 3 and 4

### (R)-2-ethyl-4-oxo-6-trifluoromethyl-3,4-dihydro-2H-quinoline-1-carboxylic Acid Ethyl Ester (3) and (S)-2-ethyl-4-oxo-6-trifluoromethyl-3,4-dihydro-2H-quinoline-1-carboxylic Acid Ethyl Ester (4)

(RS)-2-ethyl-4-oxo-6-trifluoromethyl-3,4-dihydro-2H-quinoline-1-carboxylic acid ethyl ester was resolved by chiral chromatography on a 10 cm×25 cm column packed with Chiralcel OD (Chiral Technologies Inc., Exton, Pa.). The racemic ketone (300 mg) in methanol (0.84 mL) was injected onto the column and eluted with heptane:isopropanol 99:1 at a flow rate of 275 mL/min to give the title compounds:

(R)-2-ethyl-4-oxo-6-trifluoromethyl-3,4-dihydro-2H-quinoline-1-carboxylic acid ethyl ester (3), retention time 7.82 min, [a]_{D}=−139.81° (c=0.438, chloroform).

(S)-2-ethyl-4-oxo-6-trifluoromethyl-3,4-dihydro-2H-quinoline-1-carboxylic acid ethyl ester (4), retention time 8.93 min, [a]_{D}=+139.7° (c=0.41, chloroform).

### Preparation 5

### 4-Hydrazono-6,7-dimethoxy-2-methyl-3,4-dihydro-2H-quinoline-1-carboxylic Acid Ethyl Ester

A mixture of 6,7-dimethoxy-2-methyl-4-oxo-3,4-dihydro-2H-quinoline-1-carboxylic acid ethyl ester (1.00 gm, 3.41 mmol, prepared according to the procedure described in German Patent DE 2461050), hydrazine hydrate (330 μl, 6.80 mmol) and ethanol (4.5 mL) were heated together in a crimp-topped vial at 150° C. for 30 min in a microwave oven (Emrys Optimizer, Personal Chemistry, Uppsala, Sweden). The solvent was removed under vacuum, the residue dissolved in ethanol (4.5 mL), hydrazine hydrate (330 μl, 6.80 mmol) added and the solution was heated as before at 150° C. for 30 min. The solvent was evaporated under vacuum to give the title compound as a pale yellow solid.

MS: 308.2 [M+H]⁺ found

¹H-NMR (CDCl₃) δ 7.39 (s, 1H), 7.03 (brs, 1H), 5.21 (s, 2H), 5.04 (m, 1H), 4.27 (m, 1H), 4.15 (m, 1H), 3.88 (s, 3H), 3.85 (s, 3H), 2.61 (dd, J=17, 5.81 Hz, 1H), 2.53 (dd, J=17, 1.66 Hz, 1H), 1.28 (t, J=7.47 Hz, 3H), 1.08 (d, J =6.64 Hz, 3H).

### Preparation 6

### (R)-2-Ethyl-4-hydrazono-6-trifluoromethyl-3,4-dihydro-2H-quinoline-1-carboxylic Acid Ethyl Ester

A mixture of (R)-2-ethyl-4-oxo-6-trifluoromethyl-3,4-dihydro-2H-quinoline-1-carboxylic acid ethyl ester (Preparation 3, 1.13 gm, 3.58 mmol), hydrazine hydrate (348 μl, 7.16 mmol) and ethanol (10 mL) were heated in a Dean-Stark apparatus under conditions allowing for slow distillation of the solvent. After 5 h approximately 5 mL of distillate had been collected. The solution was diluted with an equal volume of toluene and the solvent was evaporated under vacuum to give a pale green solid which was triturated and rinsed with a little hexanes to give the title compound as a nearly colorless solid (1.07 g).

MS: 330.2 [M+H]⁺ found

¹H-NMR (CDCl₃) δ 8.23 (s, 1H), 7.62 (brd, J=8.30 Hz, 1H), 7.46 (dd, J=8.30, 1.66 Hz, 1H), 5.45 (brs, 2H), 4.82 (m, 1H), 4.28 (m, 1H), 4.24 (m, 1H), 2.64 (m, 2H), 1.36 (m, 2H), 1.31 (t, J=7.47 Hz, 3H), 0.84 (t, J=7.47 Hz, 3H).

### Preparation 7

### 4-Diazo-6,7-dimethoxy-2-methyl-3,4-dihydro-2H-quinoline-1-carboxylic Acid Ethyl Ester

To a solution of 4-hydrazono-6,7-dimethoxy-2-methyl-3,4-dihydro-2H-quinoline-1-carboxylic acid ethyl ester (Preparation 5, 200 mg, 0.65 mmol) in diethyl ether (20 mL) was added manganese (IV) oxide (400 mg, activated, ˜85%, Aldrich Chemical Company, Milwaukee, Wis.). The suspension was stirred at ambient temperature under nitrogen in the dark for 30 min then the solid removed by filtration through Celite® to give the title compound as a fuchsia colored solution which was typically used immediately.

### Preparation 8

### (R)-4-Diazo-2-ethyl-6-trifluoromethyl-3,4-dihydro-2H-quinoline-1-carboxylic Acid Ethyl Ester

To a solution of (R)-2-ethyl-4-hydrazono-6-trifluoromethyl-3,4-dihydro-2H-quinoline-1-carboxylic acid ethyl ester (Preparation 6, 317 mg, 0.962 mmol) in diethyl ether (6 mL) was added manganese (IV) oxide (1.1 g, activated, ˜85%, Aldrich Chemical Company, Milwaukee, Wis.). The suspension was stirred at ambient temperature under nitrogen in the dark for 1.5 h then the solid removed by filtration through Celite®. The filtrate was diluted with toluene (15 mL) prior to evaporation to a final volume of approximately 10 mL (never to dryness) to give the title compound as a fuchsia colored solution which was typically used immediately.

### Preparations 9 and 10

### (R)-4-Chloro-2-ethyl-4-methoxycarbonecarbonyl-6-trifluoromethyl-3,4-dihydro-2H-quinoline-1-carboxylic Acid Ethyl Ester (9) and (R)-2-ethyl-4-methoxycarbonecarbonyl-6-trifluoromethyl-2H-quinoline-1-carboxylic Acid Ethyl Ester (10)

To a solution of (R)-4-diazo-2-ethyl-6-trifluoromethyl-3,4-dihydro-2H-quinoline-1-carboxylic acid ethyl ester (Preparation 8) prepared from Preparation 6 (317 mg, 0.962 mmol) as a solution in toluene as described above was added N,N-diisopropylethylamine (335 μl, 1.92 mmol) followed, dropwise, by methyl chlorooxoacetate (0.962 mmol, 88.4 μl). The mixture was allowed to stir at room temperature under nitrogen. Gas evolution was observed and the fuchsia color changed to yellow-orange within about 10 min. The solution was diluted with ethyl acetate, washed with saturated sodium hydrogen carbonate solution then water, dried over anhydrous sodium sulfate and evaporated to dryness. The residue was chromatographed on silica eluting with a hexanes:ethyl acetate gradient from 19:1 to 4:1 to give the title compounds:

(R)-4-Chloro-2-ethyl-4-methoxycarbonecarbonyl-6-trifluoromethyl-3,4-dihydro-2H-quinoline-1-carboxylic acid ethyl ester (earlier eluting diastereoisomer, 82 mg)

MS: 422.0 [M+H]⁺ found

¹H-NMR (CDCl₃) δ 7.72 (d, J=9.13 Hz, 1H), 7.58 (m, 1H), 7.54 (s, 1H), 4.57 (m, 1H), 4.27 (m, 1H), 4.25 (m, 1H), 3.95 (s, 3H), 2.85 (dd, J=14.11, 6.64 Hz, 1H), 2.77 (dd, J=14.11, 6.92 Hz, 1H), 1.61 (m, 1H), 1.52 (m, 1H), 1.30 (t, J=7.47 Hz, 3H), 0.87 (t, J=7.47 Hz, 3H).

(R)-4-Chloro-2-ethyl-4-methoxycarbonecarbonyl-6-trifluoromethyl-3,4-dihydro-2H-quinoline-1-carboxylic acid ethyl ester (later eluting diastereoisomer, 73 mg)

MS: 422.0 [M+H]⁺ found

¹H-NMR (CDCl₃) δ 7.88 (s, 1H), 7.60 (m, 2H), 7.54 (s, 1H), 4.57 (m, 1H), 4.22 (m, 1H), 4.20 (m, 1H), 3.73 (s, 3H), 3.26 (dd, J=13.8, 7.47 Hz, 1H), 2.23 (dd, J=13.8, 6.75 Hz, 1H), 1.65 (m, 1H), 1.53 (m, 1H), 1.27 (t, J=7.47 Hz, 3H), 0.89 (t, J =7.47 Hz, 3H).

(R)-2-Ethyl-4-methoxycarbonecarbonyl-6-trifluoromethyl-2H-quinoline-1-carboxylic acid ethyl ester, 133 mg

MS: 384.1 [M-H]⁻ found

¹H-NMR (CDCl₃) δ 8.27 (s, 1H), 7.71 (brd, J=8.30 Hz, 1H), 7.56 (dd, J=8.30, 1.66 Hz, 1H), 7.20 (d, J=6.64 Hz, 1H), 5.21 (m, 1H), 4.28 (m, 2H), 3.95 (s, 3H), 1.57 (m, 1H), 1.41 (m, 1H), 1.32 (t, J=7.47 Hz, 3H), 0.91 (t, J =7.47 Hz, 3H).

### Preparation 11

### 2-Ethyl-4-hydroxy-6-trifluoromethyl-3,4-dihydro-2H-quinoline-1-carboxylic Acid Ethyl Ester (Method 2)

To a solution of 2-ethyl-4-oxo-6-trifluoromethyl-3,4-dihydro-2H-quinoline-1-carboxylic acid ethyl ester (0.89 gm, 2.83 mmol) in methanol (20 mL) was added solid sodium borohydride (102 mg, 2.8 mmol). After 10 min acetone was added to quench the reaction and the mixture was stirred for 2 h before evaporating the solvent under vacuum. The residue was dissolved in methylene chloride and the solution was washed with 0.05N hydrochloric acid. The organic layer was dried over anhydrous sodium sulfate, diluted with toluene and evaporated to dryness to give the title compound as a mixture of diastereoisomers which were carried forward unseparated.

MS: 318.0 [M+H]⁺ found

(trans isomer-minor) ¹H-NMR (CDCl₃) δ 7.80 (d, J=8.30 Hz, 1H), 7.69 (s, 1H), 7.49 (dd, J=8.30, 1.66 Hz, 1H), 4.86 (m, 1H), 4.58 (m, 1H), 4.25 (m, 2H), 2.16 (m, 2H), 1.60 (m, 1H), 1.50 (m, 1H), 1.32 (t, J=7.47 Hz, 3H), 0.90 (t, J=7.47 Hz, 3H).

MS: 318.0 [M+H]⁺ found

(cis isomer-major) ¹H-NMR (CDCl₃) δ 7.73 (s, 1H), 7.58 (d, J=9.13 Hz, 1H), 7.50 (dd, J=9.13, 1.66 Hz, 1H), 4.55 (m, 1H), 4.41 (m, 1H), 4.24 (m, 2H), 2.52 (ddd, J=13.28, 7.47, 4.98 Hz, 1H), 1.67 (m, 1H), 1.60 (m, 1H), 1.48 (m, 1H), 1.30 (t, J=7.47 Hz, 3H), 0.85 (t, J=7.47 Hz, 3H).

### Preparation 12

### 4-Chloro-2-ethyl-6-trifluoromethyl-3,4-dihydro-2H-quinoline-1-carboxylic Acid Ethyl Ester (Method 1)

The product from the previous preparation (Preparation 11, 2.83 mmol) was dissolved in anhydrous methylene chloride (30 mL), cooled to 0° C. and triethylamine (1.0 mL, 7.17 mmol) followed by methanesulfonyl chloride (245 μl, 3.16 mmol) were added. After 2 h, a further aliquot of triethylamine (1.0 mL, 7.17 mmol) was added, and the mixture stirred at ambient temperature for 15 h then washed with water containing 2N hydrochloric acid (10 mL). The organic layer was dried over anhydrous sodium sulfate, evaporated to dryness and purified by chromatography on silica eluting with hexanes:acetone 25:1 to give title compound as an unequal mixture of diastereoisomers (590 mg) which typically were carried forward unseparated.

Diastereoisomer 1 (Major)

MS: 336.0 [M+H]⁺ found

¹H-NMR (CDCl₃) δ 7.69 (d, J=8.29 Hz, 1H), 7.60 (s, 1H), 7.63 (d, J=8.29 Hz, 1H), 5.12 (dd, J=6.64, 4.98 Hz, 1H), 4.60 (m, 1H), 4.27 (m, 2H), 2.60 (ddd, J=14.11, 6.64, 6.64 Hz, 1H), 2.13 (ddd, J=14.11, 6.64, 4.98 Hz, 1H), 1.64 (m, 1H), 1.54 (m, 1H), 1.31 (t, J=7.47 Hz, 3H), 0.89 (t, J=7.47 Hz, 3H).

Diastereoisomer 2 (Minor)

MS: 336.0 [M+H]⁺ found

¹H-NMR (CDCl₃) δ 7.82 (s, 1H), 7.68 (d, J=8.30 Hz, 1H), 7.51 (dd, J=8.30, 1.66 Hz, 1H), 5.08 (dd, J=5.81, 5.81 Hz, 1H), 4.55 (m, 1H), 4.25 (m, 2H), 2.71 (ddd, J=14.11, 5.81, 5.81 Hz, 1H), 2.18 (ddd, J=14.11, 5.81, 5.81 Hz, 1H), 1.74 (m, 1H), 1.60 (m, 1H), 1.31 (t, J=7.47 Hz, 3H), 0.91 (t, J=7.47 Hz, 3H).

### Preparation 13

### (R, S)-2-Ethyl-4-hydroxy-6-trifluoromethyl-3,4-dihydro-2H-quinoline-1-carboxylic Acid Ethyl Ester (Method 3)

To a solution of (R)-2-ethyl-4-oxo-6-trifluoromethyl-3,4-dihydro-2H-quinoline-1-carboxylic acid ethyl ester (1.1 gm, 3.49 mmol) in anhydrous tetrahydrofuran at 0° C. under nitrogen was added potassium triisobutylborohydride (K-Selectride®, 1M in tetrahydrofuran, 8.0 mL, 8 mmol). After 1.5 h the mixture was allowed to warm to ambient temperature, stirred for 16 h then an additional aliquot of K-Selectride® (3.49 mL, 3.49 mmol) was added. After 1.5 h the solvent was removed under vacuum, the residue was dissolved in ethyl acetate (50 mL) and this solution was washed with 2N sodium hydroxide solution (20 mL) followed by hydrogen peroxide (30%, 15 mL), dried over anhydrous magnesium sulfate and concentrated to low volume. The residue was taken up in acetonitrile and evaporated to dryness (×3) to remove residual water to give the title compound (100%), identical to the cis isomer of the product described above (Preparation 11).

### Preparation 14

### (R)-4-Chloro-2-ethyl-6-trifluoromethyl-3,4-dihydro-2H-quinoline-1-carboxylic Acid Ethyl Ester (Method 2)

(R, S)-2-ethyl-4-hydroxy-6-trifluoromethyl-3,4-dihydro-2H-quinoline-1-carboxylic acid ethyl ester (Preparation 13, 1.1 gm, 3.47 mmol) was dissolved in thionyl chloride (20 mL) at ambient temperature under nitrogen. After 1 h N,N-dimethylformamide (2 drops) was added and the mixture allowed to stir at ambient temperature for 15 h. The solvent was removed under reduced pressure and the residue was chromatographed on silica eluting with ethyl acetate-hexanes 50:1 to give the title compound as a yellow oil (405 mg) with a proton NMR spectrum very similar to that obtained for Preparation 12 except that the major and minor components of the mixture were reversed.

### EXAMPLE 1

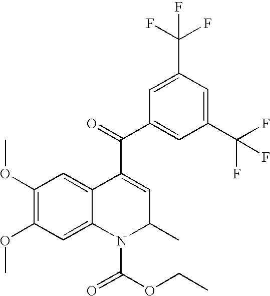

### 4-(3,5-Bis-trifluoromethyl-benzoyl)-6,7-dimethoxy-2-methyl-2H-quinoline-1-carboxylic Acid Ethyl Ester

A solution of 4-diazo-6,7-dimethoxy-2-methyl-3,4-dihydro-2H-quinoline-1-carboxylic acid ethyl ester (Preparation 7, 0.65 mmol) prepared as a solution in diethyl ether as described above was added to a solution of 3,5-bistrifluoromethylbenzoyl chloride (180 mg, 0.65 mmol) and N,N-diisopropylethylamine (120 μl, 0.65 mmol) in diethyl ether (15 mL) and allowed to stir at room temperature under nitrogen in the dark for 15 h. The solvent was removed under vacuum and the residue was chromatographed on silica eluting with hexanes:ethyl acetate 3:1 to give a partially purified product which was further purified by reverse phase chromatography (linear acetonitrile:water gradient, 55% to 100% acetonitrile, both phases containing 0.1% formic acid) to give the title compound as a lemon yellow solid (90 mg).

MS: 518.1 [M+H]⁺ found

¹H-NMR (CDCl₃) δ 8.26 (s, 2H), 8.07 (s, 1H), 7.25 (brs, 1H), 7.04 (s, 1H), 6.32 (d, J=6.64 Hz, 1H), 5.31 (m, 1H), 4.33 (m, 1H), 4.23 (m, 1H), 3.91 (s, 3H), 3.80 (s, 3H), 1.33 (t, J=7.47 Hz, 3H), 1.20 (d, J=6.64 Hz, 3H).

### EXAMPLE 2

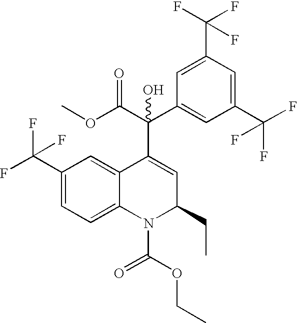

### (R)-4-[(3,5-Bis-trifluoromethyl-phenyl)-hydroxyl-methoxycarbonyl-methyl]-2-ethyl-6-trifluoromethyl-2H-quinoline-1-carboxylic Acid Ethyl Ester

A solution of 3,5-bis(trifluoromethylphenyl)magnesium bromide was prepared by dropwise addition of a solution of 3,5-bistrifluoromethylbromobenzene (0.818 mL, 4.74 mmol) in anhydrous tetrahydrofuran (0.7 mL) to a stirred suspension of magnesium powder (116 mg, 4.74 mmol) in anhydrous tetrahydrofuran (4.7 mL) at 35° C. The mixture was then heated under reflux for 1 h to give a dark colored solution. A portion of this solution (1.5 mL) was added dropwise to a solution of (R)-2-ethyl-4-methoxycarbonecarbonyl-6-trifluoromethyl-2H-quinoline-1-carboxylic acid ethyl ester (Preparation 10, 133 mg, 0.345 mmol) in anhydrous tetrahydrofuran (4 mL) at −78° C. After 30 min the mixture was warmed to 0° C. and poured into water and extracted with ethyl acetate, adding a few drops of 2N hydrochloric acid to facilitate separation of the layers. The organic layer was dried over anhydrous sodium sulfate and evaporated to dryness. This procedure was repeated in an exactly similar manner using (R)-2-ethyl-4-methoxycarbonecarbonyl-6-trifluoromethyl-2H-quinoline-1-carboxylic acid ethyl ester (Preparation 10, 235 mg, 0.609 mmol) in anhydrous tetrahydrofuran (5 mL) and adding the solution of 3,5-bis(trifluoromethylphenyl)magnesium bromide (2 mL). The combined crude product was chromatographed on silica eluting with an ethyl acetate-hexanes gradient from 5% to 30% to give the title compound as a mixture of diastereoisomers (463 mg) which were carried forward unseparated.

MS: 597.9 [M−H]⁻ found

Diastereoisomer 1: ¹H-NMR (CDCl₃) δ 7.92 (s, 2H), 7.75 (s, 1H), 7.67 (s, 1H), 7.65 (d, J=8.30 Hz, 1H), 7.34 (dd, J=8.30, 1.66 Hz, 1H), 5.97 (d, J=6.64 Hz, 1H), 5.04 (m, 1H), 4.53 (s, 1H), 4.27 (m, 2H), 3.87 (s, 3H), 1.47 (m, 1H), 1.35 (m, 1H), 1.28 (t, J=7.47 Hz, 3H), 0.85 (d, J=6.64 Hz, 3H).

MS: 597.9 [M−H]⁻ found

Diastereoisomer 2: ¹H-NMR (CDCl₃) δ 8.16 (s, 2H), 7.90 (s, 1H), 7.65 (d, J=8.30, 1H), 7.63 (s, 1H), 7.45 (dd, J=8.30, 1.66 Hz, 1H), 5.82 (d, J=6.42 Hz, 1H), 4.96 (m, 1H), 4.34 (s, 1H), 4.27 (m, 2H), 3.78 (s, 3H), 1.47 (m, 1H), 1.35 (m, 1H), 1.33 (t, J=7.47 Hz, 3H), 0.83 (d, J=7.47 Hz, 3H).

### EXAMPLES 3 AND 4

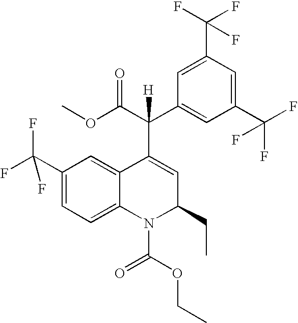

### (R, R)-4-[(3,5-Bis-trifluoromethyl-phenyl)-methoxycarbonyl-methyl]-2-ethyl-6-trifluoromethyl-2H-quinoline-1-carboxylic Acid Ethyl Ester

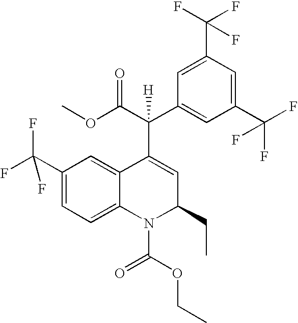

### (R, S)-4-[(3,5-Bis-trifluoromethyl-phenyl)-methoxycarbonyl-methyl]-2-ethyl-6-trifluoromethyl-2H-quinoline-1-carboxylic Acid Ethyl Ester

To a solution of (R)-4-[(3,5-bis-trifluoromethyl-phenyl)-hydroxyl-methoxycarbonyl-methyl]-2-ethyl-6-trifluoromethyl-2H-quinoline-1-carboxylic acid ethyl ester (Example 2, 125 mg, 0.208 mmol) and 2,6-di-tert-butyl-4-methylpyridine (256 mg, 1.248 mmol) in chloroform (7 mL) was added thionyl chloride (30.4 μl, 0.417 mmol). After stirring for 18 h at ambient temperature the mixture was diluted with methylene chloride, washed with water and dried over anhydrous sodium sulfate. After removal of the solvent under vacuum the residue was chromatographed on silica eluting with an ethyl acetate-hexanes gradient from 0% to 40% to give 4-[(3,5-bis-trifluoromethyl-phenyl)-chloro-methoxycarbonyl-methyl]-2-ethyl-6-trifluoromethyl-2H-quinoline-1-carboxylic acid ethyl ester as a mixture of diastereoisomers. This material (128 mg) was dissolved in a mixture of tetrahydrofuran (2 mL) and acetic acid (2 mL). Zinc dust (200 mg, 3.05 mmol) was added followed by 2N hydrochloric acid (1.5 mL). The suspension was stirred at ambient temperature for 3 h then diluted with methylene chloride and washed with water. The organic layer was dried with anhydrous sodium sulfate and evaporated to dryness under vacuum. The residue was chromatographed on silica eluting with a methylene chloride-hexanes gradient from 60% to 80% to give the title compounds:

Diastereoisomer 1: 44 mg

MS: 584.0 [M+H]⁺ found

¹H-NMR (CDCl₃) δ 7.79 (s, 1H), 7.76 (s, 2H), 7.74 (d, J=8.30 Hz, 1H), 7.47 (d, J=8.30 Hz, 1H), 7.41 (s, 1H), 6.02 (d, J=6.64 Hz, 1H), 5.12 (s, 1H), 5.01(m, 1H), 4.26 (m, 2H), 3.82 (s, 3H), 1.53 (m, 1H), 1.40 (m, 1H), 1.30 (t, J=7.47 Hz, 3H), 0.87 (d, J=7.47 Hz, 3H).

Diastereoisomer 2: 16 mg

MS: 584.0 [M+H]⁺ found

¹H-NMR (CDCl₃) δ 7.88 (s, 2H), 7.87 (s, 1H), 7.77 (d, J=8.30 Hz, 1H), 7.49 (d, J=8.3 Hz, 1H), 7.33 (s, 1H), 5.96 (d, J=5.81 Hz, 1H), 5.07 (s, 1H), 5.00 (m, 1H), 4.29 (m, 2H), 3.74 (s, 3H), 1.47 (m, 1H), 1.36 (m, 1H), 1.34 (t, J=7.47 Hz, 3H), 0.84 (d, J=7.47 Hz, 3H).

### EXAMPLES 5 AND 6

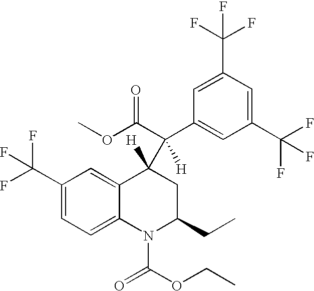

### (RS, SR, RS)-4-[(3,5-Bis-trifluoromethyl-phenyl)-methoxycarbonyl-methyl]-2-ethyl-6-trifluoromethyl-3,4-dihydro-2H-quinoline-1-carboxylic Acid Ethyl Ester

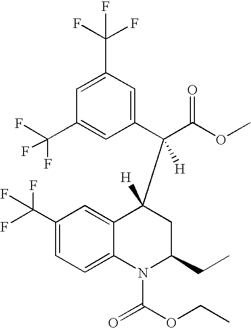

### (RS, SR, SR)-4-[(3,5-Bis-trifluoromethyl-phenyl)-methoxycarbonyl-methyl]-2-ethyl-6-trifluoromethyl-3,4-dihydro-2H-quinoline-1-carboxylic Acid Ethyl Ester

To a solution of 4-chloro-2-ethyl-6-trifluoromethyl-3,4-dihydro-2H-quinoline-1-carboxylic acid ethyl ester (Preparation 12, 275 mg, 0.82 mmol) and 3,5-bis(trifluoromethylphenyl)acetic acid methyl ester (286 mg, 1 mmol) in anhydrous dimethylformamide (2 mL) was added 1,8-diazabicyclo[5.4.0]undec-7-ene (DBU, ˜100 mg). The mixture was stirred at ambient temperature for 24 h then at 50° C. for 15 h. The mixture was poured into water, acidified by the addition of a little 2N hydrochloric acid and extracted with methylene chloride (×3). The extract was dried over anhydrous sodium sulfate, evaporated to dryness and the residue purified initially by reverse phase chromatography and finally by chromatography on silica eluting with hexanes-ethyl acetate 6:1 to give the title compounds.

(RS, SR, RS)-4-[(3,5-Bis-trifluoromethyl-phenyl)-methoxycarbonyl-methyl]-2-ethyl-6-trifluoromethyl-3,4-dihydro-2H-quinoline-1-carboxylic acid ethyl ester (13.8 mg, first eluting diastereoisomer)

MS: 586.0 [M+H]⁺ found

¹H-NMR (CDCl₃) δ 7.90 (s, 2H), 7.87 (s, 1H), 7.59 (d, J=8.30 Hz, 1H), 7.62 (d, J=8.30 Hz, 1H), 7.61 (s, 1H), 4.39-4.27 (m, 2H), 4.27-4.18 (m, 1H), 3.78 (d, J=11.61 Hz, 1H), 3.59 (m, 1H), 3.48 (s, 3H), 1.76 (ddd, J=14.10, 8.30, 3.30 Hz, 1H), 1.61-1.55 (m, 1H), 1.57-1.50 (m, 1H), 1.48-1.40 (m, 1H), 1.35 (t, J=7.47 Hz, 3H), 0.73 (t, J=7.47 Hz, 3H).

(RS, SR, SR)-4-[(3,5-Bis-trifluoromethyl-phenyl)-methoxycarbonyl-methyl]-2-ethyl-6-trifluoromethyl-3,4-dihydro-2H-quinoline-1-carboxylic acid ethyl ester (33.7mg, second eluting diastereoisomer)

MS: 586.0 [M+H]⁺ found

¹H-NMR (CDCl₃) δ 7.66 (s, 1H), 7.42 (d, J=8.30 Hz, 1H), 7.40 (s, 2H), 7.33 (dd, J=8.30, 1.66 Hz, 1H), 6.47 (d, J=1.66 Hz, 1H), 4.554.47 (m, 1H), 4.34 (m, 1H), 4.32 (m, 1H), 3.83 (d, J=11.61 Hz, 1H), 3.80 (s, 3H), 3.43 (ddd, J=11.61, 4.98, 2.49 Hz, 1H), 2.44 (ddd, J=14.11, 8.30, 2.49 Hz, 1H), 1.81 (ddd, J=14.10, 8.30, 4.98 Hz, 1H), 1.67 (m, 1H), 1.51 (m, 1H), 1.33 (t, J=7.47 Hz, 3H), 0.85 (t, J=7.47 Hz, 3H).

### EXAMPLES 7 AND 8

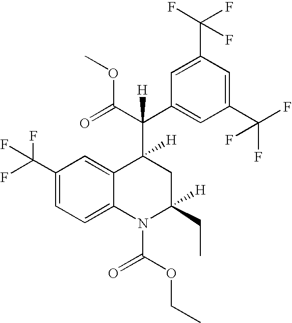

### (RS, RS, SR)-4-[(3,5-Bis-trifluoromethyl-phenyl)-methoxycarbonyl-methyl]-2-ethyl-6-trifluoromethyl-3,4-dihydro-2H-quinoline-1-carboxylic Acid Ethyl Ester

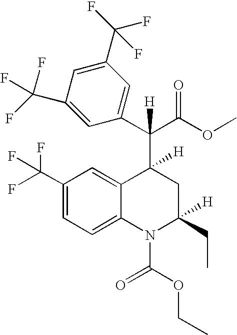

### (2S, RS, RS)-4-[(3,5-Bis-trifluoromethyl-phenyl)-methoxycarbonyl-methyl]-2-ethyl-6-trifluoromethyl-3,4-dihydro-2H-quinoline-1-carboxylic Acid Ethyl Ester

A mixture of (RS, RS)-4-[(3,5-Bis-trifluoromethyl-phenyl)-methoxycarbonyl-methyl]-2-ethyl-6-trifluoromethyl-2H-quinoline-1-carboxylic acid ethyl ester and (RS, SR)-4-[(3,5-Bis-trifluoromethyl-phenyl)-methoxycarbonyl-methyl]-2-ethyl-6-trifluoromethyl-2H-quinoline-1-carboxylic acid ethyl ester (prepared by a procedure exactly similar to that described for Examples 3 and 4 with the exception that racemic starting material was used and the mixture of diastereoisomers produced were not separated) (20 mg, 0.0342 mmol) in ethanol (5 mL) containing palladium hydroxide (20% on carbon, 20 mg) was hydrogenated in a Parr shaker (Parr Instrument Company, Moline, Ill.) at 40 psi for 5 h. The catalyst was removed by filtration through Celite® and the solvent was removed under vacuum. The residue was chromatographed on silica eluting with an ethyl acetate-hexanes gradient from 0% to 10% to give the title compounds.

(RS, RS, SR)-4-[(3,5-Bis-trifluoromethyl-phenyl)-methoxycarbonyl-methyl]-2-ethyl-6-trifluoromethyl-3,4-dihydro-2H-quinoline-1-carboxylic acid ethyl ester (first eluting diastereoisomer, 8 mg)

MS: 586.0 [M+H]⁺ found

¹H-NMR (CDCl₃) δ 7.89 (s, 1H), 7.88 (s, 2H), 7.53 (m, 2H), 7.39 (s, 1H), 4.55-4.47 (m, 1H), 4.34-4.24 (m, 2H), 4.24-4.18 (m, 1H), 4.03 (d, J=11.62 Hz, 1H), 3.77 (s, 3H), 3.38 (m, 1H), 1.75 (m, 1H), 1.49 (m, 1H), 1.38 (m, 1H), 1.31 (t, J=7.47 Hz, 3H), 0.96 (m, 1H), 0.71 (t, J=7.47 Hz, 3H).

(RS, RS, RS)-4-[(3,5-Bis-trifluoromethyl-phenyl)-methoxycarbonyl-methyl]-2-ethyl-6-trifluoromethyl-3,4-dihydro-2H-quinoline-1-carboxylic acid ethyl ester (second eluting diastereoisomer, 5 mg)

MS: 586.0 [M+H]⁺ found

¹H-NMR (CDCl₃) δ 7.93 (s, 2H), 7.84 (s, 1H), 7.53 (d, J=8.29 Hz, 1H), 7.43 (dd, J=8.29 Hz, 1H), 7.07 (s, 1H), 4.42 (m, 1H), 4.27 (d, J=9.96 Hz, 1H), 4.26 (m, 2H), 4.32 (m, 1H), 3.76 (s, 3H), 3.28 (m, 1H), 2.36 (m, 1H), 1.65 (m, 1H), 1.62 (m, 1H), 1.45 (m, 1H), 1.31 (t, J=7.47 Hz, 3H), 0.84 (t, J=7.47 Hz, 3H).

### EXAMPLES 9 AND 10

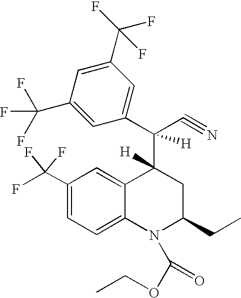

### (R, S, S)-4-[(3,5-Bis-trifluoromethyl-phenyl)-cyano-methyl]-2-ethyl-6-trifluoromethyl-3,4-dihydro-2H-quinoline-1-carboxylic Acid Ethyl Ester

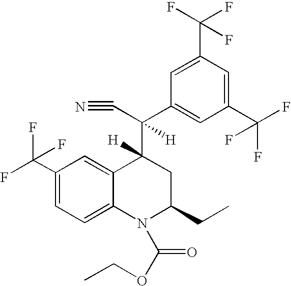

### (R, S, R)-4-[(3,5-Bis-trifluoromethyl-phenyl)-cyano-methyl]-2-ethyl-6-trifluoromethyl-3,4-dihydro-2H-quinoline-1-carboxylic Acid Ethyl Ester

Sodium hydride (60% dispersion in mineral oil, 34 mg, 0.85 mmol) was added to a solution of 3,5-bis(trifluoromethylphenyl)acetonitrile (212 mg, 0.84 mmol) in anhydrous dimethylformamide (1.5 mL). After stirring under nitrogen for 30 min at ambient temperature a solution of (R)-4-chloro-2-ethyl-6-trifluoromethyl-3,4-dihydro-2H-quinoline-1-carboxylic acid ethyl ester (Preparation 14, 188 mg, 0.56 mmol) in anhydrous dimethylformamide (2 mL) was added. The mixture was stirred at ambient temperature for 16 h then poured into water (20 mL) and extracted with diethyl ether (3×20 mL). The extract was dried over anhydrous sodium sulfate and the solvent was removed under vacuum. This material was purified by chromatography on silica eluting initially with ethyl acetate-hexanes (1:19) to obtain one diastereoisomer of the desired compound in an impure form which was further purified by reverse phase chromatography to obtain the title compound Example 9 as a clear oil (69mg). Further elution of the silica column with ethyl acetate-hexanes (1:4) provided the title compound of Example 10 as a yellow oil (54mg).

(R, S, S)-4-[(3,5-Bis-trifluoromethyl-phenyl)-cyano-methyl]-2-ethyl-6-trifluoromethyl-3,4-dihydro-2H-quinoline-1-carboxylic acid ethyl ester

MS: 553 [M+H]⁺ found

¹H-NMR (CDCl₃) δ 7.80 (s, 1H), 7.60 (d, J=8.30 Hz, 1H), 7.49 (dd, J=8.30, 1.66 Hz, 1H), 7.40 (s, 2H), 6.60 (d, J=1.66 Hz, 1H), 4.59 (m, 1H), 4.33 (m, 2H), 4.06 (d, J=9.96, 1H), 3.20 (m, 1H), 2.78 (m, 1H), 1.89 (m, 1H), 1.65 (m, 1H), 1.53 (m, 1H), 1.34 (t, J=7.47 Hz, 3H), 0.96 (m, 1H), 0.88 (t, J=7.47 Hz, 3H).

(R, S, R)-4-[(3,5-Bis-trifluoromethyl-phenyl)-cyano-methyl]-2-ethyl-6-trifluoromethyl-3,4-dihydro-2H-quinoline-1-carboxylic acid ethyl ester

MS: 553 [M+H]⁺ found

¹H-NMR (CDCl₃) δ 7.89 (s, 1H), 7.62 (s, 2H), 7.62 (d, J=8.30 Hz, 1H), 7.55 (d, J=8.30 Hz, 1H), 7.38 (s, 1H), 4.41 (m, 1H), 4.24 (m, 2H), 4.16 (m, 1H), 4.15 (d, J=8.30 Hz, 1H), 3.48 (m, 1H), 2.01 (m, 1H), 1.95 (m, 1H), 1.48 (m, 1H), 1.41 (m, 1H), 1.29 (t, J=7.47 Hz, 3H), 0.77 (t, J=7.47 Hz, 3H).

### Preparation 15

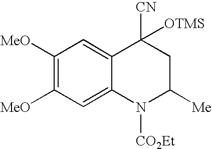

### [(R, S), (S, R)] and [(R, R), (S, S)]-4-Cyano-6,7-dimethoxy-2-methyl-4-trimethylsilanyloxy-3,4-dihydro-2H-quinoline-1-carboxylic Acid Ethyl Ester

[(RS)]-6,7-Dimethoxy-2-methyl-4-oxo-3,4-dihydro-2H-quinoline-1-carboxylic acid ethyl ester (8.02 gm, 27.3 mmol, 1 eq) and zinc iodide (0.43 gm, 1.37 mmol, 0.05 eq) were added to a dry round bottomed flask equipped with a magnetic stir bar and reflux condenser under a nitrogen atmosphere. Toluene (20 mL) was added to the flask followed by trimethylsilyl cyanide (4.40 mL, 33.0 mmol, 1.2 eq). The reaction was heated to 80° C. After 5 hr, the reaction mixture was concentrated to dryness to afford the title compounds (10.7 gm, 27.2 mmol, 100% yield) that were used without further purification.

LCMS (ESI+): 393 (MH+).

### Preparation 16

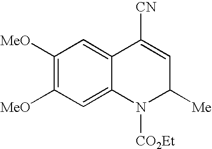

### (RS)-4-Cyano-6,7-dimethoxy-2-methyl-2H-quinoline-1-carboxylic Acid Ethyl Ester

[(R, S), (S, R)] and [(R, R), (S, S)]-4-cyano-6,7-dimethoxy-2-methyl-4-trimethylsilanyloxy-3,4-dihydro-2H-quinoline-1-carboxylic acid ethyl ester (10.7 gm, 27.37 mmol) was placed in a 100 mL round bottomed flask and dissolved in ethanol (25 mL). Hydrochloric acid in dioxane (21.0 mL of a 4.0 M solution) was added to the mixture. After 12 hr at room temperature, the reaction mixture was concentrated, quenched with a saturated aqueous sodium hydrogen carbonate solution and extracted 3 times with ethyl acetate. The combined organic layers were dried over anhydrous sodium sulfate, filtered and concentrated. Flash chromatography eluting with 80/20 hexanes/ethyl acetate the title compounds (5.49 gm, 18.1 mmol, 67% yield).

LCMS (ESI+): 303 (MH+).

¹H-NMR (CDCl₃): δ 1.13 (d, 3H), 1.33 (t, 3H), 3.90 (s, 3H), 3.92 (s, 3H), 4.30 (m, 2H), 5.25 (m, 1H), 6.65 (d, 1H), 6.91 (s, 1H), 7.24 (br s, 1H).

### Preparations 17 and 18

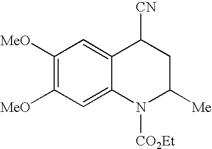

### [(R, S), ((S, R)] and [(R, R), (S, S)]-4-Cyano-6,7-dimethoxy-2-methyl-3,4-dihydro-2H-quinoline-1-carboxylic Acid Ethyl Ester

(RS)-4-Cyano-6,7-dimethoxy-2-methyl-2H-quinoline-1-carboxylic acid ethyl ester (4.99 gm, 16.5 mmol) was placed in a round bottomed flask equipped with a magnetic stir bar, dissolved in 47 mL of ethanol, and was combined with sodium borohydride (3.18 gm, 84.3 mmol, 5.1 eq). After 45 min of heating at reflux, the mixture was concentrated to dryness, quenched with water, and extracted 3 times with ethyl acetate. The organic layers were collected, dried over sodium sulfate, filtered and concentrated to afford the title compounds (4.91 gm, 16.1 mmol, 98% yield).

cis isomer:

LCMS (ESI+): 305 (MH+).

(R, S) and (S, R) ¹H-NMR (CDCl₃): δ 1.23 (d, 3H), 1.29 (t, 3H), 1.78 (m, 1H), 2.65 (m, 1H), 3.74 (m, 1H), 3.86 (s, 3H), 3.90 (s, 3H), 4.22 (m, 2H), 4.61 (m, 1H), 6.92 (s, 1H), 7.10 (s, 1H).

trans isomer:

LCMS (ESI+): 305 (MH+).

(R, R) and (S, S) ¹H-NMR (CDCl₃): δ 1.16 (d, 3H), 1.32 (t, 3H), 2.03 (m, 1H), 2.42 (m, 1H), 3.87 (s, 3H), 3.88 (s, 3H), 3.94 (m, 1H), 4.26 (m, 2H), 4.86 (m, 1H), 6.77 (s, 1H), 7.30 (s, 1H).

### Preparations 19 and 20

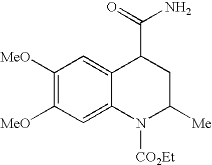

### [(R, S), (S, R)] and [(R, R), (S, S)]-4-Carbamoyl-6,7-dimethoxy-2-methyl-3,4-dihydro-2H-quinoline-1-carboxylic Acid Ethyl Ester

[(R, S), (S, R)] and [(R, R), (S, S)]-4-cyano-6,7-dimethoxy-2-methyl-3,4-dihydro-2H-quinoline-1-carboxylic acid ethyl ester (2.22 gm, 7.32 mmol, 1 eq) was dissolved in concentrated sulfuric acid (12 mL) and water (0.66 mL, 36.6 mmol, 5 eq). After 12 hr at ambient temperature the reaction was quenched into solid sodium bicarbonate, dissolved in water and extracted 3 times with ethyl acetate. Organic layers were collected, dried over anhydrous sodium sulfate, filtered, and concentrated to provide the title compounds (2.25 gm, 6.98 mmol, 95% yield).

LCMS (ESI+): 323 (MH+).

[(R, S), (S, R)]: ¹H-NMR (CDCl₃): δ 1.20 (d, 3H), 1.30 (t, 3H), 1.89 (m, 1H), 2.41 (m, 1H), 3.37 (m, 1H), 3.84 (s, 3H), 3.86 (s, 3H), 4.22 (m, 2H), 4.55 (m, 1H), 5.70 (s, 1H), 5.80 (s, 1H), 6.70 (s, 1H), 7.21 (s, 1H).

LCMS (ESI+): 323 (MH+).

[(R, R), (S, S)]: ¹H-NMR (CDCl₃): δ 1.17 (d, 3H), 1.30 (t, 3H), 1.74 (m, 1H), 2.67 (m, 1H), 3.55 (m, 1H), 3.86 (s, 3H), 3.87 (s, 3H), 4.21 (m, 2H), 4.58 (m, 1H), 5.36 (s, 1H), 5.49 (s, 1H), 6.65 (s, 1H), 7.15 (s, 1H).

### Preparations 21 and 22

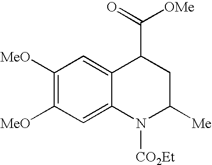

### [(R, S), (S, R)] and [(R, R), (S, S)]-6,7-Dimethoxy-2-methyl-3,4-dihydro-2H-quinoline-1,4-dicarboxylic Acid-1-ethyl Ester-4-methyl Ester

[(R, S), ((S, R)] and [(R, R), (S, S)]-4-Carbamoyl-6,7-dimethoxy-2-methyl-3,4-dihydro-2H-quinoline-1-carboxylic acid ethyl ester (2.24 gm, 6.95 mmol, 1 eq) was placed in a 100 mL round bottomed flask equipped with a stir bar and was dissolved in methylene chloride (56.5 mL). Trimethyloxonium tetrafluoroborate (1.29 gm, 8.76 mmol, 1.26 eq) was added to the solution followed by 12.2 mL more of methylene chloride. After 12 hr at ambient temperature the reaction mixture was concentrated to dryness and used without further purification. The product (2.84 gm, 6.95 mmol, 1 eq) was dissolved in water (20 mL) and stirred for several hours at room temperature. The mixture was saturated with sodium chloride solution, extracted 3 times with ethyl acetate, dried over sodium sulfate, filtered and concentrated to dryness. Flash chromatography eluting with 80/20 hexanes/ethyl acetate provided the title compounds (1.65 gm, 4.88 mmol, 70% yield).

LCMS (ESI+): 338 (MH+).

[(R, S) (S, R)] ¹H-NMR (CDCl₃): δ 1.17 (d, 3H), 1.29 (t, 3H), 1.81 (m, 1H), 2.46 (m, 1H), 3.56 (m, 1H), 3 .81 (s, 3H), 3.83 (s, 3H), 3.85 (s, 3H), 4.21 (m, 2H), 4.55 (m, 1H), 6.60 (s, 1H), 7.11 (s, 1H).

LCMS (ESI+): 338 (MH+).

[(R, R) (S, S)]: ¹H-NMR (CDCl₃): δ 1.13 (d, 3H), 1.29 (t, 3H), 1.78 (m, 1H), 2.54 (m, 1H), 3.67 (s, 3H), 3.71 (m, 1H), 3.84 (s, 3H), 3.85 (s, 3H), 4.21 (m, 2H), 4.74 (m, 1H), 6.66 (s, 1H), 7.13 (s, 1H).

### Preparations 23 and 24

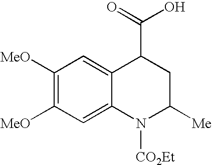

### [(R, S), (S, R)] and [(R, R), (S, S)]-6,7-Dimethoxy-2-methyl-3,4-dihydro-2H-quinoline-1,4-dicarboxylic Acid-1-ethyl Ester

[(R, S), (S, R)] and [(R, R), (S, S)]-6,7-Dimethoxy-2-methyl-3,4-dihydro-2H-quinoline-1,4-carboxylic acid ethyl ester-4-methyl ester (0.62 gm, 1.73 mmol, 1 eq) was dissolved in dioxane (12 mL) and water (12 mL) in round bottomed flask with a magnetic stir bar. Sodium hydroxide (0.13 gm, 3.48 mmol, 2 eq) was added and stirred at room temperature. After 12 hr, the reaction mixture was concentrated and partitioned between 1.0 N aqueous sodium hydroxide and diethyl ether. The aqueous layer was collected, acidified with concentrated hydrochloric acid, and extracted with ether. The organic layer was collected, dried over magnesium sulfate, filtered and concentrated to provide the title compounds (0.536 gm, 1.65 mmol, 90% yield) as a mixture of diastereoisomers.

LCMS (ESI+): 324 (MH+).

[(R, S), (S, R)]: ¹H-NMR (CDCl₃): δ 1.18 (d, 3H), 1.30 (t, 3H), 1.89 (m, 1H), 2.48 (m, 1H), 3.61 (m, 1H), 3.84 (s, 3H), 3.85 (s, 3H), 4.21 (m, 2H), 4.60 (m, 1H), 6.75 (s, 1H), 7.15 (s, 1H).

LCMS (ESI+): 324 (MH+).

[(R, R), (S, S)]: ¹H-NMR (CDCl₃): δ 1.14 (d, 3H), 1.29 (t, 3H), 1.83 (m, 1H), 2.56 (m, 1H), 3.74 (m, 1H), 3.85 (s, 6H), 4.21 (m, 2H), 4.76 (m, 1H), 6.70 (s, 1H), 7.14 (s, 1H).

### EXAMPLES 11 AND 12

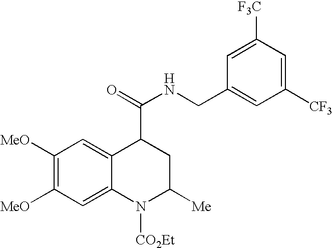

### [(R, S), (S, R)] and [(R, R), (S, S)]-4-(3,5-Bis-trifluoromethyl-benzylcarbamoyl)-6,7-dimethoxy-2-methyl-3,4-dihydro-2H-quinoline-1-carboxylic Acid Ethyl Ester

[(R, S), (S, R)] and [(R, R), (S, S)]-6,7-Dimethoxy-2-methyl-3,4-dihydro-2H-quinoline-1,4-dicarboxylic acid-1-ethyl ester (0.229 gm, 0.710 mmol) was placed in a 25 mL round bottomed flask equipped with a stir bar. Methylene chloride (7.0 mL) was added followed by the addition of 3,5-bis(trifluoromethyl)benzylamine (0.519 gm, 2.14 mmol, 3.0 eq). To this reaction, 1-hydroxybenzotriazole hydrate (0.022 gm, 0.146 mmol, 0.2 eq) and 1-(3-dimethylaminopropyl)-3-ethylcarbodiimide hydrochloride (0.232 gm, 1.21 mmol, 1.7 eq) were added. The reaction mixture was stirred at room temperature. After 12 hr, the reaction was quenched with water and extracted 3 times with ethyl acetate. The organic layers were collected, dried over sodium sulfate, filtered, concentrated to provide the title compounds (0.1225 gm, 0.223 mmol, 58% yield) as a mixture of diastereoisomers.

LCMS (ESI+): 549 (MH+).

¹H NMR [(R, R), (S, S) and (R, S) (S, R)] (CDCl₃): δ 1.16 (d, 3H), 1.24 (t, 3H), 1.80 (m, 1H), 2.66 (m, 1H), 3.64 (m, 1H), 3.81 (s, 3H), 3.86 (s, 3H), 4.11 (m, 2H), 4.29 (m, 1H), 4.56 (m, 2H), 4.70 (m, 2H), 6.05 (m, 1H), 6.40 (m, 1H), 6.65 (s, 1H), 6.60 (s, 1H), 7.13 (s, 1H), 7.66 (s, 2H), 7.75 (s, 1H), 7.80 (s, 1H). LCMS (ESI+): 549 (MH+).

### Preparations 25 and 26

### [(R, S), (S, R)] and [(R, R), (S, S)]-6,7-Dimethoxy-4-(methoxymethyl-carbamoyl)-2-methyl-3,4-dihydro-2H-quinoline-1-carboxylic Acid Ethyl Ester

[(R, S), (S, R)] and [(R, R), (S, S)]-6,7-Dimethoxy-2-methyl-3,4-dihydro-2H-quinoline-1,4-dicarboxylic acid-1-ethyl ester (0.536 gm, 1.55 mmol) was placed in a 25 mL round bottomed flask equipped with a stir bar and dissolved in methylene chloride (10 mL). The reaction mixture was cooled to 0° C., and di-iso-propylethylamine (0.18 gm, 2.0 mmol, 1.3 eq) was added followed by addition of N,O-di-methylhydroxylamine hydrochloride (1.1 eq), 4-dimethylaminopyridine (0.1 eq), and 1-(3-dimethylaminopropyl)-3-ethylcarbodiimide hydrochloride (1.2 eq). The reaction mixture was stirred at room temperature. After 12 hr, the reaction mixture was quenched with water and extracted 3 times with ethyl acetate. The organic layers were collected, dried, filtered, and concentrated to provide the title compounds (0.56 gm, 1.52 mmol, 98% yield) that were used without further purification.

LCMS (ESI+): 367 (MH+).

[(R, R), (S, S)] ¹H-NMR (CDCl₃): δ 1.10 (d, 3H), 1.28 (t, 3H), 1.83 (m, 1H), 2.41 (m, 1H), 3.16 (s, 3H), 3.38 (s, 3H), 3.80 (s, 3H), 3.82 (s, 3H), 4.06 (m, 1H), 4.21 (m, 2H), 4.84 (m 1H), 6.55 (s, 1H), 7.16 (s, 1H).

LCMS (ESI+): 367 (MH+).

[(R, S), (S, R)] ¹H-NMR (CDCl₃): δ 1.32 (d, 3H), 1.65 (t, 3H), 1.75 (m, 1H), 2.39 (m, 1H), 3.16 (s, 3H), 3.35 (s, 3H), 3.80 (s, 3H), 3.82 (s, 3H), 4.06 (m, 1H), 4.21 (m, 2H), 4.84 (m 1H), 6.45 (s, 1H), 7.05 (s, 1H).

### EXAMPLES 13 AND 14

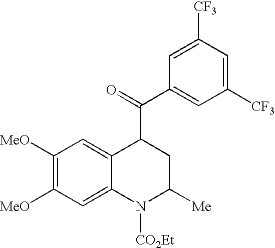

### [(R, S), (S, R)] and [(R, R), (S, S)]-4-(3,5-Bis-trifluoromethyl-benzoyl)-6,7-dimethoxy-2-methyl-3,4-dihydro-2H-quinoline-1-carboxylic Acid Ethyl Ester

[(R, S), (S, R)] and [(R, R), (S, S)]-6,7-Dimethoxy-4-(methoxy-methyl-carbamoyl)-2-methyl-3,4-dihydro-2H-quinoline-1-carboxylic acid ethyl ester (0.56 gm, 1.45 mmol, 1 eq) was dissolved in tetrahydrofuran (12 mL) in a round bottomed flask equipped with a magnetic stir bar. The reaction was cooled to 0° C. 3,5-Bis(trifluoromethyl)phenyl magnesium bromide (13.6 mL of 0.5 M solution) was added drop wise, and the reaction mixture was stirred at room temperature. After 12 hr, the reaction mixture was quenched with aqueous ammonium chloride, further saturated with NaCl and extracted 3 times with ethyl acetate. The organic layers were collected, dried over sodium sulfate, filtered and concentrated to dryness. Flash chromatography eluting with 90/10 hexanes/ethyl acetate provided the title compounds (0.556 gm, 1.07 mmol, 73% yield).

LCMS (ESI+): 520 (MH+).

[(R, S), (S, R)]: ¹H NMR (CDCl₃): δ 1.24 (d, 3H), 1.35 (t, 3H), 1.81 (m, 1H), 2.52 (m, 1H), 3.63 (s, 3H), 3.88 (s, 3H), 4.29 (m, 2H), 4.42 (m, 1H), 4.57 (m, 1H), 6.21 (s, 1H), 7.15 (s, 1H), 8.13 (s, 1H), 8.45 (s, 2H).

LCMS (ESI+): 520 (MH+).

[(R, R), (S, S)] ¹H NMR (CDCl₃): δ 1.18 (d, 3H), 1.28 (t, 3H), 1.88 (m, 1H), 2.67 (m, 1H), 3.79 (s, 3H), 3.84 (s, 3H), 4.21 (m, 2H), 4.45 (t, 1H), 4.88 (m, 1H), 6.59 (s, 1H), 7.07 (s, 1H), 7.97 (s, 1H), 8.25 (s, 2H).

### EXAMPLE 15

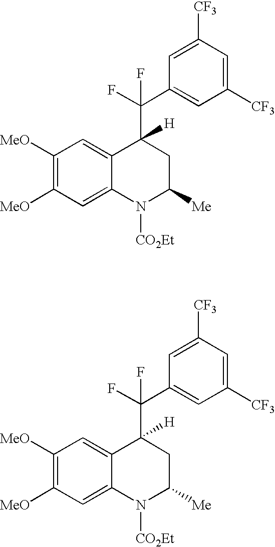

### [(R, R), (S, S)]-4-(3,5-Bis-trifluoromethyl-phenyl)-difluoro-methyl]-2-methyl-5,6-dimethoxy-3,4-dihydro-2H-quinoline-1-carboxylic Acid Ethyl Ester

[(R, R), (S, S)]-4-(3,5-Bis-trifluoromethyl-benzoyl)-2-methyl-6,7-dimethoxy-3,4-dihydro-2H-quinoline-1-carboxylic acid ethyl ester (0.062 gm, 0.120 mmol) was dissolved in methylene chloride (0.25 mL) in a vial equipped with a magnetic stir bar. [Bis(2-methoxyethyl)amino]sulfur trifluoride (0.22 mL, 1.20 mmol, 10 eq) was added to this solution and the reaction mixture was stirred at room temperature. After 12 hr, the reaction mixture was quenched with saturated aqueous ammonium chloride solution and extracted 3 times with ethyl acetate. The organic layers were collected, dried over magnesium sulfate, filtered, concentrated and purified by chromatography on silica eluting with 90/10 hexanes/ethyl acetate to provide the title compound (0.045 gm, 0.08 mmol, 66% yield).

LCMS (ESI+): 542 (MH+).

[(R, R), (S, S)]: ¹H NMR (CDCl₃): δ 1.10 (d, 3H), 1.22 (t, 3H), 1.95 (m, 1H), 2.45 (m, 1H), 3.55 (m, 1H), 3.75 (s, 3H), 3.87 (s, 3H), 4.00 (m, 1H), 4.19 (m, 1H), 4.60 (m, 1H), 6.45 (s, 1H), 6.99 (s, 1H), 7.6 (s, 2H), 7.90 (s, 1H).

### EXAMPLES 16 AND 17

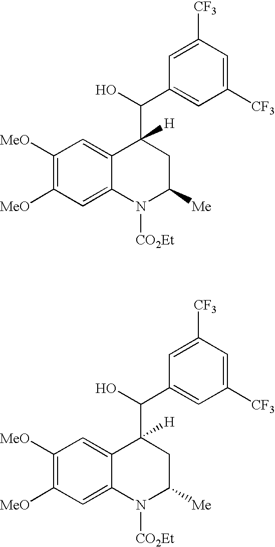

### [(R, R, R), (S, S, S)] and [(R, R, S), (S, S, R)]-4-[(3,5-Bis-trifluoromethyl-phenyl)-hydroxy-methyl]-6,7-dimethoxy-2-methyl-3,4-dihydro-2H-quinoline-1-carboxylic Acid Ethyl Ester

[(R, R), (S, S)]-4-(3,5-Bis-trifluoromethyl-benzoyl)-6,7-dimethoxy-2-methyl-3,4-dihydro-2H-quinoline-1-carboxylic acid ethyl ester (0.302 gm, 0.58 mmol, 1 eq) was placed in a round bottomed flask equipped with a magnetic stir bar. Methanol (12 mL) was added followed by the addition of sodium borohydride (0.131 gm, 3.48 mmol, 6 eq) at room temperature. After 1 hour, the reaction mixture was quenched with a brine solution, extracted 3 times with ethyl acetate and dried over sodium sulfate. The material was purified by flash chromatography eluting with 75/25 hexanes/ethyl acetate to provide the title compounds (0.271 gm, 0.52 mmol, 89% yield).

Diastereoisomer 1 (29% yield)

LCMS (ESI+): 522 (MH+).

¹H NMR (CDCl₃): δ 1.12 (d, 3H), 1.29 (t, 3H), 1.39 (m, 1H), 2.52 (m, 1H), 2.81 (br s, 1H), 2.94 (m, 1H), 3.65 (s, 3H), 3.83 (s, 3H), 4.21 (m, 2H), 4.67 (m, 1H), 5.04 (d, 1H), 6.18 (s, 1H), 7.02 (s, 1H), 7.64 (s, 2H), 7.72 (s, 1H).

Diastereoisomer 2 (60% yield)

LCMS (ESI+): 522 (MH+).

¹H NMR (CDCl₃): δ 1.14 (d, 3H), 1.34 (t, 3H), 1.56 (m, 1H), 1.86 (m, 1H), 2.97 (m, 1H), 3.87 (s, 3H), 3.88 (s, 3H), 4.26 (m, 2H), 4.49 (m, 1H), 4.77 (d, 1H), 6.70 (s, 1H), 7.09 (s, 1H), 7.84 (s, 3H).

### EXAMPLES 18 AND 19

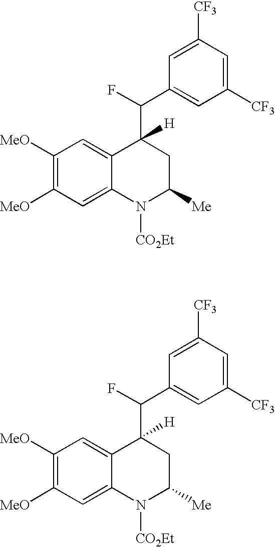

### [(R, R, R), (S, S, S) and (R, R, S), (S, S, R)]-4-[(3,5-Bis-trifluoromethyl-phenyl)-fluoro-methyl]-6,7-dimethoxy-2-methyl-3,4-dihydro-2H-quinoline-1-carboxylic Acid Ethyl Ester

[(R, R, R), (S, S, S) and (R, R, S), (S, S, R)]-4-[(3,5-Bis-trifluoromethyl-phenyl)-hydroxy-methyl]-6,7-dimethoxy-2-methyl-3,4-dihydro-2H-quinoline-1-carboxylic acid ethyl ester (0.06 gm, 0.115 mmol) was placed in a small round bottomed flask containing a magnetic stir bar and dissolved in dichloromethane (0.25 mL). To this reaction, (diethylamino)sulfur trifluoride (0.152 mL) was added dropwise at room temperature. After 2 hr, the reaction was quenched with the addition of aqueous ammonium chloride solution and extracted 3 times with EtOAc. Organics were collected, dried over Na₂SO₄, filtered and concentrated to afford the title compounds in 40% isolated yield (0.024 gm, 0.046 mmol).

LCMS (ESI+): 524 (MH+).

¹H NMR (CDCl₃): δ 1.19 (d, 3H), 1.30 (t, 3H), 2.02 (m, 1H), 2.95 (m, 1H), 3.95 (s, 3H), 3.97 (s, 3H), 4.26 (m, 2H), 4.49 (m, 1H), 6.40 (s, 1H), 6.9 (s, 1H), 7.1 (s, 1H), 7.8 (s, 3H), 7.95 (s, 1H).

### Preparation 27

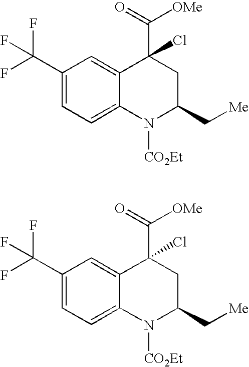

### [(R, S) and (R,R)]-4-Chloro-2-ethyl-6-trifluoromethyl-3,4-dihydro-2H-quinoline-1,4-dicarboxylic Acid-1-ethyl Ester-4-methyl Ester

(R)-2-Ethyl-4-hydrazono-6-trifluoromethyl-3,4-dihydro-2H-quinoline-1-carboxylic acid ethyl ester (Preparation 6, 1.22 gm, 3.70 mmol) was placed in a 250 mL round bottomed flask equipped with a stir bar. Diethyl ether (100 mL) was added followed by the addition of MnO₂ (22.2 mmol, 6 eq). The reaction was protected from light and stirred at room temperature for 90 min. The solution was filtered through Celite® and concentrated to a final volume of 30 mL. Additional toluene (100 mL) was added and concentrated to 40 mL. To this solution was added of di-iso-propylethylamine (3.25 mL, 18.5 mmol), followed by the addition of 20% phosgene solution in toluene (7.5 mL). The reaction mixture was stirred for 45 min before anhydrous methanol (10 mL) was added. After 2 hr, the reaction was concentrated down to 20 mL, extracted into methylene chloride and washed with 0.1 N HCl. The organic layer was collected, dried over magnesium sulfate, filtered and concentrated to dryness to obtain the title compound (1.43 gm, 3.66 mmol, 98% yield).

LCMS (ESI+): 394 (MH+).

(R, S), (R, R): ¹H NMR (CDCl₃): δ 0.95 (t, 3H), 1.25 (m, 2H), 1.30 (t, 3H), 2.60 (dd, 1H), 2.79 (dd, 1H), 3.99 (s, 3H), 4.23 (m, 2H), 4.62 (m, H), 7.61 (d, 1H), 7.70 (d, 1H).

### Preparation 28 and 29

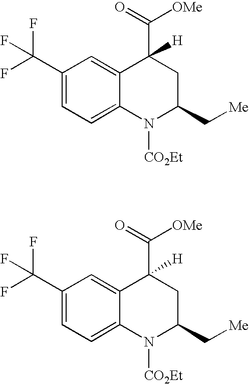

Preparation of [(R, S) and (R, R)]-2-Ethyl-6-trifluoromethyl-3,4-dihydro-2H-quinoline-1,4-dicarboxylic acid-1-ethyl ester-4-methyl ester [(R, S) and (R, R)]-4-Chloro-2-ethyl-6-trifluoromethyl-3,4-dihydro-2H-quinoline-1,4-dicarboxylic acid-1-ethyl ester-4-methyl ester (1.40 grams, 3.56 mmol) was dissolved in 30 mL of methanol before 0.15 grams of 10% Pd/C was added and the mixture was hydrogenated at 45 psi on the par shaker for 2 hours. The reaction mixture was filtered through Celite and concentrated. The crude mixture was then dissolved in dichloromethane and the organics were collected, dried over magnesium sulfate and concentrated. The product was isolated after purification by column chromatography eluting with a solution of 90:10 hexane-acetone to yield 83% (2.98 mmol) of the title compound.

(R, S): ¹H NMR (CDCl₃): δ? 0.84 (t, 3H), 1.30 (t, 3H), 1.40 (m, 2H), 1.95 (m, 1H), 2.47 (m, 1H), 3.61 (m, 1H), 3.80 (s, 3 H), 4.3 (m, 2H), 4.42 (m, 1H), 7.40 (s, 1H), 7.60 (d, 1H), 7.70 (d, 1H).

LCMS (ESI+): 360 (MH+)

(R, R): ¹H NMR (CDCl₃): δ? 0.86 (t, 3H), 1.28 (t, 3H), 1.38 (m, 2H), 2.1 (m, 1H), 2.56 (m, 1H), 3.69 (s, 3H), 3.90 (t, 1H), 4.30 (m, 2H), 4.58 (m, 1H). 7.46 (s, 1H), 7.50 (d, 1H), 7.70 (d, 1H)

LCMS (ESI+): 360 (MH+)

### Preparation 30

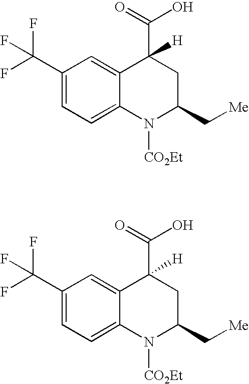

### [(R, S) and (R, R)]-2-Ethyl-6-trifluoromethyl-3,4-dihydro-2H-quinoline-1,4-dicarboxylic Acid-1-ethyl Ester

[(R, S) and (R, R)]-2-Ethyl-6-trifluoromethyl-3,4-dihydro-2H-quinoline-1,4-dicarboxylic acid-1-ethyl ester-4-methyl ester (Preparation 28 and 29, 0.070 gm, 0.195 mmol, 1 eq) was dissolved in a round bottomed flask with magnetic stir bar containing dioxane (1.5 mL) and water (1.5 mL). Sodium hydroxide (0.016 gm, 0.409mmol, 2.1 eq) was added and stirred at room temperature. After 12 hr, the reaction mixture was concentrated to a minimum volume and partitioned between 1.0 N aqueous sodium hydroxide and diethyl ether. The aqueous layer was collected, acidified with concentrated hydrochloric acid and extracted with ethyl acetate. The organic layer was collected, dried over magnesium sulfate, filtered and concentrated to provide the title compounds (0.067 gm, 0.194 mmol, 99% yield).

LCMS (ESI+): 346 (MH+).

[(R, S), (R, R)]: ¹H-NMR (CDCl₃): δ 0.84 (t, 3H), 1.23 (t, 3H), 1.39 (m, 2H), 1.94 (m, 1H), 2.59 (m, 1H), 3.84 (t, 1H), 4.19 (m, 2H), 4.62 (m, H), 7.49 (m, 2H), 7.63 (d, 1H).

### Preparation 31

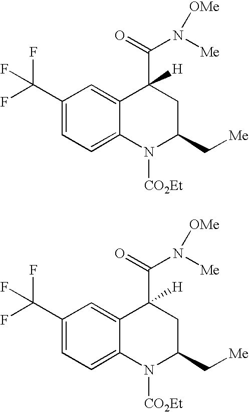

### [(R, S) and (R, R)]-2-Ethyl-4-(methoxy-methyl-carbamoyl)-6-trifluoromethyl-3,4-dihydro-2H-quinoline-1-carboxylic Acid Ethyl Ester

[(R, S) and (R, R)]-2-Ethyl-6-trifluoromethyl-3,4-dihydro-2H-quinoline-1,4-dicarboxylic acid-1-ethyl ester (0.066 gm, 0.239 mmol) was placed in a 5 mL round bottomed flask equipped with stir bar. Methylene chloride (3 mL) was added. The reaction mixture was cooled to 0° C. Di-iso-propylethylamine (0.032 gm, 0.248 mmol, 1.3 eq) was added followed by addition of N,O-dimethylhydroxylamine HCl salt (0.239 mmol), 4-dimethylaminopyridine (0.019 mmol) and 1-(3-dimethylaminopropyl)-3-ethylcarbodiimide hydrochloride (0.210 mmol). The reaction was stirred at room temperature. After 12 hr, the reaction was quenched with water and extracted 3 times with ethyl acetate. The organic layers were collected, dried over sodium sulfate, filtered, concentrated to provide the title compounds (0.065 gm, 0.167 mmol, 88% yield).

LCMS (ESI+): 389 (MH+).

[(R, S), (R, R)]: ¹H-NMR (CDCl₃): δ 0.90 (t, 3H), 1.33 (t, 3H), 1.39 (m, 2H), 2.0 (m, 1H), 2.55 (m, 1H), 3.20 (s, 3H), 3.40 (s, 3H), 4.19 (m, 2H), 4.62 (m, 1H), 7.21 (s, 1H), 7.40 (s, 2H), 7.62 (d, 2H).

### EXAMPLES 20 AND 21

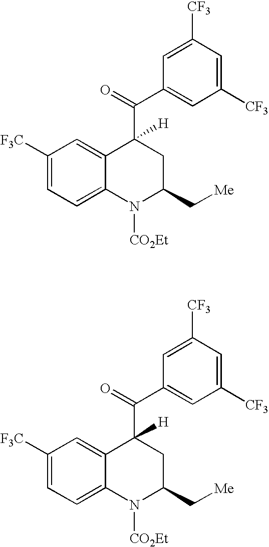

### [(R, S)] and [(R, R)]-4-(3,5-Bis-trifluoromethyl-benzoyl)-2-ethyl-6-trifluoromethyl-3,4-dihydro-2H-quinoline-1-carboxylic Acid Ethyl Ester

[(R, S), (R, R)]-2-Ethyl-4-(methoxy-methyl-carbamoyl)-6-trifluoromethyl-3,4-dihydro-2H-quinoline-1-carboxylic acid ethyl ester (0.564 gm 1.45 mmol, 1 eq) was dissolved in tetrahydrofuran (12 mL) in a round bottomed flask equipped with a stir bar. The reaction was cooled to 0° C. 3,5-Bis(trifluoromethyl)phenyl magnesium bromide (13.6 mL of 0.5M solution) was added dropwise, and the reaction mixture was stirred at room temperature. After 12 hr, the reaction mixture was quenched with aqueous ammonium chloride solution, further saturated with NaCl and extracted 3 times with ethyl acetate. The organic layers were collected, dried over sodium sulfate, filtered and concentrated to dryness. Flash chromatography eluting with 90/10 hexanes/ethyl acetate provided the title compounds (0.556 gm, 1.02 mmol, 70% yield).

trans isomer:

LCMS (ESI+): 542 (MH+).

(R, R): ¹H-NMR (CDCl₃): δ 0.98 (t, 3H), 1.29 (t, 3H), 1.52 (m, 2H), 2.05 (m, 1H), 2.64 (m, 1H), 4.24 (m, 2H), 4.66 (m, 2H), 7.40 (s, 1H), 7.55 (d, 1H), 7.60 (d, 1H), 8.05 (s, 2H), 8.24 (s, 1H).

cis isomer:

LCMS (ESI+): 542 (MH+).

(R, S): ¹H-NMR (CDCl₃): δ 0.91 (t, 3H), 1.39 (t, 3H), 1.43 (m, 2H), 1.78 (m, 1H), 1.99 (m, 1H), 2.60 (m, 1H), 4.34 (m, 2H), 4.56 (m, 2H), 7.05 (s, 1H), 7.55 (d, 1H), 7.65 (d, 1H), 8.20 (s, 1H), 8.44 (s, 2H).

### EXAMPLES 22, 23, 24 AND 25

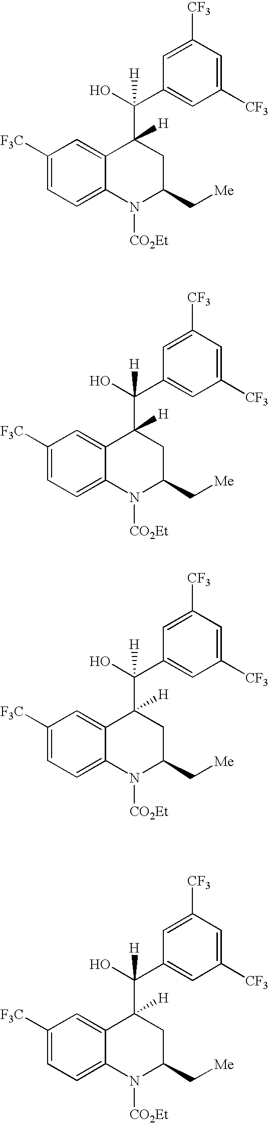

### [(R, R, R)], [(R, R, S)], [(R, S, S)], and [(R, S, R)]-4-(3,5-bis-trifluoromethyl-phenyl)-hydroxy-methyl]-2-ethyl-6-trifluoromethyl-3,4-dihydro-2H-quinoline-1-carboxylic Acid Ethyl Ester

[(R, S)] and [(R, R)]-4-(3,5-Bis-trifluoromethyl-benzoyl)-2-ethyl-6-trifluoromethyl-3,4-dihydro-2H-quinoline-1-carboxylic acid ethyl ester (0.182 gm, 0.34 mmol, 1 eq) was placed in a round bottomed flask equipped with a magnetic stir bar. Methanol (6.8 mL) was added followed by the addition of (0.077 gm, 2.05 mmol, 6 eq) sodium borohydride at room temperature. After 1 hour, the reaction mixture was quenched with brine solution, extracted 3 times with ethyl acetate and dried over sodium sulfate. The material was purified by flash chromatography eluting with 90/10 hexanes/ethyl acetate to provide the title compounds in three fractions.

(R,S,R):

LCMS (ESI+): 544 (MH+).

¹H-NMR (CDCl₃): δ 0.81 (t, 3H), 1.27 (t, 3H), 2.04 (m, 1H), 4.22 (m, 3H), 7.86 (s, 1H), 7.90 (s, 1H).

(R,S,S):

LCMS (ESI+): 544 (MH+).

¹H-NMR (CDCl₃): δ 0.73 (t, 3H), 1.09 (m, 1H), 1.27 (t, 3H), 1.43 (m, 2H), 1.67 (m, 1H), 2.44 (d, 1H), 2.87 (m, 1H), 4.24 (m, 3H), 5.17 (dd, 1H), 7.26 (s, 1H), 7.50 (m, 2H), 7.90 (s, 2H), 7.92 (s, 1H).

(R,R,R) and (R,R,S): This mixture was separated on a 10 cm by 50 cm, Chiralpak AD column eluting in heptane/IPA 98//2 at a flow rate of 275 mL/min.

(R,R,R): ¹H-NMR (CDCl₃): δ 0.77 (t, 3H), 1.31 (t, 3H), 1.41 (m, 2H), 2.21 (d, 1H), 3.19 (m, 1H), 4.23 (m, 2H), 4.37 (m, 1H), 4.83 (d, 1H), 7.65 (s, 2H), 7.79 (s, 1H).

LCMS (ESI+): 544 (MH+).

(R,R,S): ¹H-NMR (CDCl₃): δ 0.81 (t, 3H), 1.33 (t, 3H), 1.6 (m, 2H), 2.22 (d, 1H), 3.05 (m, 1H), 4.30 (m, 2H), 4.62 (m, 1H), 5.19 (d, 1H), 7.01 (s, 1H), 7.40 (s, 2H), 7.85 (s, 1H).

LCMS (ESI+): 544 (MH+).

### Preparations 32 and 33

### [(R, R), (S, S)] and [(S, R), (R, S)]-4-Aminomethyl-6,7-dimethoxy-2-methyl-3,4-dihydro-2H-quinoline-1-carboxylic Acid Ethyl Ester

Sodium borohydride (0.828 gm, 21.9 mmol) was suspended in tetrahydrofuran (13.5 mL) in a dry 50 mL round-bottomed flask equipped with stir bar. In another flask trifluoroacetic acid (1.68 mL, 21.8 mmol) was dissolved in tetrahydrofuran (5 mL). The 50 mL reaction flask was cooled to 0° C. and the trifluoroacetic acid solution was added slowly. The reaction was allowed to stir at room temperature. After 30 min, [(R, R), (S, S)] and [(S, R), (R, S)]-4-cyano-6,7-dimethoxy-2-methyl-3,4-dihydro-2H-quinoline-1-carboxylic acid ethyl ester (1.33 gm, 4.37 mmol) in tetrahydrofuran (5 mL) was added drop wise to the reaction. After 12 hr, the reaction was cooled back to 0° C. and carefully quenched with water. The aqueous layer was extracted 2 times with ethyl acetate. The organic layers were combined, dried over magnesium sulfate, filtered and concentrated. The crude product was purified by flash chromatography eluting with 97/2/1 methylene chloride/methanol/triethylamine to afford the title compounds (0.982 gm, 3.18 mmol, 73% yield).

LCMS (ESI+): 309 (MH+).

[(R, S), (S, R)] ¹H-NMR (CDCl₃): δ 1.18 (d, 3H), 1.27 (t, 3H), 2.47 (m, 2H), 2.92 (m, 1H), 3.29 (m, 1H), 3.84 (s, 3H), 3.86 (s, 3H), 4.19 (m, 2H), 4.47 (m, 1H), 6.69 (s, 1H), 7.02 (s, 1H).

LCMS (ESI+): 309 (MH+).

[(R, R), (S, S)] ¹H-NMR (CDCl₃): δ 1.15 (d, 3H), 1.31 (t, 3H), 1.76 (m, 1H), 2.18 (m, 1H), 2.94 (m, 3H), 3.85 (s, 3H), 3.86 (s, 3H), 4.22 (m, 2H), 4.61 (m, 1H), 6.69 (s, 1H), 7.12 (s, 1H).

### EXAMPLE 26

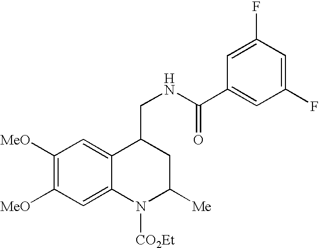

### [(R, S), (R, R)] and [(S, S), (S, R)]-4-[(3,5-Difluoro-benzoylamino)-methyl]-6,7-dimethoxy-2-methyl-3,4-dihydro-2H-quinoline-1-carboxylic Acid Ethyl Ester

To a solution of [(R, S), (R, R)] and [(S, S), (S, R)]-4-aminomethyl-6,7-dimethoxy-2-methyl-3,4-dihydro-2H-quinoline-1-carboxylic acid ethyl ester (0.012 gm, 0.039 mmol) in methylene chloride (1.0 mL) was added 3,5-difluorobenzoic acid (0.0065 gm, 0.041 mmol, 1.0 eq) followed by 1-(3-dimethylaminopropyl)-3-ethylcarbodiimide hydrochloride (0.012 gm, 0.065 mmol, 1.6 eq) and 1-hydroxybenzotriazole hydrate (0.005 gm, 0.039 mmol, 1 eq). The reaction was stirred at ambient temperature for 12 hr. The mixture was concentrated, dissolved in dimethylsulfoxide and purified by HPLC to provide the title compound (0.008 gm, 0.019 mmol).

LCMS (ESI+): 449 (MH+).

[(R, S), (R, R)] and [(S, S), (S, R)]: ¹H-NMR (CDCl₃): δ 1.15 (d, 3H), 1.30 (t, 3H), 1.79 (m, 1H), 2.16 (m, 1H), 3.16 (m, 1H), 3.63 (m, 2H), 3.82 (s, 3H), 3.87 (s, 3H), 4.22 (m, 2H), 4.63 (m, 1H), 6.13 (m, 1H), 6.63 (s, 1H), 6.92 (m, 1H), 7.13 (s, 1H), 7.19 (m, 2H).

### EXAMPLES 27 AND 28

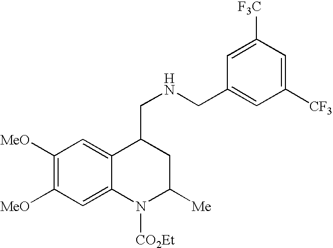

### [(R, S), (S, R)] and [(R, R), (S, S)]-4-[(3,5-Bis-trifluoromethyl-benzylamino)-methyl]-6,7-dimethoxy-2-methyl-3,4-dihydro-2H-quinoline-1-carboxylic Acid Ethyl Ester

[(R, S), (S, R)] and [(S, S), (R, R)]-4-Aminomethyl-6,7-dimethoxy-2-methyl-3,4-dihydro-2H-quinoline-1-carboxylic acid ethyl ester (0.131 gm, 0.428 mmol) was dissolved in dichloroethane (2.0 mL) in a 10 mL round bottomed flask equipped with a stir bar. To this solution, 3,5-bis(trifluoromethyl)benzaldehyde (0.071 mL, 0.431 mmol) was added followed by sodium triacetoxyborohydride (0.272 gm, 1.28 mmol). After stirring at ambient temperature for 12 hr, the reaction mixture was quenched with a 1.0 N aqueous sodium hydroxide solution and extracted 3 times with ethyl acetate. The organic layers were collected, dried over sodium sulfate, filtered and concentrated. Purification by flash chromatography eluting with 80/20 hexanes/ethyl acetate provided the title compounds (0.063 gm, 0.12 mmol, 28% yield).

LCMS (ESI+): 535 (MH+).

[(R, S) (S, R)] ¹H-NMR (CDCl₃): δ 1.09 (m, 1H), 1.17 (d, 3H), 1.28 (t, 3H), 2.47 (m, 1H), 2.61 (m, 1H), 2.83 (m, 1H), 3.16 (m, 1H), 3.83 (s, 3H), 3.84 (s, 3H), 4.02 (s, 2H), 4.20 (m, 2H), 4.47 (m, 1H), 6.81 (s, 1H), 7.01 (s, 1H), 7.82 (s, 1H), 7.87 (s, 2H).

LCMS (ESI+): 535 (MH+).

[(R, R) (S, S)] ¹H-NMR (CDCl₃): δ 1.14 (d, 3H), 1.28 (t, 3H), 1.74 (m, 1H), 2.21 (m, 1H), 2.79 (m, 2H), 2.93 (m, 1H), 3.83 (s, 3H), 3.84 (s, 3H), 3.88 (s, 2H), 4.20 (m, 2H), 4.56(s, 1H), 6.69 (s, 1H), 7.11 (s, 1H), 7.75 (s, 1H), 7.79 (s, 2H).

### EXAMPLES 29 AND 30

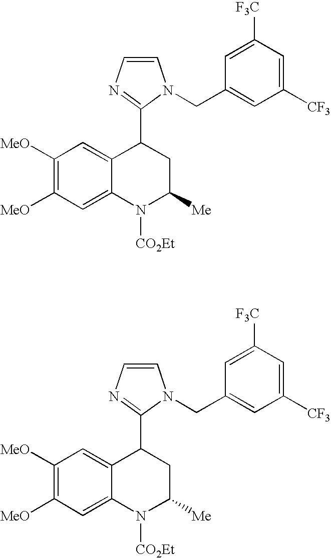

### [(R, S), (S, R)] and [(R, R), (S, S)]-4-[(3,5-Bis-trifluoromethyl-benzyl)-methoxycarbonyl-amino]-6,7-dimethoxy-2-methyl-3,4-dihydro-2H-quinoline-1-carboxylic Acid Ethyl Ester

[(R, S), (S, R)] and [(S, S), (R, R)]-4-[(3,5-Bis-trifluoromethyl-benzylamino)-methyl]-6,7-dimethoxy-2-methyl-3,4-dihydro-2H-quinoline-1-carboxylic acid ethyl ester (0.020 gm, 0.037 mmol) was dissolved in tetrahydrofuran (2.0 mL) in a 10 mL round bottomed flask equipped with a stir bar. To this solution, potassium carbonate (0.134 gm, 0.972 mmol) was added followed by the addition of methyl chloroformate (0.030 mL, 0.388 mmol). The reaction mixture was stirred at room temperature. After 12 hr, the reaction mixture was quenched with 1.0 N aqueous sodium hydroxide and extracted 3 times with ethyl acetate. The organic layers were collected, dried over sodium sulfate, filtered and concentrated. Purification by flash chromatography eluting with 65/35 hexanes/ethyl acetate provided the title compounds (0.005 gm, 0.008mmol, 30% yield).

LCMS (ESI+): 593 (MH+).

(R, S) (S, R)] ¹H-NMR (CDCl₃): δ 1.17 (d, 3H), 1.29 (t, 3H), 2.30 (m, 1H), 2.75 (m, 1H), 3.60 (m, 1H), 3.78 (s, 3H), 3.83 (s, 3H), 3.85 (s, 3H), 4.22 (m, 2H), 4.39 (m, 1H), 7.03 (s, 1H).

LCMS (ESI+): 593 (MH+).

[(R, R) (S, S)] ¹H-NMR (CDCl₃): δ 1.15 (d, 3H), 1.24 (m, 3H), 1.63 (m, 1H), 2.15 (m, 1H), 3.78 (s, 3H), 3.83 (s, 3H), 3.86 (s, 3H), 4.17 (m, 2H), 4.53 (m, 1H), 7.06 (s, 1H).

The following examples were prepared from analogous starting materials using methods analogous to those described in the examples above:

### EXAMPLE 31

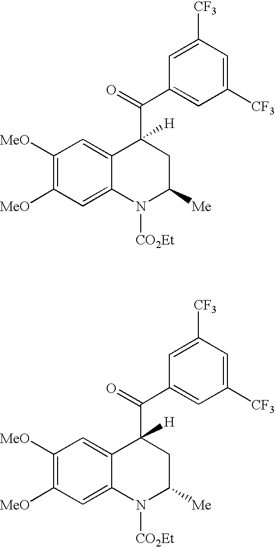

### [(R, S), (S, R)]-4-[(3,5-Bis-trifluoromethyl-benzoyl)-6,7-dimethoxy-2-methyl-3,4-dihydro-2H-quinoline-1-carboxylic Acid Ethyl Ester

¹H-NMR (CDCl₃): δ 1.2 (d, 3H), 1.35 (t, 3H), 1.95 (m, 1H), 2.55 (m, 1H), 3.60 (s, 3H), 3.97 (s, 3H), 4.20 (m, 1H), 4.22 (m, 1H), 4.40 (m, 1H), 4.62 (m, 1H), 6.2 (s, 1H), 7.1 (s, 1H) 8.19 (s, 1H), 8.45 (s, 2H).

LCMS (ESI+): 522 (MH+).

### EXAMPLE 32

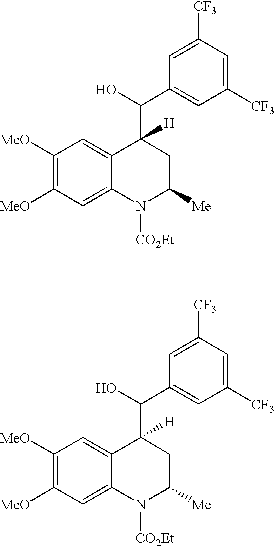

### [(R, S, S), (S, R, R), (R, S, R), (S, R, S)]-4-[(3,5-Bis-trifluoromethyl-phenyl)-hydroxy-methyl]-6,7-dimethoxy-2-methyl-3,4-dihydro-2H-quinoline-1-carboxylic Acid Ethyl Ester

¹H-NMR (CDCl₃): δ 1.13 (d, 3H), 1.34 (t, 3H), (m, 1H), 2.97 (m, 1H), 3.87 (s, 3H), 3.88 (s, 3H), 4.26 (m, 2H), 4.40 (m, 1H), 5.05 (d, 1H), 6.89 (s, 1H), 6.99 (s, 1H), 7.84 (s, 3H).

LCMS (ESI+): 522 (MH+).

### EXAMPLE 33

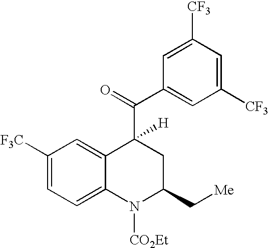

### (R, S)-4-(3,5-Bis-trifluoromethyl-benzoyl)-2-ethyl-6-trifluoromethyl-3,4-dihydro-2H-quinoline-1-carboxylic Acid Ethyl Ester

(R, S)]-cis: ¹H-NMR (CDCl₃): δ 0.91 (t, 3H), 1.39 (t, 3H), 1.43 (m, 2H), 1.78 (m, 1H), 1.99 (m, 1H), 2.60 (m, 1H), 4.34 (m, 2H), 4.56 (m, 2H), 7.05 (s, 1H), 7.55 (d, 1H), 7.65 (d, 1H), 8.20 (s, 1H), 8.44 (s, 2H).

LCMS (ESI+): 542 (MH+).

### EXAMPLE 34

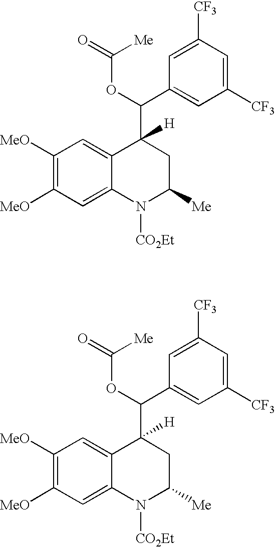

### [(R, R, S), (S, S, R), (R, R, R), (S, S, S)]-4-[Acetoxy-(3,5-Bis-trifluoromethyl-phenyl)-methyl]-6,7-dimethoxy-2-methyl-3,4-dihydro-2H-quinoline-1-carboxylic Acid Ethyl Ester

¹H-NMR (CDCl₃): δ 1.10 (d, 3H), 1.29 (t, 3H), 1.62 (m, 1H), 1.87 (m, 1H), 2.00 (s, 3H), 3.22 (m, 1H), 3.84 (s, 3H), 3.86 (s, 3H), 4.21 (m, 2H), 4.47 (m, 1H), 5.89 (d, 1H), 6.62 (s, 1H), 7.03 (s, 1H), 7.66 (s, 2H), 7.80 (s, 1H).

LCMS (ESI+): 564 (MH+).

### EXAMPLE 35

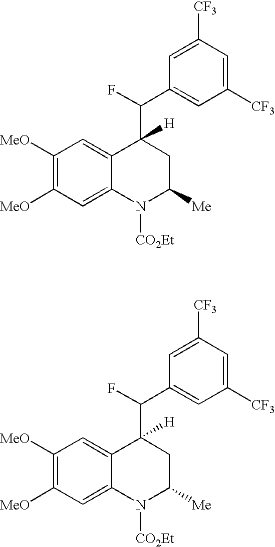

### [(R,R,R) (S,S,S) and (R,R,S) and (S,S,R)]-4-[(3,5-Bis-trifluoromethyl-phenyl)-fluoro-methyl]-6,7-dimethoxy-2-methyl-3,4-dihydro-2H-quinoline-1-carboxylic Acid Ethyl Ester

¹H-NMR (CDCl₃): δ 1.19 (d, 3H), 1.30 (t, 3H), 2.02 (m, 1H), 2.95 (m, 1H), 3.95 (s, 3H), 3.97 (s, 3H), 4.26 (m, 2H), 4.49 (m, 1H), 6.40 (s, 1H), 6.9 (s, 1H), 7.1 (s, 1H), 7.8 (s, 3 H), 7.95 (s, 1H).

LCMS (ESI+): 524 (MH+).

### EXAMPLE 36

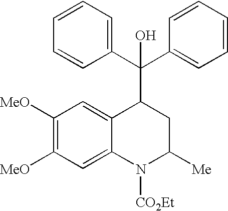

### [(R, R), (S, S), (R, S), (S, R)]-4-(Hydroxy-diphenyl-methyl)-6,7-dimethoxy-2-methyl-3,4-dihydro-2H-quinoline-1-carboxylic Acid Ethyl Ester

¹H-NMR (CDCl₃): δ 1.0 (d, 3H), 1.32 (t, 3H), 2.2 (m, 1H), 3.90 (s, 3H), 3.95 (s, 3H), 4.26 (m, 2H), 4.6 (m, 1H), 6.60 (s, 1H), 6.9 (s, 1H), 7.0 (s, 1H), 7.2-7.4 (m, 10H).

LCMS (ESI+): 444 (MH+) (minus 17 OH group).

### EXAMPLE 37

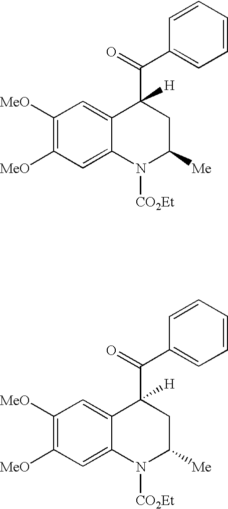

### [(R, R), (S, S)-4-benzoyl-6,7-dimethoxy-2-methyl-3,4-dihydro-2H-quinoline-1-carboxylic Acid Ethyl Ester

¹H-NMR (CDCl₃): δ 1.19 (d, 3H), 1.25 (t, 3H), 2.4 (m, 1H), 3.75 (s, 3H), 3.85 (s, 3H), 4.30 (m, 2H), 4.6 (m, 1H), 4.9 (m, 1H), 6.50 (s, 1H), 7.2 (s, 1H), 7.4 (m, 2H), 7.5 (t, 1H), 7.9 (d, 2H).

LCMS (ESI+): 384 (MH+).

### EXAMPLE 38

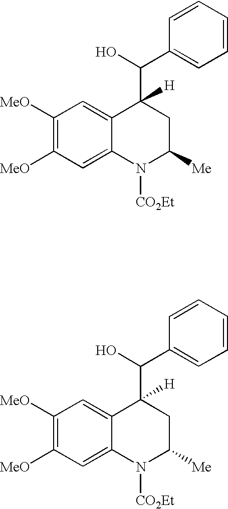

### [(R, S, S), (R, S, R), (S, R, R), (S, R, S)] 4-(Hydroxy-phenyl-methyl)-6,7-dimethoxy-2-methyl-3,4-dihydro-2H-quinoline-1-carboxylic Acid Ethyl Ester

LCMS (ESI+): 386(MH+)

Examples 39-169 in Table A were prepared as racemic mixtures using analogous methods as described above from the appropriate starting materials and have the following structure:

TABLE A

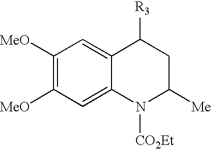

| | | | LCMS |
|---|---|---|---|
| | R³(• denotes link to | | ESI+ |
| Ex. | structure) | Compound | (MH+) |
| 39 | 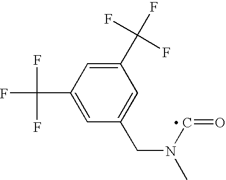 | (4-[(3,5-Bis-trifluoromethyl-benzyl)- methyl-carbamoyl]-6,7-dimethoxy- 2-methyl-3,4-dihydro-2H-quinoline- 1-carboxylic acid ethyl ester) | 563 |
| 40 | 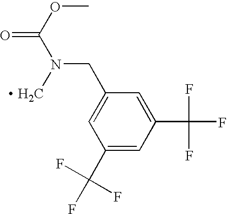 | 4-{[(3,5-Bis-trifluoromethyl-benzyl)- methoxycarbonyl-amino]-methyl}- 6,7-dimethoxy-2-methyl-3,4- dihydro-2H-quinoline-1-carboxylic acid ethyl ester | 593 |
| 41 | 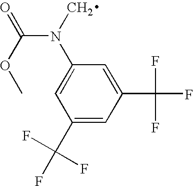 | 4-[(3,5-Bis-trifluoromethyl-phenyl)- methoxy-carbonyl-amino]-methyl}- 6,7-dimethoxy-2-methyl-3,4- dihydro-2H-quinoline-1-carboxylic acid ethyl ester | 579 |
| 42 | 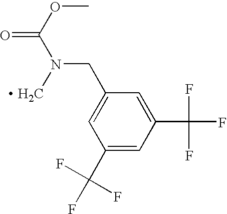 | 4-{[(3,5-Bis-trifluoromethyl-benzyl)- methoxy-carbonyl-amino]-methyl}- 6,7-dimethoxy-2-methyl-3,4- dihydro-2H-quinoline-1-carboxylic acid ethyl ester | 593 |
| 43 | 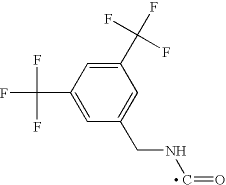 | 4-(3,5-Bis-trifluoromethyl- benzylcarbamoyl)-6,7-dimethoxy-2- methyl-3,4-dihydro-2H-quinoline-1- carboxylic acid ethyl ester | 549 |
| 44 | 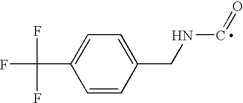 | 6,7-Dimethoxy-2-methyl-4-(4- trifluoromethyl-benzylcarbamoyl)- 3,4-dihydro-2H-quinoline-1- carboxylic acid ethyl ester | 481 |
| 45 | 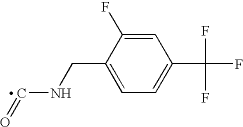 | 4-(2-Fluoro-4-trifluoromethyl- benzylcarbamoyl)-6,7-dimethoxy-2- methyl-3,4-dihydro-2H-quinoline-1- carboxylic acid ethyl ester | 499 |
| 46 | 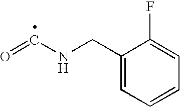 | 4-(2-Fluoro-benzylcarbamoyl)-6,7- dimethoxy-2-methyl-3,4-dihydro- 2H-quinoline-1-carboxylic acid ethyl ester | 431 |
| 47 | 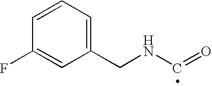 | 4-(3-Fluoro-benzylcarbamoyl)-6,7- dimethoxy-2-methyl-3,4-dihydro- 2H-quinoline-1-carboxylic acid ethyl ester | 431 |
| 48 | 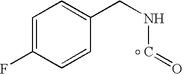 | 4-(4-Fluoro-benzylcarbamoyl)-6,7- dimethoxy-2-methyl-3,4-dihydro- 2H-quinoline-1-carboxylic acid ethyl ester | 431 |
| 49 | 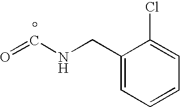 | 4-(2-Chloro-benzylcarbamoyl)-6,7- dimethoxy-2-methyl-3,4-dihydro- 2H-quinoline-1-carboxylic acid ethyl ester | 447 |
| 50 | 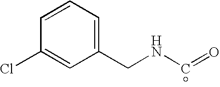 | 4-(3-Chloro-benzylcarbamoyl)-6,7- dimethoxy-2-methyl-3,4-dihydro- 2H-quinoline-1-carboxylic acid ethyl ester | 447 |
| 51 | 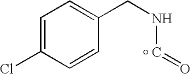 | 4-(4-Chloro-benzylcarbamoyl)-6,7- dimethoxy-2-methyl-3,4-dihydro- 2H-quinoline-1-carboxylic acid ethyl ester | 447 |
| 52 | 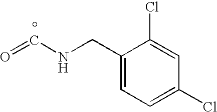 | 4-(2,4-Dichloro-benzylcarbamoyl)- 6,7-dimethoxy-2-methyl-3,4- dihydro-2H-quinoline-1-carboxylic acid ethyl ester | 481 |
| 53 | 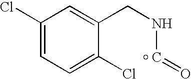 | 4-(2,5-Dichloro-benzylcarbamoyl)- 6,7-dimethoxy-2-methyl-3,4- dihydro-2H-quinoline-1-carboxylic acid ethyl ester | 481 |
| 54 | 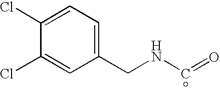 | 4-(3,4-Dichloro-benzylcarbamoyl)- 6,7-dimethoxy-2-methyl-3,4- dihydro-2H-quinoline-1-carboxylic acid ethyl ester | 481 |
| 55 | 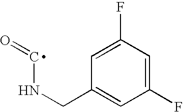 | 4-(3,5-Difluoro-benzylcarbamoyl)- 6,7-dimethoxy-2-methyl-3,4- dihydro-2H-quinoline-1-carboxylic acid ethyl ester | 449 |
| 56 | 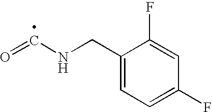 | 4-(2,4-Difluoro-benzylcarbamoyl)- 6,7-dimethoxy-2-methyl-3,4- dihydro-2H-quinoline-1-carboxylic acid ethyl ester | 449 |
| 57 | 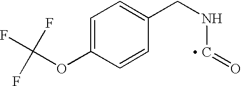 | 6,7-Dimethoxy-2-methyl-4-(4- trifluoromethoxy- benzylcarbamoyl)-3,4-dihydro-2H- quinoline-1-carboxylic acid ethyl ester | 497 |
| 58 | 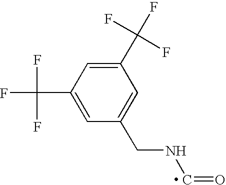 | 4-(3,5-Bis-trifluoromethyl- benzylcarbamoyl)-6,7-dimethoxy- 2-methyl-3,4-dihydro-2H- quinoline-1-carboxylic acid ethyl ester | 549 |
| 59 | 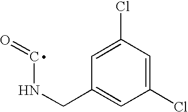 | 4-(3,5-Dichloro-benzylcarbamoyl)- 6,7-dimethoxy-2-methyl-3,4- dihydro-2H-quinoline-1-carboxylic acid ethyl ester | 481 |
| 60 | 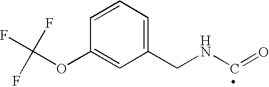 | 6,7-Dimethoxy-2-methyl-4-(3- trifluoromethoxy- benzylcarbamoyl)-3,4-dihydro-2H- quinoline-1-carboxylic acid ethyl ester | 497 |
| 61 | 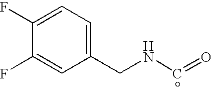 | 4-(3,4-Difluoro-benzylcarbamoyl)- 6,7-dimethoxy-2-methyl-3,4- dihydro-2H-quinoline-1-carboxylic acid ethyl ester | 449 |
| 62 | 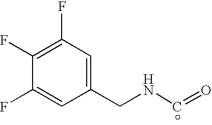 | 6,7-Dimethoxy-2-methyl-4-(3,4,5- trifluoro-benzylcarbamoyl)-3,4- dihydro-2H-quinoline-1-carboxylic acid ethyl ester | 467 |
| 63 | 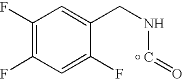 | 6,7-Dimethoxy-2-methyl-4-(2,4,5- trifluoro-benzylcarbamoyl)-3,4- dihydro-2H-quinoline-1-carboxylic acid ethyl ester | 467 |
| 64 | 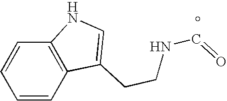 | 4-[2-(1H-Indol-3-yl)- ethylcarbamoyl]-6,7-dimethoxy-2- methyl-3,4-dihydro-2H-quinoline- 1-carboxylic acid ethyl ester | 466 |
| 65 | 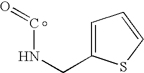 | 6,7-Dimethoxy-2-methyl-4- [(thiophen-2-ylmethyl)-carbamoyl]- 3,4-dihydro-2H-quinoline-1- carboxylic acid ethyl ester | 419 |
| 66 | 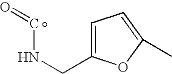 | 6,7-Dimethoxy-2-methyl-4-[(5- methyl-furan-2-ylmethyl)- carbamoyl]-3,4-dihydro-2H- quinoline-1-carboxylic acid ethyl ester | 417 |
| 67 | 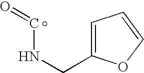 | 4-[(Furan-2-ylmethyl)-carbamoyl]- 6,7-dimethoxy-2-methyl-3,4- dihydro-2H-quinoline-1-carboxylic acid ethyl ester | 403 |
| 68 | 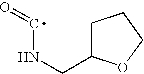 | 6,7-Dimethoxy-2-methyl-4- [(tetrahydro-furan-2-ylmethyl)- carbamoyl]-3,4-dihydro-2H- quinoline-1-carboxylic acid ethyl ester | 407 |
| 69 | 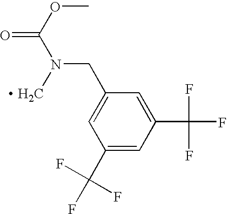 | 4-{[(3,5-Bis-trifluoromethyl-benzyl)- methoxy-carbonyl-amino]-methyl}- 6,7-dimethoxy-2-methyl-3,4- dihydro-2H-quinoline-1-carboxylic acid ethyl ester | 593 |
| 70 | 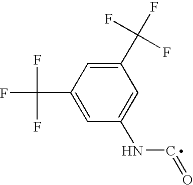 | 4-(3,5-Bis-trifluoromethyl- phenylcarbamoyl)-6,7-dimethoxy- 2-methyl-3,4-dihydro-2H- quinoline-1-carboxylic acid ethyl ester | 535 |
| 71 | 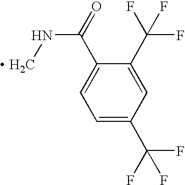 | 4-[(2,4-Bis-trifluoromethyl- benzoylamino)-methyl]-6,7- dimethoxy-2-methyl-3,4-dihydro- 2H-quinoline-1-carboxylic acid ethyl ester | 549 |
| 72 | 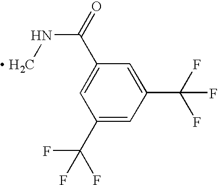 | 4-[(3,5-Bis-trifluoromethyl- benzoylamino)-methyl]-6,7- dimethoxy-2-methyl-3,4-dihydro- 2H-quinoline-1-carboxylic acid ethyl ester | 549 |
| 73 | 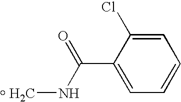 | 4-[(2-Chloro-benzoylamino)- methyl]-6,7-dimethoxy-2-methyl- 3,4-dihydro-2H-quinoline-1- carboxylic acid ethyl ester | 447 |
| 74 | 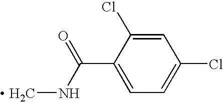 | 4-[(2,4-Dichloro-benzoylamino)- methyl]-6,7-dimethoxy-2-methyl- 3,4-dihydro-2H-quinoline-1- carboxylic acid ethyl ester | 481 |
| 75 | 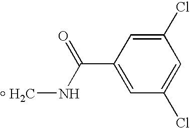 | 4-[(3,5-Dichloro-benzoylamino)- methyl]-6,7-dimethoxy-2-methyl- 3,4-dihydro-2H-quinoline-1- carboxylic acid ethyl ester | 481 |
| 76 | 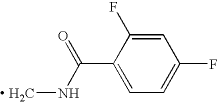 | 4-[(2,4-Difluoro-benzoylamino)- methyl]-6,7-dimethoxy-2-methyl- 3,4-dihydro-2H-quinoline-1- carboxylic acid ethyl ester | 449 |
| 77 | 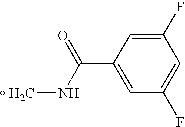 | 4-[(3,5-Difluoro-benzoylamino)- methyl]-6,7-dimethoxy-2-methyl- 3,4-dihydro-2H-quinoline-1- carboxylic acid ethyl ester | 449 |
| 78 | 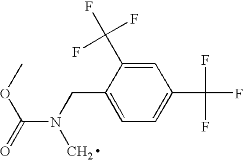 | 4-{[(2,4-Bis-trifluoromethyl- benzyl)-methoxycarbonyl-amino]- methyl}-6,7-dimethoxy-2-methyl- 3,4-dihydro-2H-quinoline-1- carboxylic acid ethyl ester | 593 |
| 79 | 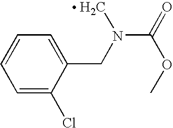 | 4-{[(2-Chloro-benzyl)- methoxycarbonyl-amino]-methyl}- 6,7-dimethoxy-2-methyl-3,4- dihydro-2H-quinoline-1-carboxylic acid ethyl ester | 491 |
| 80 | 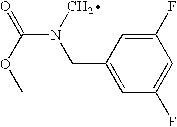 | 4-{[(3,5-Difluoro-benzyl)- methoxycarbonyl-amino]-methyl}- 6,7-dimethoxy-2-methyl-3,4- dihydro-2H-quinoline-1-carboxylic acid ethyl ester | 493 |
| 81 | 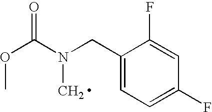 | 4-{[(2,4-Difluoro-benzyl)- methoxycarbonyl-amino]-methyl}- 6,7-dimethoxy-2-methyl-3,4- dihydro-2H-quinoline-1-carboxylic acid ethyl ester | 493 |
| 82 | 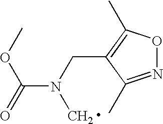 | 4-{[(3,5-Dimethyl-isoxazol-4- ylmethyl)-methoxycarbonyl- amino]-methyl}-6,7-dimethoxy-2- methyl-3,4-dihydro-2H-quinoline-1- carboxylic acid ethyl ester | 476 |
| 83 | 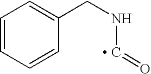 | 4-Benzylcarbamoyl-6,7-dimethoxy- 2-methyl-3,4-dihydro-2H-quinoline- 1-carboxylic acid ethyl ester | 413 |
| 84 | 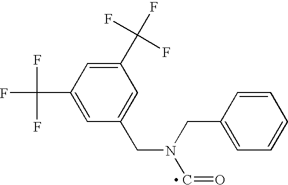 | 4-[Benzyl-(3,5-bis-trifluoromethyl- benzyl)-carbamoyl]-6,7-dimethoxy- 2-methyl-3,4-dihydro-2H- quinoline-1-carboxylic acid ethyl ester | 639 |
| 85 | 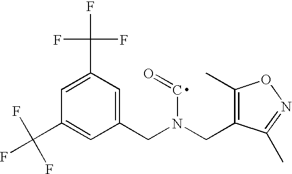 | 4-[(3,5-Bis-trifluoromethyl-benzyl)- (3,5-dimethyl-isoxazol-4-ylmethyl)- carbamoyl]-6,7-dimethoxy-2- methyl-3,4-dihydro-2H-quinoline-1- carboxylic acid ethyl ester | 658 |
| 86 | 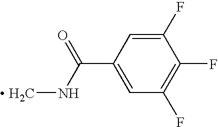 | 6,7-Dimethoxy-2-methyl-4-[(3,4,5- trifluoro-benzoylamino)-methyl]- 3,4-dihydro-2H-quinoline-1- carboxylic acid ethyl ester | 467 |
| 87 | 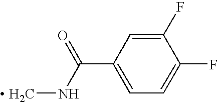 | 4-[(3,4-Difluoro-benzoylamino)- methyl]-6,7-dimethoxy-2-methyl- 3,4-dihydro-2H-quinoline-1- carboxylic acid ethyl ester | 449 |
| 88 | 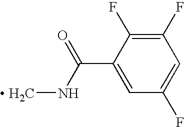 | 6,7-Dimethoxy-2-methyl-4-[(2,3,5- trifluoro-benzoylamino)-methyl]- 3,4-dihydro-2H-quinoline-1- carboxylic acid ethyl ester | 467 |
| 89 | 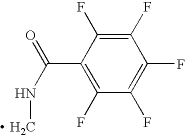 | 6,7-Dimethoxy-2-methyl-4- [(2,3,4,5,6- pentafluorobenzoylamino)-methyl]- 3,4-dihydro-2H-quinoline-1- carboxylic acid ethyl ester | 503 |
| 90 | 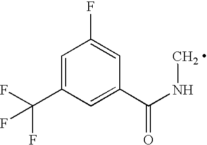 | 4-[(3-Fluoro-5-trifluoromethyl- benzoylamino)-methyl]-6,7- dimethoxy-2-methyl-3,4-dihydro- 2H-quinoline-1-carboxylic acid ethyl ester | 499 |
| 91 | 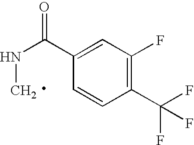 | 4-[(3-Fluoro-4-trifluoromethyl- benzoylamino)-methyl]-6,7- dimethoxy-2-methyl-3,4-dihydro- 2H-quinoline-1-carboxylic acid ethyl ester | 499 |
| 92 | 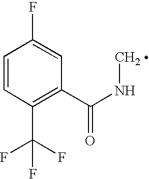 | 4-[(5-Fluoro-2-trifluoromethyl- benzoylamino)-methyl]-6,7- dimethoxy-2-methyl-3,4-dihydro- 2H-quinoline-1-carboxylic acid ethyl ester | 499 |
| 93 | 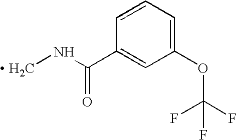 | 6,7-Dimethoxy-2-methyl-4-[(3- trifluoromethoxy-benzoylamino)- methyl]-3,4-dihydro-2H-quinoline- 1-carboxylic acid ethyl ester | 497 |
| 94 | 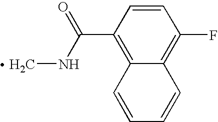 | 4-{[(4-Fluoro-naphthalene-1- carbonyl)-amino]-methyl}-6,7- dimethoxy-2-methyl-3,4-dihydro- 2H-quinoline-1-carboxylic acid ethyl ester | 481 |
| 95 | 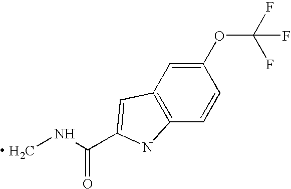 | 6,7-Dimethoxy-2-methyl-4-{[(5- trifluoromethoxy-1H-indole-2- carbonyl)-amino]-methyl}-3,4- dihydro-2H-quinoline-1-carboxylic acid ethyl ester | 536 |
| 96 | 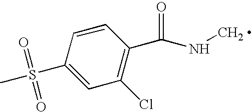 | 4-[(2-Chloro-4-methanesulfonyl- benzoylamino)-methyl]-6,7- dimethoxy-2-methyl-3,4-dihydro- 2H-quinoline-1-carboxylic acid ethyl ester | 525 |
| 97 | 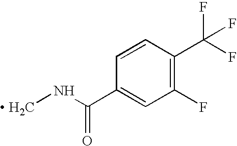 | 4-[(3-Fluoro-4-trifluoromethyl- benzoylamino)-methyl]-6,7- dimethoxy-2-methyl-3,4-dihydro- 2H-quinoline-1-carboxylic acid ethyl ester | 499 |
| 98 | 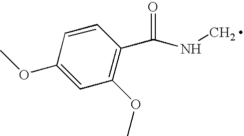 | 4-[(2,4-Dimethoxy-benzoylamino)- methyl]-6,7-dimethoxy-2-methyl- 3,4-dihydro-2H-quinoline-1- carboxylic acid ethyl ester | 473 |
| 99 | 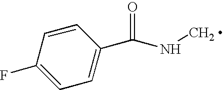 | 4-[(4-Fluoro-benzoylamino)- methyl]-6,7-dimethoxy-2-methyl- 3,4-dihydro-2H-quinoline-1- carboxylic acid ethyl ester | 431 |
| 100 | 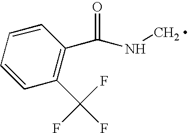 | 6,7-Dimethoxy-2-methyl-4-[(2- trifluoromethyl-benzoylamino)- methyl]-3,4-dihydro-2H-quinoline- 1-carboxylic acid ethyl ester | 481 |
| 101 | 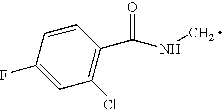 | 4-[(2-Chloro-4-fluoro- benzoylamino)-methyl]-6,7- dimethoxy-2-methyl-3,4-dihydro- 2H-quinoline-1-carboxylic acid ethyl ester | 465 |
| 102 | 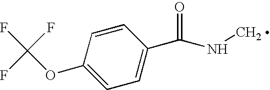 | 6,7-Dimethoxy-2-methyl-4-[(4- trifluoromethoxy-benzoylamino)- methyl]-3,4-dihydro-2H-quinoline- 1-carboxylic acid ethyl ester | 497 |
| 103 | 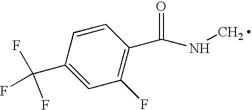 | 4-[(2-Fluoro-4-trifluoromethyl- benzoylamino)-methyl]-6,7- dimethoxy-2-methyl-3,4-dihydro- 2H-quinoline-1-carboxylic acid ethyl ester | 499 |
| 104 | 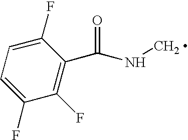 | 6,7-Dimethoxy-2-methyl-4-[(2,3,6- trifluoro-benzoylamino)-methyl]- 3,4-dihydro-2H-quinoline-1- carboxylic acid ethyl ester | 467 |
| 105 | 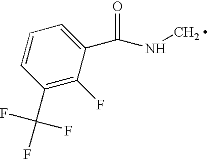 | 4-[(2-Fluoro-3-trifluoromethyl- benzoylamino)-methyl]-6,7- dimethoxy-2-methyl-3,4-dihydro- 2H-quinoline-1-carboxylic acid ethyl ester | 499 |
| 106 | 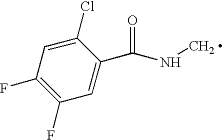 | 4-[(2-Chloro-4,5-difluoro- benzoylamino)-methyl]-6,7- dimethoxy-2-methyl-3,4-dihydro- 2H-quinoline-1- carboxylic acid ethyl ester | 483 |
| 107 | 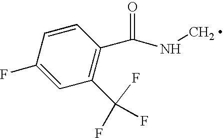 | 4-[(4-Fluoro-2-trifluoromethyl- benzoylamino)-methyl]-6,7- dimethoxy-2-methyl-3,4-dihydro- 2H-quinoline-1-carboxylic acid ethyl ester | 499 |
| 108 | 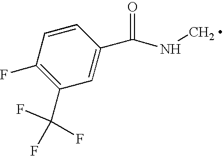 | 4-[(4-Fluoro-3-trifluoromethyl- benzoylamino)-methyl]-6,7- dimethoxy-2-methyl-3,4-dihydro- 2H-quinoline-1-carboxylic acid ethyl ester | 499 |
| 109 | 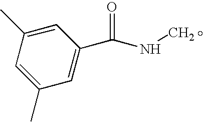 | 4-[(3,5-Dimethyl-benzoylamino)- methyl]-6,7-dimethoxy-2-methyl- 3,4-dihydro-2H-quinoline-1- carboxylic acid ethyl ester | 441 |
| 110 | 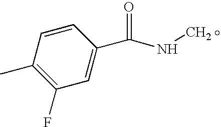 | 4-[(3-Fluoro-4-methyl- benzoylamino)-methyl]-6,7- dimethoxy-2-methyl-3,4-dihydro- 2H-quinoline-1-carboxylic acid ethyl ester | 445 |
| 111 | 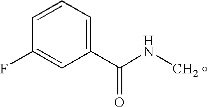 | 4-[(3-Fluoro-benzoylamino)- methyl]-6,7-dimethoxy-2-methyl- 3,4-dihydro-2H-quinoline-1- carboxylic acid ethyl ester | 431 |
| 112 | 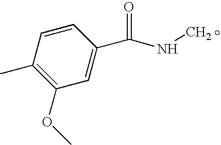 | 6,7-Dimethoxy-4-[(3-methoxy-4- methyl-benzoylamino)-methyl]-2- methyl-3,4-dihydro-2H-quinoline- 1-carboxylic acid ethyl ester | 457 |
| 113 | 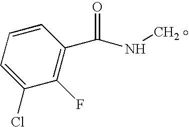 | 4-[(3-Chloro-2-fluoro- benzoylamino)-methyl]-6,7- dimethoxy-2-methyl-3,4-dihydro- 2H-quinoline-1-carboxylic acid ethyl ester | 465 |
| 114 | 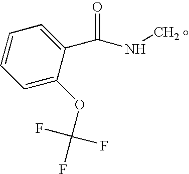 | 6,7-Dimethoxy-2-methyl-4-[(2- trifluoromethoxy-benzoylamino)- methyl]-3,4-dihydro-2H-quinoline- 1-carboxylic acid ethyl ester | 497 |
| 115 | 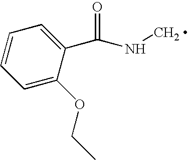 | 4-[(3-Ethoxy-benzoylamino)- methyl]-6,7-dimethoxy-2-methyl- 3,4-dihydro-2H-quinoline-1- carboxylic acid ethyl ester | 457 |
| 116 | 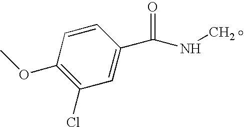 | 4-[(3-Chloro-4-methoxy- benzoylamino)-methyl]-6,7- dimethoxy-2-methyl-3,4-dihydro- 2H-quinoline-1-carboxylic acid ethyl ester | 477 |
| 117 | 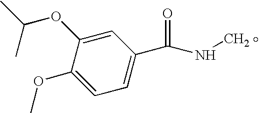 | 4-[(3-Isopropoxy-4-methoxy- benzoylamino)-methyl]-6,7- dimethoxy-2-methyl-3,4-dihydro- 2H-quinoline-1-carboxylic acid ethyl ester | 501 |
| 118 | 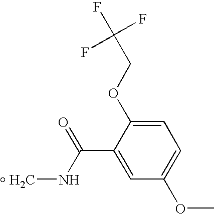 | 6,7-Dimethoxy-4-{[5-methoxy-2- (2,2,2-trifluoro-ethoxy)- benzoylamino]-methyl}-2-methyl- 3,4-dihydro-2H-quinoline-1- carboxylic acid ethyl ester | 541 |
| 119 | 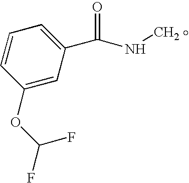 | 4-[(3-Difluoromethoxy- benzoylamino)-methyl]-6,7- dimethoxy-2-methyl-3,4-dihydro- 2H-quinoline-1-carboxylic acid ethyl ester | 479 |
| 120 | 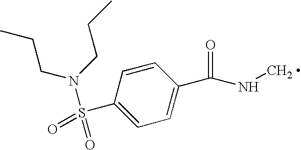 | 4-[(4-Dipropylsulfamoyl- benzoylamino)-methyl]-6,7- dimethoxy-2-methyl-3,4-dihydro- 2H-quinoline-1-carboxylic acid ethyl ester | 576 |
| 121 | 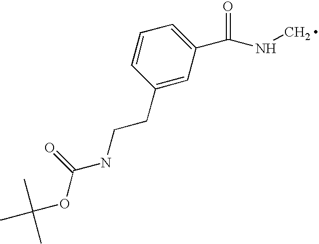 | 4-{[3-(2-tert-Butoxycarbonylamino- ethyl)-benzoylamino]-methyl}-6,7- dimethoxy-2-methyl-3,4-dihydro- 2H-quinoline-1-carboxylic acid ethyl ester | 556 |
| 122 | 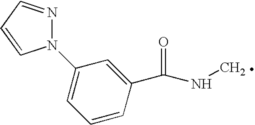 | 6,7-Dimethoxy-2-methyl-4-[(3- pyrazol-1-yl-benzoylamino)- methyl]-3,4-dihydro-2H-quinoline-1- carboxylic acid ethyl ester | 479 |
| 123 | 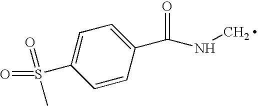 | 4-[(4-Methanesulfonyl- benzoylamino)-methyl]-6,7- dimethoxy-2-methyl-3,4-dihydro- 2H-quinoline-1-carboxylic acid ethyl ester | 491 |
| 124 | 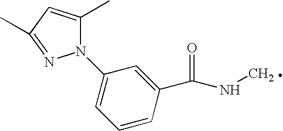 | 4-{[3-(3,5-Dimethyl-pyrazol-1-yl)- benzoylamino]-methyl}-6,7- dimethoxy-2-methyl-3,4-dihydro- 2H-quinoline-1-carboxylic acid ethyl ester | 507 |
| 125 | 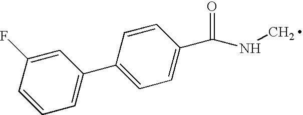 | 4-{[(3′-Fluoro-biphenyl-4-carbonyl)- amino]-methyl}-6,7-dimethoxy-2- methyl-3,4-dihydro-2H-quinoline-1- carboxylic acid ethyl ester | 507 |
| 126 | 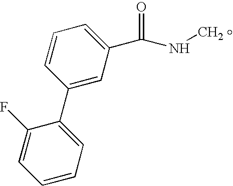 | 4-{[(2′-Fluoro-biphenyl-3-carbonyl)- amino]-methyl}-6,7-dimethoxy-2- methyl-3,4-dihydro-2H-quinoline-1- carboxylic acid ethyl ester | 507 |
| 127 | 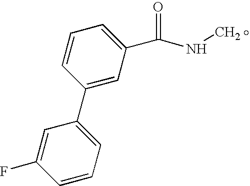 | 4-{[(3′-Fluoro-biphenyl-3-carbonyl)- amino]-methyl}-6,7-dimethoxy-2- methyl-3,4-dihydro-2H-quinoline-1- carboxylic acid ethyl ester | 507 |
| 128 | 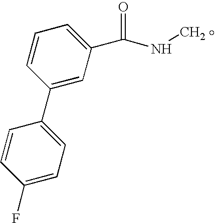 | 4-{[(4′-Fluoro-biphenyl-3-carbonyl)- amino]-methyl}-6,7-dimethoxy-2- methyl-3,4-dihydro-2H-quinoline-1- carboxylic acid ethyl ester | 507 |
| 129 | 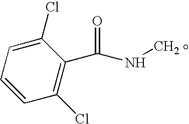 | 4-[(2,6-Dichloro-benzoylamino)- methyl]-6,7-dimethoxy-2-methyl- 3,4-dihydro-2H-quinoline-1- carboxylic acid ethyl ester | 481 |
| 130 | 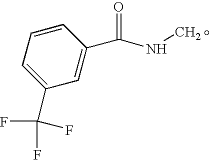 | 6,7-Dimethoxy-2-methyl-4-[(3- trifluoromethyl-benzoylamino)- methyl]-3,4-dihydro-2H-quinoline-1- carboxylic acid ethyl ester | 481 |
| 131 | 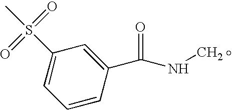 | 4-[(3-Methanesulfonyl- benzoylamino)-methyl]-6,7- dimethoxy-2-methyl-3,4-dihydro- 2H-quinoline-1-carboxylic acid ethyl ester | 491 |
| 132 | 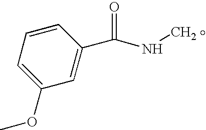 | 6,7-Dimethoxy-4-[(3-methoxy- benzoylamino)-methyl]-2-methyl- 3,4-dihydro-2H-quinoline-1- carboxylic acid ethyl ester | 443 |
| 133 | 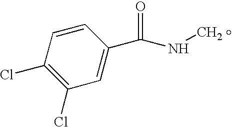 | 4-[(3,4-Dichloro-benzoylamino)- methyl]-6,7-dimethoxy-2-methyl- 3,4-dihydro-2H-quinoline-1- carboxylic acid ethyl ester | 481 |
| 134 | 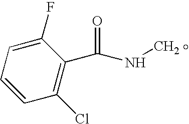 | 4-[(2-Chloro-6-fluoro- benzoylamino)-methyl]-6,7- dimethoxy-2-methyl-3,4-dihydro- 2H-quinoline-1-carboxylic acid ethyl ester | 465 |
| 135 | 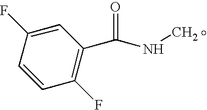 | 4-[(2,5-Difluoro-benzoylamino)- methyl]-6,7-dimethoxy-2-methyl- 3,4-dihydro-2H-quinoline-1- carboxylic acid ethyl ester | 449 |
| 136 | 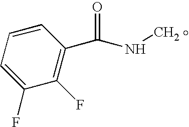 | 4-[(2,3-Difluoro-benzoylamino)- methyl]-6,7-dimethoxy-2-methyl- 3,4-dihydro-2H-quinoline-1- carboxylic acid ethyl ester | 449 |
| 137 | 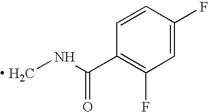 | 4-[(2,4-Difluoro-benzoylamino)- methyl]-6,7-dimethoxy-2-methyl- 3,4-dihydro-2H-quinoline-1- carboxylic acid ethyl ester | 449 |
| 138 | 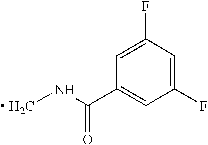 | 4-[(3,5-Difluoro-benzoylamino)- methyl]-6,7-dimethoxy-2-methyl- 3,4-dihydro-2H-quinoline-1- carboxylic acid ethyl ester | 449 |
| 139 | 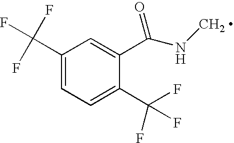 | 4-[(2,5-Bis-trifluoromethyl- benzoylamino)-methyl]-6,7- dimethoxy-2-methyl-3,4-dihydro- 2H-quinoline-1-carboxylic acid ethyl ester | 449 |
| 140 | 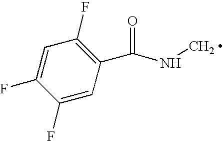 | 6,7-Dimethoxy-2-methyl-4-[(2,4,5- trifluoro-benzoylamino)-methyl]- 3,4-dihydro-2H-quinoline-1- carboxylic acid ethyl ester | 467 |
| 141 | 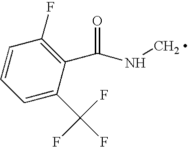 | 4-[(2-Fluoro-6-trifluoromethyl- benzoylamino)-methyl]-6,7- dimethoxy-2-methyl-3,4-dihydro- 2H-quinoline-1-carboxylic acid ethyl ester | 499 |
| 142 | 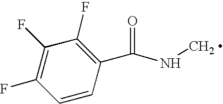 | 6,7-Dimethoxy-2-methyl-4-[(2,3,4- trifluoro-benzoylamino)-methyl]- 3,4-dihydro-2H-quinoline-1- carboxylic acid ethyl ester | 467 |
| 143 | 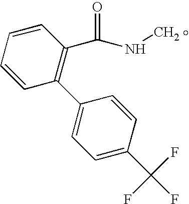 | 6,7-Dimethoxy-2-methyl-4-{[(4′- trifluoromethyl-biphenyl-2- carbonyl)-amino]-methyl}-3,4- dihydro-2H-quinoline-1-carboxylic acid ethyl ester | 557 |
| 144 | 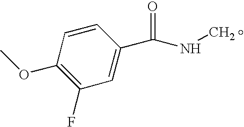 | 4-[(3-Fluoro-4-methoxy- benzoylamino)-methyl]-6,7- dimethoxy-2-methyl-3,4-dihydro- 2H-quinoline-1-carboxylic acid ethyl ester | 461 |
| 145 | 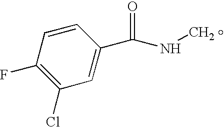 | 4-[(3-Chloro-4-fluoro- benzoylamino)-methyl]-6,7- dimethoxy-2-methyl-3,4-dihydro- 2H-quinoline-1-carboxylic acid ethyl ester | 465 |
| 146 | 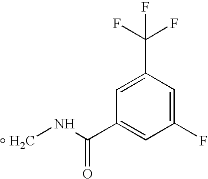 | 4-[(3-Fluoro-5-trifluoromethyl- benzoylamino)-methyl]-6,7- dimethoxy-2-methyl-3,4-dihydro- 2H-quinoline-1-carboxylic acid ethyl ester | 499 |
| 147 | 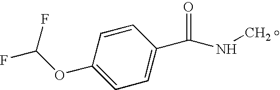 | 4-[(4-Difluoromethoxy- benzoylamino)-methyl]-6,7- dimethoxy-2-methyl-3,4-dihydro- 2H-quinoline-1-carboxylic acid ethyl ester | 479 |
| 148 | 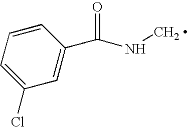 | 4-[(3-Chloro-benzoylamino)- methyl]-6,7-dimethoxy-2-methyl- 3,4-dihydro-2H-quinoline-1- carboxylic acid ethyl ester | 447 |
| 149 | 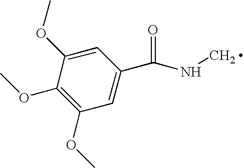 | 6,7-Dimethoxy-2-methyl-4-[(3,4,5- trimethoxy-benzoylamino)-methyl]- 3,4-dihydro-2H-quinoline-1- carboxylic acid ethyl ester | 503 |
| 150 | 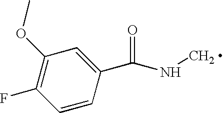 | 4-[(4-Fluoro-3-methoxy- benzoylamino)-methyl]-6,7- dimethoxy-2-methyl-3,4-dihydro- 2H-quinoline-1-carboxylic acid ethyl ester | 461 |
| 151 | 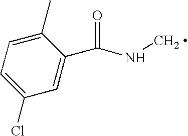 | 4-[(5-Chloro-2-methyl- benzoylamino)-methyl]-6,7- dimethoxy-2-methyl-3,4-dihydro- 2H-quinoline-1-carboxylic acid ethyl ester | 461 |
| 152 | 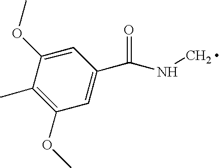 | 4-[(3,5-Dimethoxy-4-methyl- benzoylamino)-methyl]-6,7- dimethoxy-2-methyl-3,4-dihydro- 2H-quinoline-1-carboxylic acid ethyl ester | 487 |
| 153 | 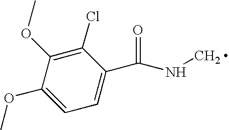 | 4-[(2-Chloro-3,4-dimethoxy- benzoylamino)-methyl]-6,7- dimethoxy-2-methyl-3,4-dihydro- 2H-quinoline-1-carboxylic acid ethyl ester | 507 |
| 154 | 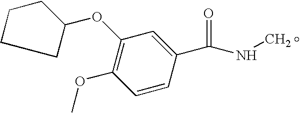 | 4-[(3-Cyclopentyloxy-4-methoxy- benzoylamino)-methyl]-6,7- dimethoxy-2-methyl-3,4-dihydro- 2H-quinoline-1-carboxylic acid ethyl ester | 527 |
| 155 | 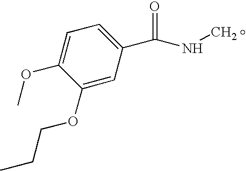 | 6,7-Dimethoxy-4-[(4-methoxy-3- propoxy-benzoylamino)-methyl]-2- methyl-3,4-dihydro-2H-quinoline-1- carboxylic acid ethyl ester | 501 |
| 156 | 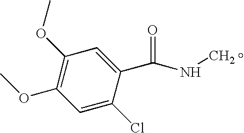 | 4-[(2-Chloro-4,5-dimethoxy- benzoylamino)-methyl]-6,7- dimethoxy-2-methyl-3,4-dihydro- 2H-quinoline-1-carboxylic acid ethyl ester | 507 |
| 157 | 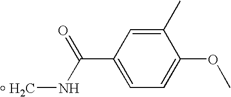 | 6,7-Dimethoxy-4-[(4-methoxy-3- methyl-benzoylamino)-methyl]-2- methyl-3,4-dihydro-2H-quinoline-1- carboxylic acid ethyl ester | 457 |
| 158 | 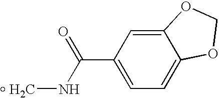 | 4-{[(Benzo[1,3]dioxole-5-carbonyl)- amino]-methyl}-6,7-dimethoxy-2- methyl-3,4-dihydro-2H-quinoline-1- carboxylic acid ethyl ester | 457 |
| 159 | 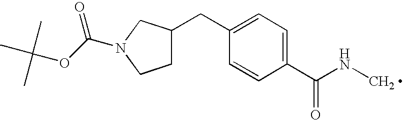 | 4-{[4-(1-tert-Butoxycarbonyl- pyrrolidin-3-yloxy)-benzoylamino]- methyl}-6,7-dimethoxy-2-methyl- 3,4-dihydro-2H-quinoline-1- carboxylic acid ethyl | 498 |
| 160 | 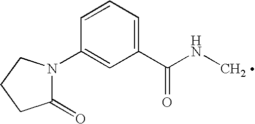 | 6,7-Dimethoxy-2-methyl-4-{[3-(2- oxo-pyrrolidin-1-yl)-benzoylamino]- methyl}-3,4-dihydro-2H-quinoline- 1-carboxylic acid ethyl ester | 496 |
| 161 | 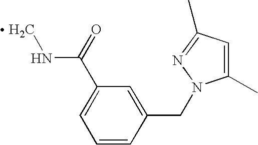 | 4-{[3-(3,5-Dimethyl-pyrazol-1- ylmethyl)-benzoylamino]-methyl}- 6,7-dimethoxy-2-methyl-3,4- dihydro-2H-quinoline-1-carboxylic acid ethyl ester | 521 |
| 162 | 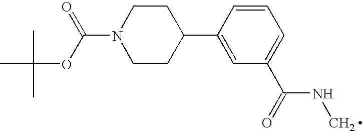 | 4-{[3-(1-tert-Butoxycarbonyl- piperidin-4-yl)-benzoylamino]- methyl}-6,7-dimethoxy-2-methyl- 3,4-dihydro-2H-quinoline-1- carboxylic acid ethyl ester | 496 |
| 163 | 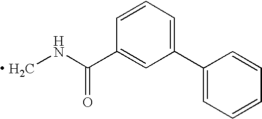 | 4-{[(Biphenyl-3-carbonyl)-amino]- methyl}-6,7-dimethoxy-2-methyl- 3,4-dihydro-2H-quinoline-1- carboxylic acid ethyl ester | 489 |
| 164 | 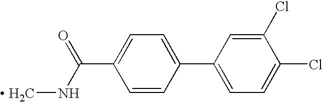 | 4-{[(3′,4′-Dichloro-biphenyl-4- carbonyl)-amino]-methyl}-6,7- dimethoxy-2-methyl-3,4-dihydro- 2H-quinoline-1-carboxylic acid ethyl ester | 557 |
| 165 | 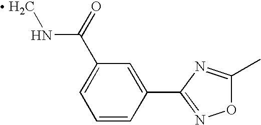 | 6,7-Dimethoxy-2-methyl-4-{[3-(5- methyl-[1,2,4]oxadiazol-3-yl)- benzoylamino]-methyl}-3,4-dihydro- 2H-quinoline-1-carboxylic acid ethyl ester | 495 |
| 166 | 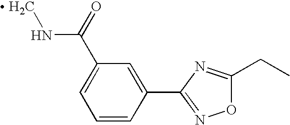 | 4-{[3-(5-Ethyl-[1,2,4]oxadiazol-3-yl)- benzoylamino]-methyl}-6,7- dimethoxy-2-methyl-3,4-dihydro- 2H-quinoline-1-carboxylic acid ethyl ester | 509 |
| 167 | 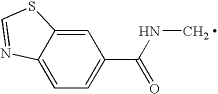 | 4-{[(Benzothiazole-6-carbonyl)- amino]-methyl}-6,7-dimethoxy-2- methyl-3,4-dihydro-2H-quinoline-1- carboxylic acid ethyl ester | 470 |
| 168 | 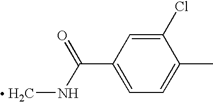 | 4-[(3-Chloro-4-methyl- benzoylamino)-methyl]-6,7- dimethoxy-2-methyl-3,4-dihydro- 2H-quinoline-1-carboxylic acid ethyl ester | 461 |
| 169 | 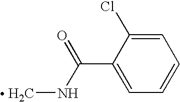 | 4-[(2-Chloro-benzoylamino)- methyl]-6,7-dimethoxy-2-methyl- 3,4-dihydro-2H-quinoline-1- carboxylic acid ethyl ester | 447 |

### Preparation 34

### 3-Ethyl-6,7-dimethyl-3,4-dihydro-1H-quinoxalin-2-one

A mixture of 5,6-dimethyl phenylenediamine (22.45 gm, 165 mmol, 1 eq), 2-ketobutyric acid (16.83 gm, 165 mmol, 1 eq) and ethanol (75 mL) were irradiated in the Milestone microwave (Milestone Laboratories, Sorisole, Italy) for 5 min at 180° C. The solid product upon cooling was filtered and washed with ethanol. Concentration of the filtrate and further crystallization provided the desired quinoxalin-2-one (18.45 gm, 55.2%).

¹H-NMR (dmso-d6): δ 1.17 (t, J=7.05 Hz, 3H), 2.25 (s, 3H), 2.26 (s, 3H), 2.74 (q, J=7.05 Hz, 2H), 7.00 (s, 1H), 7.48 (s, 1H), 12.15 (brs, 1H).

ESI-MS: 203 (MH+).

### Preparation 35

### 3-Chloro-2-ethyl-6,7-dimethyl-1,2-dihydro-quinoxaline

3-Ethyl-6,7-dimethyl-3,4-dihydro-1H-quinoxalin-2-one (18.45 g, 91.2 mmol) was dissolved in 180 mL phosphorus oxychloride and the mixture was heated overnight at 110° C. under a drying tube charged with potassium hydroxide and Drierite®. After cooling, the phosphorus (V) oxychloride was carefully distilled off, and residue was carefully quenched with ice, followed by saturated sodium hydrogen carbonate. The aqueous suspension was extracted several times with methylene chloride. The combined organics were washed 1 time with brine, dried over anhydrous sodium sulfate, filtered, and evaporated to give the title compound (19.3 gm, 81%), which was carried forward without further purification.

¹H-NMR (dmso-d6): δ 1.30 (t, J=7.47 Hz, 3H), 3.04 (q, J=7.47 Hz, 2H), 7.74 , (s, 1H), 7.83 (s, 1H).

### Preparation 36

### 2-Ethyl-6,7-dimethyl-1,2,3,4-tetrahydro-quinoxaline

The residue containing 3-chloro-2-ethyl-6,7-dimethyl-1,2-dihydro-quinoxaline was dissolved in 200 mL acetic acid and 18.2 g sodium acetate was added (222 mmol, 3 eq). After flushing with nitrogen, the vessel was charged with palladium on carbon (10%, 7.86 gm, 0.1 eq Pd, 7.39 mmol). The reaction was subjected to hydrogenation at 45 psi for 5 hr whereupon hydrogen uptake ceased. The reaction was filtered through Celite® and evaporated. The residue was azeotroped 3 times with heptane to remove further acetic acid. The residue was then partitioned between ethyl acetate and saturated aqueous sodium hydrogen carbonate. The organic layer was washed 3 times with saturated aqueous sodium hydrogen carbonate, 1 time with brine, dried over anhydrous sodium sulfate, filtered and evaporated. Flash chromatography eluting with a 20-50% ethyl acetate/hexanes gradient provided the desired quinoxaline (10.56 gm, 63% yield.) as a fluffy pinkish solid.

¹H-NMR (dmso-d6): δ 0.89 (t, J=7.46 Hz, 3H), 1.35 (m, 2H), 1.89, (s, 3H), 1.94 (s, 3H), 2.75 (m, 1H), 2.96 (m, 1H), 3.14 (m, 1H), 6.12, (s, 1H), 6.17 (s, 1H).

### Preparation 37

### 3-Ethyl-6,7-dimethyl-3,4-dihydro-2H-quinoxaline-1-carboxylic Acid Tert-butyl Ester

2-Ethyl-6,7-dimethyl-1,2,3,4-tetrahydro-quinoxaline (10.56 g, 55.6 mmol, 1 eq) was dissolved in anhydrous methylene chloride and cooled to −30° C. in an ethylene glycol/dry ice bath. A solution of di-tert-butyl dicarbonate (12.13 gm, 55.6 mmol, 1 eq) in methylene chloride (50 mL) was added dropwise, and the reaction was allowed to slowly warm to room temperature overnight. The reaction was evaporated to dryness and repartitioned between ethyl acetate and 0.1M HCl. The organic layer was washed 3 times with 0.1M HCl, 1 time with saturated sodium hydrogen carbonate, 1 time with brine, dried over anhydrous sodium sulfate, filtered and evaporated. Silica chromatography eluting with 10% ethyl acetate/hexane provided the desired compound (10.35 g, 64%).

¹H-NMR (dmso-d6): δ 0.90 (t, J=7.47 Hz, 3H), 1.34 (m, 2H), 1.41 (s, 9H), 2.02 (s, 6H), 3.09 (m, 1H), 3.24 (m, 1H), 3.58 (m, 1H), 5.74 (s, 1H), 6.34 (s, 1H), 7.05 (brs, 1H).

ESI-MS: 290 (M+), 235 (MH+-isobutylene).

### Preparation 38

### 2-Ethyl-6,7-dimethyl-2,3-dihydro-quinoxaline-1,4-dicarboxylic Acid 4-tert-butyl Ester 1-ethyl Ester

A solution of 3-ethyl-6,7-dimethyl-3,4-dihydro-2H-quinoxaline-1-carboxylic acid tert-butyl ester (10.35 gm, 35.7 mmol, 1 eq) and 4-dimethylaminopyridine (436 mg, 3.57 mmol, 0.1 eq) in anhydrous pyridine (250 mL) was cooled to 0° C. and ethyl chloroformate (17.0 mL, 178.3 mmol, 5 eq) was added dropwise. The reaction was allowed to warm to room temperature overnight. The solvents were evaporated under vacuum and azeotroped 3 times with heptanes. The residue, after drying under vacuum, was partitioned between ethyl acetate and 0.1M HCl. The organic layer was extracted with 0.1M HCl until the extracts were acidic, then was washed 1 time with saturated aqueous sodium hydrogen carbonate, 1 time with water, 1 time with brine. The organic layer was dried over anhydrous sodium sulfate, filtered and evaporated to provide the desired compound (100%).

¹H-NMR (dmso-d6): δ 0.77 (t, J=7.05 Hz, 3H), 1.20 (t, J=7.05 Hz, 3H), 1.25 (m, 2H), 1.44 (s, 9H), 2.13 (s, 6H), 3.55 (dd, J=13.35, 4.98 Hz, 1H), 3.78 (dd, J=13.1, 3.32 Hz, 1H), 4.12 (m, 2H), 4.39 (m, 1H), 7.41 (s, 1H), 7.44 (s, 1H).

ESI-MS: 307 (MH+-isobutylene), 263 (MH+-boc).

### Preparation 39

### 2-Ethyl-6,7-dimethyl-3,4-dihydro-2H-quinoxaline-1-carboxylic Acid Ethyl Ester

To 2-Ethyl-6,7-dimethyl-2,3-dihydro-quinoxaline-1,4-dicarboxylic acid 4-tert-butyl ester 1-ethyl ester (12.93 g) was added trifluoroacetic acid (200 mL), the mixture was stirred until a solution had formed and then evaporated to dryness under vacuum. The residue was azeotroped 3 times with heptane and dried under vacuum. The residual oil was then partitioned between methylene chloride and saturated aqueous sodium bicarbonate. The aqueous layer was extracted 3 times with methylene chloride. The combined methylene chloride extracts were dried over anhydrous sodium sulfate, filtered and evaporated, giving the desired quinoxaline (100%).

¹H-NMR (dmso-d6): δ 0.77 (t, J=7.47 Hz, 3H), 1.19 (t, J=7.05 Hz, 3H), 1.24 (m, 2H), 2.02 (s, 6H), 3.14 (m, 2H), 4.08 (m, 2H), 4.24 (m, 1H), 5.72 (m, 1H), 7.11 (brs, 1H).

ESI-MS: 263 (MH+).

This racemic quinoxaline was subjected to preparative chiral separations using a ChiralcelOD 10×25 cm chiral preparative column. The eluent was 5% ethanol in heptane, at a flow rate of 275 mL/min, observing at 300 nm. The sample was loaded on the column using 2:1 methanol/dichloromethane. Retention time of the S-enantiomer was 16 min, the R-enantiomer was 22 min. A 25 g sample was subjected to these conditions, resulting in 10 g of the R-enantiomer, 99.4% ee, and 11 g of the S-enantiomer, 95.1% ee.

### EXAMPLES 170, 171, 172 AND 173

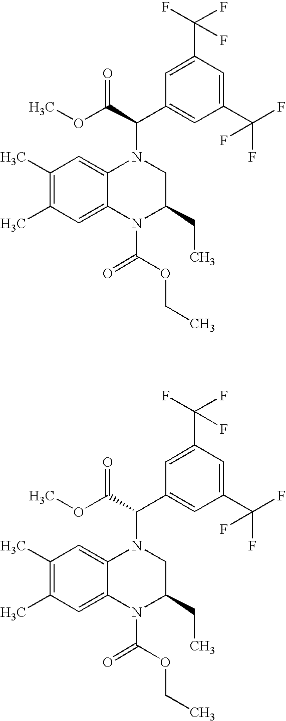

### (R,R, R,S, S,R and S,S)-4-[(3,5-Bis-trifluoromethyl-phenyl)-methoxycarbonyl-methyl]-2-ethyl-6,7-dimethyl-3,4-dihydro-2H-quinoxaline-1-carboxylic Acid Ethyl Ester

Method A:

A mixture of (RS)-2-ethyl-6,7-dimethyl-3,4-dihydro-2H-quinoxaline-1-carboxylic acid ethyl ester (652 mg, 1 eq, 2.5 mmol), bromo-4-(3,5-bis-trifluoromethyl-phenyl)-acetic acid methyl ester (1.0 gm, 1.1 eq, 2.74 mmol) and 2,6-lutidine (0.87 mL, 3 eq, 7.47 mmol) in dimethylformamide (3 mL) were heated at 140° C. for 20 min by microwave irradiation in an Emrys Optimizer (Personal Chemistry, Uppsala, Sweden). The mixture was partitioned between methylene chloride and water, and the phases were separated. The aqueous phase was extracted 3 times with methylene chloride, and the combined organic extracts were washed 2 times with water, 1 time with brine, dried over anhydrous sodium sulfate, filtered and evaporated. Chromatography on silica gel using 10% ethyl acetate in hexanes as eluant provided the title compounds as a mixture of two diastereoisomers (900 mg, 66%).

Method B:

A mixture of (RS)-2-ethyl-6,7-dimethyl-3,4-dihydro-2H-quinoxaline-1-carboxylic acid ethyl ester (652 mg, 1 eq, 2.5 mmol), bromo-4-(3,5-bis-trifluoromethyl-phenyl)-acetic acid methyl ester (1.0 gm, 1.1 eq, 2.74 mmol) and 2,6-lutidine (0.87 mL, 3 eq, 7.47 mmol) in dimethylformamide (3 mL) were stirred at room temperature for 24 hours. The mixture was partitioned between methylene chloride and water, and the phases were separated. The aqueous phase was extracted 3 times with methylene chloride, and the combined organic extracts were washed 2 times with water, 1 time with brine, dried over anhydrous sodium sulfate, filtered and evaporated. Chromatography on silica gel using 10% ethyl acetate in hexanes as eluant provided the title compounds as a mixture of two diastereoisomers (900 mg, 66%).

Diastereoisomer 1 ¹H-NMR (CDCl₃): δ 0.71 (t, J=7.57 Hz, 3H), 1.30 (t, J=7.05 Hz, 3H), 1.46 (m, 2H), 2.18 (s, 3H), 2.20 (s, 3H), 2.77 (dd, J=9.54, 2.08 Hz, 1H), 3.37 (dd, J=11.38, 3.32 Hz, 1H) 3.82 (s, 3H), 4.20 (m, 2H), 4.26 (m, 1H), 5.82 (s, 1H), 6.58 (s, 1H), 7.49 (brs, 1H) 7.75 (s, 2H), 7.88 (s, 1H).

LCMS (ESI+): 547 (MH+)

Diastereoisomer 2: ¹H-NMR (CDCl₃): δ 0.87 (t, J=7.47 Hz, 3H), 1.28 (t, J=6.64 Hz, 3H), 1.43 (m, 2H), 2.18 (s, 3H), 3.11 (dd, J=11.35, 4.98 Hz, 1H), 3.22 (dd, J=10.79, 1.66 Hz, 1H), 3.83 (s, 3H), 4.19 (m, 2H), 4.44 (m, 1H), 5.66 (s, 1H), 6.41 (s, 1H), 7.28 (brs, 1H), 7.73 (s, 2H), 7.86 (s, 1H).

LCMS (ESI+): 547 (MH+).

This racemic mixture of diastereoisomers could be further resolved by chiral HPLC on a Pirkle Covalent (S,S)Whelk-O 1 column (Regis Technologies, Inc., Morton Grove, Ill.) (5×25 cm) eluting at 100 mL/min with 5% ethanol/heptane to provide three fractions:

Isomer 1, Retention time=18 min

Isomers 2 and 3, Retention time=25 min

Isomer 4, Retention time=37 min

### EXAMPLES 174 AND 175

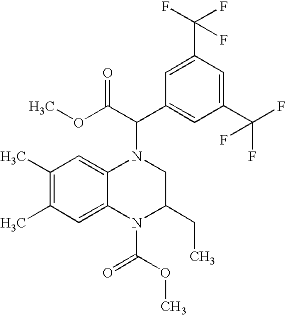

### Preparation of 4-[(3,5-Bis-trifluoromethyl-phenyl)-methoxycarbonyl-methyl]-2-ethyl-6,7-dimethyl-3,4-dihydro-2H-quinoxaline-1-carboxylic Acid Methyl Ester

This compound was prepared using the procedure as described above for 4-[(3,5-bis-trifluoromethyl-phenyl)-methoxycarbonyl-methyl]-2-ethyl-6,7-dimethyl-3,4-dihydro-2H-quinoxaline-1-carboxylic acid ethyl ester, but using methyl chloroformate in the place of ethyl chloroformate.

Diastereoisomer 1 ¹H-NMR (CDCl₃): δ 0.86 (t, J=7.47 Hz, 3H), 1.43 (m, 2H), 2.18 (s, 6H), 3.10 (dd, J=11.48, 4.98 Hz, 1H), 3.22 (dd, J=11.17 Hz 1.66 Hz, 1H), 3.75 (s, 3H), 4.43 (m, 1H), 3.82 (s, 3H), 5.66 (s, 1H), 6.41 (s, 1H), 7.22 (brs, 1H), 7.72 (s, 2H), 7.86 (s, 1H).

LCMS (ESI+): 532 (M+).

Diastereoisomer 2 ¹H-NMR (CDCl₃): δ 0.71 (t, J=7.47 Hz, 3H), 1.19 (m, 1H), 1.43 (m, 1H), 2.19 (s, 3H), 2.20 (s, 3H), 2.77 (dd, J=11.47, 1.65 Hz, 1H), 3.38 (dd, J=11.30, 3.74 Hz, 1H), 3.77 (s, 3H), 3.81 (s, 3H), 4.38 (m, 1H), 5.83 (s, 1H), 6.58 (s, 1H), 7.37 (s, 1H), 7.75 (s, 2H), 7.88 (s, 1H).

LCMS (ESI+): 532 (M+).

### EXAMPLES 176 AND 177

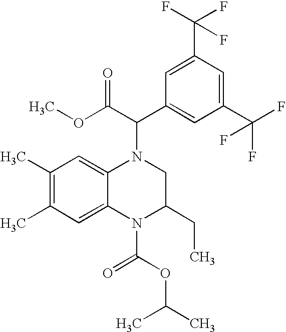

### Preparation of 4-[(3,5-Bis-trifluoromethyl-phenyl)-methoxycarbonyl-methyl]-2-ethyl-6,7-dimethyl-3,4-dihydro-2H-quinoxaline-1-carboxylic Acid Isopropyl Ester

This compound was prepared using the procedure as described above for compound 4-[(3,5-Bis-trifluoromethyl-phenyl)-methoxycarbonyl-methyl]-2-ethyl-6,7-dimethyl-3,4-dihydro-2H-quinoxaline-1-carboxylic acid ethyl ester, but using isopropyl chloroformate in the place of ethyl chloroformate.

Diastereoisomer 1 ¹H-NMR (CDCl₃): δ 0.86 (t, J=7.47 Hz, 3H), 1.16 (dd, J=6.22, 6.22 Hz, 3H), 1.26 (dd, J=15.07, 6.22 Hz, 3H), 1.50 (m, 2H), 2.17 (s, 6H), 3.10, (m, 1H), 3.2 (m, 1H), 3.82 (s, 3H), 4.42 (m, 1H), 5.00 (m, 1H), 5.65 (s, 1H), 6.40 (s, 1H), 7.31 (brs, 1H), 7.64 (s, 1H), 7.74 (s, 1H).).

LCMS (ESI+): 560 (M+).

Diastereoisomer 2 ¹H-NMR (CDCl₃): δ 0.71 (t, J=7.47, 3H), 1.16 (m, 1H), 1.26 (d, J=6.23 Hz), 1.29 (d, J=5.81 Hz), 1.44 (m, 1H), 2.18 (s, 3H), 2.20 (s, 3H), 2.76 (dd, J=11.52, 2.07 Hz, 1H), 3.37 (dd, J=11.52, 3.32 Hz), 3.82 (s, 3H), 4.38 (m, 1H), 5.01 (m, 1H), 5.82 (s, 1H), 6.58 (s, 1H), 7.47 (brs, 1H), 7.75 (s, 2H), 7.88 (s, 1H).

LCMS (ESI+): 561 (MH+).

### Preparation 40

### (3,5-Bis-trifluoromethyl-phenyl)-bromo-acetonitrile

To a solution of (3,5-bis-trifluoromethyl-phenyl)-acetonitrile (2.0 g, 7.9 mmol) in carbon tetrachloride (20 mL) was added dibenzoyl peroxide (0.076 g, 0.3 mmol) and N-bromosucinnimide (1.4 g, 7.9 mmol). This reaction mixture was refluxed for 24 h then diluted with methylene chloride and washed with brine. The organic layer was dried and concentrated to afford the crude product which was purified by silica gel chromatography using 2% ethyl acetate in hexanes as eluant to afford the title compound (2.0 g, 75%).

LCMS (ESI+): 333 (MH+).

### EXAMPLE 178

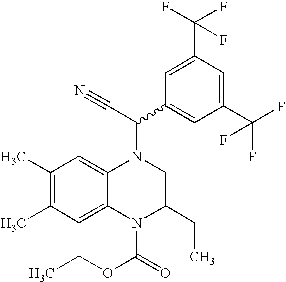

### 4-[(3,5-Bis-trifluoromethyl-phenyl)-cyano-methyl]-2-ethyl-6,7-dimethyl-3,4-dihydro-2H-quinoxaline-1-carboxylic Acid Ethyl Ester

Method A:

A mixture of 2-ethyl-6,7-dimethyl-3,4-dihydro-2H-quinoxaline-1-carboxylic acid ethyl ester (1 g, 1 eq, 3.81 mmol), 3,5-bis-(trifluoromethyl-phenyl)-bromo-acetonitrile (Preparation 40, 1.27 gm, 1 eq, 3.81 mmol) and potassium carbonate (1.58 gm, 3 eq, 11.43 mmol) in acetonitrile (5 mL) was subjected to microwave irradiation in the Emrys Optimizer (Personal Chemistry, Uppsala, Sweden) at 140° C. for 20 min. The reaction was partitioned between ethyl acetate and water, and the phases were separated. The aqueous phase was extracted 3 times with ethyl acetate, and the combined organic extracts were washed 2 times with water, 1 time with brine, dried over anhydrous sodium sulfate, filtered and evaporated. Chromatography on silica gel using a gradient of 10-30% ethyl acetate in hexanes as eluant provided the desired nitrile as a mixture of two diastereoisomers (1.5:1) (900 mg, 29%).

Method B:

A mixture of 2-ethyl-6,7-dimethyl-3,4-dihydro-2H-quinoxaline-1-carboxylic acid ethyl ester (1 g, 1 eq, 3.81 mmol), 3,5-bis-(trifluoromethyl-phenyl)-bromo-acetonitrile (Preparation 40, 1.27 gm, 1 eq, 3.81 mmol) and 2,6-lutidine (3 eq, 11.43 mmol) in N,N-dimethylformamide (5 mL) was stirred at room temperature for 24 hours. The reaction was partitioned between ethyl acetate and water, and the phases were separated. The aqueous phase was extracted 3 times with ethyl acetate, and the combined organic extracts were washed 2 times with water, 1 time with brine, dried over anhydrous sodium sulfate, filtered and evaporated. Chromatography on silica gel using a gradient of 10-30% ethyl acetate in hexanes as eluant provided the desired nitrile as a mixture of two diastereoisomers (1.5:1) (900 mg, 29%).

LCMS (ESI+): 514 (MH+)

### EXAMPLE 179

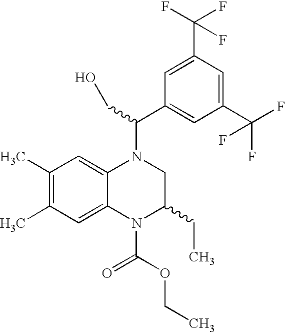

### Preparation of 4-[1-(3,5-Bis-trifluoromethyl-phenyl)-2-hydroxy-ethyl]-2-ethyl-6,7-dimethyl-3,4-dihydro-2H-quinoxaline-1-carboxylic Acid Ethyl Ester

To a solution of the 4-[(3,5-bis-trifluoromethyl-phenyl)-methoxycarbonyl-methyl]-2-ethyl-6,7-dimethyl-3,4-dihydro-2H-quinoxaline-1-carboxylic acid ethyl ester (0.14 g, 0.26 mmol) in tetrahydrofuran (5 mL) at −78° C. was added a solution of lithium aluminum hydride (1M in tetrahydrofuran, 0.16 mL) dropwise. This solution was slowly warmed to room temperature over 3 h. The reaction mixture was quenched with sodium sulfate hexahydrate (2 gm) and the mixture was stirred for 30 min. The reaction mixture was filtered and concentrated to afford the crude product (mixture of diastereoisomers) which was purified by silica gel chromatography eluting with 10% ethyl acetate in hexanes to give the title compound (0.08 g, 70%).

¹H-NMR (CDCl₃): δ 0.71 (t, J=7.57 Hz, 3H), 0.95 (t, J=7.05 Hz, 3H), 1.26 (m, 2H), 2.20 (bs, 6H), 2.97 (dd, 1H), 3.25 (m, 1H), 3.37 (m, 1H), 3.85 (m, 2H), 4.15 (m, 3H), 4.5 (bm, 1H), 4.62(m, 1H), 4.70 (m, 1H), 5.82 (s, 1H), 6.58 (s, 1H), 7.49 (brs, 1H) 7.75 (s, 2H), 7.88 (s, 1H).

LCMS (ESI+): 518 (MH+)

### EXAMPLE 180

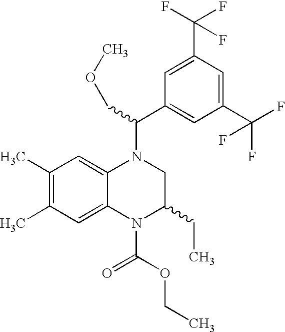

### Preparation of 4-[1-(3,5-Bis-trifluoromethyl-phenyl)-2-methoxy-ethyl]-2-ethyl-6,7-dimethyl-3,4-dihydro-2H-quinoxaline-1-carboxylic Acid Ethyl Ester

To a solution of 4-[1-(3,5-bis-trifluoromethyl-phenyl)-2-hydroxy-ethyl]-2-ethyl-6,7-dimethyl-3,4-dihydro-2H-quinoxaline-1-carboxylic acid ethyl ester (0.044 gm, 0.13 mmol) in anhydrous dimethylformamide (93 mL), was added sodium hydride (60% suspension, 0.005 gm, 0.27 mmol) and the mixture was stirred for 20 min. Excess of methyl iodide was added and the reaction was stirred for 1 h and quenched with saturated ammonium chloride. The mixture was extracted with ether (3×20 mL) and concentrated. Silica gel chromatography of the crude product using a gradient of 10-30% ethyl acetate in hexanes as eluant provided the desired product as an oil (0.015 gm, 35%).

¹H-NMR (CDCl₃): δ 0.71 (t, J=7.57 Hz, 3H), 1.30 (t, J=7.05 Hz, 3H), 1.46 (m, 2H), 2.20 (brs, 6H), 2.97 (dd, 1H), 3.25 (m, 1H), 3.37 (m, 1H), 3.4 (s, 3H), 3.85 (m, 2H), 4.26 (m, 1H), 4.5 (bm, 1H), 4.62(m, 1H), 4.70 (m, 1H), 5.82 (s, 1H), 6.58 (s, 1H), 7.49 (brs, 1H), 7.75 (s, 2H), 7.88 (s, 1H).

LCMS (ESI+): 532 (MH+).

### EXAMPLE 181

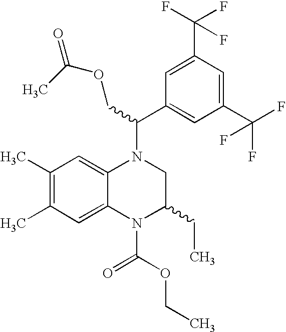

### Preparation of 4-[2-Acetoxy-1-(3,5-bis-trifluoromethyl-phenyl)-ethyl]-2-ethyl-6,7-dimethyl-3,4-dihydro-2H-quinoxaline-1-carboxylic Acid Ethyl Ester

4-[1-(3,5-Bis-trifluoromethyl-phenyl)-2-hydroxy-ethyl]-2-ethyl-6,7-dimethyl-3,4-dihydro-2H-quinoxaline-1-carboxylic acid ethyl ester was dissolved in a 1:1 mixture of acetic anhydride and pyridine (4 mL) and the mixture was stirred at ambient temperature for 10 h. The solvent was removed in vacuo and the residue was purified by silica gel chromatography to afford the desired acetate (25 mg, 58%; mixture of diastereoisomers)

¹H-NMR (CDCl₃): δ 0.71 (t, J=7.57 Hz, 3H), 1.30 (t, J=7.05 Hz, 3H), 1.46 (m, 2H), 1.98 (s, 3H) 2.18 (s, 3H), 2.20 (s, 3H), 2.77 (dd, 1H), 3.05 (m, 1H), 3.37 (m, 1H), 4.20 (m, 2H), 4.26 (m, 1H), 4.5 (m, 1H), 4.62(m, 1H), 4.70 (m, 1H), 5.82 (s, 1H), 6.58 (s, 1H), 7.49 (brs, 1H), 7.75 (s, 2H), 7.88 (s, 1H).

LCMS (ESI+): 560 (MH+)

### EXAMPLE 182

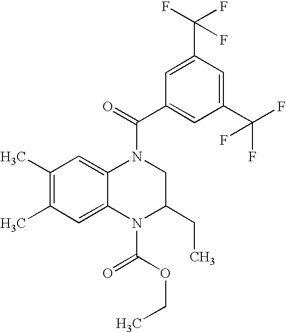

### Preparation of 4-(3,5-Bis-trifluoromethyl-benzoyl)-2-ethyl-6,7-dimethyl-3,4-dihydro-2H-quinoxaline-1-carboxylic Acid Ethyl Ester

A room-temperature solution of 2-ethyl-6,7-dimethyl-3,4-dihydro-2H-quinoxaline-1-carboxylic acid ethyl ester (0.1 gm, 1 eq, 0.38 mmol) in 5 mL methylene chloride was treated with the acid chloride formed by combination of 3,5-bis-(trifluoromethyl)benzoic acid (147 mg, 1.5 eq, 0.57 mmol), oxalyl chloride (0.05 mL, 1.5 eq, 0.57 mmol) and a drop of anhydrous dimethylformamide. The reaction was allowed to stir overnight at room temperature, at which time the reaction was quenched with saturated sodium hydrogen carbonate and extracted 3 times with methylene chloride. The combined organic extracts were washed 1 time with 1M HCl, 1 time with saturated sodium hydrogen carbonate, 1 time with brine, dried over anhydrous sodium sulfate, filtered and evaporated. Chromatography on silica gel using a 10-20% ethyl acetate in hexanes gradient provided 4-(3,5-Bis-trifluoromethyl-benzoyl)-2-ethyl-6,7-dimethyl-3,4-dihydro-2H-quinoxaline-1-carboxylic acid ethyl ester (130 mg, 68%).

¹H-NMR (CDCl₃): δ 0.91 (t, J=7.47 Hz, 3H), 1.30 (t, J=7.04 Hz, 3H), 1.50 (m, 1H), 1.60 (m, 1H), 1.94 (s, 3H), 2.20 (s, 3H), 3.39 (m, 1H), 4.26 (q, J=6.22 Hz, 2H), 4.68 (m, 2H), 6.29 (brs, 1H), 7.41 (brs, 1H), 7.78 (s, 2H), 7.85 (s, 1H).

FIA-MS (APCl+): 503 (MH+).

### Preparation 41

### 6-Trifluoromethyl-1,2,3,4-tetrahydro-quinoxaline

This desired quinoxaline was prepared using the method described by V. Krchnak et. al. (Tetrahedron Lett. 42, 2443-2446 (2001)), using 3-(R)-amino-pentan-1-ol and 2-fluoro-5-trifluoromethyl-nitrobenzene.

¹H-NMR (dmso-d6): δ 1.06 (t, J=7.48 Hz, 3H), 1.73 (m, J=7.90 Hz, 2H), 3.19 (dd, J=9.96 Hz, 1H), 3.68 (dd, J=9.55 Hz, 1H), 3.71 (m, 1H), 6.98 (d, J=8.3 Hz, 1H), 7.25 (dd, 1H), 7.43 (s, 1H).

ESI-MS: 231 (MH+).

### EXAMPLE 183

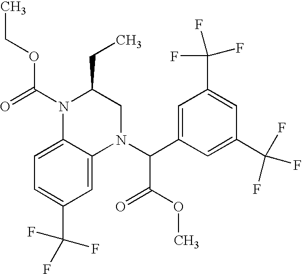

### 4-[(3,5-Bis-trifluoromethyl-phenyl)-methoxycarbonyl-methyl]-2-ethyl-6-trifluoromethyl-3,4-dihydro-2H-quinoxaline-1-carboxylic Acid Ethyl Ester

A solution of 6-trifluoromethyl-1,2,3,4-tetrahydro-quinoxaline (Preparation 41, 0.01 g, 0.04 mmol, 1 eq), 3,5-bis-trifluoromethyl-phenyl)-methoxycarbonyl-methyl bromide (0.017 g, 0.044 mmol, 1.1 eq), and 2,6-lutidine (0.014 g, 0.12 mmol 3 eq) in dimethylformamide (0.5 mL) were irradiated in the Milestone microwave (Milestone Laboratories, Sorisole, Italy) for 10 min at 140° C. The solvent was removed under reduced pressure to yield a dark oil which was used without further purification.

ESI-MS: 542 (MH⁺).

This residue was combined with 2,6-lutidine (0.018 g, 0.16 mmol, 2 eq) and ethyl chloroformate (0.0175 g, 0.16 mmol, 2 eq) in dimethylformamide (0.5 mL) and heated at 140° C. for 10 min by irradiation in a Milestone microwave (Milestone Laboratories, Sorisole, Italy). The solution was purified by preparative HPLC eluting with water 0.1% NH₄OH, 70-0% with acetonitrile, 0.1% NH₄OH, 6 min gradient time, and 8 min run time to yield the title compound.

ESI-MS: 587 (MH+)

General Procedure for Parallel Synthesis of Amides:

The required benzoic acids (0.06 mmol, 1 eq) were combined with PS-PPh₃ (115 mg, 1.51 mmol/g, 3 eq, 0.18 mmol) (Argonaut Technologies, Inc., Foster City, Calif.) in septum-capped 2 dram vials. To this solid mixture was added dichloroethane (1 mL) via a Tecan US (Research Triangle Park, N.C.) liquid handler. To this suspension was added via the Tecan a solution of trichloroacetonitrile (0.0072 mL, 1.2 eq, 0.07 mmol) in dichloroethane (1 mL) and the reactions were gently agitated by means of an orbital shaker for 3 h. To this solution was added PS-DIPAM (56 mg, 3.2 mmol/g, 3 eq, 0.18 mmol) (Polymer Laboratories, Amherst, Mass.) and a solution of 2-ethyl-6,7-dimethyl-3,4-dihydro-2H-quinoxaline-1-carboxylic acid ethyl ester (15 mg, 0.06 mmol, 1.0 eq) in dichloroethane (1 mL) was added via the Tecan. The reactions were agitated overnight. The reactions were filtered through filter tubes using the Tecan liquid handler, and the resins were washed 3 times with dichloroethane. The solutions were evaporated using a Genevac Mega660 centrifugal evaporator (Genevac Ltd. Suffolk, UK) and the residues were redissolved in dimethyl sulfoxide (0.2 mL) and purified using the Shimadzu preparative HPLC system (Shimadzu Corporation, Kyoto, Japan) eluting with a 30-100% acetonitrile/water/0.1% formic acid gradient on a 19×50 mm Waters Symmetry Column (Waters Corp, Milford, Mass.) 8 min run, 6 min gradient, 25 mL/min, UV triggered collection, observing at 210 nm. The product-containing fractions were evaporated to dryness using the Genevac Mega660 centrifugal evaporator (Genevac Ltd., Suffolk, UK).

Using the appropriate starting materials, Examples 184-409 were made as racemic mixtures in an analogous manner to Example 183:

### EXAMPLE 184

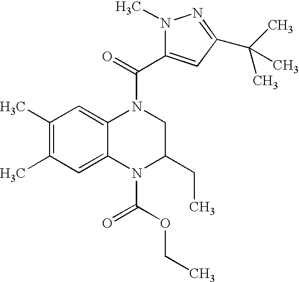

### 4-(5-tert-Butyl-2-methyl-2H-pyrazole-3-carbonyl )-2-ethyl-6,7-dimethyl-3,4-dihydro-2H-quinoxaline-1-carboxylic Acid Ethyl Ester

LC-MS (ESI+): 427 (MH+).

### EXAMPLE 185

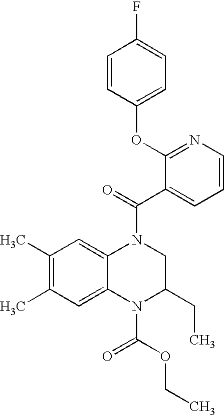

### 2-Ethyl-4-[2-(4-fluoro-phenoxy)-pyridine-3-carbonyl]-6,7-dimethyl-3,4-dihydro-2H-quinoxaline-1-carboxylic Acid Ethyl Ester

LC-MS (ESI+): (MH+).

### EXAMPLE 186

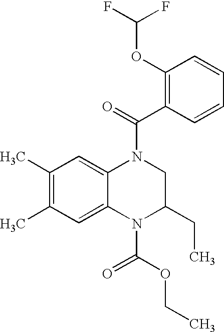

### 4-(2-Difluoromethoxy-benzoyl)-2-ethyl-6,7-dimethyl-3,4-dihydro-2H-quinoxaline-1-carboxylic Acid Ethyl Ester

LC-MS (ESI+): 478 (MH+).

### EXAMPLE 187

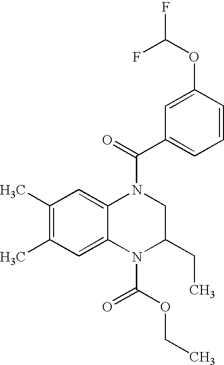

### 4-(3-Difluoromethoxy-benzoyl)-2-ethyl-6,7-dimethyl-3,4-dihydro-2H-quinoxaline-1-carboxylic Acid Ethyl Ester

LC-MS (ESI+): 433 (MH+).

### EXAMPLE 188

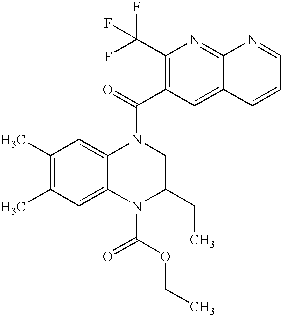

### 2-Ethyl-6,7-dimethyl-4-(2-trifluoromethyl-[1,8]naphthyridine-3-carbonyl)-3,4-dihydro-2H-quinoxaline-1-carboxylic Acid Ethyl Ester

LC-MS (ESI+): 487 (MH+).

### EXAMPLE 189

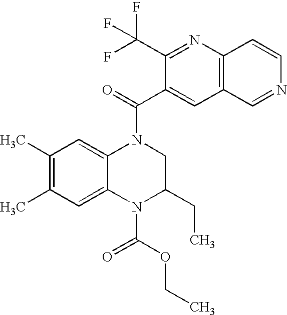

### 2-Ethyl-6,7-dimethyl-4-(2-trifluoromethyl-[1,6]naphthyridine-3-carbonyl)-3,4-dihydro-2H-quinoxaline-1-carboxylic Acid Ethyl Ester

LC-MS (ESI+): 487 (MH+).

### EXAMPLE 190

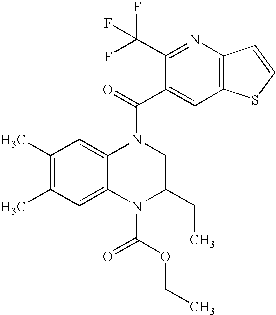

### 2-Ethyl-6,7-dimethyl-4-(5-trifluoromethyl-thieno[3,2-b]pyridine-6-carbonyl)-3,4-dihydro-2H-quinoxaline-1-carboxylic Acid Ethyl Ester

LC-MS (ESI+): 492 (MH+).

### EXAMPLE 191

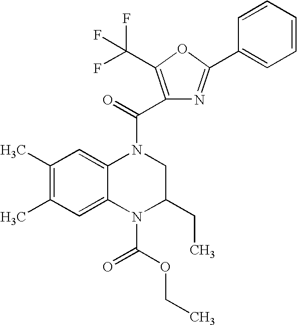

### 2-Ethyl-6,7-dimethyl-4-(2-phenyl-5-trifluoromethyl-oxazole-4-carbonyl)-3,4-dihydro-2H-quinoxaline-1-carboxylic Acid Ethyl Ester

LC-MS (ESI+): 502 (MH+).

### EXAMPLE 192

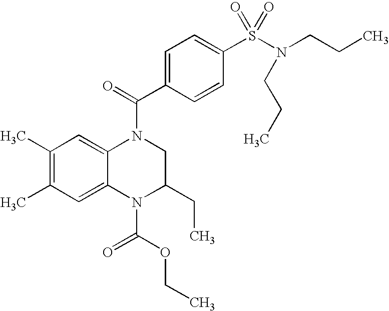

### 4-(4-Dipropylsulfamoyl-benzoyl)-2-ethyl-6,7-dimethyl-3,4-dihydro-2H-quinoxaline-1-carboxylic Acid Ethyl Ester

LC-MS (ESI+): 530 (MH+).

### EXAMPLE 193

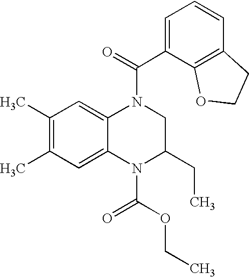

### 4-(2,3-Dihydro-benzofuran-7-carbonyl)-2-ethyl-6,7-dimethyl-3,4-dihydro-2H-quinoxaline-1-carboxylic Acid Ethyl Ester

LC-MS (ESI+): 409 (MH+).

### EXAMPLE 194

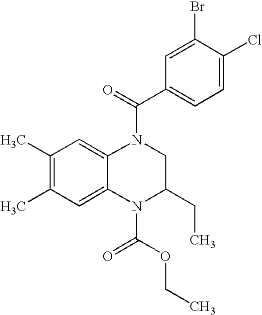

### 4-(3-Bromo-4-chloro-benzoyl)-2-ethyl-6,7-dimethyl-3,4-dihydro-2H-quinoxaline-1-carboxylic Acid Ethyl Ester

LC-MS (ESI+): 480 (MH+).

### EXAMPLE 195

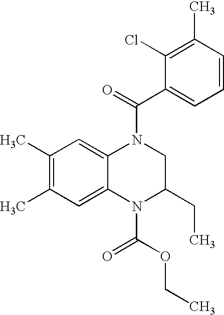

### 4-(2-Chloro-3-methyl-benzoyl)-2-ethyl-6,7-dimethyl-3,4-dihydro-2H-quinoxaline-1-carboxylic Acid Ethyl Ester

LC-MS (ESI+): 415 (MH+).

### EXAMPLE 196

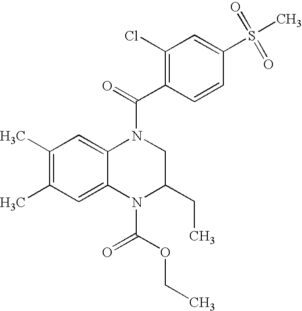

### 4-(2-Chloro-4-methanesulfonyl-benzoyl)-2-ethyl-6,7-dimethyl-3,4-dihydro-2H-quinoxaline-1-carboxylic Acid Ethyl Ester

LC-MS (ESI+): 479 (MH+).

### EXAMPLE 197

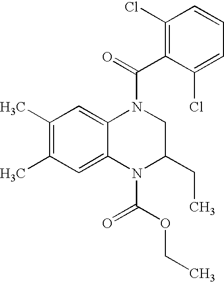

### 4-(2,6-Dichloro-benzoyl)-2-ethyl-6,7-dimethyl-3,4-dihydro-2H-quinoxaline-1-carboxylic Acid Ethyl Ester

LC-MS (ESI+): 436 (MH+).

### EXAMPLE 198

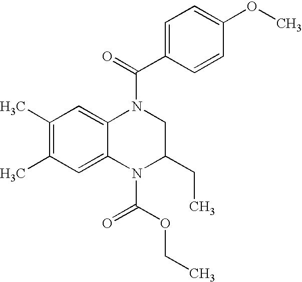

### 2-Ethyl-4-(4-methoxy-benzoyl)-6,7-dimethyl-3,4-dihydro-2H-quinoxaline-1-carboxylic Acid Ethyl Ester

LC-MS (ESI+): 397 (MH+).

### EXAMPLE 199

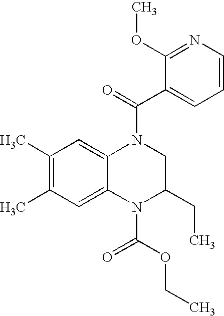

### 2-Ethyl-4-(2-methoxy-pyridine-3-carbonyl)-6,7-dimethyl-3,4-dihydro-2H-quinoxaline-1-carboxylic Acid Ethyl Ester

LC-MS (ESI+): 398 (MH+).

### EXAMPLE 200

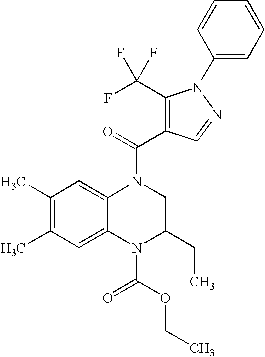

### 2-Ethyl-6,7-dimethyl-4-(1-phenyl-5-trifluoromethyl-1H-pyrazole-4-carbonyl)-3,4-dihydro-2H-quinoxaline-1-carboxylic Acid Ethyl Ester

LC-MS (ESI+): 501 (MH+).

### EXAMPLE 201

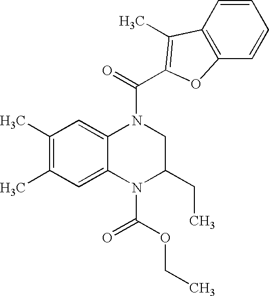

### 2-Ethyl-6,7-dimethyl-4-(3-methyl-benzofuran-2-carbonyl)-3,4-dihydro-2H-quinoxaline-1-carboxylic Acid Ethyl Ester

LC-MS (ESI+): 421(MH+).

### EXAMPLE 202

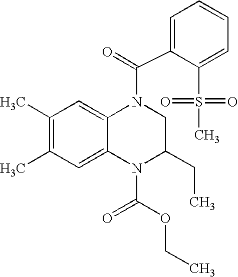

### 2-Ethyl-4-(2-methanesulfonyl-benzoyl)-6,7-dimethyl-3,4-dihydro-2H-quinoxaline-1-carboxylic Acid Ethyl Ester

LC-MS (ESI+): 445 (MH+).

### EXAMPLE 203

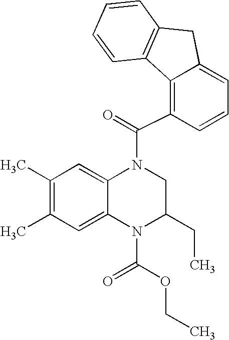

### 2-Ethyl-4-(9H-fluorene-4-carbonyl)-6,7-dimethyl-3,4-dihydro-2H-quinoxaline-1-carboxylic Acid Ethyl Ester

LC-MS (ESI+): 455 (MH+).

### EXAMPLE 204

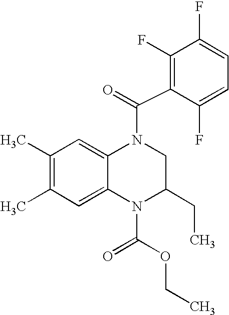

### 2-Ethyl-6,7-dimethyl-4-(2,3,6-trifluoro-benzoyl)-3,4-dihydro-2H-quinoxaline-1-carboxylic Acid Ethyl Ester

LC-MS (ESI+): 421 (MH+).

### EXAMPLE 205

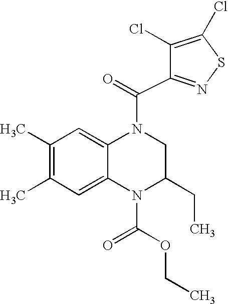

### 4-(4,5-Dichloro-isothiazole-3-carbonyl)-2-ethyl-6,7-dimethyl-3,4-dihydro-2H-quinoxaline-1-carboxylic Acid Ethyl Ester

LC-MS (ESI+): 443 (MH+).

### EXAMPLE 206

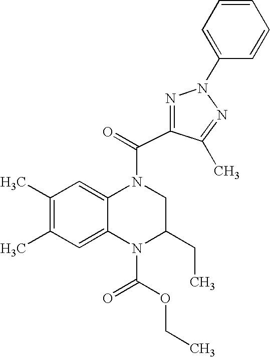

### 2-Ethyl-6,7-dimethyl-4-(5-methyl-2-phenyl-2H[1,2,3]triazole-4-carbonyl)-3,4-dihydro-2H-quinoxaline-1-carboxylic Acid Ethyl Ester

LC-MS (ESI+): 448 (MH+).

### EXAMPLE 207

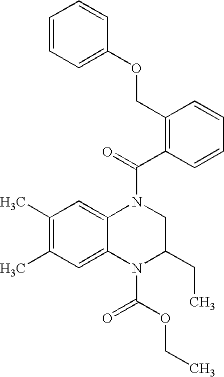

### 2-Ethyl-6,7-dimethyl-4-(2-phenoxymethyl-benzoyl)-3,4-dihydro-2H-quinoxaline-1-carboxylic Acid Ethyl Ester

LC-MS (ESI+): 473 (MH+).

### EXAMPLE 208

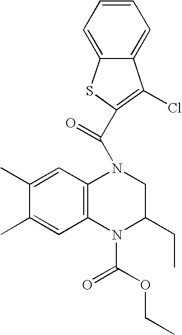

### 4-(3-Chloro-benzo[b]thiophene-2-carbonyl)-2-ethyl-6,7-dimethyl-3,4-dihydro-2H-quinoxaline-1-carboxylic Acid Ethyl Ester

LC-MS (ESI+): 457 (MH+).

### EXAMPLE 209

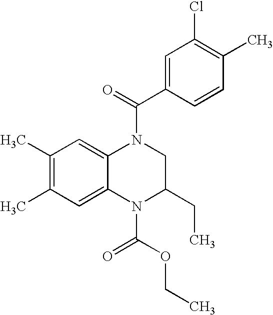

### 4-(3-Chloro-4-methyl-benzoyl)-2-ethyl-6,7-dimethyl-3,4-dihydro-2H-quinoxaline-1-carboxylic Acid Ethyl Ester

LC-MS (ESI+): 415 (MH+).

### EXAMPLE 210

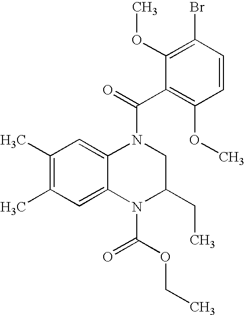

### 4-(3-Bromo-2,6-dimethoxy-benzoyl)-2-ethyl-6,7-dimethyl-3,4-dihydro-2H-quinoxaline-1-carboxylic Acid Ethyl Ester

LC-MS (ESI+): 506 (MH+).

### EXAMPLE 211

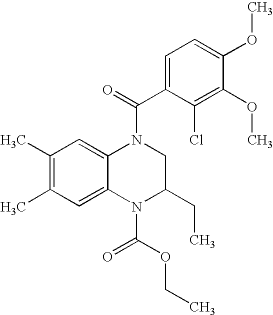

### 4-(2-Chloro-3,4-dimethoxy-benzoyl)-2-ethyl-6,7-dimethyl-3,4-dihydro-2H-quinoxaline-1-carboxylic Acid Ethyl Ester

LC-MS (ESI+): 461 (MH+).

### EXAMPLE 212

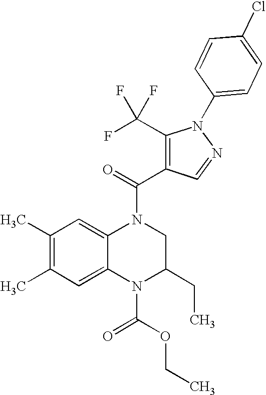

### 4-[1-(4-Chloro-phenyl)-5-trifluoromethyl-1H-pyrazole-4-carbonyl]-2-ethyl-6,7-dimethyl-3,4-dihydro-2H-quinoxaline-1-carboxylic Acid Ethyl Ester

LC-MS (ESI+): 535 (MH+).

### EXAMPLE 213

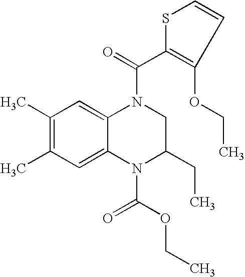

### 4-(3-Ethoxy-thiophene-2-carbonyl)-2-ethyl-6,7-dimethyl-3,4-dihydro-2H-quinoxaline-1-carboxylic Acid Ethyl Ester

LC-MS (ESI+): 417 (MH+).

### EXAMPLE 214

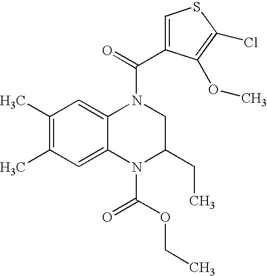

### 4-(5-Chloro-4-methoxy-thiophene-3-carbonyl)-2-ethyl-6,7-dimethyl-3,4-dihydro-2H-quinoxaline-1-carboxylic Acid Ethyl Ester

LC-MS (ESI+): 437 (MH+).

### EXAMPLE 215

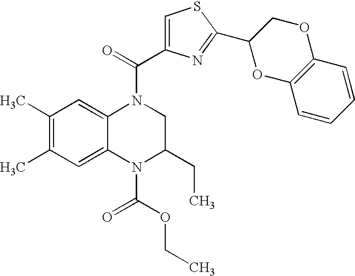

### 4-[2-(2,3-Dihydro-benzo[1,4]dioxin-2-yl)-thiazole-4-carbonyl]-2-ethyl-6,7-dimethyl-3,4-dihydro-2H-quinoxaline-1-carboxylic Acid Ethyl Ester

LC-MS (ESI+): 508 (MH+).

### EXAMPLE 216

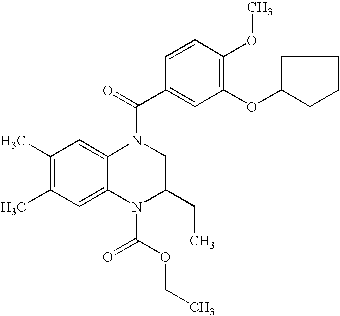

### 4-(3-Cyclopentyloxy-4-methoxy-benzoyl)-2-ethyl-6,7-dimethyl-3,4-dihydro-2H-quinoxaline-1-carboxylic Acid Ethyl Ester

LC-MS (ESI+): 481 (MH+).

### EXAMPLE 217

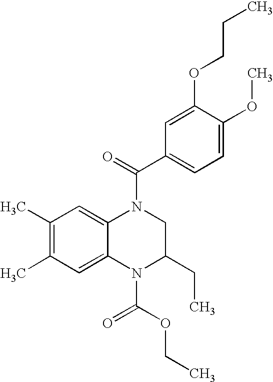

### 2-Ethyl-4-(4-methoxy-3-propoxy-benzoyl)-6,7-dimethyl-3,4-dihydro-2H-quinoxaline-1-carboxylic Acid Ethyl Ester

LC-MS (ESI+): 455 (MH+).

### EXAMPLE 218

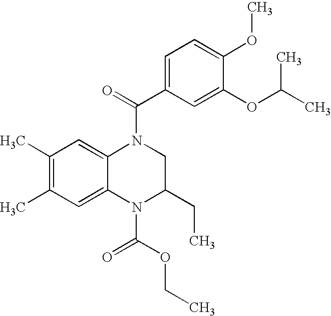

### 2-Ethyl-4-(3-isopropoxy-4-methoxy-benzoyl)-6,7-dimethyl-3,4-dihydro-2H-quinoxaline-1-carboxylic Acid Ethyl Ester

LC-MS (ESI+): 455 (MH+).

### EXAMPLE 219

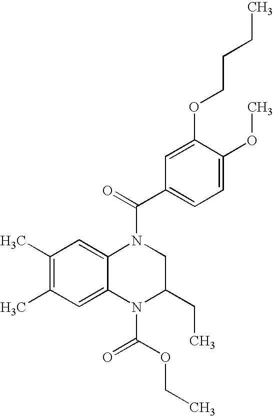

### 4-(3-Butoxy-4-methoxy-benzoyl)-2-ethyl-6,7-dimethyl-3,4-dihydro-2H-quinoxaline-1-carboxylic Acid Ethyl Ester

LC-MS (ESI+): 469 (MH+).

### EXAMPLE 220

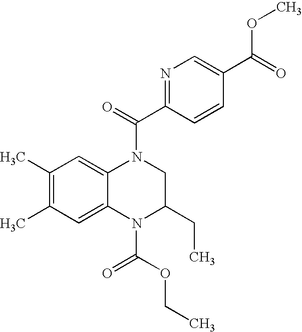

### 2-Ethyl-4-(5-methoxycarbonyl-pyridine-2-carbonyl)-6,7-dimethyl-3,4-dihydro-2H-quinoxaline-1-carboxylic Acid Ethyl Ester

LC-MS (ESI+): 426 (MH+).

### EXAMPLE 221

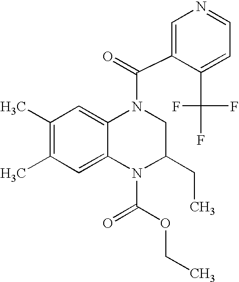

### 2-Ethyl-6,7-dimethyl-4-(4-trifluoromethyl-pyridine-3-carbonyl)-3,4-dihydro-2H-quinoxaline-1-carboxylic Acid Ethyl Ester

LC-MS (ESI+): 436 (MH+).

### EXAMPLE 222

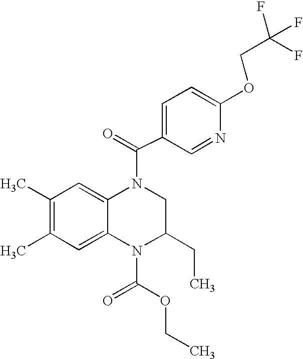

### 2-Ethyl-6,7-dimethyl-4-[6-(2,2,2-trifluoro-ethoxy)-pyridine-3-carbonyl]-3,4-dihydro-2H-quinoxaline-1-carboxylic Acid Ethyl Ester

LC-MS (ESI+): 466 (MH+).

### EXAMPLE 223

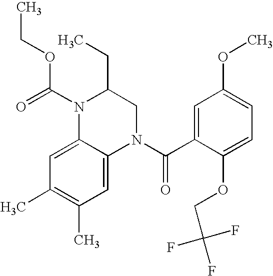

### 2-Ethyl-4-[5-methoxy-2-(2,2,2-trifluoro-ethoxy)-benzoyl]-6,7-dimethyl-3,4-dihydro-2H-quinoxaline-1-carboxylic Acid Ethyl Ester

LC-MS (ESI+): 495 (MH+).

### EXAMPLE 224

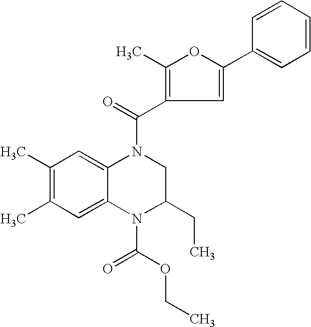

### 2-Ethyl-6,7-dimethyl-4-(2-methyl-5-phenyl-furan-3-carbonyl)-3,4-dihydro-2H-quinoxaline-1-carboxylic Acid Ethyl Ester

LC-MS (ESI+): 447 (MH+).

### EXAMPLE 225

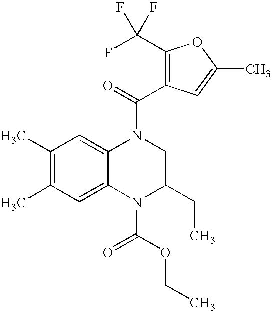

### 2-Ethyl-6,7-dimethyl-4-(5-methyl-2-trifluoromethyl-furan-3-carbonyl)-3,4-dihydro-2H-quinoxaline-1-carboxylic Acid Ethyl Ester

LC-MS (ESI+): 439 (MH+).

### EXAMPLE 226

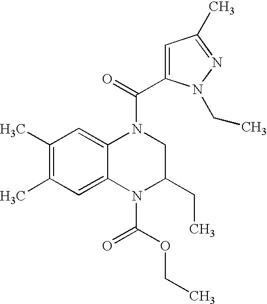

### 2-Ethyl-4-(2-ethyl-5-methyl-2H-pyrazole-3-carbonyl)-6,7-dimethyl-3,4-dihydro-2H-quinoxaline-1-carboxylic Acid Ethyl Ester

LC-MS (ESI+): 399 (MH+).

### EXAMPLE 227

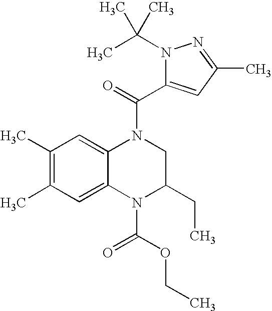

### 4-(2-tert-Butyl-5-methyl-2H-pyrazole-3-carbonyl)-2-ethyl-6,7-dimethyl-3,4-dihydro-2H-quinoxaline-1-carboxylic Acid Ethyl Ester

LC-MS (ESI+): 427 (MH+).

### EXAMPLE 228

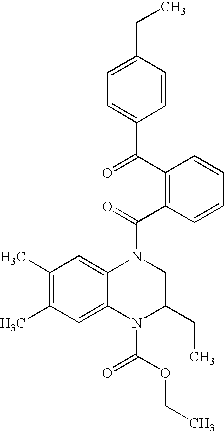

### 2-Ethyl-4-[2-(4-ethyl-benzoyl)-benzoyl]-6,7-dimethyl-3,4-dihydro-2H-quinoxaline-1-carboxylic Acid Ethyl Ester

LC-MS (ESI+): 499 (MH+).

### EXAMPLE 229

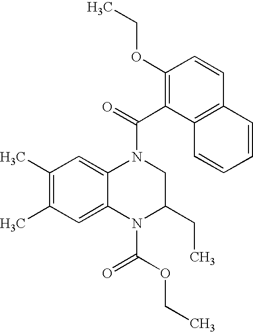

### 4-(2-Ethoxy-naphthalene-1-carbonyl)-2-ethyl-6,7-dimethyl-3,4-dihydro-2H-quinoxaline-1-carboxylic Acid Ethyl Ester

LC-MS (ESI+): 461 (MH+).

### EXAMPLE 230

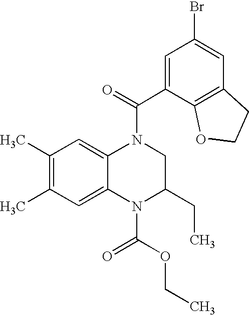

### 4-(5-Bromo-2,3-dihydro-benzofuran-7-carbonyl)-2-ethyl-6,7-dimethyl-3,4-dihydro-2H-quinoxaline-1-carboxylic Acid Ethyl Ester

LC-MS (ESI+): 488 (MH+).

### EXAMPLE 231

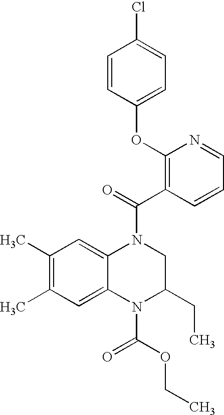

### 4-[2-(4-Chloro-phenoxy)-pyridine-3-carbonyl]-2-ethyl-6,7-dimethyl-3,4-dihydro-2H-quinoxaline-1-carboxylic Acid Ethyl Ester

LC-MS (ESI+): 494 (MH+).

### EXAMPLE 232

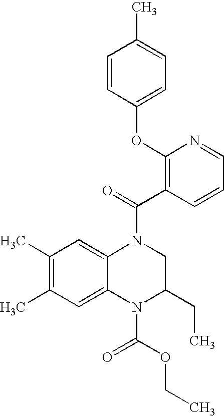

### 2-Ethyl-6,7-dimethyl-4-(2-p-tolyloxy-pyridine-3-carbonyl)-3,4-dihydro-2H-quinoxaline-1-carboxylic Acid Ethyl Ester

LC-MS (ESI+): 474 (MH+).

### EXAMPLE 233

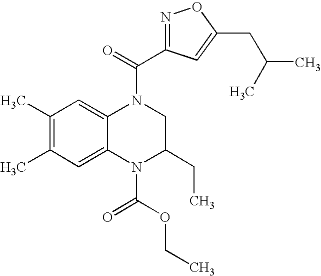

### 2-Ethyl-4-(5-isobutyl-isoxazole-3-carbonyl)-6,7-dimethyl-3,4-dihydro-2H-quinoxaline-1-carboxylic Acid Ethyl Ester

LC-MS (ESI+): 414 (MH+).

### EXAMPLE 234

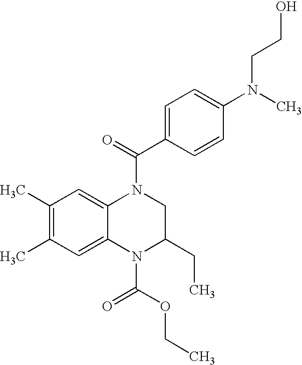

### 2-Ethyl-4-{4-[(2-hydroxy-ethyl)-methyl-amino]-benzoyl}-6,7-dimethyl-3,4-dihydro-2H-quinoxaline-1-carboxylic Acid Ethyl Ester

LC-MS (ESI+): 440 (MH+).

### EXAMPLE 235

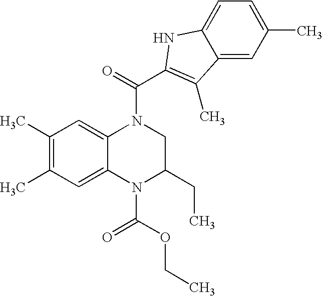

### 4-(3,5-Dimethyl-1H-indole-2-carbonyl)-2-ethyl-6,7-dimethyl-3,4-dihydro-2H-quinoxaline-1-carboxylic Acid Ethyl Ester

LC-MS (ESI+): 434 (MH+).

### EXAMPLE 236

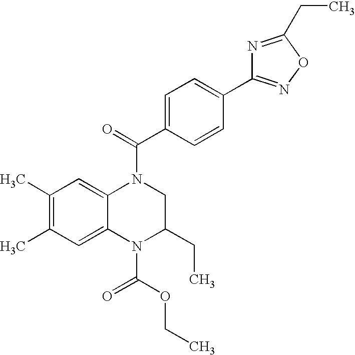

### 2-Ethyl-4-[4-(5-ethyl-[1,2,4]oxadiazol-3-yl)-benzoyl]-6,7-dimethyl-3,4-dihydro-2H-quinoxaline-1-carboxylic Acid Ethyl Ester

LC-MS (ESI+): 463 (MH+).

### EXAMPLE 237

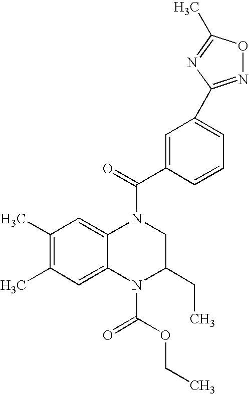

### 2-Ethyl-6,7-dimethyl-4-[3-(5-methyl-[1,2,4]oxadiazol-3-yl)-benzoyl]-3,4-dihydro-2H-quinoxaline-1-carboxylic Acid Ethyl Ester

LC-MS (ESI+): 449 (MH+).

### EXAMPLE 238

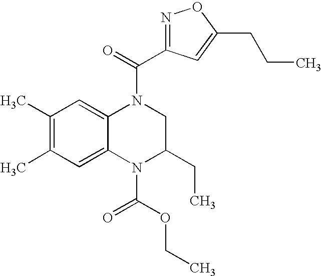

### 2-Ethyl-6,7-dimethyl-4-(5-propyl-isoxazole-3-carbonyl)-3,4-dihydro-2H-quinoxaline-1-carboxylic Acid Ethyl Ester

LC-MS (ESI+): 400 (MH+).

### EXAMPLE 239

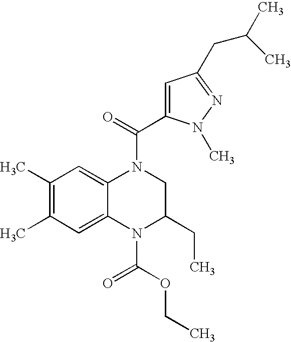

### 2-Ethyl-4-(5-isobutyl-2-methyl-2H-pyrazole-3-carbonyl)-6,7-dimethyl-3,4-dihydro-2H-quinoxaline-1-carboxylic Acid Ethyl Ester

LC-MS (ESI+): 427 (MH+).

### EXAMPLE 240

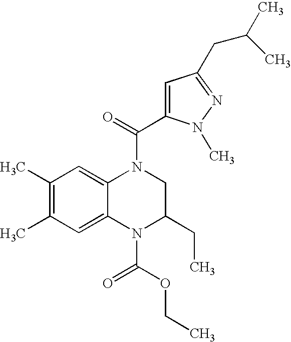

### 2-Ethyl-4-(5-methoxymethyl-furan-2-carbonyl)-6,7-dimethyl-3,4-dihydro-2H-quinoxaline-1-carboxylic Acid Ethyl Ester

LC-MS (ESI+): 401 (MH+).

### EXAMPLE 241

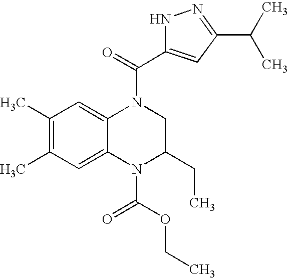

### 2-Ethyl-4-(5-isopropyl-2H-pyrazole-3-carbonyl)-6,7-dimethyl-3,4-dihydro-2H-quinoxaline-1-carboxylic Acid Ethyl Ester

LC-MS (ESI+): 399 (MH+).

### EXAMPLE 242

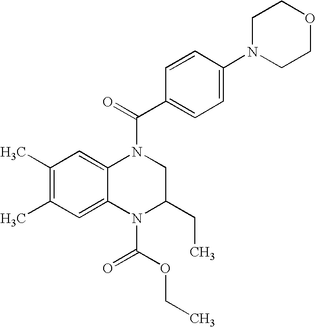

### 2-Ethyl-6,7-dimethyl-4-(4-morpholin-4-yl-benzoyl)-3,4-dihydro-2H-quinoxaline-1-carboxylic Acid Ethyl Ester

LC-MS (ESI+): 452 (MH+).

### EXAMPLE 243

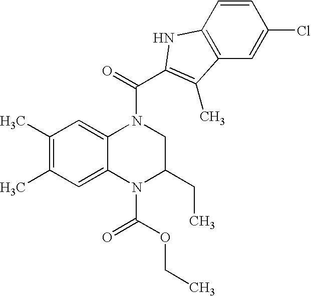

### 4-(5-Chloro-3-methyl-1H-indole-2-carbonyl)-2-ethyl-6,7-dimethyl-3,4-dihydro-2H-quinoxaline-1-carboxylic Acid Ethyl Ester

LC-MS (ESI+): 454 (MH+).

### EXAMPLE 244

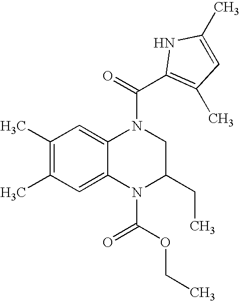

### 4-(3,5-Dimethyl-1H-pyrrole-2-carbonyl)-2-ethyl-6,7-dimethyl-3,4-dihydro-2H-quinoxaline-1-carboxylic Acid Ethyl Ester

LC-MS (ESI+): 384 (MH+).

### EXAMPLE 245

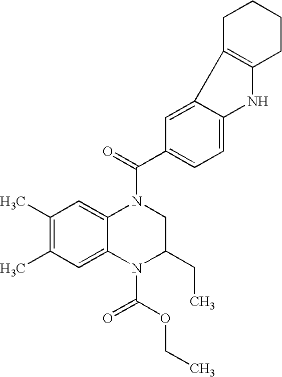

### 2-Ethyl-6,7-dimethyl-4-(6,7,8,9-tetrahydro-5H-carbazole-3-carbonyl)-3,4-dihydro-2H-quinoxaline-1-carboxylic Acid Ethyl Ester

LC-MS (ESI+): 460 (MH+).

### EXAMPLE 246

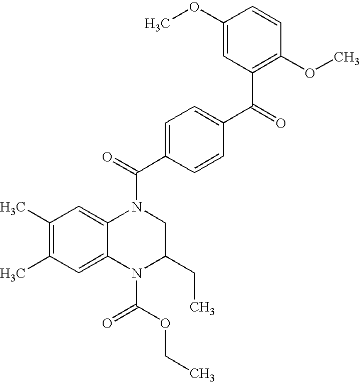

### 4-[4-(2,5-Dimethoxy-benzoyl)-benzoyl]-2-ethyl-6,7-dimethyl-3,4-dihydro-2H-quinoxaline-1-carboxylic Acid Ethyl Ester

LC-MS (ESI+): 531 (MH+).

### EXAMPLE 247

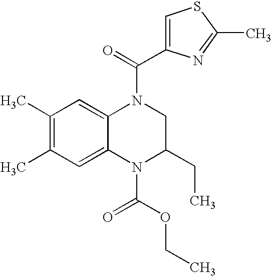

### 2-Ethyl-6,7-dimethyl-4-(2-methyl-thiazole-4-carbonyl)-3,4-dihydro-2H-quinoxaline-1-carboxylic Acid Ethyl Ester

LC-MS (ESI+): 388 (MH+).

### EXAMPLE 248

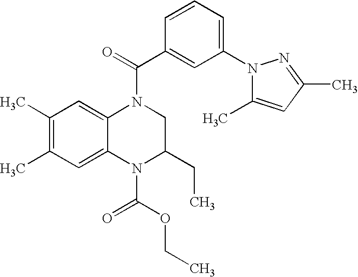

### 4-[3-(3,5-Dimethyl-pyrazol-1-yl)-benzoyl]-2-ethyl-6,7-dimethyl-3,4-dihydro-2H-quinoxaline-1-carboxylic Acid Ethyl Ester

LC-MS (ESI+): 461 (MH+).

### EXAMPLE 249

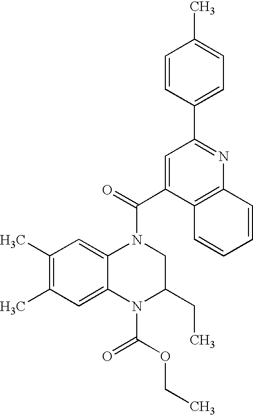

### 2-Ethyl-6,7-dimethyl-4-(2-p-tolyl-quinoline-4-carbonyl)-3,4-dihydro-2H-quinoxaline-1-carboxylic Acid Ethyl Ester

LC-MS (ESI+): 508 (MH+).

### EXAMPLE 250

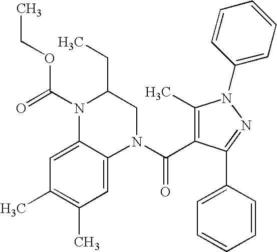

### 2-Ethyl-6,7-dimethyl-4-(5-methyl-1,3-diphenyl-1H-pyrazole-4-carbonyl)-3,4-dihydro-2H-quinoxaline-1-carboxylic Acid Ethyl Ester

LC-MS (ESI+): 523 (MH+).

### EXAMPLE 251

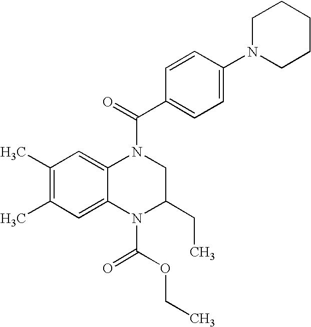

### 2-Ethyl-6,7-dimethyl-4-(4-piperidin-1-yl-benzoyl)-3,4-dihydro-2H-quinoxaline-1-carboxylic Acid Ethyl Ester

LC-MS (ESI+): 450 (MH+).

### EXAMPLE 252

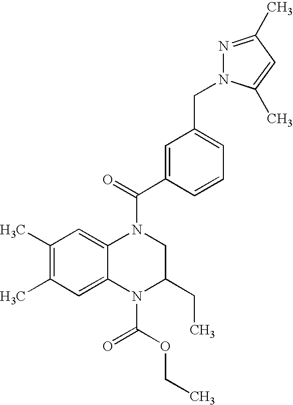

### 4-[3-(3,5-Dimethyl-pyrazol-1-ylmethyl)-benzoyl]-2-ethyl-6,7-dimethyl-3,4-dihydro-2H-quinoxaline-1-carboxylic Acid Ethyl Ester

LC-MS (ESI+): 475 (MH+).

### EXAMPLE 253

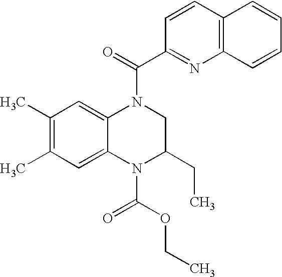

### 2-Ethyl-6,7-dimethyl-4-(quinoline-2-carbonyl)-3,4-dihydro-2H-quinoxaline-1-carboxylic Acid Ethyl Ester

LC-MS (ESI+): 418 (MH+).

### EXAMPLE 254

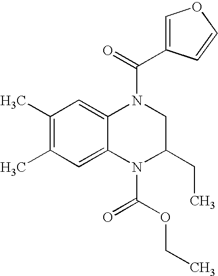

### 2-Ethyl-4-(furan-3-carbonyl)-6,7-dimethyl-3,4-dihydro-2H-quinoxaline-1-carboxylic Acid Ethyl Ester

LC-MS (ESI+): 357 (MH+).

### EXAMPLE 255

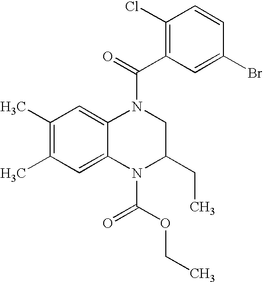

### 4-(5-Bromo-2-chloro-benzoyl)-2-ethyl-6,7-dimethyl-3,4-dihydro-2H-quinoxaline-1-carboxylic Acid Ethyl Ester

LC-MS (ESI+): 480 (MH+).

### EXAMPLE 256

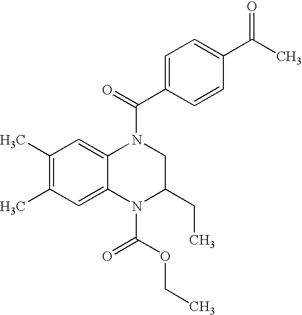

### 4-(4-Acetyl-benzoyl)-2-ethyl-6,7-dimethyl-3,4-dihydro-2H-quinoxaline-1-carboxylic Acid Ethyl Ester

LC-MS (ESI+): 409 (MH+).

### EXAMPLE 257

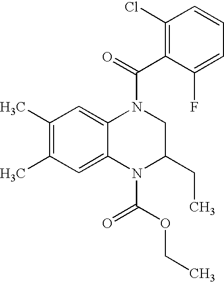

### 4-(2-Chloro-6-fluoro-benzoyl )-2-ethyl-6,7-dimethyl-3,4-dihydro-2H-quinoxaline-1-carboxylic Acid Ethyl Ester

LC-MS (ESI+): 419 (MH+).

### EXAMPLE 258

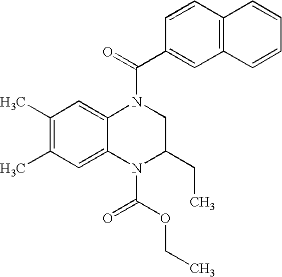

### 2-Ethyl-6,7-dimethyl-4-(naphthalene-2-carbonyl)-3,4-dihydro-2H-quinoxaline-1-carboxylic Acid Ethyl Ester

LC-MS (ESI+): 417 (MH+).

### EXAMPLE 259

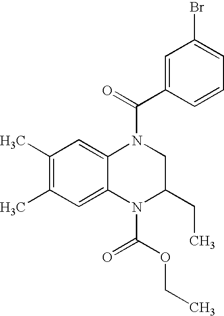

### 4-(3-Bromo-benzoyl)-2-ethyl-6,7-dimethyl-3,4-dihydro-2H-quinoxaline-1-carboxylic Acid Ethyl Ester

LC-MS (ESI+): 446 (MH+).

### EXAMPLE 260

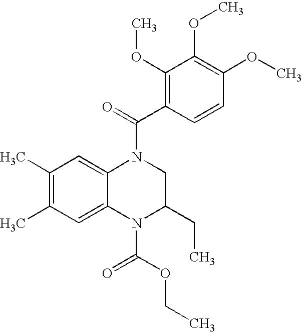

### 2-Ethyl-6,7-dimethyl-4-(2,3,4-trimethoxy-benzoyl)-3,4-dihydro-2H-quinoxaline-1-carboxylic Acid Ethyl Ester

LC-MS (ESI+): 457 (MH+).

### EXAMPLE 261

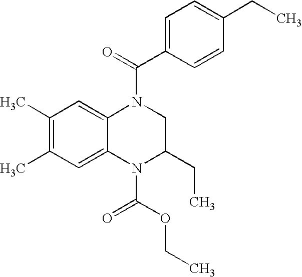

### 2-Ethyl-4-(4-ethyl-benzoyl)-6,7-dimethyl-3,4-dihydro-2H-quinoxaline-1-carboxylic Acid Ethyl Ester

LC-MS (ESI+): 395 (MH+).

### EXAMPLE 262

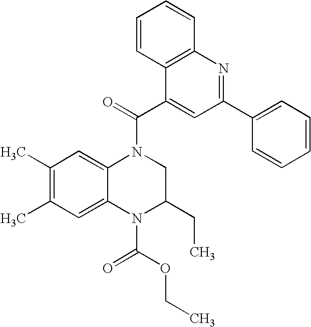

### 2-Ethyl-6,7-dimethyl-4-(2-phenyl-quinoline-4-carbonyl)-3,4-dihydro-2H-quinoxaline-1-carboxylic Acid Ethyl Ester

LC-MS (ESI+): 494 (MH+).

### EXAMPLE 263

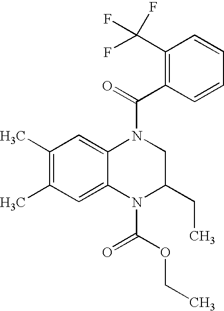

### 2-Ethyl-6,7-dimethyl-4-(2-trifluoromethyl-benzoyl)-3,4-dihydro-2H-quinoxaline-1-carboxylic Acid Ethyl Ester

LC-MS (ESI+): 435 (MH+).

### EXAMPLE 264

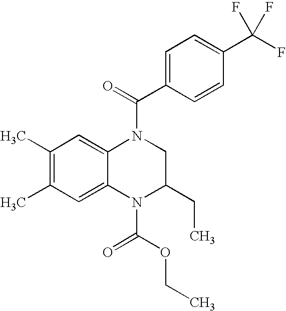

### 2-Ethyl-6,7-dimethyl-4-(4-trifluoromethyl-benzoyl)-3,4-dihydro-2H-quinoxaline-1-carboxylic Acid Ethyl Ester

LC-MS (ESI+): 435 (MH+).

### EXAMPLE 265

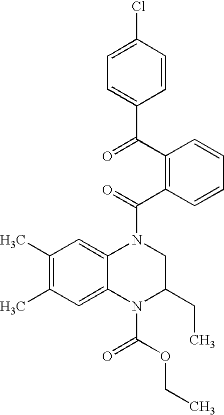

### 4-[2-(4-Chloro-benzoyl)-benzoyl]-2-ethyl-6,7-dimethyl-3,4-dihydro-2H-quinoxaline-1-carboxylic Acid Ethyl Ester

LC-MS (ESI+): 506 (MH+).

### EXAMPLE 266

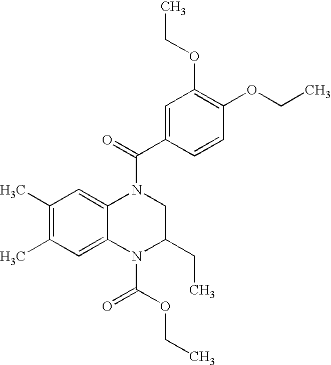

### 4-(3,4-Diethoxy-benzoyl)-2-ethyl-6,7-dimethyl-3,4-dihydro-2H-quinoxaline-1-carboxylic Acid Ethyl Ester

LC-MS (ESI+): 455 (MH+).

### EXAMPLE 267

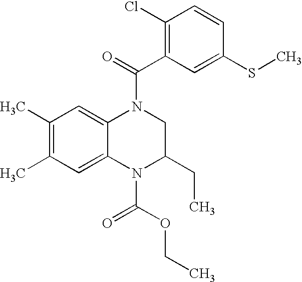

### 4-(2-Chloro-5-methylsulfanyl-benzoyl)-2-ethyl-6,7-dimethyl-3,4-dihydro-2H-quinoxaline-1-carboxylic Acid Ethyl Ester

LC-MS (ESI+): 448 (MH+).

### EXAMPLE 268

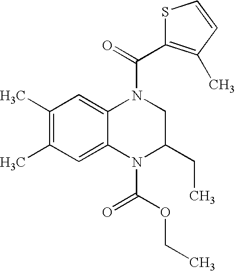

### 2-Ethyl-6,7-dimethyl-4-(3-methyl-thiophene-2-carbonyl)-3,4-dihydro-2H-quinoxaline-1-carboxylic Acid Ethyl Ester

LC-MS (ESI+): 387 (MH+).

### EXAMPLE 269

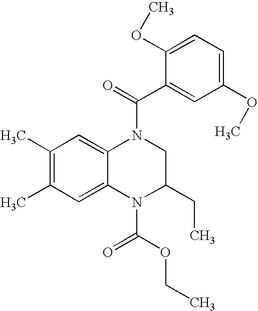

### 4-(2,5-Dimethoxy-benzoyl)-2-ethyl-6,7-dimethyl-3,4-dihydro-2H-quinoxaline-1-carboxylic Acid Ethyl Ester

LC-MS (ESI+): 427 (MH+).

### EXAMPLE 270

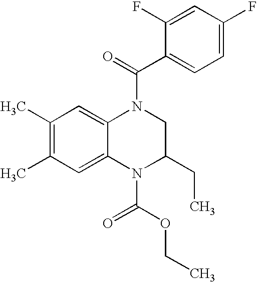

### 4-(2,4-Difluoro-benzoyl)-2-ethyl-6,7-dimethyl-3,4-dihydro-2H-quinoxaline-1-carboxylic Acid Ethyl Ester

LC-MS (ESI+): 403 (MH+).

### EXAMPLE 271

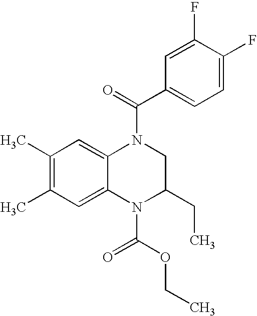

### 4-(3,4-Difluoro-benzoyl)-2-ethyl-6,7-dimethyl-3,4-dihydro-2H-quinoxaline-1-carboxylic Acid Ethyl Ester

LC-MS (ESI+): 403 (MH+).

### EXAMPLE 272

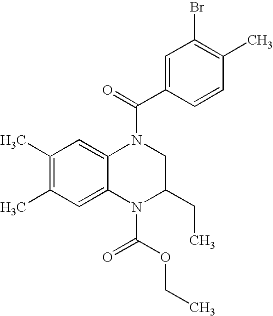

### 4-(3-Bromo-4-methyl-benzoyl)-2-ethyl-6,7-dimethyl-3,4-dihydro-2H-quinoxaline-1-carboxylic Acid Ethyl Ester

LC-MS (ESI+): 460 (MH+).

### EXAMPLE 273

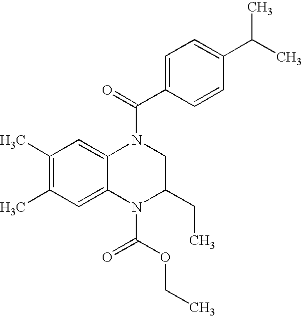

### 2-Ethyl-4-(4-isopropyl-benzoyl)-6,7-dimethyl-3,4-dihydro-2H-quinoxaline-1-carboxylic Acid Ethyl Ester

LC-MS (ESI+): 409 (MH+).

### EXAMPLE 274

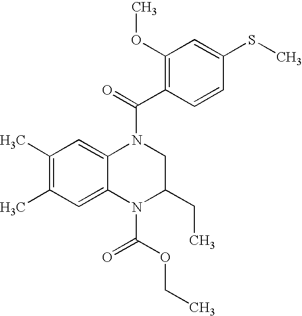

### 2-Ethyl-4-(2-methoxy-4-methylsulfanyl-benzoyl)-6,7-dimethyl-3,4-dihydro-2H-quinoxaline-1-carboxylic Acid Ethyl Ester

LC-MS (ESI+): 443 (MH+).

### EXAMPLE 275

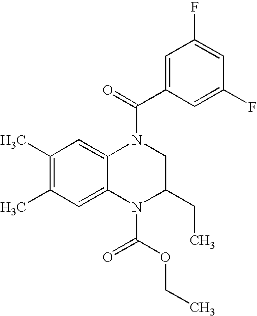

### 4-(3,5-Difluoro-benzoyl)-2-ethyl-6,7-dimethyl-3,4-dihydro-2H-quinoxaline-1-carboxylic Acid Ethyl Ester

LC-MS (ESI+): 403 (MH+).

### EXAMPLE 276

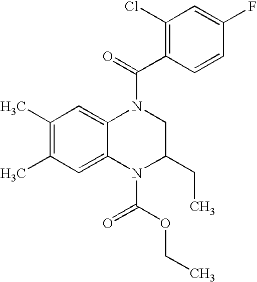

### 4-(2-Chloro-4-fluoro-benzoyl )-2-ethyl-6,7-dimethyl-3,4-dihydro-2H-quinoxaline-1-carboxylic Acid Ethyl Ester

LC-MS (ESI+): 419 (MH+).

### EXAMPLE 277

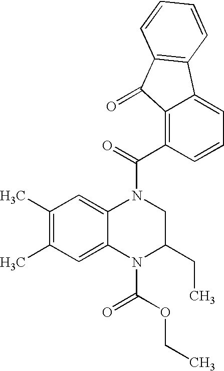

### 2-Ethyl-6,7-dimethyl-4-(9-oxo-9H-fluorene-1-carbonyl)-3,4-dihydro-2H-quinoxaline-1-carboxylic Acid Ethyl Ester

LC-MS (ESI+): 469 (MH+).

### EXAMPLE 278

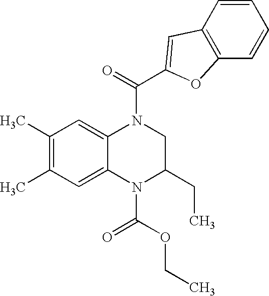

### 4-(Benzofuran-2-carbonyl)-2-ethyl-6,7-dimethyl-3,4-dihydro-2H-quinoxaline-1-carboxylic Acid Ethyl Ester

LC-MS (ESI+): 407 (MH+).

### EXAMPLE 279

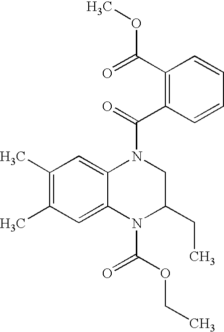

### 2-Ethyl-4-(2-methoxycarbonyl-benzoyl)-6,7-dimethyl-3,4-dihydro-2H-quinoxaline-1-carboxylic Acid Ethyl Ester

LC-MS (ESI+): 425 (MH+).

### EXAMPLE 280

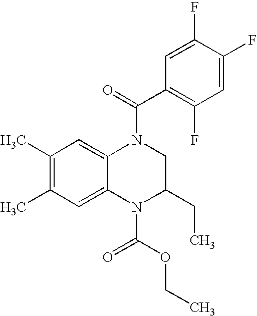

### 2-Ethyl-6,7-dimethyl-4-(2,4,5-trifluoro-benzoyl)-3,4-dihydro-2H-quinoxaline-1-carboxylic Acid Ethyl Ester

LC-MS (ESI+): 421 (MH+).

### EXAMPLE 281

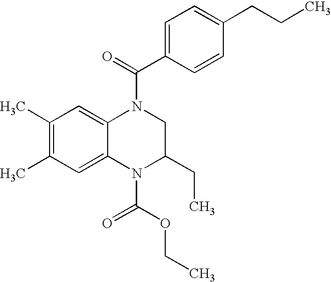

### 2-Ethyl-6,7-dimethyl-4-(4-propyl-benzoyl)-3,4-dihydro-2H-quinoxaline-1-carboxylic Acid Ethyl Ester

LC-MS (ESI+): 409 (MH+).

### EXAMPLE 282

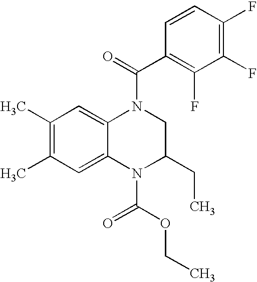

### 2-Ethyl-6,7-dimethyl-4-(2,3,4-trifluoro-benzoyl)-3,4-dihydro-2H-quinoxaline-1-carboxylic Acid Ethyl Ester

LC-MS (ESI+): 421 (MH+).

### EXAMPLE 283

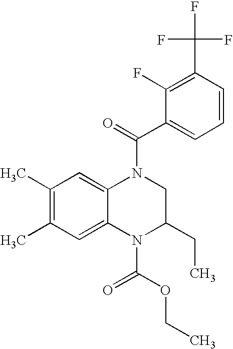

### 2-Ethyl-4-(2-fluoro-3-trifluoromethyl-benzoyl)-6,7-dimethyl-3,4-dihydro-2H-quinoxaline-1-carboxylic Acid Ethyl Ester

LC-MS (ESI+): 453 (MH+).

### EXAMPLE 284

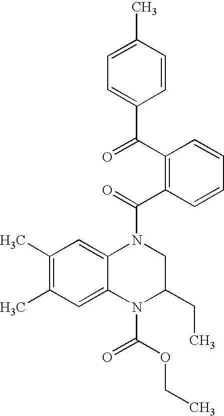

### 2-Ethyl-6,7-dimethyl-4-[2-(4-methyl-benzoyl)-benzoyl]-3,4-dihydro-2H-quinoxaline-1-carboxylic Acid Ethyl Ester

LC-MS (ESI+): 485 (MH+).

### EXAMPLE 285

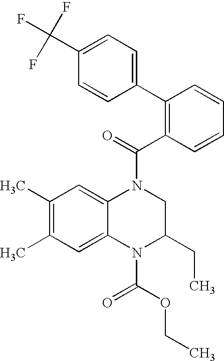

### 2-Ethyl-6,7-dimethyl-4-(4′-trifluoromethyl-biphenyl-2-carbonyl)-3,4-dihydro-2H-quinoxaline-1-carboxylic Acid Ethyl Ester

LC-MS (ESI+): 511 (MH+).

### EXAMPLE 286

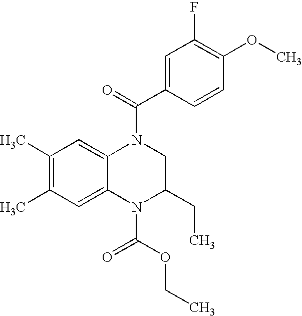

### 2-Ethyl-4-(3-fluoro-4-methoxy-benzoyl)-6,7-dimethyl-3,4-dihydro-2H-quinoxaline-1-carboxylic Acid Ethyl Ester

LC-MS (ESI+): 415 (MH+).

### EXAMPLE 287

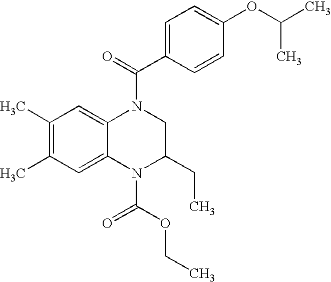

### 2-Ethyl-4-(4-isopropoxy-benzoyl)-6,7-dimethyl-3,4-dihydro-2H-quinoxaline-1-carboxylic Acid Ethyl Ester

LC-MS (ESI+): 425 (MH+).

### EXAMPLE 288

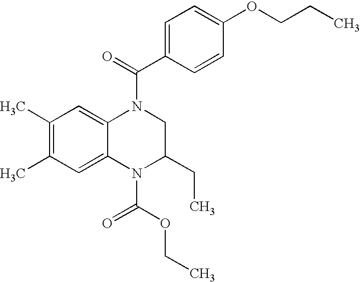

### 2-Ethyl-6,7-dimethyl-4-(4-propoxy-benzoyl)-3,4-dihydro-2H-quinoxaline-1-carboxylic Acid Ethyl Ester

LC-MS (ESI+): 425 (MH+).

### EXAMPLE 289

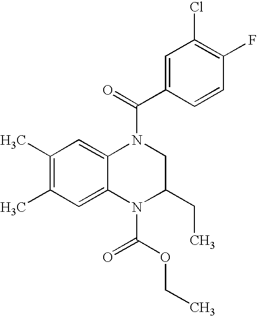

### 4-(3-Chloro-4-fluoro-benzoyl)-2-ethyl-6,7-dimethyl-3,4-dihydro-2H-quinoxaline-1-carboxylic Acid Ethyl Ester

LC-MS (ESI+): 419 (MH+).

### EXAMPLE 290

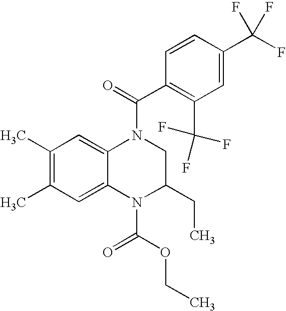

### 4-(2,4-Bis-trifluoromethyl-benzoyl)-2-ethyl-6,7-dimethyl-3,4-dihydro-2H-quinoxaline-1-carboxylic Acid Ethyl Ester

LC-MS (ESI+): 503 (MH+).

### EXAMPLE 291

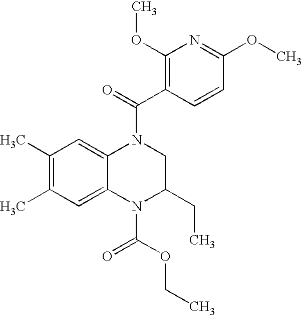

### 4-(2,6-Dimethoxy-pyridine-3-carbonyl)-2-ethyl-6,7-dimethyl-3,4-dihydro-2H-quinoxaline-1-carboxylic Acid Ethyl Ester

LC-MS (ESI+): 428 (MH+).

### EXAMPLE 292

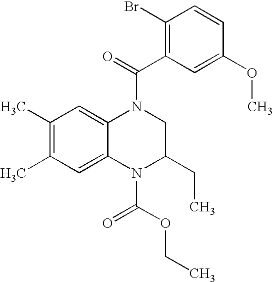

### 4-(2-Bromo-5-methoxy-benzoyl)-2-ethyl-6,7-dimethyl-3,4-dihydro-2H-quinoxaline-1-carboxylic Acid Ethyl Ester

LC-MS (ESI+): 476 (MH+).

### EXAMPLE 293

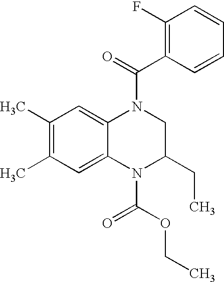

### 2-Ethyl-4-(2-fluoro-benzoyl)-6,7-dimethyl-3,4-dihydro-2H-quinoxaline-1-carboxylic Acid Ethyl Ester

LC-MS (ESI+): 385 (MH+).

### EXAMPLE 294

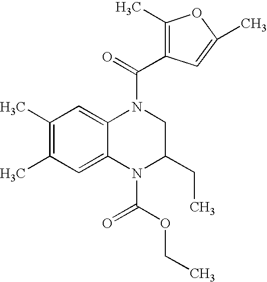

### 4-(2,5-Dimethyl-furan-3-carbonyl)-2-ethyl-6,7-dimethyl-3,4-dihydro-2H-quinoxaline-1-carboxylic Acid Ethyl Ester

LC-MS (ESI+): 385 (MH+).

### EXAMPLE 295

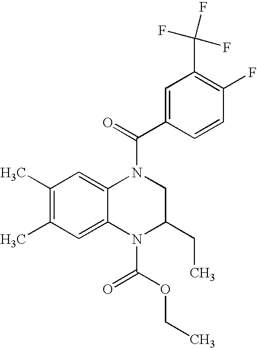

### 2-Ethyl-4-(4-fluoro-3-trifluoromethyl-benzoyl)-6,7-dimethyl-3,4-dihydro-2H-quinoxaline-1-carboxylic Acid Ethyl Ester

LC-MS (ESI+): 453 (MH+).

### EXAMPLE 296

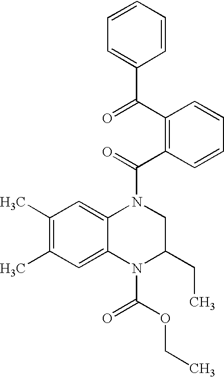

### 4-(2-Benzoyl-benzoyl)-2-ethyl-6,7-dimethyl-3,4-dihydro-2H-quinoxaline-1-carboxylic Acid Ethyl Ester

LC-MS (ESI+): 471 (MH+).

### EXAMPLE 297

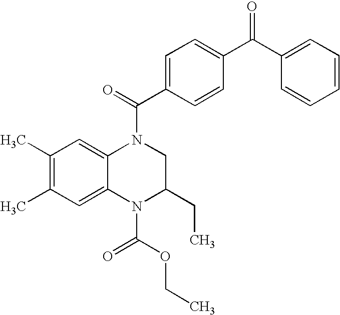

### 4-(4-Benzoyl-benzoyl)-2-ethyl-6,7-dimethyl-3,4-dihydro-2H-quinoxaline-1-carboxylic Acid Ethyl Ester

LC-MS (ESI+): 471 (MH+).

### EXAMPLE 298

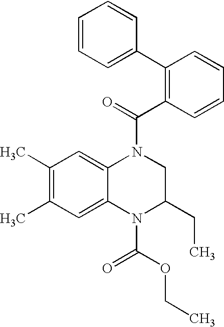

### 4-(Biphenyl-2-carbonyl)-2-ethyl-6,7-dimethyl-3,4-dihydro-2H-quinoxaline-1-carboxylic Acid Ethyl Ester

LC-MS (ESI+): 443 (MH+).

### EXAMPLE 299

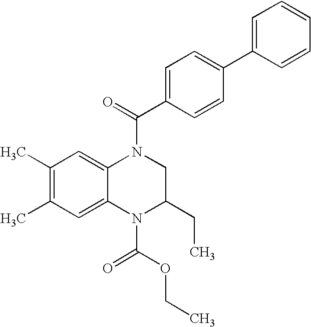

### 4-(Biphenyl-4-carbonyl)-2-ethyl-6,7-dimethyl-3,4-dihydro-2H-quinoxaline-1-carboxylic Acid Ethyl Ester

LC-MS (ESI+): 443 (MH+).

### EXAMPLE 300

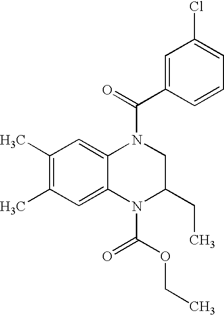

### 4-(3-Chloro-benzoyl)-2-ethyl-6,7-dimethyl-3,4-dihydro-2H-quinoxaline-1-carboxylic Acid Ethyl Ester

LC-MS (ESI+): 401 (MH+).

### EXAMPLE 301

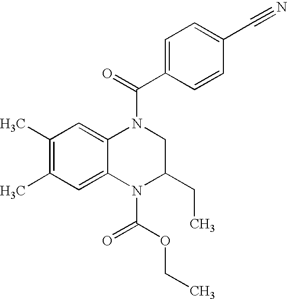

### 4-(4-Cyano-benzoyl)-2-ethyl-6,7-dimethyl-3,4-dihydro-2H-quinoxaline-1-carboxylic Acid Ethyl Ester

LC-MS (ESI+): 392 (MH+).

### EXAMPLE 302

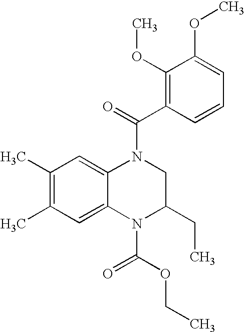

### 4-(2,3-Dimethoxy-benzoyl)-2-ethyl-6,7-dimethyl-3,4-dihydro-2H-quinoxaline-1-carboxylic Acid Ethyl Ester

LC-MS (ESI+): 427 (MH+).

### EXAMPLE 303

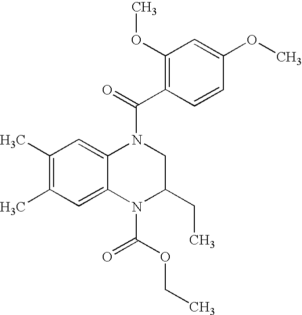

### 4-(2,4-Dimethoxy-benzoyl)-2-ethyl-6,7-dimethyl-3,4-dihydro-2H-quinoxaline-1-carboxylic Acid Ethyl Ester

LC-MS (ESI+): 427 (MH+).

### EXAMPLE 304

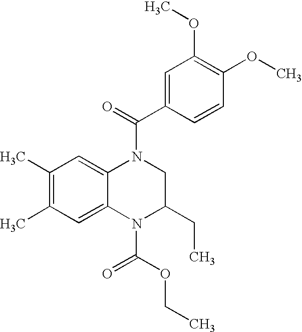

### 4-(3,4-Dimethoxy-benzoyl)-2-ethyl-6,7-dimethyl-3,4-dihydro-2H-quinoxaline-1-carboxylic Acid Ethyl Ester

LC-MS (ESI+): 427 (MH+).

### EXAMPLE 305

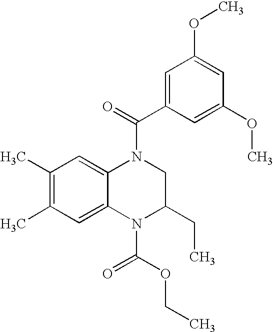

### 4-(3,5-Dimethoxy-benzoyl)-2-ethyl-6,7-dimethyl-3,4-dihydro-2H-quinoxaline-1-carboxylic Acid Ethyl Ester

LC-MS (ESI+): 427 (MH+).

### EXAMPLE 306

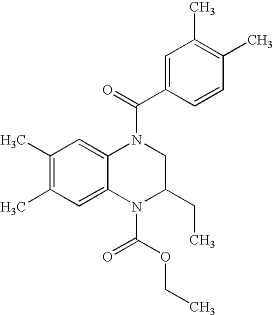

### 4-(3,4-Dimethyl-benzoyl)-2-ethyl-6,7-dimethyl-3,4-dihydro-2H-quinoxaline-1-carboxylic Acid Ethyl Ester

LC-MS (ESI+): 395 (MH+).

### EXAMPLE 307

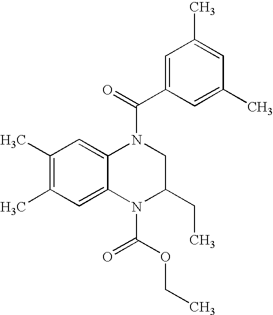

### 4-(3,5-Dimethyl-benzoyl)-2-ethyl-6,7-dimethyl-3,4-dihydro-2H-quinoxaline-1-carboxylic Acid Ethyl Ester

LC-MS (ESI+): 395 (MH+).

### EXAMPLE 308

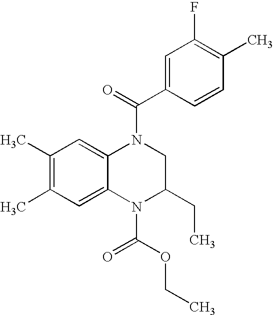

### 2-Ethyl-4-(3-fluoro-4-methyl-benzoyl)-6,7-dimethyl-3,4-dihydro-2H-quinoxaline-1-carboxylic Acid Ethyl Ester

LC-MS (ESI+): 399 (MH+).

### EXAMPLE 309

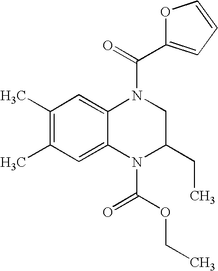

### 2-Ethyl-4-(furan-2-carbonyl)-6,7-dimethyl-3,4-dihydro-2H-quinoxaline-1-carboxylic Acid Ethyl Ester

LC-MS (ESI+): 357 (MH+).

### EXAMPLE 310

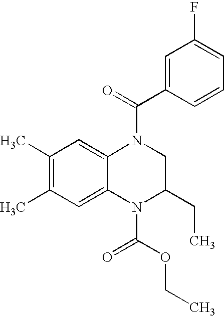

### 2-Ethyl-4-(3-fluoro-benzoyl)-6,7-dimethyl-3,4-dihydro-2H-quinoxaline-1-carboxylic Acid Ethyl Ester

LC-MS (ESI+): 385 (MH+).

### EXAMPLE 311

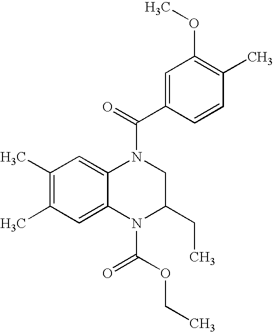

### 2-Ethyl-4-(3-methoxy-4-methyl-benzoyl)-6,7-dimethyl-3,4-dihydro-2H quinoxaline-1-carboxylic Acid Ethyl Ester

LC-MS (ESI+): 411 (MH+).

### EXAMPLE 312

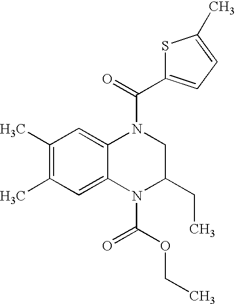

### 2-Ethyl-6,7-dimethyl-4-(5-methyl-thiophene-2-carbonyl)-3,4-dihydro-2H-quinoxaline-1-carboxylic Acid Ethyl Ester

LC-MS (ESI+): 387 (MH+).

### EXAMPLE 313

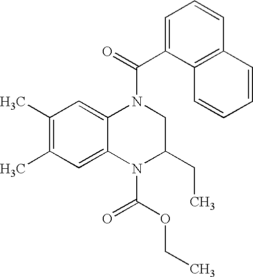

### 2-Ethyl-6,7-dimethyl-4-(naphthalene-1-carbonyl)-3,4-dihydro-2H-quinoxaline-1-carboxylic Acid Ethyl Ester

LC-MS (ESI+): 417 (MH+).

### EXAMPLE 314

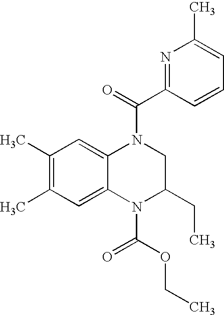

### 2-Ethyl-6,7-dimethyl-4-(6-methyl-pyridine-2-carbonyl)-3,4-dihydro-2H-quinoxaline-1-carboxylic Acid Ethyl Ester

LC-MS (ESI+): 382 (MH+).

### EXAMPLE 315

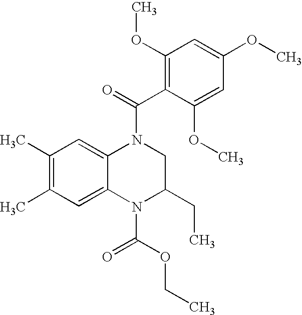

### 2-Ethyl-6,7-dimethyl-4-(2,4,6-trimethoxy-benzoyl)-3,4-dihydro-2H-quinoxaline-1-carboxylic Acid Ethyl Ester

LC-MS (ESI+): 457 (MH+).

### EXAMPLE 316

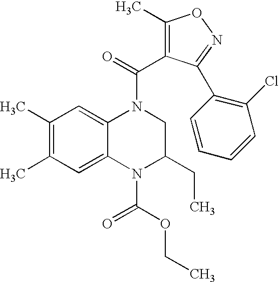

### 4-[3-(2-Chloro-phenyl)-5-methyl-isoxazole-4-carbonyl]-2-ethyl-6,7-dimethyl-3,4-dihydro-2H-quinoxaline-1-carboxylic Acid Ethyl Ester

LC-MS (ESI+): 482 (MH+).

### EXAMPLE 317

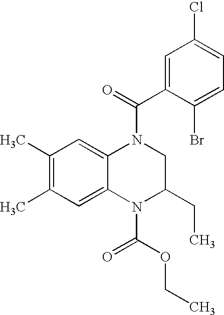

### 4-(2-Bromo-5-chloro-benzoyl)-2-ethyl-6,7-dimethyl-3,4-dihydro-2H-quinoxaline-1-carboxylic Acid Ethyl Ester

LC-MS (ESI+): 480 (MH+).

### EXAMPLE 318

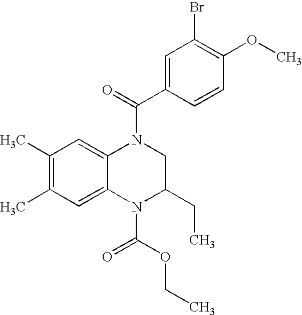

### 4-(3-Bromo-4-methoxy-benzoyl)-2-ethyl-6,7-dimethyl-3,4-dihydro-2H-quinoxaline-1-carboxylic Acid Ethyl Ester

LC-MS (ESI+): 476 (MH+).

### EXAMPLE 319

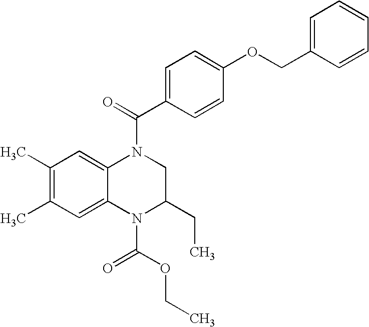

### 4-(4-Benzyloxy-benzoyl)-2-ethyl-6,7-dimethyl-3,4-dihydro-2H-quinoxaline-1-carboxylic Acid Ethyl Ester

LC-MS (ESI+): 473 (MH+).

### EXAMPLE 320

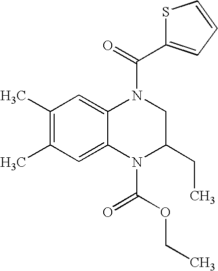

### 2-Ethyl-6,7-dimethyl-4-(thiophene-2-carbonyl)-3,4-dihydro-2H-quinoxaline-1-carboxylic Acid Ethyl Ester

LC-MS (ESI+): 373 (MH+).

### EXAMPLE 321

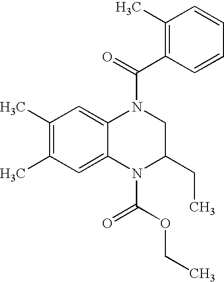

### 2-Ethyl-6,7-dimethyl-4-(2-methyl-benzoyl)-3,4-dihydro-2H-quinoxaline-1-carboxylic Acid Ethyl Ester

LC-MS (ESI+): 381 (MH+).

### EXAMPLE 322

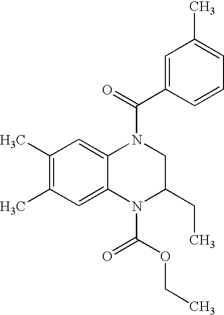

### 2-Ethyl-6,7-dimethyl-4-(3-methyl-benzoyl)-3,4-dihydro-2H-quinoxaline-1-carboxylic Acid Ethyl Ester

LC-MS (ESI+): 381 (MH+).

### EXAMPLE 323

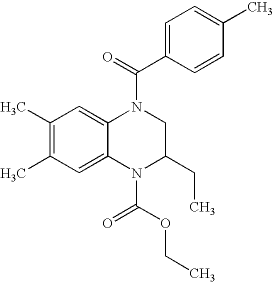

### 2-Ethyl-6,7-dimethyl-4-(4-methyl-benzoyl)-3,4-dihydro-2H-quinoxaline-1-carboxylic Acid Ethyl Ester

LC-MS (ESI+): (MH+).

### EXAMPLE 324

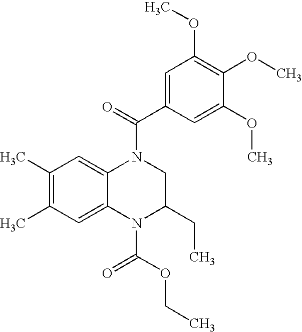

### 2-Ethyl-6,7-dimethyl-4-(3,4,5-trimethoxy-benzoyi)-3,4-dihydro-2H-quinoxaline-1-carboxylic Acid Ethyl Ester

LC-MS (ESI+): 457 (MH+).

### EXAMPLE 325

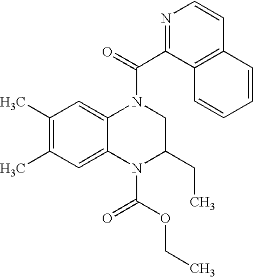

### 2-Ethyl-4-(isoquinoline-1-carbonyl)-6,7-dimethyl-3,4-dihydro-2H-quinoxaline-1-carboxylic Acid Ethyl Ester

LC-MS (ESI+): 418 (MH+).

### EXAMPLE 326

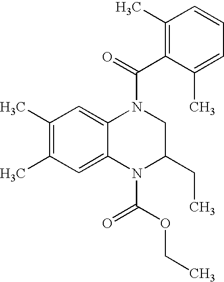

### 4-(2,6-Dimethyl-benzoyl)-2-ethyl-6,7-dimethyl-3,4-dihydro-2H-quinoxaline-1-carboxylic Acid Ethyl Ester

LC-MS (ESI+): 395 (MH+).

### EXAMPLE 327

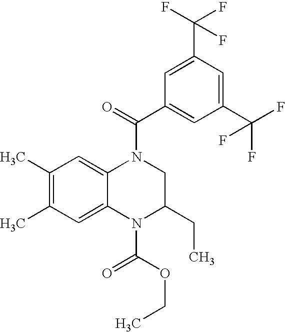

### 4-(3,5-Bis-trifluoromethyl-benzoyl)-2-ethyl-6,7-dimethyl-3,4-dihydro-2H-quinoxaline-1-carboxylic Acid Ethyl Ester

LC-MS (ESI+): 503 (MH+).

### EXAMPLE 328

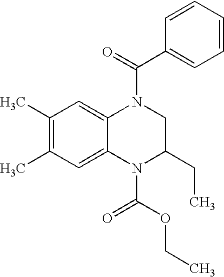

### 4-Benzoyl-2-ethyl-6,7-dimethyl-3,4-dihydro-2H-quinoxaline-1-carboxylic Acid Ethyl Ester

LC-MS (ESI+): 367 (MH+).

### EXAMPLE 329

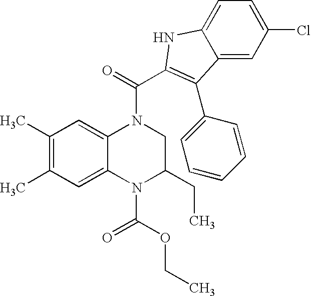

### 4-(5-Chloro-3-phenyl-1H-indole-2-carbonyl)-2-ethyl-6,7-dimethyl-3,4-dihydro-2H-quinoxaline-1-carboxylic Acid Ethyl Ester

LC-MS (ESI+): 517 (MH+).

### EXAMPLE 330

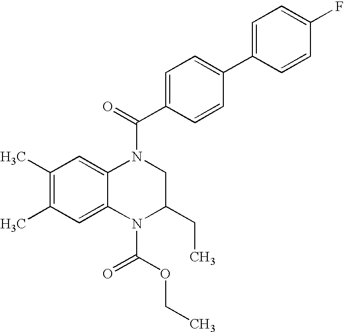

### 2-Ethyl-4-(4′-fluoro-biphenyl-4-carbonyl)-6,7-dimethyl-3,4-dihydro-2H-quinoxaline-1-carboxylic Acid Ethyl Ester

LC-MS (ESI+): 461 (MH+).

### EXAMPLE 331

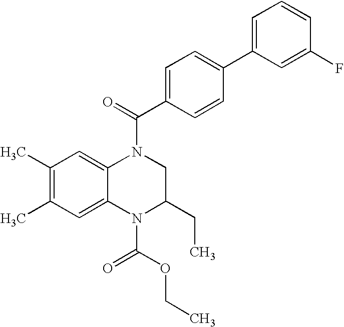

### 2-Ethyl-4-(3′-fluoro-biphenyl-4-carbonyl)-6,7-dimethyl-3,4-dihydro-2H-quinoxaline-1-carboxylic Acid Ethyl Ester

LC-MS (ESI+): 461 (MH+).

### EXAMPLE 332

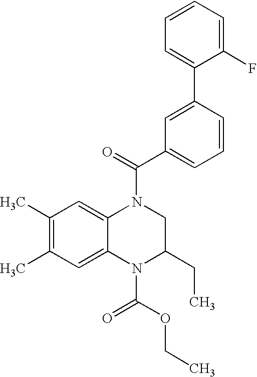

### 2-Ethyl-4-(2′-fluoro-biphenyl-3-carbonyl)-6,7-dimethyl-3,4-dihydro-2H-quinoxaline-1-carboxylic Acid Ethyl Ester

LC-MS (ESI+): 461 (MH+).

### EXAMPLE 333

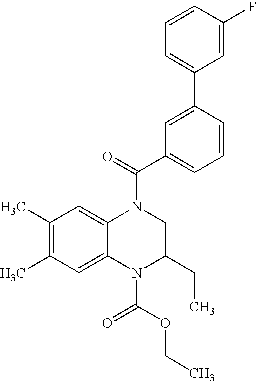

### 2-Ethyl-4-(3′-fluoro-biphenyl-3-carbonyl)-6,7-dimethyl-3,4-dihydro-2H-quinoxaline-1-carboxylic Acid Ethyl Ester

LC-MS (ESI+): 461 (MH+).

### EXAMPLE 334

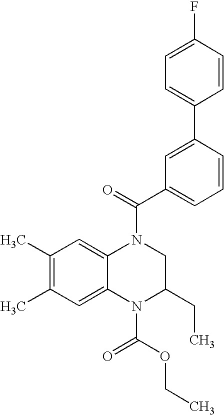

### 2-Ethyl-4-(4′-fluoro-biphenyl-3-carbonyl)-6,7-dimethyl-3,4-dihydro-2H-quinoxaline-1-carboxylic Acid Ethyl Ester

LC-MS (ESI+): 461 (MH+).

### EXAMPLE 335

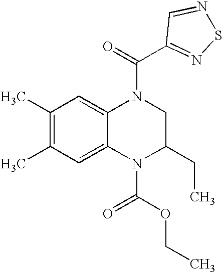

### 2-Ethyl-6,7-dimethyl-4-([1,2,5]thiadiazole-3-carbonyl)-3,4-dihydro-2H-quinoxaline-1-carboxylic Acid Ethyl Ester

LC-MS (ESI+): 375 (MH+).

### EXAMPLE 336

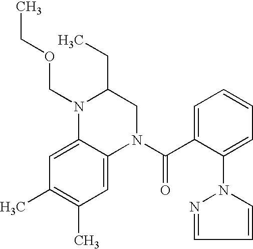

### 2-Ethyl-6,7-dimethyl-4-(2-pyrazol-1-yl-benzoyl)-3,4-dihydro-2H-quinoxaline-1-carboxylic Acid Ethyl Ester

LC-MS (ESI+): 433 (MH+).

### EXAMPLE 337

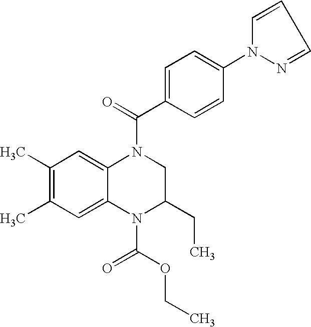

### 2-Ethyl-6,7-dimethyl-4-(4-pyrazol-1-yl-benzoyl)-3,4-dihydro-2H-quinoxaline-1-carboxylic Acid Ethyl Ester

LC-MS (ESI+): 433 (MH+).

### EXAMPLE 338

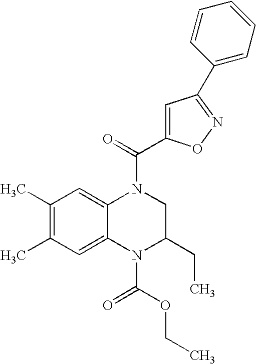

### 2-Ethyl-6,7-dimethyl-4-(3-phenyl-isoxazole-5-carbonyl)-3,4-dihydro-2H-quinoxaline-1-carboxylic Acid Ethyl Ester

LC-MS (ESI+): 434 (MH+).

### EXAMPLE 339

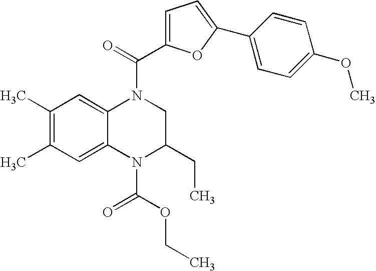

### 2-Ethyl-4-[5-(4-methoxy-phenyl)-furan-2-carbonyl]-6,7-dimethyl-3,4-dihydro-2H-quinoxaline-1-carboxylic Acid Ethyl Ester

LC-MS (ESI+): 463 (MH+).

### EXAMPLE 340

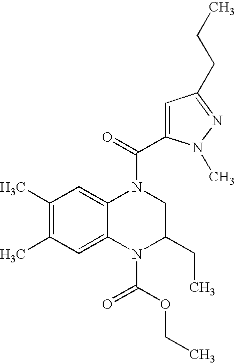

### 2-Ethyl-6,7-dimethyl-4-(2-methyl-5-propyl-2H-pyrazole-3-carbonyl)-3,4-dihydro-2H-quinoxaline-1-carboxylic Acid Ethyl Ester

LC-MS (ESI+): 413(MH+).

### EXAMPLE 341

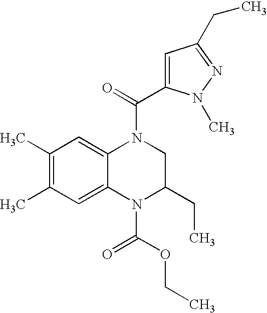

### 2-Ethyl-4-(5-ethyl-2-methyl-2H-pyrazole-3-carbonyl)-6,7-dimethyl-3,4-dihydro-2H-quinoxaline-1-carboxylic Acid Ethyl Ester

LC-MS (ESI+): 399 (MH+).

### EXAMPLE 342

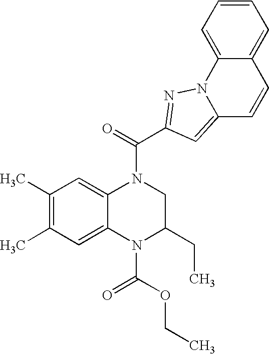

### 2-Ethyl-6,7-dimethyl-4-(pyrazolo[1,5-a]quinoline-2-carbonyl)-3,4-dihydro-2H-quinoxaline-1-carboxylic Acid Ethyl Ester

LC-MS (ESI+): 457 (MH+).

### EXAMPLE 343

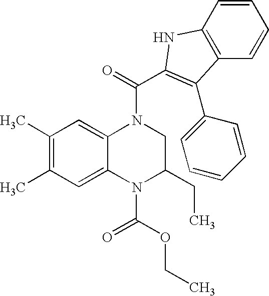

### 2-Ethyl-6,7-dimethyl-4-(3-phenyl-1H-indole-2-carbonyl)-3,4-dihydro-2H-quinoxaline-1-carboxylic Acid Ethyl Ester

LC-MS (ESI+): 482 (MH+).

### EXAMPLE 344

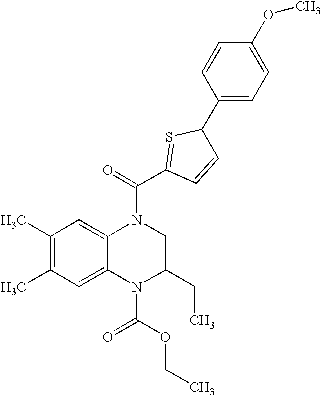

### 2-Ethyl-4-[5-(4-methoxy-phenyl)-thiophene-2-carbonyl]-6,7-dimethyl-3,4-dihydro-2H-quinoxaline-1-carboxylic Acid Ethyl Ester

LC-MS (ESI+): 479 (MH+).

### EXAMPLE 345

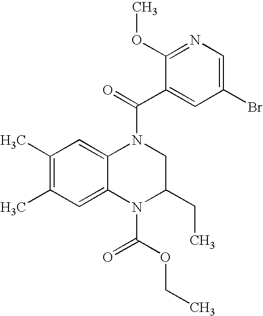

### 4-(5-Bromo-2-methoxy-pyridine-3-carbonyl)-2-ethyl-6,7-dimethyl-3,4-dihydro-2H-quinoxaline-1-carboxylic Acid Ethyl Ester

LC-MS (ESI+): 477 (MH+).

### EXAMPLE 346

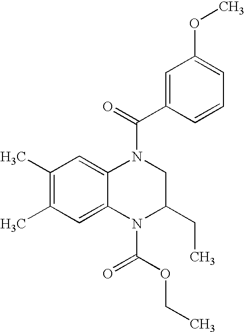

### 2-Ethyl-4-(3-methoxy-benzoyl)-6,7-dimethyl-3,4-dihydro-2H-quinoxaline-1-carboxylic Acid Ethyl Ester

LC-MS (ESI+): 397 (MH+).

### EXAMPLE 347

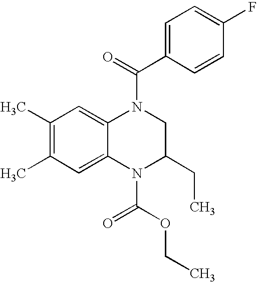

### 2-Ethyl-4-(4-fluoro-benzoyl)-6,7-dimethyl-3,4-dihydro-2H-quinoxaline-1-carboxylic Acid Ethyl Ester

LC-MS (ESI+): 385 (MH+).

### EXAMPLE 348

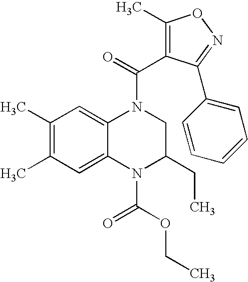

### 2-Ethyl-6,7-dimethyl-4-(5-methyl-3-phenyl-isoxazole-4-carbonyl)-3,4-dihydro-2H-quinoxaline-1-carboxylic Acid Ethyl Ester

LC-MS (ESI+): 448 (MH+).

### EXAMPLE 349

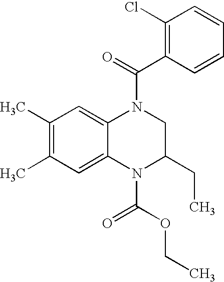

### 4-(2-Chloro-benzoyl)-2-ethyl-6,7-dimethyl-3,4-dihydro-2H-quinoxaline-1-carboxylic Acid Ethyl Ester

LC-MS (ESI+): 401 (MH+).

### EXAMPLE 350

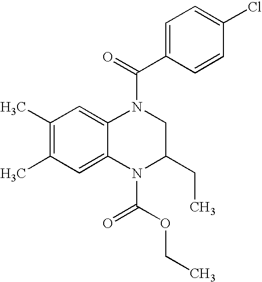

### 4-(4-Chloro-benzoyl)-2-ethyl-6,7-dimethyl-3,4-dihydro-2H-quinoxaline-1-carboxylic Acid Ethyl Ester

LC-MS (ESI+): 401 (MH+).

### EXAMPLE 351

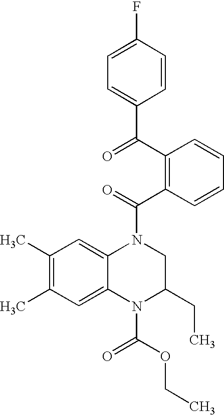

### 2-Ethyl-4-[2-(4-fluoro-benzoyl)-benzoyl]-6,7-dimethyl-3,4-dihydro-2H-quinoxaline-1-carboxylic Acid Ethyl Ester

LC-MS (ESI+): 489 (MH+).

### EXAMPLE 352

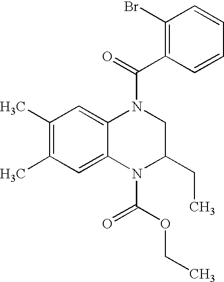

### 4-(2-Bromo-benzoyl)-2-ethyl-6,7-dimethyl-3,4-dihydro-2H-quinoxaline-1-carboxylic Acid Ethyl Ester

LC-MS (ESI+): 446 (MH+).

### EXAMPLE 353

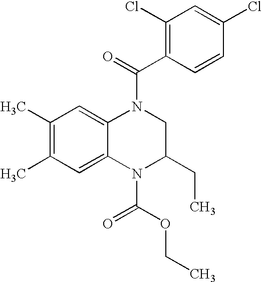

### 4-(2,4-Dichloro-benzoyl)-2-ethyl-6,7-dimethyl-3,4-dihydro-2H-quinoxaline-1-carboxylic Acid Ethyl Ester

LC-MS (ESI+): 436 (MH+).

### EXAMPLE 354

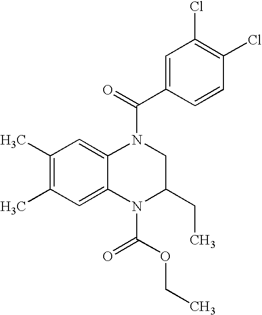

### 4-(3,4-Dichloro-benzoyl)-2-ethyl-6,7-dimethyl-3,4-dihydro-2H-quinoxaline-1-carboxylic Acid Ethyl Ester

LC-MS (ESI+): 436 (MH+).

### EXAMPLE 355

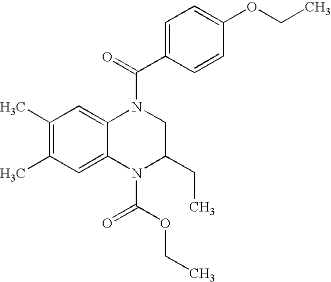

### 4-(4-Ethoxy-benzoyl)-2-ethyl-6,7-dimethyl-3,4-dihydro-2H-quinoxaline-1-carboxylic Acid Ethyl Ester

LC-MS (ESI+): 411 (MH+).

### EXAMPLE 356

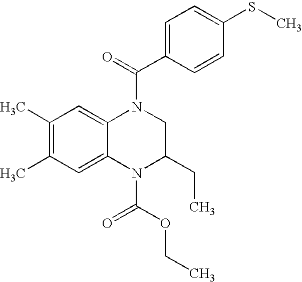

### 2-Ethyl-6,7-dimethyl-4-(4-methylsulfanyl-benzoyl)-3,4-dihydro-2H-quinoxaline-1-carboxylic Acid Ethyl Ester

LC-MS (ESI+): 413 (MH+).

### EXAMPLE 357

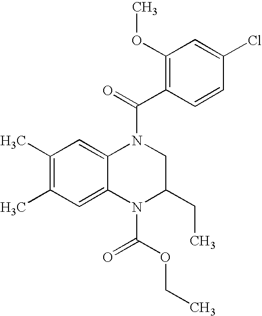

### 4-(4-Chloro-2-methoxy-benzoyl)-2-ethyl-6,7-dimethyl-3,4-dihydro-2H-quinoxaline-1-carboxylic Acid Ethyl Ester

LC-MS (ESI+): 431 (MH+).

### EXAMPLE 358

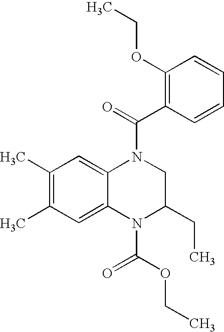

### 4-(2-Ethoxy-benzoyl)-2-ethyl-6,7-dimethyl-3,4-dihydro-2H-quinoxaline-1-carboxylic Acid Ethyl Ester

LC-MS (ESI+): 411 (MH+).

### EXAMPLE 359

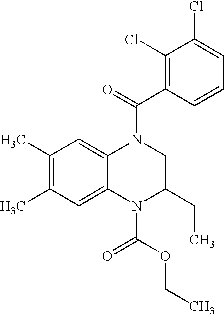

### 4-(2,3-Dichloro-benzoyl)-2-ethyl-6,7-dimethyl-3,4-dihydro-2H-quinoxaline-1-carboxylic Acid Ethyl Ester

LC-MS (ESI+): 436 (MH+).

### EXAMPLE 360

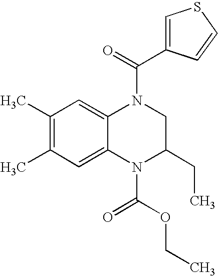

### 2-Ethyl-6,7-dimethyl-4-(thiophene-3-carbonyl)-3,4-dihydro-2H-quinoxaline-1-carboxylic Acid Ethyl Ester

LC-MS (ESI+): 373 (MH+).

### EXAMPLE 361

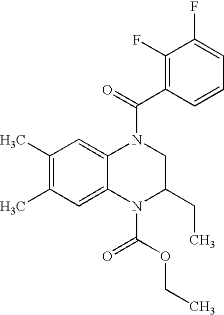

### 4-(2,3-Difluoro-benzoyl)-2-ethyl-6,7-dimethyl-3,4-dihydro-2H-quinoxaline-1-carboxylic Acid Ethyl Ester

LC-MS (ESI+): 403 (MH+).

### EXAMPLE 362

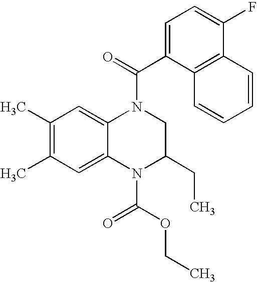

### 2-Ethyl-4-(4-fluoro-naphthalene-1-carbonyl)-6,7-dimethyl-3,4-dihydro-2H-quinoxaline-1-carboxylic Acid Ethyl Ester

LC-MS (ESI+): 435 (MH+).

### EXAMPLE 363

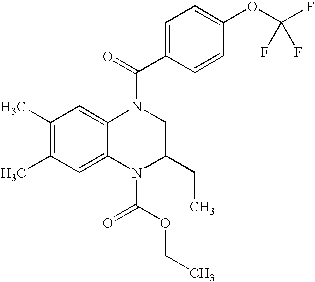

### 2-Ethyl-6,7-dimethyl-4-(4-trifluoromethoxy-benzoyl)-3,4-dihydro-2H-quinoxaline-1-carboxylic Acid Ethyl Ester

LC-MS (ESI+): 451 (MH+).

### EXAMPLE 364

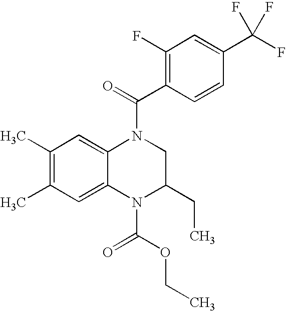

### 2-Ethyl-4-(2-fluoro-4-trifluoromethyl-benzoyl)-6,7-dimethyl-3,4-dihydro-2H-quinoxaline-1-carboxylic Acid Ethyl Ester

LC-MS (ESI+): 453 (MH+).

### EXAMPLE 365

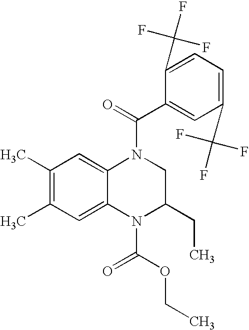

### 4-(2,5-Bis-trifluoromethyl-benzoyl)-2-ethyl-6,7-dimethyl-3,4-dihydro-2H-quinoxaline-1-carboxylic Acid Ethyl Ester

LC-MS (ESI+): 503 (MH+).

### EXAMPLE 366

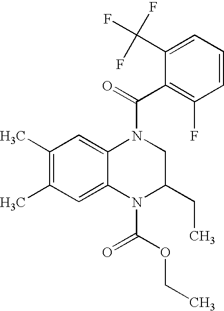

### 2-Ethyl-4-(2-fluoro-6-trifluoromethyl-benzoyl)-6,7-dimethyl-3,4-dihydro-2H-quinoxaline-1-carboxylic Acid Ethyl Ester

LC-MS (ESI+): 453 (MH+).

### EXAMPLE 367

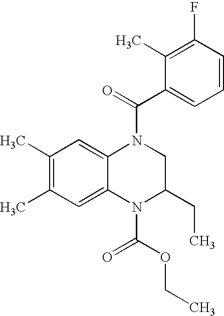

### 2-Ethyl-4-(3-fluoro-2-methyl-benzoyl)-6,7-dimethyl-3,4-dihydro-2H-quinoxaline-1-carboxylic Acid Ethyl Ester

LC-MS (ESI+): 399 (MH+).

### EXAMPLE 368

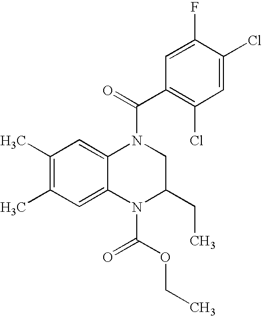

### 4-(2,4-Dichloro-5-fluoro-benzoyl)-2-ethyl-6,7-dimethyl-3,4-dihydro-2H-quinoxaline-1-carboxylic Acid Ethyl Ester

LC-MS (ESI+): 454 (MH+).

### EXAMPLE 369

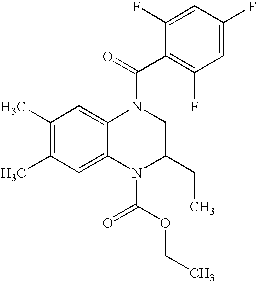

### 2-Ethyl-6,7-dimethyl-4-(2,4,6-trifluoro-benzoyl)-3,4-dihydro-2H-quinoxaline-1-carboxylic Acid Ethyl Ester

LC-MS (ESI+): 421 (MH+).

### EXAMPLE 370

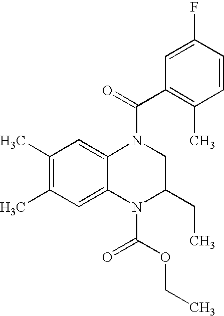

### 2-Ethyl-4-(5-fluoro-2-methyl-benzoyl)-6,7-dimethyl-3,4-dihydro-2H-quinoxaline-1-carboxylic Acid Ethyl Ester

LC-MS (ESI+): 399 (MH+).

### EXAMPLE 371

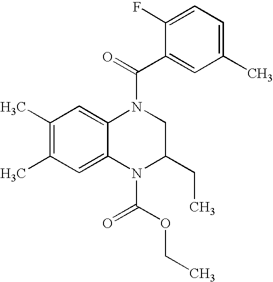

### 2-Ethyl-4-(2-fluoro-5-methyl-benzoyl)-6,7-dimethyl-3,4-dihydro-2H-=quinoxaline-1-carboxylic Acid Ethyl Ester

LC-MS (ESI+): 399 (MH+).

### EXAMPLE 372

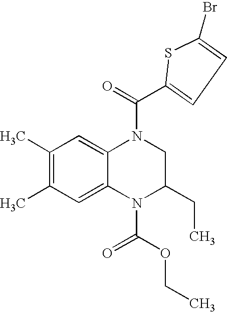

### 4-(5-Bromo-thiophene-2-carbonyl)-2-ethyl-6,7-dimethyl-3,4-dihydro-2H-quinoxaline-1-carboxylic Acid Ethyl Ester

LC-MS (ESI+): 452 (MH+).

### EXAMPLE 373

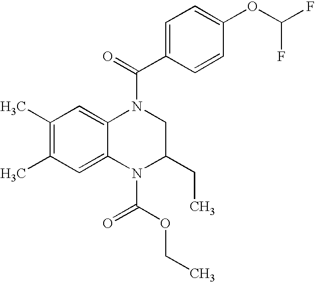

### 4-(4-Difluoromethoxy-benzoyl)-2-ethyl-6,7-dimethyl-3,4-dihydro-2H-quinoxaline-1-carboxylic Acid Ethyl Ester

LC-MS (ESI+): 433 (MH+).

### EXAMPLE 374

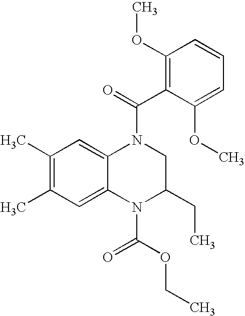

### 4-(2,6-Dimethoxy-benzoyl)-2-ethyl-6,7-dimethyl-3,4-dihydro-2H-quinoxaline-1-carboxylic Acid Ethyl Ester

LC-MS (ESI+): 427 (MH+).

### EXAMPLE 375

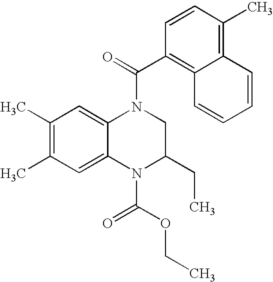

### 2-Ethyl-6,7-dimethyl-4-(4-methyl-naphthalene-1-carbonyl)-3,4-dihydro-2H-quinoxaline-1-carboxylic Acid Ethyl Ester

LC-MS (ESI+): 431 (MH+).

### EXAMPLE 376

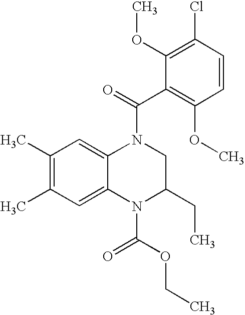

### 4-(3-Chloro-2,6-dimethoxy-benzoyl)-2-ethyl-6,7-dimethyl-3,4-dihydro-2H-quinoxaline-1-carboxylic Acid Ethyl Ester

LC-MS (ESI+): 461 (MH+).

### EXAMPLE 377

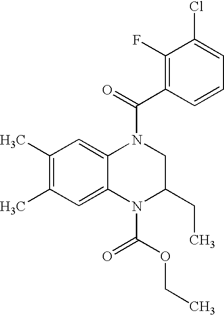

### 4-(3-Chloro-2-fluoro-benzoyl)-2-ethyl-6,7-dimethyl-3,4-dihydro-2H-quinoxaline-1-carboxylic Acid Ethyl Ester

LC-MS (ESI+): 419 (MH+).

### EXAMPLE 378

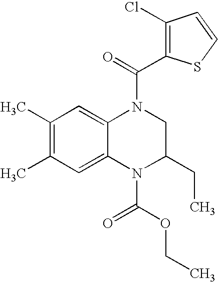

### 4-(3-Chloro-thiophene-2-carbonyl)-2-ethyl-6,7-dimethyl-3,4-dihydro-2H-quinoxaline-1-carboxylic Acid Ethyl Ester

LC-MS (ESI+): 407 (MH+).

### EXAMPLE 379

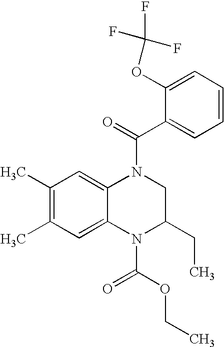

### 2-Ethyl-6,7-dimethyl-4-(2-trifluoromethoxy-benzoyl)-3,4-dihydro-2H-quinoxaline-1-carboxylic Acid Ethyl Ester

LC-MS (ESI+): 451 (MH+).

### EXAMPLE 380

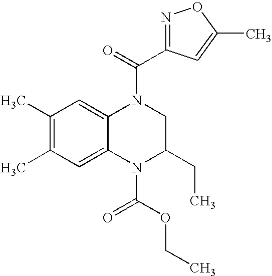

### 2-Ethyl-6,7-dimethyl-4-(5-methyl-isoxazole-3-carbonyl)-3,4-dihydro-2H-quinoxaline-1-carboxylic Acid Ethyl Ester

LC-MS (ESI+): 372 (MH+).

### EXAMPLE 381

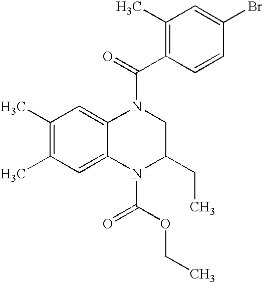

### 2-Ethyl-6,7-dimethyl-4-(3-methyl-furan-2-carbonyl)-3,4-dihydro-2H-quinoxaline-1-carboxylic Acid Ethyl Ester

LC-MS (ESI+): 371 (MH+).

### EXAMPLE 382

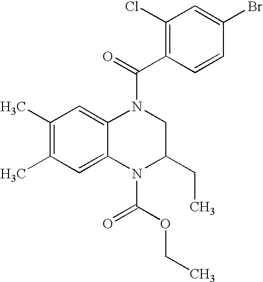

### 4-(4-Bromo-2-methyl-benzoyl)-2-ethyl-6,7-dimethyl-3,4-dihydro-2H-quinoxaline-1-carboxylic Acid Ethyl Ester

LC-MS (ESI+): 460 (MH+).

### EXAMPLE 383

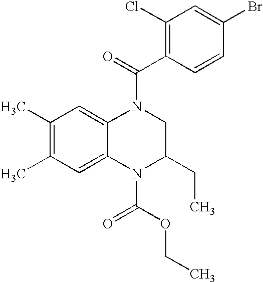

### 4-(4-Bromo-2-chloro-benzoyl)-2-ethyl-6,7-dimethyl-3,4-dihydro-2H-quinoxaline-1-carboxylic Acid Ethyl Ester

LC-MS (ESI+): 480 (MH+).

### EXAMPLE 384

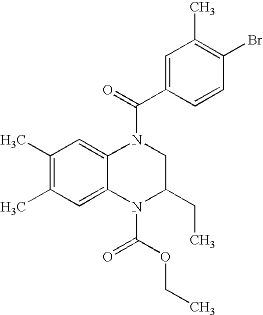

### 4-(4-Bromo-3-methyl-benzoyl)-2-ethyl-6,7-dimethyl-3,4-dihydro-2H-quinoxaline-1-carboxylic Acid Ethyl Ester

LC-MS (ESI+): 460 (MH+).

### EXAMPLE 385

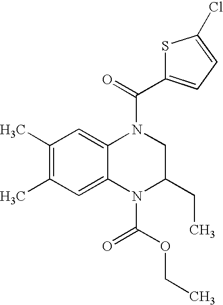

### 4-(5-Chloro-thiophene-2-carbonyl)-2-ethyl-6,7-dimethyl-3,4-dihydro-2H-quinoxaline-1-carboxylic Acid Ethyl Ester

LC-MS (ESI+): 407 (MH+).

### EXAMPLE 386

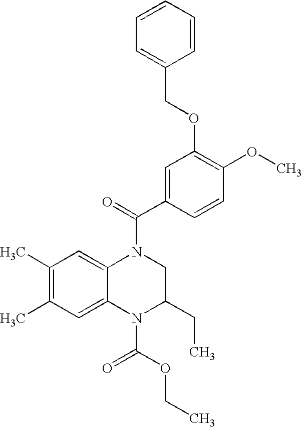

### 4-(3-Benzyloxy-4-methoxy-benzoyl)-2-ethyl-6,7-dimethyl-3,4-dihydro-2H-quinoxaline-1-carboxylic Acid Ethyl Ester

LC-MS (ESI+): 503 (MH+).

### EXAMPLE 387

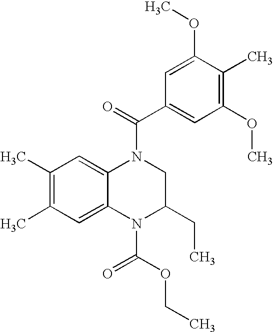

### 4-(3,5-Dimethoxy-4-methyl-benzoyl)-2-ethyl-6,7-dimethyl-3,4-dihydro-2H-quinoxaline-1-carboxylic Acid Ethyl Ester

LC-MS (ESI+): 441 (MH+).

### EXAMPLE 388

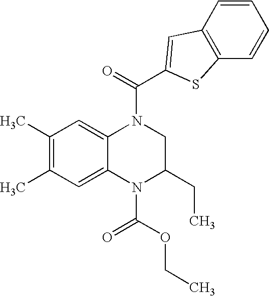

### 4-(Benzo[b]thiophene-2-carbonyl)-2-ethyl-6,7-dimethyl-3,4-dihydro-2H-quinoxaline-1-carboxylic Acid Ethyl Ester

LC-MS (ESI+): 423 (MH+).

### EXAMPLE 389

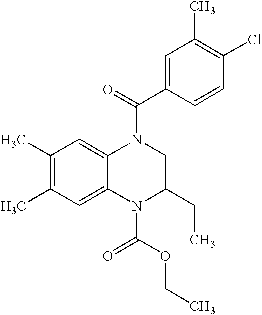

### 4-(4-Chloro-3-methyl-benzoyl)-2-ethyl-6,7-dimethyl-3,4-dihydro-2H-quinoxaline-1-carboxylic Acid Ethyl Ester

LC-MS (ESI+): 415 (MH+).

### EXAMPLE 390

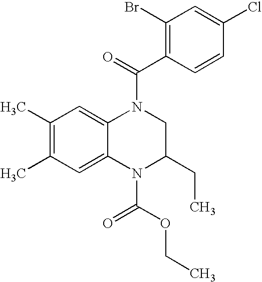

### 4-(2-Bromo-4-chloro-benzoyl)-2-ethyl-6,7-dimethyl-3,4-dihydro-2H-quinoxaline-1-carboxylic Acid Ethyl Ester

LC-MS (ESI+): 480 (MH+).

### EXAMPLE 391

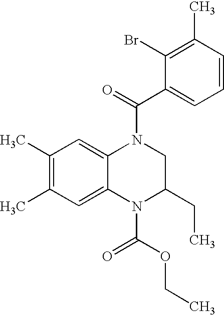

### 4-(2-Bromo-3-methyl-benzoyl)-2-ethyl-6,7-dimethyl-3,4-dihydro-2H-quinoxaline-1-carboxylic Acid Ethyl Ester

LC-MS (ESI+): 460 (MH+).

### EXAMPLE 392

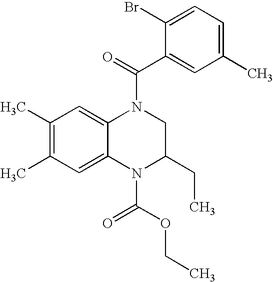

### 4-(2-Bromo-5-methyl-benzoyl)-2-ethyl-6,7-dimethyl-3,4-dihydro-2H-uinoxaline-1-carboxylic Acid Ethyl Ester

LC-MS (ESI+): 460 (MH+).

### EXAMPLE 393

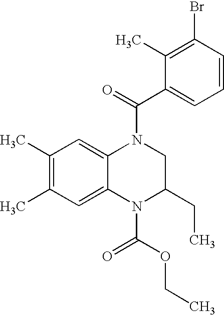

### 4-(3-Bromo-2-methyl-benzoyl)-2-ethyl-6,7-dimethyl-3,4-dihydro-2H-quinoxaline-1-carboxylic Acid Ethyl Ester

LC-MS (ESI+): 460 (MH+).

### EXAMPLE 394

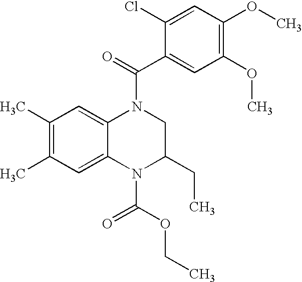

### 4-(2-Chloro-4,5-dimethoxy-benzoyl)-2-ethyl-6,7-dimethyl-3,4-dihydro-2H-quinoxaline-1-carboxylic Acid Ethyl Ester

LC-MS (ESI+): 461 (MH+).

### EXAMPLE 395

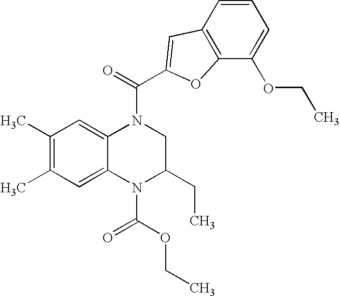

### 4-(7-Ethoxy-benzofuran-2-carbonyl)-2-ethyl-6,7-dimethyl-3,4-dihydro-2H-quinoxaline-1-carboxylic Acid Ethyl Ester

LC-MS (ESI+): 451 (MH+).

### EXAMPLE 396

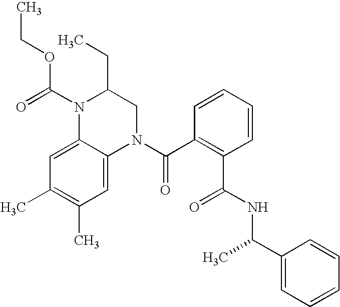

### 2-Ethyl-6,7-dimethyl-4-[2-(1-phenyl-ethylcarbamoyl)-benzoyl]-3,4-dihydro-2H-quinoxaline-1-carboxylic Acid Ethyl Ester

LC-MS (ESI+): 514 (MH+).

### EXAMPLE 397

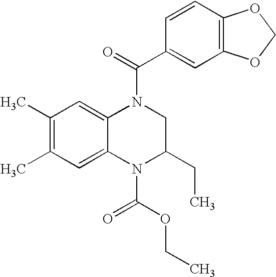

### 4-(Benzo[1,3]dioxole-5-carbonyl)-2-ethyl-6,7-dimethyl-3,4-dihydro-2H-quinoxaline-1-carboxylic Acid Ethyl Ester

LC-MS (ESI+): 411 (MH+).

### EXAMPLE 398

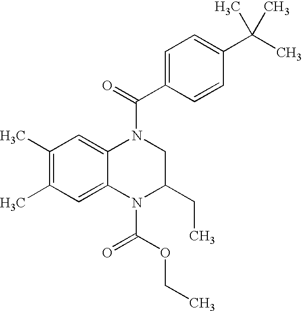

### 4-(4-tert-Butyl-benzoyl)-2-ethyl-6,7-dimethyl-3,4-dihydro-2H-quinoxaline-1-carboxylic Acid Ethyl Ester

LC-MS (ESI+): 423 (MH+).

### EXAMPLE 399

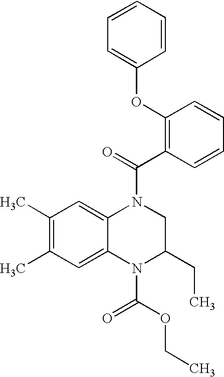

### 2-Ethyl-6,7-dimethyl-4-(2-phenoxy-benzoyl)-3,4-dihydro-2H-quinoxaline-1-carboxylic Acid Ethyl Ester

LC-MS (ESI+): 459 (MH+).

### EXAMPLE 400

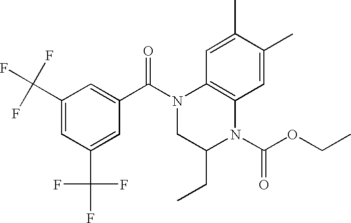

### 4-(3,5-Bis-trifluoromethyl-benzoyl)-2-ethyl-6,7-dimethyl-3,4-dihydro-2H-quinoxaline-1-carboxylic Acid Ethyl Ester

LC-MS (ESI+): 503 (MH+).

### EXAMPLE 401

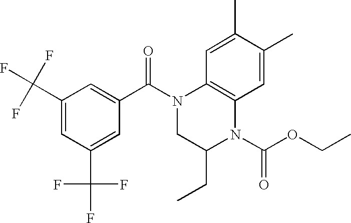

### 4-(3,5-Bis-trifluoromethyl-benzoyl)-2-ethyl-6,7-dimethyl-3,4-dihydro-2H-quinoxaline-1-carboxylic Acid Ethyl Ester

LC-MS (ESI+): 503 (MH+).

### EXAMPLE 402

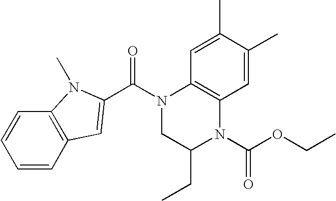

### 2-Ethyl-6,7-dimethyl-4-(1-methyl-1H-indole-2-carbonyl)-3,4-dihydro-2H-quinoxaline-1-carboxylic Acid Ethyl Ester

LC-MS (ESI+): 420 (MH+).

### EXAMPLE 403

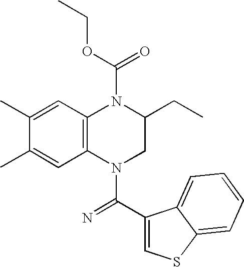

### 4-(Benzo[b]thiophene-3-carbonyl)-2-ethyl-6,7-dimethyl-3,4-dihydro-2H-quinoxaline-1-carboxylic Acid Ethyl Ester

LC-MS (ESI+): 423 (MH+).

### EXAMPLE 404

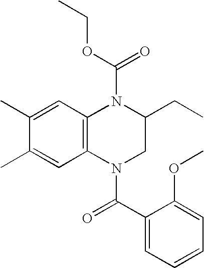

### 2-Ethyl-4-(2-methoxy-benzoyl)-6,7-dimethyl-3,4-dihydro-2H-quinoxaline-1-carboxylic Acid Ethyl Ester

LC-MS (ESI+): 3977 (MH+).

### EXAMPLE 405

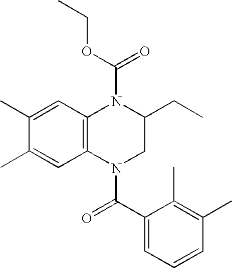

### 4-(2,3-Dimethyl-benzoyl)-2-ethyl-6,7-dimethyl-3,4-dihydro-2H-quinoxaline-1-carboxylic Acid Ethyl Ester

LC-MS (ESI+): 395 (MH+).

### EXAMPLE 406

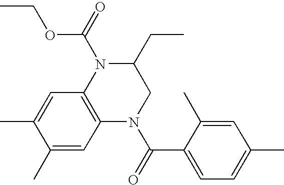

### 4-(2,4-Dimethyl-benzoyl)-2-ethyl-6,7-dimethyl-3,4-dihydro-2H-quinoxaline-1-carboxylic Acid Ethyl Ester

LC-MS (ESI+): 395 (MH+).

### EXAMPLE 407

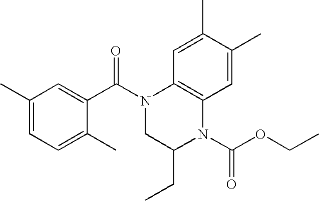

### 4-(2,5-Dimethyl-benzoyl)-2-ethyl-6,7-dimethyl-3,4-dihydro-2H-quinoxaline-1-carboxylic Acid Ethyl Ester

LC-MS (ESI+): 395 (MH+).

### EXAMPLE 408

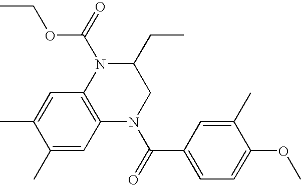

### 2-Ethyl-4-(4-methoxy-3-methyl-benzoyl)-6,7-dimethyl-3,4-dihydro-2H-quinoxaline-1-carboxylic Acid Ethyl Ester

LC-MS (ESI+): 411 (MH+).

### EXAMPLE 409

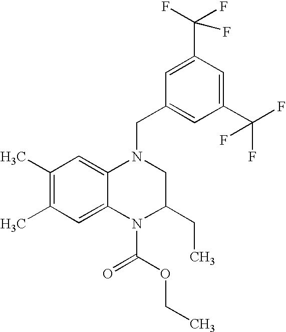

### Preparation of 4-(3,5-Bis-trifluoromethyl-benzyl)-2-ethyl-6,7-dimethyl-3,4-dihydro-2H-quinoxaline-1-carboxylic Acid Ethyl Ester

A solution of 2-ethyl-6,7-dimethyl-3,4-dihydro-2H-quinoxaline-1-carboxylic acid ethyl ester (0.05 g, 1 eq, 0.19 mmol), 3,5-bis-(trifluoromethyl)benzyl bromide (0.038 mL, 2.2 eq, 0.42 mmol) and triethylamine (0.062 mL, 3 eq, 0.57 mmol) in acetonitrile (2 mL) was treated with catalytic potassium iodide and subjected to two ten minute cycles on the Emrys Optimizer (Personal Chemistry, Uppsala, Sweden) chemical microwave at 180° C. The reaction was evaporated to dryness and purified on the Shimadzu Corporation (Kyoto, Japan) preparative HPLC system using a 40-100% acetonitrile/water 6 min gradient, 8 min total run time to provide the title compound (0.0045 mg, 5%).

¹HNMR (CDCl3): δ 0.90 (t, J=7.47 Hz, 3H), 1.31 (t, J=7.05 Hz, 3H), 1.46 (m, 2H), 2.11 (s, 3H), 2.16 (s, 3H), 3.17 (dd, J=11.28, 1.24 Hz, 1H), 3.49 (dd, J=11.16, 4.15 Hz, 1H), 4.19 (m, 1H), 4.28 (m, 1H), 4.46 (d, J=17.43 Hz), 4.52 (m, 1H), 4.66 (d, J=17.01 Hz, 1H), 6.31 (s, 1H), 7.29 (brs, 1H), 7.71 (s, 2H), 7.78 (s, 1H).

LCMS (ESI+): 489 (MH+).

The prophetic examples 410-427 may be prepared in optically enriched form by resolution of the corresponding racemate indicated, or an intermediate in its synthesis, using methods analogous to those described herein:

### EXAMPLE 410

### (Stereo-Isomers of Example 2)

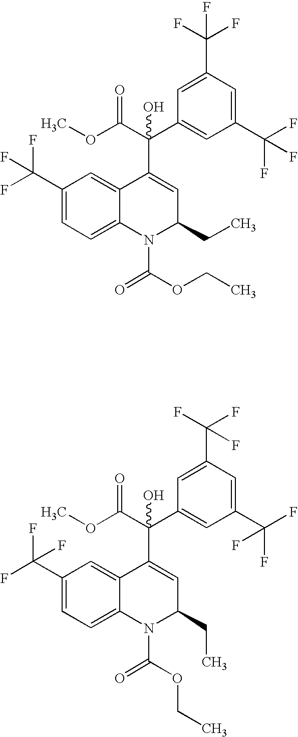

(R, R)-4-[(3,5-Bis-trifluoromethyl-phenyl)-hydroxyl-methoxycarbonyl-methyl]-2-ethyl-6-trifluoromethyl-2H-quinoline-1-carboxylic acid ethyl ester;

(R, S)-4-[(3,5-Bis-trifluoromethyl-phenyl)-hydroxyl-methoxycarbonyl-methyl]-2-ethyl-6-trifluoromethyl-2H-quinoline-1-carboxylic acid ethyl ester;

(S, R)-4-[(3,5-Bis-trifluoromethyl-phenyl)-hydroxyl-methoxycarbonyl-methyl]-2-ethyl-6-trifluoromethyl-2H-quinoline-1-carboxylic acid ethyl ester; or

(S, S)-4-[(3,5-Bis-trifluoromethyl-phenyl)-hydroxyl-methoxycarbonyl-methyl]-2-ethyl-6-trifluoromethyl-2H-quinoline-1-carboxylic acid ethyl ester;

or a pharmaceutically acceptable salt of said compound.

### EXAMPLE 411

### (Stereo-Isomers of Examples 3 and 4)

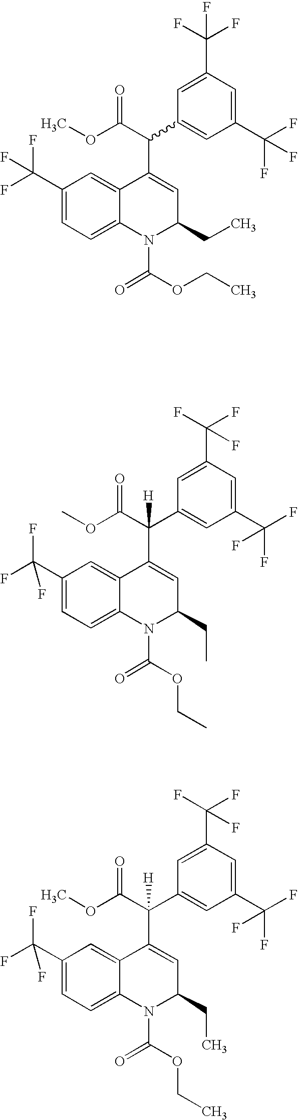

(S, R)-4-[(3,5-Bis-trifluoromethyl-phenyl)-methoxycarbonyl-methyl]-2-ethyl-6-trifluoromethyl-2H-quinoline-1-carboxylic acid ethyl ester; or

(S, S)-4-[(3,5-Bis-trifluoromethyl-phenyl)-methoxycarbonyl-methyl]-2-ethyl-6-trifluoromethyl-2H-quinoline-1-carboxylic acid ethyl ester; or a pharmaceutically acceptable salt of said compound.

### EXAMPLE 412

### (Stereo-Isomers of Examples 5, 6, 7 and 8)

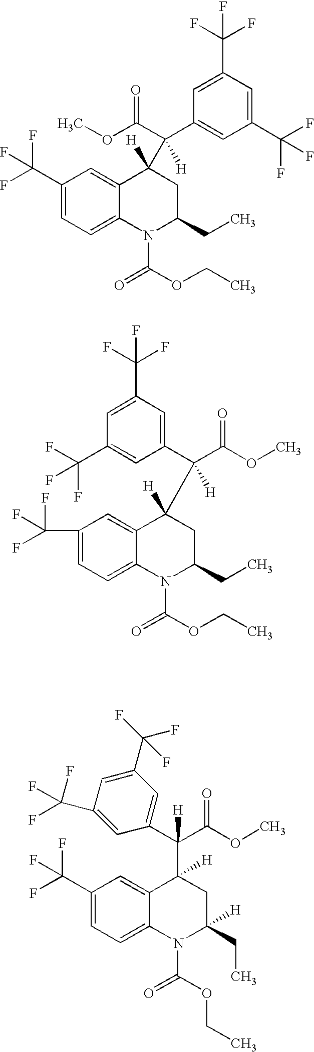

(R, S, R)-4-[(3,5-Bis-trifluoromethyl-phenyl)-methoxycarbonyl-methyl]-2-ethyl-6-trifluoromethyl-3,4-dihydro-2H-quinoline-1-carboxylic acid ethyl ester;

(R, S, S)-4-[(3,5-Bis-trifluoromethyl-phenyl)-methoxycarbonyl-methyl]-2-ethyl-6-trifluoromethyl-3,4-dihydro-2H-quinoline-1-carboxylic acid ethyl ester;

(R, R, S)-4-[(3,5-Bis-trifluoromethyl-phenyl)-methoxycarbonyl-methyl]-2-ethyl-6-trifluoromethyl-3,4-dihydro-2H-quinoline-1-carboxylic acid ethyl ester;

(R, R, R)-4-[(3,5-Bis-trifluoromethyl-phenyl)-methoxycarbonyl-methyl]-2-ethyl-6-trifluoromethyl-3,4-dihydro-2H-quinoline-1-carboxylic acid ethyl ester;

(S, S, R)-4-[(3,5-Bis-trifluoromethyl-phenyl)-methoxycarbonyl-methyl]-2-ethyl-6-trifluoromethyl-3,4-dihydro-2H-quinoline-1-carboxylic acid ethyl ester;

(S, S, S)-4-[(3,5-Bis-trifluoromethyl-phenyl)-methoxycarbonyl-methyl]-2-ethyl-6-trifluoromethyl-3,4-dihydro-2H-quinoline-1-carboxylic acid ethyl ester;

(S, R, S)-4-[(3,5-Bis-trifluoromethyl-phenyl)-methoxycarbonyl-methyl]-2-ethyl-6-trifluoromethyl-3,4-dihydro-2H-quinoline-1-carboxylic acid ethyl ester; or

(S, R, R)-4-[(3,5-Bis-trifluoromethyl-phenyl)-methoxycarbonyl-methyl]-2-ethyl-6-trifluoromethyl-3,4-dihydro-2H-quinoline-1-carboxylic acid ethyl ester; or

a pharmaceutically acceptable salt of said compound.

### EXAMPLE 413

### (Stereo-Isomers of Examples 9 and 10)

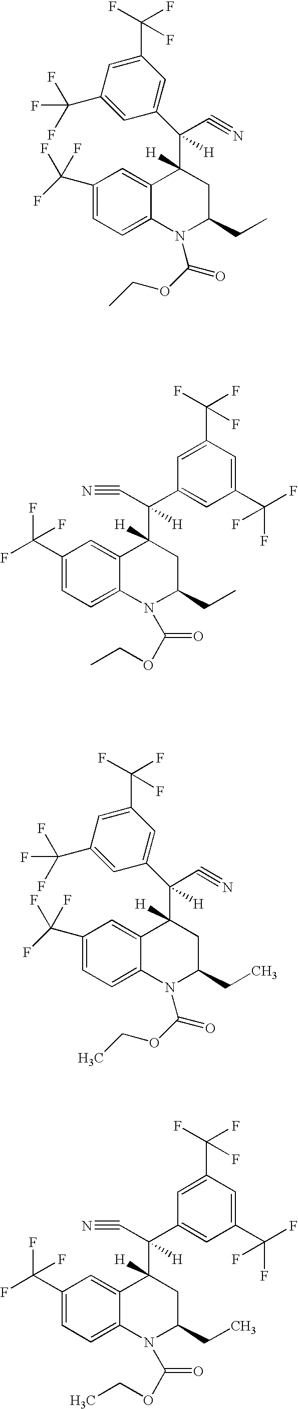

(R, R, R)-4-[(3,5-Bis-trifluoromethyl-phenyl)-cyano-methyl]-2-ethyl-6-trifluoromethyl-3,4-dihydro-2H-quinoline-1-carboxylic acid ethyl ester;

(R, R, S)-4-[(3,5-Bis-trifluoromethyl-phenyl)-cyano-methyl]-2-ethyl-6-trifluoromethyl-3,4-dihydro-2H-quinoline-1-carboxylic acid ethyl ester;

(R, S, R)-4-[(3,5-Bis-trifluoromethyl-phenyl)-cyano-methyl]-2-ethyl-6-trifluoromethyl-3,4-dihydro-2H-quinoline-1-carboxylic acid ethyl ester;

(R, S, S)-4-[(3,5-Bis-trifluoromethyl-phenyl)-cyano-methyl]-2-ethyl-6-trifluoromethyl-3,4-dihydro-2H-quinoline-1-carboxylic acid ethyl ester;

(S, S, S)-4-[(3,5-Bis-trifluoromethyl-phenyl)-cyano-methyl]-2-ethyl-6-trifluoromethyl-3,4-dihydro-2H-quinoline-1-carboxylic acid ethyl ester;

(S, S, R)-4-[(3,5-Bis-trifluoromethyl-phenyl)-cyano-methyl]-2-ethyl-6-trifluoromethyl-3,4-dihydro-2H-quinoline-1-carboxylic acid ethyl ester;

(S, R, R)-4-[(3,5-Bis-trifluoromethyl-phenyl)-cyano-methyl]-2-ethyl-6-trifluoromethyl-3,4-dihydro-2H-quinoline-1-carboxylic acid ethyl ester;

(S, R, R)-4-[(3,5-Bis-trifluoromethyl-phenyl)-cyano-methyl]-2-ethyl-6-trifluoromethyl-3,4-dihydro-2H-quinoline-1-carboxylic acid ethyl ester;

4-[(3,5-Bis-trifluoromethyl-phenyl)-cyano-methyl]-2-ethyl-6-trifluoromethyl-2H-quinoline-1-carboxylic acid ethyl ester;

(R,S)-4-[(3,5-Bis-trifluoromethyl-phenyl)-cyano-methyl]-2-ethyl-6-trifluoromethyl-2H-quinoline-1-carboxylic acid ethyl ester;

(R,R)-4-[(3,5-Bis-trifluoromethyl-phenyl)-cyano-methyl]-2-ethyl-6-trifluoromethyl-2H-quinoline-1-carboxylic acid ethyl ester;

(S,R)-4-[(3,5-Bis-trifluoromethyl-phenyl)-cyano-methyl]-2-ethyl-6-trifluoromethyl-2H-quinoline-1-carboxylic acid ethyl ester; or

(S,S)-4-[(3,5-Bis-trifluoromethyl-phenyl)-cyano-methyl]-2-ethyl-6-trifluoromethyl-2H-quinoline-1-carboxylic acid ethyl ester;

or a pharmaceutically acceptable salt of said compound.

### EXAMPLE 414

### (Stereo-Isomers of Examples 13 and 14)

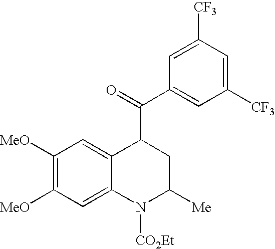

(R,S)-4-[(3,5-bis-trifluoromethyl-benzoyl)]-6,7-dimethoxy-2-methyl-3,4-dihydro-2H-quinoline-1-carboxylic acid ethyl ester;

(R,R)-4-[(3,5-Bis-trifluoromethyl-benzoyl)]-6,7-dimethoxy-2-methyl-3,4-dihydro-2H-quinoline-1-carboxylic acid ethyl ester;

(S,R)-4-[(3,5-bis-trifluoromethyl-benzoyl)]-6,7-dimethoxy-2-methyl-3,4-dihydro-2H-quinoline-1-carboxylic acid ethyl ester; or

(S,S)-4-[(3,5-Bis-trifluoromethyl-benzoyl)]-6,7-dimethoxy-2-methyl-3,4-dihydro-2H-quinoline-1-carboxylic acid ethyl ester;

or a pharmaceutically acceptable salt thereof.

### EXAMPLE 415

### (Stereo-Isomers of Examples 18, 19 and 35)

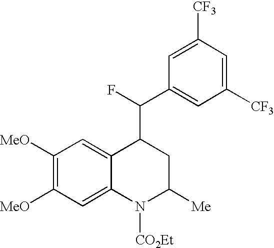

(R,R,R)-4-[(3,5-bis-trifluoromethyl-phenyl)-fluoro-methyl]-6,7-dimethoxy-2-methyl-3,4-dihydro-2H-quinoline-1-carboxylic acid ethyl ester;

(R,R,S)-4-[(3,5-bis-trifluoromethyl-phenyl)-fluoro-methyl]-6,7-dimethoxy-2-methyl-3,4-dihydro-2H-quinoline-1-carboxylic acid ethyl ester;

(R,S,R)-4-[(3,5-bis-trifluoromethyl-phenyl)-fluoro-methyl]-6,7-dimethoxy-2-methyl-3,4-dihydro-2H-quinoline-1-carboxylic acid ethyl ester;

(R,S,S)-4-[(3,5-bis-trifluoromethyl-phenyl)-fluoro-methyl]-6,7-dimethoxy-2-methyl-3,4-dihydro-2H-quinoline-1-carboxylic acid ethyl ester;

(S,R,R)-4-[(3,5-bis-trifluoromethyl-phenyl)-fluoro-methyl]-6,7-dimethoxy-2-methyl-3,4-dihydro-2H-quinoline-1-carboxylic acid ethyl ester;

(S,R,S)-4-[(3,5-bis-trifluoromethyl-phenyl)-fluoro-methyl]-6,7-dimethoxy-2-methyl-3,4-dihydro-2H-quinoline-1-carboxylic acid ethyl ester;

(S,S,R)-4-[(3,5-bis-trifluoromethyl-phenyl)-fluoro-methyl]-6,7-dimethoxy-2-methyl-3,4-dihydro-2H-quinoline-1-carboxylic acid ethyl ester; or

(S,S,S)-4-[(3,5-bis-trifluoromethyl-phenyl)-fluoro-methyl]-6,7-dimethoxy-2-methyl-3,4-dihydro-2H-quinoline-1-carboxylic acid ethyl ester;

or a pharmaceutically acceptable salt of said compound.

### EXAMPLE 416

### (Stereo-Isomers of Examples 20 and 21)

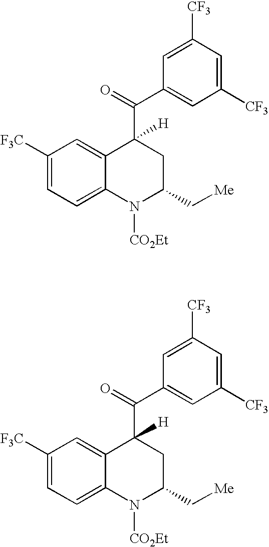

(S,R)-4-[(3,5-bis-trifluoromethyl-benzoyl)]-2-ethyl-6-trifluoromethyl-3,4-dihydro-2H-quinoline-1-carboxylic acid ethyl ester; or

(S,S)-4-[(3,5-bis-trifluoromethyl-benzoyl)]-2-ethyl-6-trifluoromethyl-3,4-dihydro-2H-quinoline-1-carboxylic acid ethyl ester;

or a pharmaceutically acceptable salt of said compound.

### EXAMPLE 417

### (Stereo-Isomers of Examples 16 and 17)

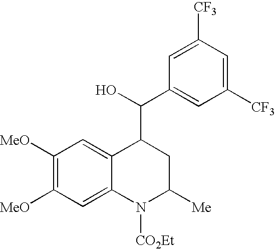

(R,R,R)-4-[(3,5-bis-trifluoromethyl-phenyl)-hydroxy-methyl]-6,7-dimethoxy-2-methyl-3,4-dihydro-2H-quinoline-1-carboxylic acid ethyl ester;

(R,R,S)-4-[(3,5-bis-trifluoromethyl-phenyl)-hydroxy-methyl]-6,7-dimethoxy-2-methyl-3,4-dihydro-2H-quinoline-1-carboxylic acid ethyl ester;

(R,S,R)-4-[(3,5-bis-trifluoromethyl-phenyl)-hydroxy-methyl]-6,7-dimethoxy-2-methyl-3,4-dihydro-2H-quinoline-1-carboxylic acid ethyl ester;

(R,S,S)-4-[(3,5-bis-trifluoromethyl-phenyl)-hydroxy-methyl]-6,7-dimethoxy-2-methyl-3,4-dihydro-2H-quinoline-1-carboxylic acid ethyl ester;

(S,R,R)-4-[(3,5-bis-trifluoromethyl-phenyl)-hydroxy-methyl]-6,7-dimethoxy-2-methyl-3,4-dihydro-2H-quinoline-1-carboxylic acid ethyl ester;

(S,R,S)-4-[(3,5-bis-trifluoromethyl-phenyl)-hydroxy-methyl]-6,7-dimethoxy-2-methyl-3,4-dihydro-2H-quinoline-1-carboxylic acid ethyl ester;

(S,S,R)-4-[(3,5-bis-trifluoromethyl-phenyl)-hydroxy-methyl]-6,7-dimethoxy-2-methyl-3,4-dihydro-2H-quinoline-1-carboxylic acid ethyl ester; or

(S,S,S)-4-[(3,5-bis-trifluoromethyl-phenyl)-hydroxy-methyl]-6,7-dimethoxy-2-methyl-3,4-dihydro-2H-quinoline-1-carboxylic acid ethyl ester;

or a pharmaceutically acceptable salt of said compound.

### EXAMPLE 418

### (Stereo-Isomers of Example 34)

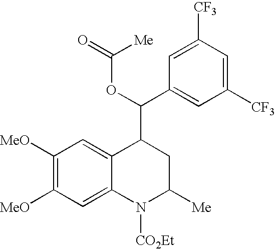

(R,S,S)-4-[Acetoxy-(3,5-Bis-trifluoromethyl-phenyl)-methyl]-6,7-dimethoxy-2-methyl-3,4-dihydro-2H-quinoline-1-carboxylic acid ethyl ester;

(R,R,S)-4-[Acetoxy-(3,5-Bis-trifluoromethyl-phenyl)-methyl]-6,7-dimethoxy-2-methyl-3,4-dihydro-2H-quinoline-1-carboxylic acid ethyl ester;

(R,S,R)-4-[Acetoxy-(3,5-Bis-trifluoromethyl-phenyl)-methyl]-6,7-dimethoxy-2-methyl-3,4-dihydro-2H-quinoline-1-carboxylic acid ethyl ester;

(R,R,R)-4-[Acetoxy-(3,5-Bis-trifluoromethyl-phenyl)-methyl]-6,7-dimethoxy-2-methyl-3,4-dihydro-2H-quinoline-1-carboxylic acid ethyl ester;

(S,S,S)-4-[Acetoxy-(3,5-Bis-trifluoromethyl-phenyl)-methyl]-6,7-dimethoxy-2-methyl-3,4-dihydro-2H-quinoline-1-carboxylic acid ethyl ester;

(S,R,S)-4-[Acetoxy-(3,5-Bis-trifluoromethyl-phenyl)-methyl]-6,7-dimethoxy-2-methyl-3,4-dihydro-2H-quinoline-1-carboxylic acid ethyl ester;

(S,S,R)-4-[Acetoxy-(3,5-Bis-trifluoromethyl-phenyl)-methyl]-6,7-dimethoxy-2-methyl-3,4-dihydro-2H-quinoline-1-carboxylic acid ethyl ester; or

(S,R,R)-4-[Acetoxy-(3,5-Bis-trifluoromethyl-phenyl)-methyl]-6,7-dimethoxy-2-methyl-3,4-dihydro-2H-quinoline-1-carboxylic acid ethyl ester;

or a pharmaceutically acceptable salt of said compound.

### EXAMPLE 419

### (Stereo-Isomers of Examples 22, 23, 24 and 25)

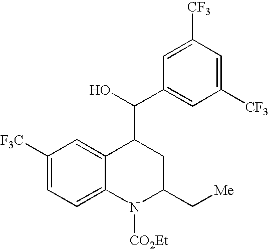

(R,S,S)-4-[(3,5-bis-trifluoromethyl-phenyl)-hydroxy-methyl]-2-ethyl-6-trifluoromethyl-3,4-dihydro-2H-quinoline-1-carboxylic acid ethyl ester;

(R,S,R)-4-[(3,5-bis-trifluoromethyl-phenyl)-hydroxy-methyl]-2-ethyl-6-trifluoromethyl-3,4-dihydro-2H-quinoline-1-carboxylic acid ethyl ester;

(R,R,S)-4-[(3,5-bis-trifluoromethyl-phenyl)-hydroxy-methyl]-2-ethyl-6-trifluoromethyl-3,4-dihydro-2H-quinoline-1-carboxylic acid ethyl ester;

(R,R,R)-4-[(3,5-bis-trifluoromethyl-phenyl)-hydroxy-methyl]-2-ethyl-6-trifluoromethyl-3,4-dihydro-2H-quinoline-1-carboxylic acid ethyl ester;

(S,S,S)-4-[(3,5-bis-trifluoromethyl-phenyl)-hydroxy-methyl]-2-ethyl-6-trifluoromethyl-3,4-dihydro-2H-quinoline-1-carboxylic acid ethyl ester;

(S,S,R)-4-[(3,5-bis-trifluoromethyl-phenyl)-hydroxy-methyl]-2-ethyl-6-trifluoromethyl-3,4-dihydro-2H-quinoline-1-carboxylic acid ethyl ester;

(S,R,S)-4-[(3,5-bis-trifluoromethyl-phenyl)-hydroxy-methyl]-2-ethyl-6-trifluoromethyl-3,4-dihydro-2H-quinoline-1-carboxylic acid ethyl ester; or

(S,R,R)-4-[(3,5-bis-trifluoromethyl-phenyl)-hydroxy-methyl]-2-ethyl-6-trifluoromethyl-3,4-dihydro-2H-quinoline-1-carboxylic acid ethyl ester;

or a pharmaceutically acceptable salt of said compound.

### EXAMPLE 420

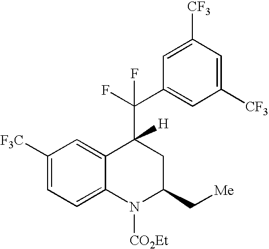

4-[(3,5-Bis-trifluoromethyl-phenyl)-difluoro-methyl]-2-ethyl-6-trifluoromethyl-3,4-dihydro-2H-quinoline-1-carboxylic acid ethyl ester;

(R,S)-4-[(3,5-Bis-trifluoromethyl-phenyl)-difluoro-methyl]-2-ethyl-6-trifluoromethyl-3,4-dihydro-2H-quinoline-1-carboxylic acid ethyl ester;

(R,R)-4-[(3,5-Bis-trifluoromethyl-phenyl)-difluoro-methyl]-2-ethyl-6-trifluoromethyl-3,4-dihydro-2H-quinoline-1-carboxylic acid ethyl ester;

(S,R)-4-[(3,5-Bis-trifluoromethyl-phenyl)-difluoro-methyl]-2-ethyl-6-trifluoromethyl-3,4-dihydro-2H-quinoline-1-carboxylic acid ethyl ester; or

(S,S)-4-[(3,5-Bis-trifluoromethyl-phenyl)-difluoro-methyl]-2-ethyl-6-trifluoromethyl-3,4-dihydro-2H-quinoline-1-carboxylic acid ethyl ester;

or a pharmaceutically acceptable salt of said compound.

### EXAMPLE 421

### (Stereo-Isomers of Example 15)

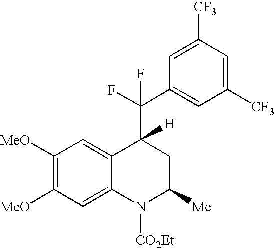

(R,R)-4-[(3,5-bis-trifluoromethyl-phenyl )-difluoro-methyl]-2-methyl-6,7-dimethoxy-3,4-dihydro-2H-quinoline-1-carboxylic acid ethyl ester;

(R,S)-4-[(3,5-bis-trifluoromethyl-phenyl)-difluoro-methyl]-2-methyl-6,7-dimethoxy-3,4-dihydro-2H-quinoline-1-carboxylic acid ethyl ester;

(S,R)-4-[(3,5-bis-trifluoromethyl-phenyl)-difluoro-methyl]-2-methyl-6,7-dimethoxy-3,4-dihydro-2H-quinoline-1-carboxylic acid ethyl ester; or

(S,S)-4-[(3,5-bis-trifluoromethyl-phenyl)-difluoro-methyl]-2-methyl-6,7-dimethoxy-3,4-dihydro-2H-quinoline-1-carboxylic acid ethyl ester;

or a pharmaceutically acceptable salt of said compound.

### EXAMPLE 422

### (Stereo-Isomers of Example 182)

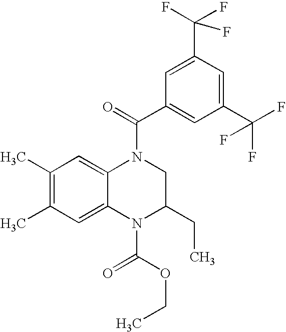

(R)-4-[(3,5-Bis-trifluoromethyl-benzoyl)]-2-ethyl-6,7-dimethyl-3,4-dihydro-2H-quinoxaline-1-carboxylic acid ethyl ester; or

(S)-4-[(3,5-Bis-trifluoromethyl-benzoyl)]-2-ethyl-6,7-dimethyl-3,4-dihydro-2H-quinoxaline-1-carboxylic acid ethyl ester;

or a pharmaceutically acceptable salt of said compound.

### EXAMPLE 423

### (Stereo-Isomers of Example 178)

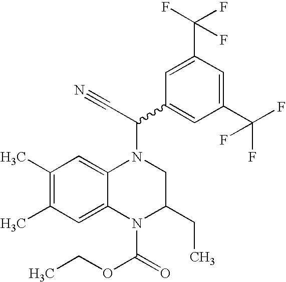

(R,R)-4-[(3,5-Bis-trifluoromethyl-phenyl)-cyano-methyl]-2-ethyl-6,7-dimethyl-3,4-dihydro-2H-quinoxaline-1-carboxylic acid ethyl ester;

(R,S)-4-[(3,5-Bis-trifluoromethyl-phenyl)-cyano-methyl]-2-ethyl-6,7-dimethyl-3,4-dihydro-2H-quinoxaline-1-carboxylic acid ethyl ester;

(S,R)-4-[(3,5-Bis-trifluoromethyl-phenyl)-cyano-methyl]-2-ethyl-6,7-dimethyl-3,4-dihydro-2H-quinoxaline-1-carboxylic acid ethyl ester; or

(S,S)-4-[(3,5-Bis-trifluoromethyl-phenyl)-cyano-methyl]-2-ethyl-6,7-dimethyl-3,4-dihydro-2H-quinoxaline-1-carboxylic acid ethyl ester;

or a pharmaceutically acceptable salt of said compound.

### EXAMPLE 424

### (Stereo-Isomers of Examples 170, 171, 172, and 173)

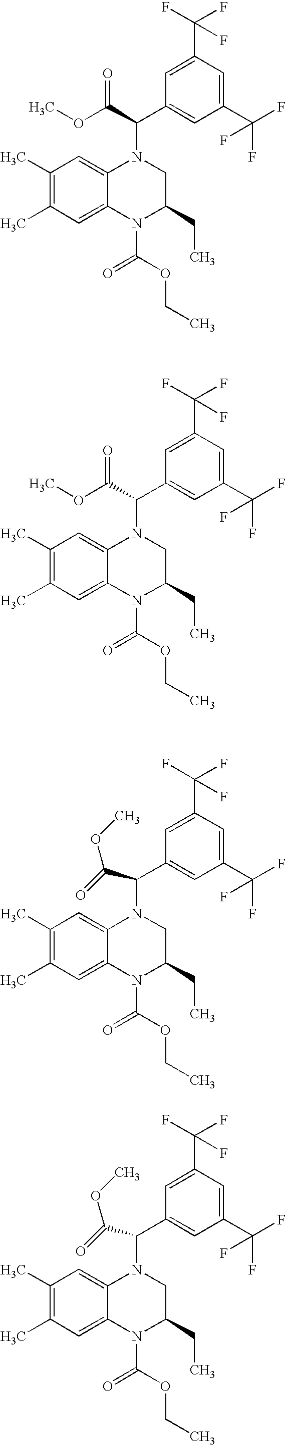

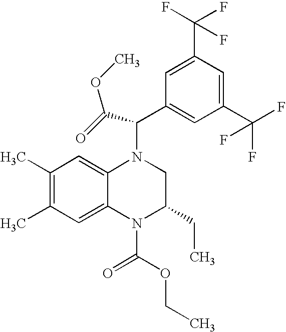

(R,R)-4-[(3,5-Bis-trifluoromethyl-phenyl)-methoxycarbonyl-methyl]-2-ethyl-6,7-dimethyl-3,4-dihydro-2H-quinoxaline-1-carboxylic acid ethyl ester;

(R,S)-4-[(3,5-Bis-trifluoromethyl-phenyl)-methoxycarbonyl-methyl]-2-ethyl-6,7-dimethyl-3,4-dihydro-2H-quinoxaline-1-carboxylic acid ethyl ester;

(S,R)-4-[(3,5-Bis-trifluoromethyl-phenyl)-methoxycarbonyl-methyl]-2-ethyl-6,7-dimethyl-3,4-dihydro-2H-quinoxaline-1-carboxylic acid ethyl ester; or

(S,S)-4-[(3,5-Bis-trifluoromethyl-phenyl)-methoxycarbonyl-methyl]-2-ethyl-6,7-dimethyl-3,4-dihydro-2H-quinoxaline-1-carboxylic acid ethyl ester;

or a pharmaceutically acceptable salt of said compound.

### EXAMPLE 425

### (Stereo-Isomers of Examples 174 and 175)

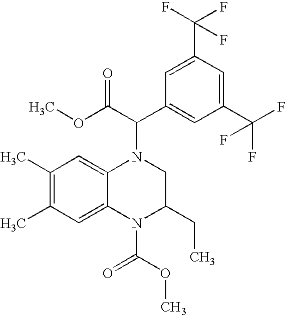

(R,R)-4-[(3,5-Bis-trifluoromethyl-phenyl)-methoxycarbonyl-methyl]-2-ethyl-6,7-dimethyl-3,4-dihydro-2H-quinoxaline-1-carboxylic acid methyl ester;

(R,S)-4-[(3,5-Bis-trifluoromethyl-phenyl)-methoxycarbonyl-methyl]-2-ethyl-6,7-dimethyl-3,4-dihydro-2H-quinoxaline-1-carboxylic acid methyl ester;

(S,R)-4-[(3,5-Bis-trifluoromethyl-phenyl)-methoxycarbonyl-methyl]-2-ethyl-6,7-dimethyl-3,4-dihydro-2H-quinoxaline-1-carboxylic acid methyl ester; or

(S,S)-4-[(3,5-Bis-trifluoromethyl-phenyl)-methoxycarbonyl-methyl]-2-ethyl-6,7-dimethyl-3,4-dihydro-2H-quinoxaline-1-carboxylic acid methyl ester;

or a pharmaceutically acceptable salt of said compound.

### EXAMPLE 426

### (Stereo-Isomers of Examples 176 and 177)

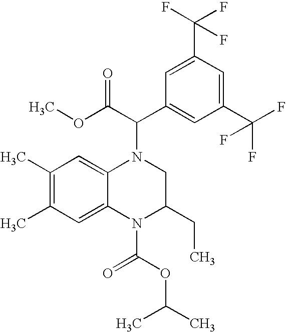

(R,R)-4-[(3,5-Bis-trifluoromethyl-phenyl)-methoxycarbonyl-methyl]-2-ethyl-6,7-dimethyl-3,4-dihydro-2H-quinoxaline-1-carboxylic acid isopropyl ester;

(R,S)-4-[(3,5-Bis-trifluoromethyl-phenyl)-methoxycarbonyl-methyl]-2-ethyl-6,7-dimethyl-3,4-dihydro-2H-quinoxaline-1-carboxylic acid isopropyl ester;

(S,R)-4-[(3,5-Bis-trifluoromethyl-phenyl)-methoxycarbonyl-methyl]-2-ethyl-6,7-dimethyl-3,4-dihydro-2H-quinoxaline-1-carboxylic acid isopropyl ester; or

(S,S)-4-[(3,5-Bis-trifluoromethyl-phenyl)-methoxycarbonyl-methyl]-2-ethyl-6,7-dimethyl-3,4-dihydro-2H-quinoxaline-1-carboxylic acid isopropyl ester;

or a pharmaceutically acceptable salt of said compound.

### EXAMPLE 427

### (Stereo-Isomers of Example 183)

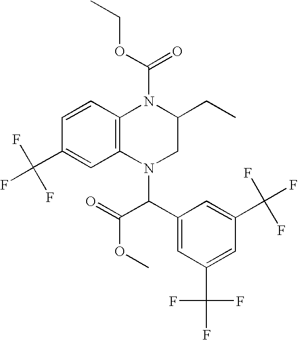

(R,R)-4-[(3,5-Bis-trifluoromethyl-phenyl)-methoxycarbonyl-methyl]-2-ethyl-6-trifluoromethyl-3,4-dihydro-2H-quinoxaline-1-carboxylic acid ethyl ester;

(R,S)-4-[(3,5-Bis-trifluoromethyl-phenyl)-methoxycarbonyl-methyl]-2-ethyl-6-trifluoromethyl-3,4-dihydro-2H-quinoxaline-1-carboxylic acid ethyl ester;

(S,R)-4-[(3,5-Bis-trifluoromethyl-phenyl)-methoxycarbonyl-methyl]-2-ethyl-6-trifluoromethyl-3,4-dihydro-2H-quinoxaline-1-carboxylic acid ethyl ester; or

(S,S)-4-[(3,5-Bis-trifluoromethyl-phenyl)-methoxycarbonyl-methyl]-2-ethyl-6-trifluoromethyl-3,4-dihydro-2H-quinoxaline-1-carboxylic acid ethyl ester;

or a pharmaceutically acceptable salt of said compound.

### EXAMPLES 428 AND 429

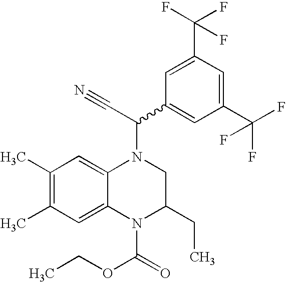

### (R,S)-4-[(3,5-Bis-trifluoromethyl-phenyl)-cyano-methyl]-2-ethyl-6,7-dimethyl-3,4-dihydro-2H-quinoxaline-1-carboxylic Acid Ethyl Ester and (R,R)-4-[(3,5-Bis-trifluoromethyl-phenyl)-cyano-methyl]-2-ethyl-6,7-dimethyl-3,4-dihydro-2H-quinoxaline-1-carboxylic Acid Ethyl Ester

A mixture of 2-ethyl-6,7-dimethyl-3,4-dihydro-2(R)-H-quinoxaline-1-carboxylic acid ethyl ester (1 g, 1 eq, 3.81 mmol), 3,5-bis-(trifluoromethyl-phenyl)-bromo-acetonitrile (Preparation 40, 1.27 gm, 1 eq, 3.81 mmol) and 2,6-lutidine (3 eq, 11.43 mmol) in N,N-dimethylformamide (5 mL) was stirred at room temperature for 24 hours. The reaction was partitioned between ethyl acetate and water, and the phases were separated. The aqueous phase was extracted 3 times with ethyl acetate, and the combined organic extracts were washed 2 times with water, 1 time with brine, dried over anhydrous sodium sulfate, filtered and evaporated. Chromatography on silica gel using a gradient of 10-30% ethyl acetate in hexanes as eluant provided the desired nitrile diastereoisomers (1.5:1) (1.0 g. and 0.7 g, 60%).

Isomer 1:

LCMS (ESI+): 514 (MH+)

¹H-NMR (CDCl₃) δ 0.88 (t, J=7.5 Hz, 3H), 1.29 (t, J=7.1 Hz, 3H), 1.49 (m, 2H), 2.21 (s, 3H), 2.22 (s, 3H), 3.03 (dd, J₁=11.0 Hz, J₂=2.9 Hz, 1H), 3.19 (dd, J₁=11.0 Hz, J₂=5.4 Hz, 1H), 4.19 (m, 1H), 4.27 (m, 1H), 4.50 (brm, 1H), 6.05 (s, 1H), 6.59 (s, 1H), 7.33 (brs, 1H), 7.94 (s, 1H), 7.97 (s, 2H).

Isomer2:

LCMS (ESI+): 514 (MH+)

¹H-NMR (CDCl₃) δ 0.76 (t, J=7.48 Hz, 3H), 1.31 (t, J=7.06 Hz, 3H), 1.52 (m, 2H), 2.21 (s, 3H), 2.23 (s, 3H), 2.75 (dd, J₁=11.0 Hz, J₂=2.1 Hz, 1H), 3.29 (dd, J₁=11.0 Hz, J₂=3.73 Hz, 1H), 4.19-4.30 (m, 2H), 4.52 (brm, 1H), 6.19 (s, 1H), 6.70 (s, 1H), 7.47 (brs, 1H), 7.96 (s, 1H), 8.00 (s, 2H).

### Preparation 42

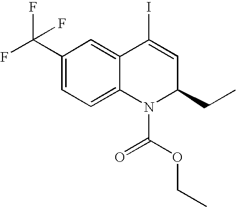

### (R)-2-Ethyl-4-iodo-6-trifluoromethyl-2H-quinoline-1-carboxylic Acid Ethyl Ester

The title compound was prepared using the general procedure described by D. H. R. Barton et al. (Tetrahedron Letters 1983 24, 1605). To a solution of iodine (3.0 gm, 9.1 mmol) in anhydrous tetrahydrofuran (30 mL) under nitrogen was slowly added a solution of 1,1,3,3-tetramethylguanidine (63.7 mmol, 8 mL) in anhydrous tetrahydrofuran (30 mL). The mixture was stirred at room temperature for 10 min before addition of (R)-2-ethyl-4-hydrazono-6-trifluoromethyl-3,4-dihydro-2H-quinoline-1-carboxylic acid ethyl ester (Preparation 6, 9.1 mmol, 3 gm) in anhydrous tetrahydrofuran (30 mL). After 15 min the solvent was removed under vacuum and the residue heated under nitrogen at 85° C. for 90 min. The residue was dissolved in ethyl acetate, washed with 2N hydrochloric acid, aqueous sodium sulfite solution (2.5%), saturated sodium hydrogen carbonate solution and dried over anhydrous sodium sulfate. The crude product was purified by chromatography on silica gel eluting with hexanes/ethyl acetate from 19:1 to 85:15 to give the title compound as a yellow solid (2.8 gm, 72%).

MS: 426.3 [M+H]⁺ found.

¹H-NMR (CDCl₃) δ 7.69 (brs, 1H), 7.61 (brd, J=8.14 Hz, 1H), 7.49 (brd, J=8.14 Hz, 1H), 6.85 (d, J=6.64 Hz, 1H), 4.87 (m, 1H), 4.27 (m, 2H), 4.15 (m, 1H), 1.50 (m, 1H), 1.38 (m, 1H), 1.32 (t, J=7.47 Hz, 3H), 0.87 (t, J=7.47 Hz, 3H).

### EXAMPLE 430

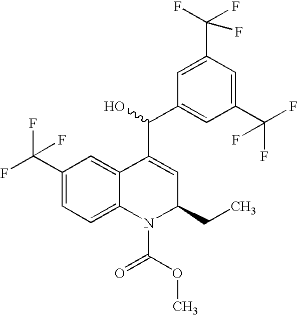

### (RS, RS) and (RS, SR)-4-[(3,5-Bis-trifluoromethyl-phenyl)-hydroxy-methyl]-2-ethyl-6-trifluoromethyl-2H-quinoline-1-carboxylic Acid Ethyl Ester

To a solution of (RS)-2-ethyl-4-iodo-6-trifluoromethyl-2H-quinoline-1-carboxylic acid ethyl ester (814 mg, 1.91 mmol, prepared as described above for the (R) isomer except that racemic starting material was used) in anhydrous tetrahydrofuran (4 mL) under nitrogen at −78° C. was added dropwise n-butyllithium (2.5M in hexanes, 2.87 mmol, 1.15 mL). After 5 min 3,5-bis(trifluoromethyl)benzaldehyde (6.06 mmol, 1 mL) was added dropwise. After 45 min at −78° C. the mixture was allowed to warm to room temperature and after 1 hr water was added. The mixture was acidified by addition of 2N hydrochloric acid and extracted with ethyl acetate. The organic solution was washed with water, dried over anhydrous sodium sulfate and evaporated under vacuum. The residue was purified by chromatography on silica gel eluting with hexanes/ethyl acetate from 9:1 to 4:1 then further purified by chromatography on silica gel eluting with dichloromethane to give the title compounds as a mixture of diastereoisomers (60 mg).

MS: 540.3 [M−H]⁺ found.

### EXAMPLE 431

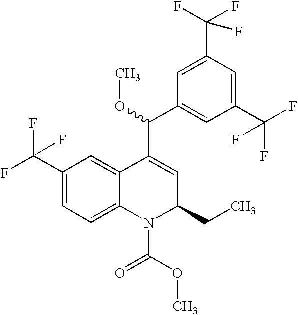

### (RS, RS) and (RS, SR)-4-[(3,5-Bis-trifluoromethyl-phenyl)-methoxy-methyl]-2-ethyl-6-trifluoromethyl-2H-quinoline-1-carboxylic Acid Ethyl Ester

To a solution of the diastereoisomeric mixture (RS, RS) and (RS, SR)-4-[(3,5-bis-trifluoromethyl-phenyl)-hydroxy-methyl]-2-ethyl-6-trifluoromethyl-2H-quinoline-1-carboxylic acid ethyl ester (Example 430, 32 mg, 0.059 mmol) in dimethylsulfoxide (3 mL) under nitrogen was added powdered potassium hydroxide (0.236 mmol, 13 mg) followed immediately by iodomethane (0.118 mmol, 7.4 μL). The mixture was stirred at room temperature for 2 hr then diluted with 2N hydrochloric acid. The mixture was extracted with ethyl acetate, the organic layer washed with water and saturated sodium chloride solution, dried over anhydrous sodium sulfate and evaporated under vacuum. The crude product was purified by chromatography on silica gel eluting with hexanes then hexanes/ethyl acetate 19:1 then 9:1 then 85:15 to give the title compound (13 mg) as a mixture of diastereoisomers.

### EXAMPLE 432

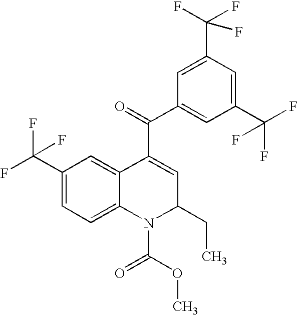

### (RS)-4-(3,5-Bis-trifluoromethyl-benzoyl)-2-ethyl-6-trifluoromethyl-2H-quinoline-1-carboxylic Acid Ethyl Ester

To a solution of the diastereoisomeric mixture (RS, RS) and (RS, SR)-4-[(3,5-bis-trifluoromethyl-phenyl)-hydroxy-methyl]-2-ethyl-6-trifluoromethyl-2H-quinoline-1-carboxylic acid ethyl ester (Example 430, 11 mg, 0.020 mmol) in anhydrous diethyl ether (1 mL) was added manganese (IV) oxide (22 mg, activated, ˜85%, Aldrich Chemical Company, Milwaukee, Wis.). The suspension was stirred at ambient temperature for 90 min. A second aliquot of manganese (IV) oxide (20 mg) was added and stirring was continued for a further 1 hr before a third aliquot of manganese (IV) oxide (30 mg) was added. After 10 min the solid was removed by filtration through Celite®, the solvent was removed under vacuum and the residue was chromatographed on Baker Silica Gel (1 gm, 40 μm) (J. T. Baker, Phillipsburg, N.J.) eluting with hexanes then hexanes/ethyl acetate 19:1 then 9:1 then 85:15 then 4:1 to give the title compound (6.6 mg).

MS: 540.3 [M+H]⁺ found.

¹H-NMR (CDCl₃) δ 8.27 (s, 2H), 8.11 (s, 1H), 7.79 (brd, J=8.3 Hz, 1H), 7.77 (brs, 1H), 7.57 (brd, J=8.3 Hz, 1H), 6.56 (d, J=6.64 Hz, 1H), 5.18 (m, 1H), 4.31 (m, 2H), 1.64 (m, 1H), 1.55 (m, 1H), 1.33 (t, J=7.47 Hz, 3H), 0.96 (t, J=7.47 Hz, 3H).

### EXAMPLES 433 AND 434

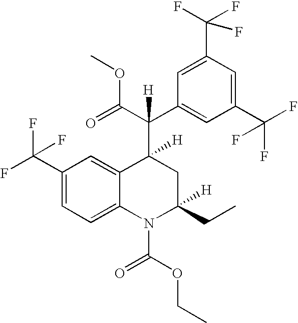

### (R, R, S)-4-[(3,5-Bis-trifluoromethyl-phenyl)-methoxycarbonyl-methyl]-2-ethyl-6-trifluoromethyl-3,4-dihydro-2H-quinoline-1-carboxylic Acid Ethyl Ester

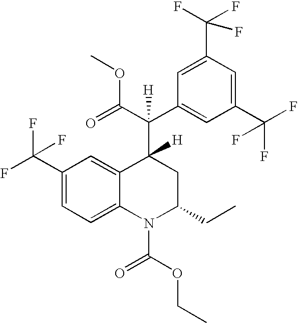

### (S, S, R)-4-[(3,5-Bis-trifluoromethyl-phenyl)-methoxycarbonyl-methyl]-2-ethyl-6-trifluoromethyl-3,4-dihydro-2H-quinoline-1-carboxylic Acid Ethyl Ester

(RS, RS, SR)-4-[(3,5-Bis-trifluoromethyl-phenyl)-methoxycarbonyl-methyl]-2-ethyl-6-trifluoromethyl-3,4-dihydro-2H-quinoline-1-carboxylic acid ethyl ester (Example 7) was resolved on a Pirkle Covalent (S,S)Whelk-O 1 column (Regis Technologies, Inc., Morton Grove, Ill.) (5×25 cm) eluting at 100 mL/min with 5% ethanol/heptane to provide two fractions:

First eluting: (S, S, R)-4-[(3,5-Bis-trifluoromethyl-phenyl)-methoxycarbonyl-methyl]-2-ethyl-6-trifluoromethyl-3,4-dihydro-2H-quinoline-1-carboxylic acid ethyl ester;

MS: 585.8 [M+H]⁺ found.

Second eluting: (R, R, S)-4-[(3,5-Bis-trifluoromethyl-phenyl)-methoxycarbonyl-methyl]-2-ethyl-6-trifluoromethyl-3,4-dihydro-2H-quinoline-1-carboxylic acid ethyl ester;

MS: 586.2 [M+H]⁺ found.

### EXAMPLES 435 AND 436

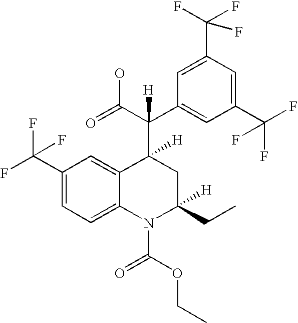

### (R, R, S)-4-[(3,5-Bis-trifluoromethyl-phenyl)-carboxy-methyl]-2-ethyl-6-trifluoromethyl-3,4-dihydro-2H-quinoline-1-carboxylic Acid Ethyl Ester

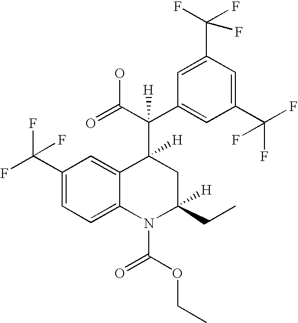

### (R, R, R)-4-[(3,5-Bis-trifluoromethyl-phenyl)-carboxy-methyl]-2-ethyl-6-trifluoromethyl-3,4-dihydro-2H-quinoline-1-carboxylic Acid Ethyl Ester

A mixture of (R, R, S)-4-[(3,5-bis-trifluoromethyl-phenyl)-methoxycarbonyl-methyl]-2-ethyl-6-trifluoromethyl-3,4-dihydro-2H-quinoline-1-carboxylic acid ethyl ester (Example 433, 98 mg, 0.167 mmol), aqueous sodium hydroxide (1N, 1 mL, 1 mmol) and anhydrous tetrahydrofuran (2.4 mL) was stirred at room temperature for 5 days before adding 2N hydrochloric acid to acid pH. The mixture was extracted with ethyl acetate, the organic solution washed with water (×3) and dried over anhydrous sodium sulfate. The solvent was removed under vacuum and the residue chromatographed on Baker Silica Gel (1 gm, 40 μm) (J. T. Baker, Phillipsburg, N.J.) eluting with a hexanes-ethyl acetate gradient from 0% to 80% ethyl acetate to give the title compounds:

First eluting: (R, R, S)-4-[(3,5-Bis-trifluoromethyl-phenyl)-carboxy-methyl]-2-ethyl-6-trifluoromethyl-3,4-dihydro-2H-quinoline-1-carboxylic acid ethyl ester (67 mg);

MS: 572.4 [M+H]⁺ found.

¹H-NMR (CDCl₃) δ 7.90 (s, 1H), 7.89 (s, 2H), 7.54 (m, 2H), 7.46 (brs, 1H), 4.28 (m, 2H), 4.22 (m, 1H), 4.08 (d, J=11.2 Hz, 1H), 3.37 (m, 1H), 1.76 (m, 1H), 1.48 (m, 1H), 1.38 (m, 1H), 1.31 (t, J=7.05 Hz, 3H), 0.95 (m, 1H), 0.70 (t, J=7.47 Hz, 3H).

Second eluting: (R, R, R)-4-[(3,5-Bis-trifluoromethyl-phenyl)-carboxy-methyl]-2-ethyl-6-trifluoromethyl-3,4-dihydro-2H-quinoline-1-carboxylic acid ethyl ester (29.5 mg)

MS: 572.3 [M+H]⁺ found

¹H-NMR (CDCl₃) δ 7.94 (s, 2H), 7.83 (s, 1H), 7.50 (d, J=8.14 Hz, 1H), 7.41 (brd, J=8.14 Hz, 1H), 7.01 (brs, 1H), 4.42 (m, 1H), 4.22 (m, 2H), 4.21 (m, 1H), 3.32 (m, 1H), 2.45 (m, 1H), 1.58 (m, 1H), 1.41 (m, 1H), 1.41 (m, 1H), 1.28 (t, J=7.47 Hz, 3H), 0.79 (t, J=7.47 Hz, 3H).

### EXAMPLE 437

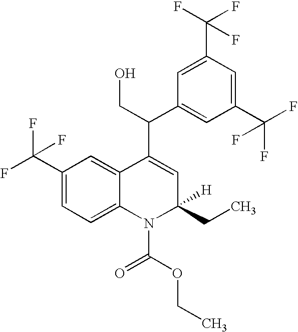

### (R, R) and (R, S)-4-[1-(3,5-Bis-trifluoromethyl-phenyl)-2-hydroxy-ethyl]-2-ethyl-6-trifluoromethyl-2H-quinoline-1-carboxylic Acid Ethyl Ester

To a solution of (R,R)-4-[(3,5-bis-trifluoromethyl-phenyl)-methoxycarbonyl-methyl]-2-ethyl-6-trifluoromethyl-2H-quinoline-1-carboxylic acid ethyl ester and (R, S)-4-[(3,5-bis-trifluoromethyl-phenyl)-methoxycarbonyl-methyl]-2-ethyl-6-trifluoromethyl-2H-quinoline-1-carboxylic acid ethyl ester (Examples 3 and 4, used as the mixture obtained prior to separation, 329 mg, 0.563 mmol) in anhydrous tetrahydrofuran (6 mL) under nitrogen at −40° C. was added dropwise a solution of lithium aluminum hydride (1M in tetrahydrofuran, 845 μL, 0.845 mmol). After 30 min an excess of ethyl acetate was added to quench the reaction and the mixture was allowed to warm to room temperature. The mixture was shaken with water/ethyl acetate, the organic layer washed with water, dried over anhydrous sodium sulfate and evaporated under vacuum. The crude product was purified by chromatography on silica gel eluting with hexanes then hexanes/ethyl acetate 9:1 then 4:1 then 7:3 to give the title compounds as a mixture of diastereoisomers (243 mg).

MS: 556.3 [M+H]⁺ found.

### EXAMPLE 438

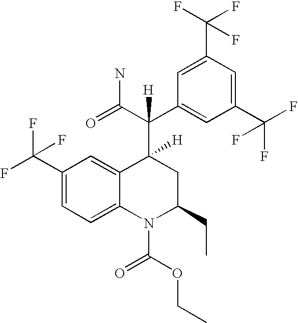

### (R, R, S)-4-[(3,5-Bis-trifluoromethyl-phenyl)-carbamoyl-methyl]-2-ethyl-6-trifluoromethyl-3,4-dihydro-2H-quinoline-1-carboxylic Acid Ethyl Ester

A mixture of (R, R, S)-4-[(3,5-bis-trifluoromethyl-phenyl)-carboxy-methyl]-2-ethyl-6-trifluoromethyl-3,4-dihydro-2H-quinoline-1-carboxylic acid ethyl ester (Example 435, 110 mg, 0.193 mmol) and thionyl chloride (1 mL) was stirred at room temperature under nitrogen for 3 days then the excess thionyl chloride was removed under vacuum. To the residue was added a solution of ammonia in dioxane (0.5M, 6 mL, 3 mmol). After 12 hr the mixture was diluted with ethyl acetate, washed with water (×2) and the organic layer was dried over anhydrous sodium sulfate then evaporated to dryness under vacuum. The crude product was purified by chromatography on silica gel eluting with dichloromethane/ethyl acetate 39:1 then 19:1 to give the title compound (103 mg).

MS: 571.3 [M+H]⁺ found.

¹H-NMR (CDCl₃) δ7.88 (s, 1H), 7.88 (s, 2H), 7.51 (m, 2H), 7.46 (brs, 1H), 5.80 (brs, 1H), 5.53 (brs, 1H), 4.26 (m, 1H), 4.26 (m, 1H), 4.19 (m, 1H), 3.82 (d, J=10.79 Hz, 1H), 3.45 (m, 1H), 1.68 (m, 1H), 1.49 (m, 1H), 1.39 (m, 1H), 1.29 (t, J=7.06 Hz, 3H), 0.95 (m, 1H), 0.70 (t, J=7.47 Hz, 3H).

The two following compounds were prepared by an analogous procedure utilizing, respectively, methylamine and dimethylamine in place of ammonia:

### EXAMPLE 439

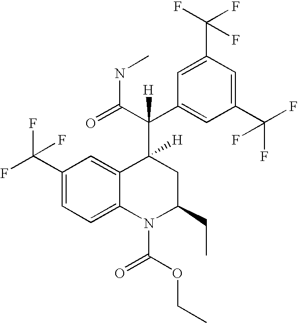

### (R, R, S)-4-[(3,5-Bis-trifluoromethyl-phenyl)-methylcarbamoyl-methyl]-2-ethyl-6-trifluoromethyl-3,4-dihydro-2H-quinoline-1-carboxylic Acid Ethyl Ester

MS: 585.3 [M+H]⁺ found.

### EXAMPLE 440

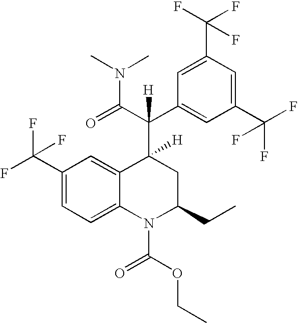

### (R, R, S)-4-[(3,5-Bis-trifluoromethyl-phenyl)-dimethylcarbamoyl-methyl]-2-ethyl-6-trifluoromethyl-3,4-dihydro-2H-quinoline-1-carboxylic Acid Ethyl Ester

MS: 599.3 [M+H]⁺ found.

### EXAMPLES 441 AND 442

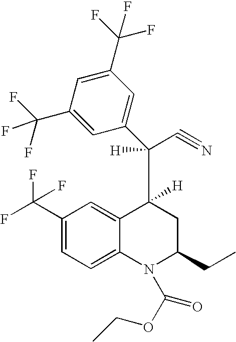

### (R, R, S)-4-[(3,5-Bis-trifluoromethyl-phenyl)-cyano-methyl]-2-ethyl-6-trifluoromethyl-3,4-dihydro-2H-quinoline-1-carboxylic Acid Ethyl Ester

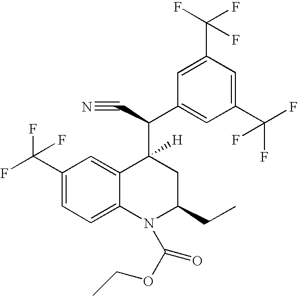

### (R, R, R)-4-[(3,5-Bis-trifluoromethyl-phenyl)-cyano-methyl]-2-ethyl-6-trifluoromethyl-3,4-dihydro-2H-quinoline-1-carboxylic Acid Ethyl Ester

To a solution of (R, R, S)-4-[(3,5-bis-trifluoromethyl-phenyl)-carbamoyl-methyl]-2-ethyl-6-trifluoromethyl-3,4-dihydro-2H-quinoline-1-carboxylic acid ethyl ester (Example 438, 30 mg, 0.053 mmol) in anhydrous dichloromethane (1 mL) was added (methoxycarbonylsulfamoyl)triethylammonium hydroxide (Burgess reagent, 37 mg, 0.157 mmol). The mixture was stirred at room temperature for 72 hr under nitrogen then evaporated under vacuum. The crude product was purified by chromatography on silica gel eluting with hexanes then hexanes/ethyl acetate 19:1 then 9:1 to give the title compounds:

First eluting compound: (R, R, R)-4-[(3,5-bis-trifluoromethyl-phenyl)-cyano-methyl]-2-ethyl-6-trifluoromethyl-3,4-dihydro-2H-quinoline-1-carboxylic acid ethyl ester (8mg);

MS: 553.3 [M+H]⁺ found;

¹H-NMR (CDCl₃) δ 7.96 (s, 1H), 7.90 (s, 2H), 7.60 (m, 2H), 7.56 (brs, 1H), 4.88 (d, J=3.32 Hz, 1H), 4.28 (m, 1H), 4.22 (m, 1H), 4.22 (m, 1H), 2.93 (m, 1H), 2.22 (m, 1H), 1.63 (m, 1H), 1.62 (m, 1H), 1.51 (m, 1H), 1.27 (t, J=7.47 Hz, 3H), 0.83 (t, J=7.47 Hz, 3H).

Second eluting compound: (R, R, S)-4-[(3,5-bis-trifluoromethyl-phenyl)-cyano-methyl]-2-ethyl-6-trifluoromethyl-3,4-dihydro-2H-quinoline-1-carboxylic acid ethyl ester (20 mg);

MS: 553.3 [M+H]⁺ found;

¹H-NMR (CDCl₃) δ 7.96 (s, 1H), 7.92 (s, 2H), 7.59 (brs, 1H), 7.57 (m, 2H), 4.36 (m, 1H), 4.30 (m, 1H), 4.27 (d, J=8.3 Hz, 1H), 4.22 (m, 1H), 3.23 (m, 1H), 2.08 (m, 1H), 1.55 (m, 1H), 1.43 (m, 1H), 1.30 (t, J=7.47 Hz, 3H), 1.27 (m, 1H), 0.79 (t, J=7.47 Hz, 3H).

### Preparation 43

### (R)-4-Bromo-2-ethyl-6-trifluoromethyl-3,4-dihydro-2H-quinoline-1-carboxylic Acid Ethyl Ester

To a solution of (R, S)-2-ethyl-4-hydroxy-6-trifluoromethyl-3,4-dihydro-2H-quinoline-1-carboxylic acid ethyl ester (Preparation 13, 3.29 gm, 10.37 mmol) in dichloromethane (25 mL) at ambient temperature under nitrogen was added pyridine (1.58 mL) followed dropwise by a solution of phosphorus (III) bromide (1.1 mL) in dichloromethane (10 mL). The mixture was allowed to stir at ambient temperature for 15 h then partitioned between water and dichloromethane. The organic layer was washed with saturated sodium hydrogen carbonate solution (2×15 mL), dried over anhydrous sodium sulfate and the solvent was removed under reduced pressure to give the title compound as a yellow oil (3.79 gm) containing an approximately 5:1 mixture of diastereoisomers and a variable amount of (R)-2-ethyl-6-trifluoromethyl-2H-quinoline-1-carboxylic acid ethyl ester formed by elimination of hydrogen bromide. The crude bromide was used directly without further purification or stored in the refrigerator to arrest further decomposition.

MS: 379, 381 [M].⁺ found (GC-MS).

### EXAMPLES 443 AND 444

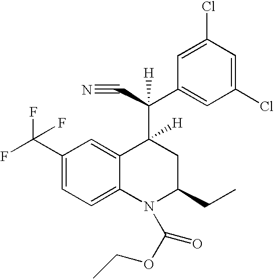

### (R, R, R)-4-[Cyano-(3,5-dichloro-phenyl)-methyl]-2-ethyl-6-trifluoromethyl-3,4-dihydro-2H-quinoline-1-carboxylic Acid Ethyl Ester

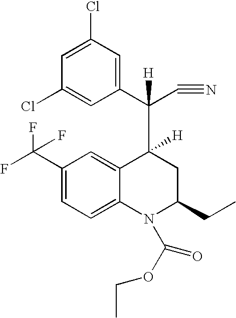

### (R, R, S)-4-[Cyano-(3,5-dichloro-phenyl)-methyl]-2-ethyl-6-trifluoromethyl-3,4-dihydro-2H-quinoline-1-carboxylic Acid Ethyl Ester

To a solution of 3,5-dichlorophenylacetonitrile (134 mg, 0.72 mmol, prepared according to the procedure described in WO00/58292) in anhydrous N,N-dimethylformamide (1 mL) was added sodium hydride (60% mineral oil dispersion, 0.925 mmol, 37 mg) and the mixture was stirred at room temperature for 30 min. A solution of (R)-4-bromo-2-ethyl-6-trifluoromethyl-3,4-dihydro-2H-quinoline-1-carboxylic acid ethyl ester (Preparation 43, 250 mg, mixture of isomers as prepared above) in anhydrous N,N-dimethylformamide (1.5 mL) was added and the mixture was stirred at room temperature for 5 min. Water was added and the mixture was extracted with diethyl ether (3×20 mL) and the organic extract was diluted with heptane and evaporated to dryness to give the crude product as a yellow oil (˜400 mg). Initial purification was achieved by purified by chromatography on silica gel eluting with hexanes/ethyl acetate 9:1. Fractions containing the title compounds were further purified using radial chromatography (Chromatron model 7924T, Harrison Research, Palo Alto, Calif.) with a 2 mm silica gel rotor eluting with hexanes/ethyl acetate 9:1 to give the title compounds:

First Eluting Compound:

(R, R, R)-4-[cyano-(3,5-dichloro-phenyl)-methyl]-2-ethyl-6-trifluoromethyl-3,4-dihydro-2H-quinoline-1-carboxylic acid ethyl ester (14 mg);

¹H-NMR (CDCl₃) δ 7.59 (s, 2H), 7.54 (s, 1H), 7.43 (t, J=1.95 Hz, 1H), 7.35 (d, J=1.95 Hz, 2H), 4.69 (d, J=3.52 Hz, 1H), 4.28 (m, 1H), 4.22 (m, 1H), 4.22 (m, 1H), 2.90 (m, 1H), 2.27 (m, 1H), 1.61 (m, 1H), 1.50 (m, 1H), 1.49 (m, 1H), 1.29 (t, J=7.03 Hz, 3H), 0.84 (t, J=7.42 Hz, 3H).

Second Eluting Compound:

(R, R, S)-4-[cyano-(3,5-dichloro-phenyl)-methyl]-2-ethyl-6-trifluoromethyl-3,4-dihydro-2H-quinoline-1-carboxylic acid ethyl ester (5 mg);

MS: 485.2 [M+H]⁺ found;

¹H-NMR (CDCl₃) δ 7.59 (s, 1H), 7.55 (s, 2H), 7.42 (t, J=1.66 Hz, 1H), 7.33 (d, J=1.66 Hz, 2H), 4.34 (m, 1H), 4.27 (m, 1H), 4.22 (m, 1H), 4.01 (d, J=9.13 Hz, 1H), 3.13 (m, 1H), 2.07 (m, 1H), 1.53 (m, 1H), 1.42 (m, 1H), 1.30 (t, J=7.47 Hz, 3H), 1.16 (m, 1H), 0.78 (t, J=7.47 Hz, 3H).

### EXAMPLE 445

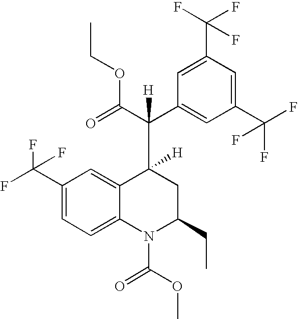

### (R, R, S)-4-[(3,5-Bis-trifluoromethyl-phenyl)-ethoxycarbonyl-methyl]-2-ethyl-6-trifluoromethyl-3,4-dihydro-2H-quinoline-1-carboxylic Acid Ethyl Ester

A solution of (R, R, S)-4-[(3,5-bis-trifluoromethyl-phenyl)-carboxy-methyl]-2-ethyl-6-trifluoromethyl-3,4-dihydro-2H-quinoline-1-carboxylic acid ethyl ester (Example 435, 33 mg, 0.058 mmol) in anhydrous ethanol (5 mL) containing concentrated sulfuric acid (4 drops) was heated under reflux for 18 hr then the solvent was evaporated under vacuum. The residue was partitioned between water and ethyl acetate, the organic layer was washed with water, dried over anhydrous sodium sulfate and evaporated under vacuum. The residue was purified by chromatography on silica gel eluting with hexanes then a hexanes/ethyl acetate gradient from 9:1 to 4:6 to give the title compound (17.5 mg) as a gum.

MS: 600.6 [M+H]⁺ found.

¹H-NMR (CDCl₃) δ 7.88 (s, 2H), 7.88 (s, 1H), 7.53 (m, 2H), 7.44 (brs, 1H), 4.35 (m, 1H), 4.27 (m, 2H), 4.22 (m, 1H), 4.06 (m, 1H), 4.01 (d, J=11.62 Hz, 1H), 3.37 (m, 1H), 1.73 (m, 1H), 1.48 (m, 1H), 1.38 (m, 1H), 1.30 (t, J=7.06 Hz, 3H), 1.27 (t, J=7.06 Hz, 3H), 0.94 (m, 1H), 0.70 (t, J=7.47 Hz, 3H).

### EXAMPLE 446

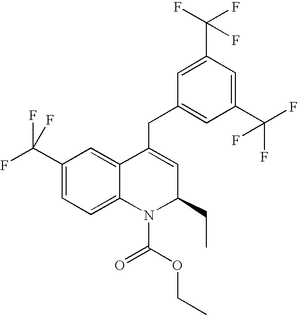

### (R)-4-(3,5-Bis-trifluoromethyl-benzyl)-2-ethyl-6-trifluoromethyl-2H-quinoline-1-carboxylic Acid Ethyl Ester

To a solution of (R, R)-4-[(3,5-bis-trifluoromethyl-phenyl)-methoxycarbonyl-methyl]-2-ethyl-6-trifluoromethyl-2H-quinoline-1-carboxylic acid ethyl ester and (R, S)-4-[(3,5-bis-trifluoromethyl-phenyl)-methoxycarbonyl-methyl]-2-ethyl-6-trifluoromethyl-2H-quinoline-1-carboxylic acid ethyl ester (Examples 3 and 4, used as the mixture obtained prior to separation, 148 mg, 0.253 mmol) in tetrahydrofuran (3 mL) under nitrogen was added aqueous sodium hydroxide solution (1M, 250 μL, 0.25 mmol). After stirring at room temperature for 72 hr an additional aliquot of aqueous sodium hydroxide solution (1M, 100 μL, 0.1 mmol) was added. After 72 hr the mixture was acidified by addition of 2N hydrochloric acid and extracted with ethyl acetate. The organic layer was dried over anhydrous sodium sulfate, evaporated under vacuum and the residue was purified by chromatography on silica gel eluting with dichloromethane/hexanes 1:1 then 3:1 followed by dichloromethane then dichloromethane/methanol 9:1 then a hexanes/ethyl acetate gradient from 9:1 to 4:6 to give the title compound (4.5 mg).

MS: 526.3 [M+H]⁺ found.

¹H-NMR (CDCl₃) δ 7.76 (s, 1H), 7.74 (m, 1H), 7.68 (s, 2H), 7.48 (m, 1H), 7.42 (brs, 1H), 5.76 (d, J=5.81 Hz, 1H), 4.93 (m, 1H), 4.27 (m, 2H), 3.92 (m, 1H), 3.88(m, 1H), 1.48 (m, 1H), 1.38 (m, 1H), 1.32 (t, J=7.47 Hz, 3H), 0.85 (t, J=7.47 Hz, 3H).

### EXAMPLE 447

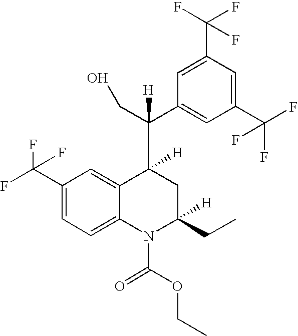

### (R, R, S)-4-[1-(3,5-Bis-trifluoromethyl-phenyl)-2-hydroxy-ethyl]-2-ethyl-6-trifluoromethyl-3,4-dihydro-2H-quinoline-1-carboxylic Acid Ethyl Ester

To a solution of (R, R, S)-4-[(3,5-bis-trifluoromethyl-phenyl)-carboxy-methyl]-2-ethyl-6-trifluoromethyl-3,4-dihydro-2H-quinoline-1-carboxylic acid ethyl ester (Example 435, 67 mg, 0.117 mmol) in tetrahydrofuran (1 mL) under nitrogen was added borane-dimethylsulfide complex (22.2 μL, 0.234 mmol). After 24 hr aqueous sodium hydroxide (1M, 4 drops) was added to quench the reaction and the mixture was allowed to stir for 1 hr. The mixture was acidified by addition of 2N hydrochloric acid, stirred for 10 min and extracted with ethyl acetate. The organic layer was washed with water, dried over anhydrous sodium sulfate and the solvent was evaporated under vacuum. The crude product was purified by chromatography on silica gel eluting with hexanes then hexanes/ethyl acetate 19:1 then 9:1 then 4:1 then 3:7 and finally 2:3 to give the title compound (54 mg).

MS: 558.3 [M+H]⁺ found.

¹H-NMR (CDCl₃) δ 7.83 (s, 1H), 7.80 (s, 2H), 7.60 (brs, 1H), 7.52 (m, 2H), 4.27 (m, 1H), 4.24 (m, 2H), 4.20 (m, 1H), 4.00 (m, 1H), 3.54 (m, 1H), 2.84 (m, 1H), 1.82 (m, 1H), 1.63 (brs, 1H), 1.46 (m, 1H), 1.32 (m, 1H), 1.31 (t, J=7.05 Hz, 3H), 0.87 (m, 1H), 0.69 (t, J=7.47 Hz, 3H).

### Preparation 44

### Methyl (3,5-dichloro)-phenylacetate

A mixture of 3,5-dichlorophenylacetonitrile (2 gm, prepared according to the procedure described in WO00/58292), ethanol (25 mL), potassium hydroxide (3.95 gm) and water (10 mL) was heated under reflux for 4 hr then evaporated to dryness under vacuum. The residue was partitioned between water (20 mL) and diethyl ether and the aqueous layer was acidified to pH1 by addition of concentrated hydrochloric acid. The mixture was extracted with diethyl ether (3×25 mL), the organic layer dried over anhydrous sodium sulfate and evaporated under vacuum. The carboxylic acid was dissolved in methanol (20 mL) and trimethylsilyldiazomethane (2M solution in hexanes, 30 mL, Aldrich Chemical Company, Milwaukee, Wis.) was added slowly. After 1 hr the solvent was removed under vacuum, the residue dissolved in diethyl ether, washed with aqueous sodium carbonate solution (2M), dried over anhydrous sodium sulfate and evaporated to dryness to give the title compound (2.1 gm).

¹H-NMR (CDCl₃) δ 7.27 (s, 1H), 7.17 (s, 2H), 3.71 (s, 3H), 3.57 (s, 2H).

### EXAMPLES 448, 449, 450 AND 451

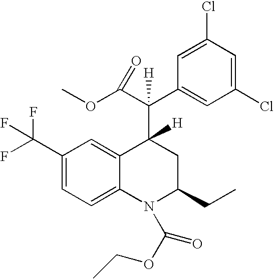

### (R, S, R)-4-[(3,5-Dichloro-phenyl)-methoxycarbonyl-methyl]-2-ethyl-6-trifluoromethyl-3,4-dihydro-2H-quinoline-1-carboxylic Acid Ethyl Ester

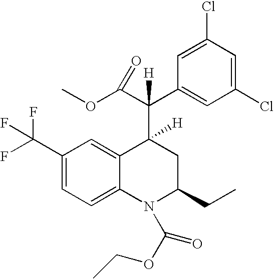

### (R, R, S)-4-[(3,5-Dichloro-phenyl)-methoxycarbonyl-methyl]-2-ethyl-6-trifluoromethyl-3,4-dihydro-2H-quinoline-1-carboxylic Acid Ethyl Ester

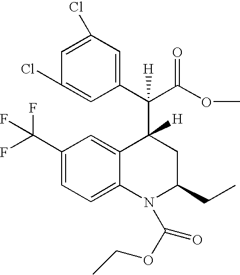

### (R, S, S)-4-[(3,5-Dichloro-phenyl)-methoxycarbonyl-methyl]-2-ethyl-6-trifluoromethyl-3,4-dihydro-2H-quinoline-1-carboxylic Acid Ethyl Ester

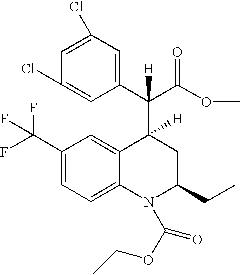

### (R, R, R)-4-[(3,5-Dichloro-phenyl)-methoxycarbonyl-methyl]-2-ethyl-6-trifluoromethyl-3,4-dihydro-2H-quinoline-1-carboxylic Acid Ethyl Ester

To a solution of methyl (3,5-dichloro)-phenylacetate (311 mg, 1.42 mmol) in anhydrous N,N-dimethylformamide (3 mL) was added sodium hydride (60% mineral oil dispersion, 1.93 mmol, 77 mg) and the mixture was stirred at room temperature for 30 min. A solution of (R)-4-bromo-2-ethyl-6-trifluoromethyl-3,4-dihydro-2H-quinoline-1-carboxylic acid ethyl ester (490 mg, mixture of isomers, Preparation 43) in anhydrous N,N-dimethylformamide (5 mL) was added and the mixture was stirred at room temperature for 5 min. Water was added and the mixture was extracted with diethyl ether (3×20 mL) and the organic extract was diluted with heptane and evaporated to dryness to give the crude product as a yellow oil (˜460 mg). Purification was achieved using radial chromatography (Chromatron model 7924T, Harrison Research, Palo Alto, Calif.) with a 4 mm silica gel rotor eluting with hexanes/ethyl acetate 9:1 and subsequently rechromatographing appropriate fractions eluting with dichloromethane/hexanes 45:55 to give the title compounds:

(R, S, R)-4-[(3,5-dichloro-phenyl)-methoxycarbonyl-methyl]-2-ethyl-6-trifluoromethyl-3,4-dihydro-2H-quinoline-1-carboxylic acid ethyl ester (10 mg);

MS: 518.3 [M+H]⁺ found;

¹H-NMR (CDCl₃) δ 7.55 (brd, J=8.30 Hz, 1H), 7.49 (brd, J=8.30 Hz, 1H), 7.48 (brs, 1H), 7.33 (m, 2H), 7.33 (m, 1H), 4.33 (m, 1H), 4.31 (m, 1H), 4.24 (m, 1H), 3.56 (d, J=11.62 Hz, 1H), 3.52 (m, 1H), 3.46 (s, 3H), 1.86 (m, 1H), 1.53 (m, 1H), 1.47 (m, 2H), 1.34 (t, J=7.47 Hz, 3H), 0.74 (t, J=7.47 Hz, 3H).

(R, R, S)-4-[(3,5-dichloro-phenyl)-methoxycarbonyl-methyl]-2-ethyl-6-trifluoromethyl-3,4-dihydro-2H-quinoline-1-carboxylic acid ethyl ester (24 mg);

MS: 518.3 [M+H]⁺ found;

¹H-NMR (CDCl₃) δ 7.52 (m, 1H), 7.50 (m, 1H), 7.35 (t, J=1.66 Hz, 1H), 7.32 (brs, 1H), 7.30 (d, J=1.66 Hz, 2H), 4.30 (m, 1H), 4.27 (m, 1H), 4.20 (m, 1H), 3.78 (d, J=11.61 Hz, 1H), 3.74 (s, 3H), 3.28 (m, 1H), 1.85 (m, 1H), 1.49 (m, 1H), 1.39 (m, 1H), 1.30 (t, J=7.47 Hz, 3H), 0.91 (m, 1H), 0.72 (t, J=7.47 Hz, 3H).

(R, S, S)-4-[(3,5-dichloro-phenyl)-methoxycarbonyl-methyl]-2-ethyl-6-trifluoromethyl-3,4-dihydro-2H-quinoline-1-carboxylic acid ethyl ester (29 mg);

MS: 518.3 [M+H]⁺ found;

¹H-NMR (CDCl₃) δ 7.43 (d, J=8.30 Hz, 1H), 7.36 (dd, J=8.30, 1.66 Hz, 1H), 7.15 (t, J=1.66, 1H), 6.88 (d, J=1.66 Hz, 2H), 6.69 (brs, 1H), 4.45 (m, 1H), 4.32 (m, 1H), 4.31 (m, 1H), 3.75 (s, 3H), 3.62 (d, J=10.79 Hz, 1H), 3.40 (m, 1H), 2.88 (m, 1H), 1.75 (m, 1H), 1.64 (m, 1H), 1.48 (m, 1H), 1.34 (t, J=7.47 Hz, 3H), 0.83 (t, J=7.47 Hz, 3H).

(R, R, R)-4-[(3,5-dichloro-phenyl)-methoxycarbonyl-methyl]-2-ethyl-6-trifluoromethyl-3,4-dihydro-2H-quinoline-1-carboxylic acid ethyl ester (18 mg);

MS: 518.3 [M+H]⁺ found;

¹H-NMR (CDCl₃) δ 7.51 (brd, J=8.59 Hz, 1H), 7.43 (brd, J=8.59 Hz, 1H), 7.33 (m, 2H), 7.31 (m, 1H), 7.10 (brs, 1H), 4.40 (m, 1H), 4.28 (m, 1H), 4.23 (m, 1H), 4.06 (d, J=10.15 Hz, 1H), 3.74 (s, 3H), 3.24 (m, 1H), 2.32 (m, 1H), 1.64 (m, 1H), 1.47 (m, 1H), 1.37 (m, 1H), 1.32 (t, J=7.02 Hz, 3H), 0.83 (t, J=7.47 Hz, 3H).

### EXAMPLE 452

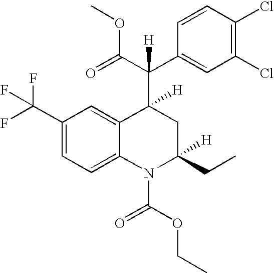

### (R, R, S)-4-[(3,4-Dichloro-phenyl)-methoxycarbonyl-methyl]-2-ethyl-6-trifluoromethyl-3,4-dihydro-2H-quinoline-1-carboxylic Acid Ethyl Ester

To a solution of methyl (3,4-dichloro)-phenylacetate (1.22 gm, 5.57 mmol) in anhydrous N,N-dimethylformamide (5 mL) was added sodium hydride (60% mineral oil dispersion, 7 mmol, 280 mg) and the mixture was stirred at room temperature for 5 min. A solution of (R)-4-bromo-2-ethyl-6-trifluoromethyl-3,4-dihydro-2H-quinoline-1-carboxylic acid ethyl ester (798 mg, mixture of isomers, Preparation 43) in anhydrous N,N-dimethylformamide (4 mL) was added and the mixture was stirred at room temperature for 15 min. Water was added and the mixture was acidified by addition of 2N hydrochloric acid then extracted with dichloromethane (×3) and the organic extract was dried over anhydrous sodium sulfate and evaporated to dryness to give a yellow oil. This was dissolved in tetrahydrofuran (10 mL) and water (5 mL) and aqueous sodium hydroxide (2N, 10 mL) was added. The mixture was stirred at room temperature for 24 hr then partitioned between hydrochloric acid (0.1N) and dichloromethane. The organic extract was dried over anhydrous sodium sulfate and evaporated to dryness to give a mixture of carboxylic acids. This material was purified by chromatography on silica gel eluting with hexanes/ethyl acetate 5:1 then 4:1. Fractions containing the first carboxylic acid to elute (with the desired R, R, S stereochemistry) were combined, evaporated to dryness under vacuum and dissolved in methanol (25 mL). Trimethylsilyldiazomethane (2M solution in hexanes, Aldrich Chemical Company, Milwaukee, Wis.) was added slowly until effervescence ceased and a yellow color persisted. This was discharged by addition of a small amount of acetic acid then the solvent was removed under vacuum and the residue was purified by chromatography on silica gel eluting with a hexanes/dichloromethane gradient from 2:1 to 1:2. Final purification was achieved using reverse phase HPLC using the Shimadzu preparative HPLC system (Shimadzu Corporation, Kyoto, Japan) eluting with a 30-100% acetonitrile/water/0.1% formic acid gradient on a 19×50 mm Waters Symmetry Column (Waters Corp, Milford, Mass.) 8 min run, 6 min gradient, 25 ml/min, UV triggered collection, observing at 210 nm. The product-containing fractions were evaporated to dryness to give the title compound (75 mg).

MS: 518.3 [M+H]⁺ found.

¹H-NMR (CDCl₃) δ 7.52 (m, 1H), 7.51 (m, 1H), 7.50 (m, 1H), 7.47 (d, J=8.3 Hz, 1H), 7.34 (brs, 1H), 7.25 (dd, J=8.3, 2.49 Hz, 1H), 4.28 (m, 1H), 4.27 (m, 1H), 4.20 (m, 1H), 3.80 (d, J=11.62 Hz, 1H), 3.73 (s, 3H), 3.29 (m, 1H), 1.86 (m, 1H), 1.50 (m, 1H), 1.37 (m, 1H), 1.30 (t, J=7.47 Hz, 3H), 0.90 (m, 1H), 0.71 (t, J=7.47 Hz, 3H).

### EXAMPLE 453

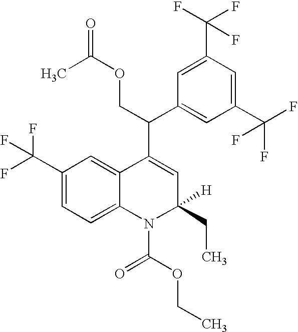

### (R, S) and (R, R)-4-[2-Acetoxy-1-(3,5-bis-trifluoromethyl-phenyl)-ethyl]-2-ethyl-6-trifluoromethyl-2H-quinoline-1-carboxylic Acid Ethyl Ester

To a solution of the diastereoisomeric mixture (R, R) and (R, S)-4-[1-(3,5-bis-trifluoromethyl-phenyl)-2-hydroxy-ethyl]-2-ethyl-6-trifluoromethyl-2H-quinoline-1-carboxylic acid ethyl ester (Example 437, 57 mg, 0.102 mmol) in dichloromethane (1 mL) under nitrogen was added triethylamine (43 μL, 0.306 mmol) followed by acetyl chloride (9 μL, 0.132 mmol). After 3 hr the mixture was partitioned between water and dichloromethane, the organic layer was separated, dried over anhydrous sodium sulfate and evaporated under vacuum. The residue was purified by chromatography on silica gel eluting with hexanes then hexanes/ethyl acetate 19:1 then 9:1 then 4:1 then 3:7 and finally 2:3 to give the title compound as a mixture of diastereoisomers (51 mg).

MS: 598.3 [M+H]⁺ found.

### EXAMPLE 454

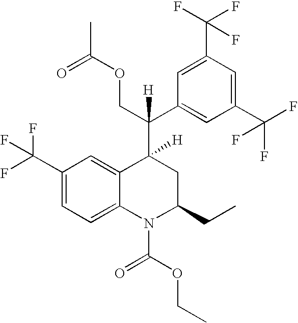

### (R, R, S)-4-[2-Acetoxy-1-(3,5-bis-trifluoromethyl-phenyl)-ethyl]-2-ethyl-6-trifluoromethyl-3,4-dihydro-2H-quinoline-1-carboxylic Acid Ethyl Ester

To a solution of (R, R, S)-4-[1-(3,5-bis-trifluoromethyl-phenyl)-2-hydroxy-ethyl]-2-ethyl-6-trifluoromethyl-3,4-dihydro-2H-quinoline-1-carboxylic acid ethyl ester (Example 447, 50 mg, 0.0896 mmol) in dichloromethane (1 mL) under nitrogen was added triethylamine (37 μL, 0.269 mmol) followed by acetyl chloride (8.3 μL, 0.116 mmol). After 30 min the mixture was partitioned between water and dichloromethane, the organic layer was separated, dried over anhydrous sodium sulfate and evaporated under vacuum. The residue was purified by chromatography on silica gel eluting with hexanes then hexanes/ethyl acetate 19:1 then 9:1 then 4:1 to give the title compound (41 mg).

MS: 600.4 [M+H]⁺ found.

¹H-NMR (CDCl₃) δ 7.88 (s, 1H), 7.85 (s, 1H), 7.73 (s, 2H), 7.64 (m, 1H), 7.62 (m, 1H), 4.80 (dd, J=11.62, 4.15 Hz, 1H), 4.29 (m, 1H), 4.25 (m, 1H), 4.23 (m, 1H), 4.20 (m, 1H), 3.72 (ddd, J=9.13, 9.13, 3.32 Hz, 1H), 2.79 (m, 1H), 1.97 (s, 3H), 1.84 (m, 1H), 1.46 (m, 1H), 1.32 (m, 1H), 1.31 (t, J=7.47 Hz, 3H), 0.87 (m, 1H), 0.69 (t, J=7.47 Hz, 3H).

### EXAMPLE 455

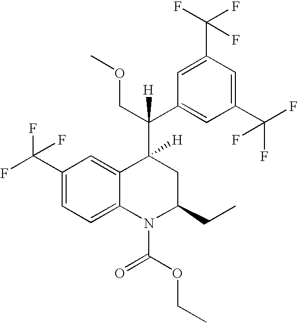

### (R, R, S)-4-[1-(3,5-Bis-trifluoromethyl-phenyl)-2-methoxy-ethyl]-2-ethyl-6-trifluoromethyl-3,4-dihydro-2H-quinoline-1-carboxylic Acid Ethyl Ester

To a solution of (R, R, S)-4-[1-(3,5-bis-trifluoromethyl-phenyl)-2-hydroxy-ethyl]-2-ethyl-6-trifluoromethyl-3,4-dihydro-2H-quinoline-1-carboxylic acid ethyl ester (Example 447, 20 mg, 0.035 mmol) in tetrahydrofuran (1 mL) under nitrogen was added sodium hydride (60% mineral oil dispersion, 0.043 mmol, 1.7 mg) followed after 5 min by iodomethane (2 drops). After stirring for 16 hr additional aliquots of sodium hydride (2 mg) and iodomethane (3 drops) were added. The mixture was stirred at room temperature for 48 hr then diluted with ethyl acetate. The mixture was washed with water, the organic layer separated, dried over anhydrous sodium sulfate and evaporated under vacuum. The crude product was purified by chromatography on silica gel eluting with hexanes then hexanes/ethyl acetate 19:1 then 9:1 to give the title compound (19 mg).

MS: 572.5 [M+H]⁺ found.

¹H-NMR (CDCl₃) δ 7.81 (s, 1H), 7.76 (s, 2H), 7.61 (brs, 1H), 7.52 (m, 2H), 4.27 (m, 1H), 4.24 (m, 1H), 4.21 (m, 1H), 3.89 (dd, J=9.96, 3.32 Hz, 1H), 3.74 (dd, J=9.13, 7.47 Hz, 1H), 3.55 (m, 1H), 3.29 (s, 3H), 2.85 (m, 1H), 1.80 (m, 1H), 1.47 (m, 1H), 1.32 (m, 1H), 1.31 (t, J=7.47 Hz, 3H), 0.87 (m, 1H), 0.69 (t, J=7.47 Hz, 3H).

### EXAMPLE 456

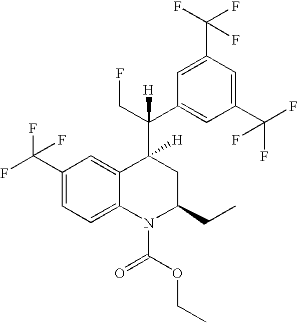

### (R, R, S)-4-[1-(3,5-Bis-trifluoromethyl-phenyl)-2-fluoro-ethyl]-2-ethyl-6-trifluoromethyl-3,4-dihydro-2H-quinoline-1-carboxylic Acid Ethyl Ester

To a solution of (R, R, S)-4-[1-(3,5-bis-trifluoromethyl-phenyl)-2-hydroxy-ethyl]-2-ethyl-6-trifluoromethyl-3,4-dihydro-2H-quinoline-1-carboxylic acid ethyl ester (Example 447, 20 mg, 0.035 mmol) in dichloromethane (1 mL) under nitrogen was added diethylaminosulfur trifluoride (47 μL, 0.358 mmol). The mixture was stirred at room temperature for 3 hr then a further aliquot of diethylaminosulfur trifluoride (47 μL, 0.358 mmol) was added. After 1 hr the mixture was partitioned between water and dichloromethane, the organic layer was separated, dried over anhydrous sodium sulfate and evaporated under vacuum. The residue was initially purified by chromatography on silica gel eluting with hexanes/ethyl acetate 19:1 then 19:1 then 9:1 and finally purified by reverse phase hplc using the Shimadzu preparative HPLC system (Shimadzu Corporation, Kyoto, Japan) eluting with a 30-100% acetonitrile/water/0.1% formic acid gradient on a 19×50 mm Waters Symmetry Column (Waters Corp, Milford, Mass.) 8 min run, 6 min gradient, 25 ml/min, UV triggered collection, observing at 210 nm. The product-containing fractions were evaporated to dryness to give the title compound (3 mg).

MS: 560.3 [M+H]⁺ found.

¹H-NMR (CDCl₃) δ 7.86 (s, 1H), 7.80 (s, 2H), 7.55 (m, 2H), 7.54 (s, 1H), 4.93 (ddd, J=46.47, 9.96, 4.14 Hz, 1H), 4.82 (ddd, J=46.47, 9.96, 6.64 Hz, 1H), 4.28 (m, 1H), 4.27 (m, 1H), 4.23 (m, 1H), 3.72 (m, 1H), 2.91 (m, 1H), 1.88 (m, 1H), 1.48 (m, 1H), 1.34 (m, 1H), 1.32 (t, J=7.47 Hz, 3H), 0.92 (m, 1H), 0.71 (t, J=7.47 Hz, 3H).

### EXAMPLE 457

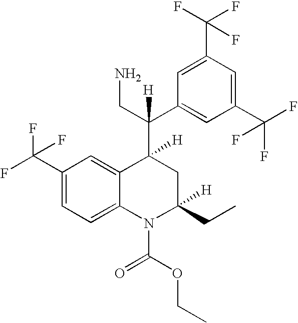

### (R, R, S)-4-[1-(3,5-Bis-trifluoromethyl-phenyl)-2-amino-ethyl]-2-ethyl-6-trifluoromethyl-3,4-dihydro-2H-quinoline-1-carboxylic Acid Ethyl Ester

To a solution of (R, R, S)-4-[(3,5-bis-trifluoromethyl-phenyl)-carbamoyl-5 methyl]-2-ethyl-6-trifluoromethyl-3,4-dihydro-2H-quinoline-1-carboxylic acid ethyl ester (Example 438, 25 mg, 0.0438 mmol) in tetrahydrofuran (3 mL) under nitrogen was added borane-dimethylsulfide complex (8.3 μL, 0.0876 mmol). The mixture was heated at 70° C. for 48 hr then water (2.5 mL) and saturated aqueous sodium carbonate solution (1 mL) was added. The mixture was heated at 70° C. for 1 hr then partitioned between water and dichloromethane. The organic layer was evaporated to dryness under vacuum and the residue dissolved in diethyl ether (5 mL) and 2N hydrochloric acid (1 mL) added. After 24 hr the mixture was diluted with water, sodium carbonate solution added and the mixture was extracted with dichloromethane. The organic layer was dried over anhydrous sodium sulfate and the solvent was evaporated under vacuum. The crude product was purified by chromatography on silica gel eluting with hexanes/ethyl acetate 3:1 then ethyl acetate give the title compound (18.5 mg).

MS: 557.4 [M+H]⁺ found.

¹H-NMR (CDCl₃) δ 7.84 (s, 1H), 7.77 (s, 2H), 7.58 (brs, 1H), 7.52 (m, 2H), 4.27 (m, 1H), 4.23 (m, 1H), 4.22 (m, 1H), 3.44 (dd, J=13.28, 3.32 Hz, 1H), 3.35 (m, 1H), 3.09 (dd, J=13.28, 9.13 Hz, 1H), 2.76 (m, 1H), 1.76 (m, 1H), 1.45 (m, 1H), 1.34 (m, 1H), 1.31 (t, J=7.47 Hz, 3H), 0.84 (m, 1H), 0.69 (t, J=7.47 Hz, 3H).

### EXAMPLES 458, 459, 460 AND 461

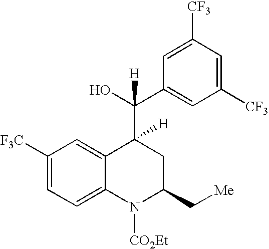

### [(R, S, S)]-4-[(3,5-bis-trifluoromethyl-phenyl)-hydroxy-methyl]-2-ethyl-6-trifluoromethyl-3,4-dihydro-2H-quinoline-1-carboxylic Acid Ethyl Ester

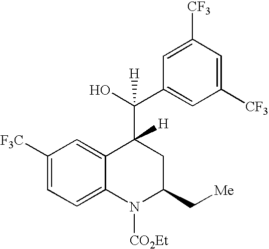

### [(R, R, R)]-4-[(3,5-bis-trifluoromethyl-phenyl)-hydroxy-methyl]-2-ethyl-6-trifluoromethyl-3,4-dihydro-2H-quinoline-1-carboxylic Acid Ethyl Ester

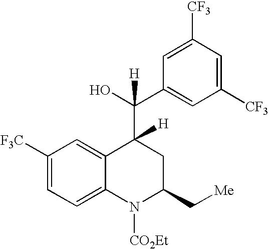

### [(R, R, S)]-4-[(3,5-bis-trifluoromethyl-phenyl)-hydroxy-methyl]-2-ethyl-6-trifluoromethyl-3,4-dihydro-2H-quinoline-1-carboxylic Acid Ethyl Ester

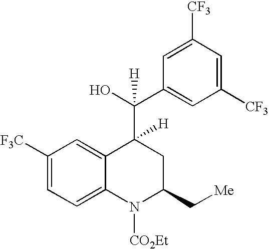

### [(R, S, R)]-4-[(3,5-bis-trifluoromethyl-phenyl)-hydroxy-methyl]-2-ethyl-6-trifluoromethyl-3,4-dihydro-2H-quinoline-1-carboxylic Acid Ethyl Ester

These compounds (previously prepared in Examples 22, 23, 24 and 25) were prepared in optically enriched form by resolution of the corresponding racemate indicated, or an intermediate in its synthesis, using methods analogous to those described herein.

### EXAMPLES 462, 463, 464 AND 465

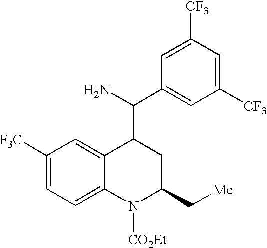

### Preparation of [(R, R, R)], [(R, R, S)], [(R, S, S)], and [(R, S, R)]-4-[Amino-(3,5-bis-trifluoromethyl-phenyl)-methyl]-2-ethyl-6-trifluoromethyl-3,4-dihydro-2H-quinoline-1-carboxylic Acid Ethyl Ester

General procedure to make amine compounds: [(R, R, R)]-4-(3,5-bis-trifluoromethyl-phenyl)-hydroxy-methyl]-2-ethyl-6-trifluoromethyl-3,4-dihydro-2H-quinoline-1-carboxylic acid ethyl ester (0.527 grams, 0.971 mmol, 1 eq) was placed in a round bottomed flask equipped with a magnetic stir bar. Methylene chloride (20 mL) was added followed by the addition of triethylamine (0.456 mL, 3.37 mmol, 3.37 eq) and mesyl chloride (0.150 mL, 1.94 mmol, 2.0 eq) at room temperature and the reaction mixture was stirred overnight. The reaction mixture was quenched with water, extracted 4 times with ethyl acetate. The organic layer was washed with 0.1M HCl, followed by saturated bicarbonate solution and dried over sodium sulfate. The solution was filtered, concentrated and dried on the high vacuum to provide [(R, R, R)]-4-[(3,5-bis-trifluoromethyl-phenyl)-methanesulfonyloxy-methyl]-2-ethyl-6-trifluoromethyl-3,4-dihydro-2H-quinoline-1-carboxylic acid ethyl ester (0.599 grams, 99% yield) as a white solid, MS (ES+) m/z=622 (M+1).

[(R, R, R)]-4-[(3,5-bis-trifluoromethyl-phenyl)-methanesulfonyloxy-methyl]-2-ethyl-6-trifluoromethyl-3,4-dihydro-2H-quinoline-1-carboxylic acid ethyl ester (0.313 grams, 0.504 mmol, 1 eq) was placed in a round bottomed flask equipped with a magnetic stir bar. DMF (12 mL) was added followed by the addition of sodium azide (0.201 grams, 0.3.10 mmol, 6.1 eq). The reaction mixture was heated to 70° C. for 12 hours. The reaction mixture was cooled to room temperature, diluted into 200 mL of EtOAc and washed 4 times with brine and water. The EtOAc was collected, dried over sodium sulfate, filtered and concentrated. The material was purified on a Biotage flash 40s to provide [(R, R, S)]-4-[Azido-(3,5-bis-trifluoromethyl-phenyl)-methyl]-2-ethyl-6-trifluoromethyl-3,4-dihydro-2H-quinoline-1-carboxylic acid ethyl ester 0.202 grams, 71% yield) MS (ES+) m/z=569 (M+1).

[(R, R, S)]-4-[Azido-(3,5-bis-trifluoromethyl-phenyl)-methyl]-2-ethyl-6-trifluoromethyl-3,4-dihydro-2H-quinoline-1-carboxylic acid ethyl ester (0.202 grams, 0.356 mol, 1 eq) was placed in a round bottomed flask and equipped with a magnetic stir bar and reflux condenser. NH₄CO₂H (0.226 grams, 3.58 mol, 10.1 eq) and Pd/C (0.113 grams, 0.107 mol, 0.30 eq) was added followed by the addition of a 2:1 solution of methanol and ethyl acetate (8.80 mL). The reaction mixture was refluxed for 2 hours and then filtered through Celite®. The fitrate was concentrated but not to dryness and partitioned between ethyl acetate and aqueous sodium bicarbonate solution. The organic layer was collected, dried over sodium sulfate, filtered and concentrated. The crude material was purified on Biotage Flash 40 M to provide the desired compound, [(R, R, S)], 4-[Amino-(3,5-bis-trifluoromethyl-phenyl)-methyl]-2-ethyl-6-trifluoromethyl-3,4-dihydro-2H-quinoline-1-carboxylic acid ethyl ester (0.162 grams, 83% yield) MS (ES+) m/z=543 (M+1).

¹H NMR (CDCl₃): δ 0.82 (t, 3H), 1.32 (t, 3H), 1.34-1.57 (m, 3H), 2.53 (m, 1H), 3.00 (m, 1H), 4.28 (q, 2H), 4.50 (d, 1H), 4.54 (m, 1H), 7.03 (s, 1H), 7.42 (d, 1H), 7.60 (d, 1H), 7.66 (s, 2H), 7.72 (s, 1H).

MS (ES+) m/z=543 (M+1).

These additional compounds were prepared using analogous procedures to those described herein:

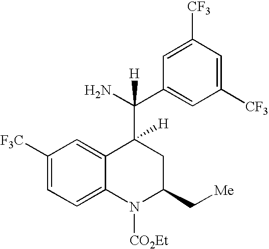

### [(R, S, S)], 4-[Amino-(3,5-bis-trifluoromethyl-phenyl)-methyl]-2-ethyl-6-trifluoromethyl-3,4-dihydro-2H-quinoline-1-carboxylic Acid Ethyl Ester

¹H NMR (CDCl₃): δ 0.71 (t, 3H), 1.00 (m, 1H), 1.29 (t, 3H), 1.33-1.52 (m, 2H), 1.67 (m, 1H & H₂O), 2.70 (m, 1H), 4.15-4.31 (m, 3H), 4.47 (d, 1H), 7.51 (m, 2H), 7.84 (s, 1H), 7.89 (s, 2H), 7.91 (s, 1H);

MS (ES+) m/z=543 (M+1).

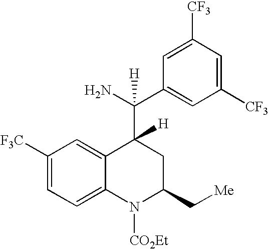

### [(R, R, R)], 4-[Amino-(3,5-bis-trifluoromethyl-phenyl)-methyl]-2-ethyl-6-trifluoromethyl-3,4-dihydro-2H-quinoline-1-carboxylic Acid Ethyl Ester

HNMR (CDCl₃): δ 0.75 (t, 3H), 1.33 (t, 3H), 1.37-1.62 (m, 3H), 1.82 (m, 1H), 2.98 (m, 1H), 4.17-4.36 (m, 4H), 7.51 (s, 1H), 7.53-7.61 (m, 2H), 7.83 (s, 1H), 7.86 (s, 2H);

MS (ES+) m/z=543 (M+1).

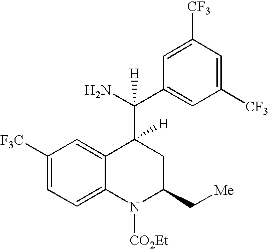

### [(R, S, R)], 4-[Amino-(3,5-bis-trifluoromethyl-phenyl)-methyl]-ethyl-6-2-trifluoromethyl-3,4-dihydro-2H-quinoline-1-carboxylic Acid Ethyl Ester

HNMR (CDCl₃): δ 0.82 (t, 3H), 1.27 (t, 3H), 1.40-1.64 (m, 3H), 2.07 (m, 1H), 2.76 (m, 1H), 4.09-4.27 (m, 3H), 5.07 (m, 1H), 7.55 (q, 2H), 7.66 (s, 1H), 7.83 (s, 1H), 7.96 (s, 2H);

MS (ES+) m/z=543 (M+1).

### Preparation 45

### Benzhydrylidene-(3,5-bis-trifluoromethyl-benzyl)-amine

A solution of 3,5-bis-trifluoromethyl-benzylamine (75 gm, 0.308 mol) and benzophenone imine (53.5 mL, 57.78 gm, 0.319 mol) in diisopropyl ether (375 mL) was heated under reflux for 1 hr. The mixture was then concentrated under vacuum to a volume of 100 mL, isopropanol (250 mL) was added and the volume reduced to 150 mL at atmospheric pressure. After stirring overnight the crystalline product was isolated by filtration and washed with isopropanol (25 mL) to give the title compound (92%).

### Preparation 46

### [(R, R, S)]-4-[(Benzhydrylidene-amino)-(3,5-bis-trifluoromethyl-phenyl)-methyl]-2-ethyl-6-trifluoromethyl-3,4-dihydro-2H-quinoline-1-carboxylic Acid Ethyl Ester

To a solution of (2R)-4-chloro-2-ethyl-6-trifluoromethyl-3,4-dihydro-2H-quinoline-1-carboxylic acid ethyl ester (Preparation 14, 50 gm, 0.149 mol) and benzhydrylidene-(3,5-bis-trifluoromethyl-benzyl)-amine (60.7 gm, 0.149 mol) in N,N-dimethylformamide (150 mL) was added a solution of sodium hexamethyldisilazide (1M in tetrahydrofuran, 164 mL, 0.164 mol) under nitrogen. The mixture was stirred at 20° C. to 30° C. for 3 hr then water (500 mL) followed by diisopropyl ether (750 mL) was added. The upper layer was separated and concentrated under vacuum to a volume of 250 mL. Hexane (750 mL) was added and the mixture concentrated to a volume of 250 mL. Hexane (750 mL) was added and the mixture concentrated to a volume of 250 mL. The precipitate was collected by filtration to give the title compound (26.3 gm, 37.2 mmol) as a crystalline solid. The mother liquor contained [(R, R, R)]-4-[(benzhydrylidene-amino)-(3,5-bis-trifluoromethyl-phenyl)-methyl]-2-ethyl-6-trifluoromethyl-3,4-dihydro-2H-quinoline-1-carboxylic acid ethyl ester which could be isolated by chromatography on silica gel if desired.

### EXAMPLE 466

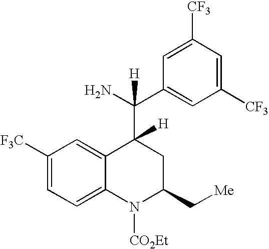

### [(R, R, S)]-4-[Amino-(3,5-bis-trifluoromethyl-phenyl)-methyl]-2-ethyl-6-trifluoromethyl-3,4-dihydro-2H-quinoline-1-carboxylic Acid Ethyl Ester (Method 2)

To a suspension of [(R, R, S)]-4-[(benzhydrylidene-amino)-(3,5-bis-trifluoromethyl-phenyl)-methyl]-2-ethyl-6-trifluoromethyl-3,4-dihydro-2H-quinoline-1-carboxylic acid ethyl ester (40 gm, 56.6 mmol) in isopropanol (200 mL) was added methanesulfonic acid (3.7 mL, 57 mmol) and water (1 mL). The mixture was heated until the solid dissolved then allowed to cool and granulate. The mixture was concentrated to a volume of about 50 mL, diisopropyl ether (200 mL) was added and the suspension was stirred at 0° C. for 4 hr. The crystalline solid was collected by filtration, washed with diisopropyl ether (2×100 mL) and dried in air to give the mesylate salt of the title compound (36.1 gm, 100%). This material (32 gm, 50 mmol) was suspended in water (300 mL) and diethyl ether (300 mL) followed by aqueous sodium hydroxide (1N, 52 mL, 52 mmol) were added with stirring. The upper layer was separated and the aqueous layer extracted with diethyl ether (2×100 mL). The combined ether layers were dried over anhydrous magnesium sulfate and evaporated under vacuum to give the title compound (27.1 gm, 100%) as a clear colorless oil.

### EXAMPLE 467, 468, 469, AND 470

The following compounds were prepared from starting materials and procedures analogous to those described above, particularly in Examples 18, 19, and 456.

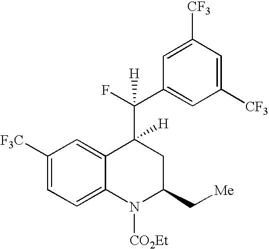

[(R, S, R)]-4-(3,5-bis-trifluoromethyl-phenyl)-fluoro-methyl]-2-ethyl-6-trifluoromethyl-3,4-dihydro-2H-quinoline-1-carboxylic acid ethyl ester

¹H-NMR (CDCl₃): δ 7.92 (s, 1H), 7.83 (s, 2H), 7.67 (s, 1H), 7.57 (s, 2H), 6.5 (d, 1H), 4.24 (m, 3H), 2.91 (dd, 1H), 2.08 (m, 1H), 1.51 (m, 3H), 1.27 (t, 3H), 0.80 (t, 3H);

MS: 546.3 [M+H]+ found.

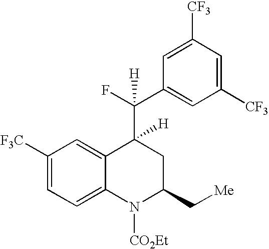

[(R, S, S)]-4-(3,5-bis-trifluoromethyl-phenyl)-fluoro-methyl]-2-ethyl-6-trifluoromethyl-3,4-dihydro-2H-quinoline-1-carboxylic acid ethyl ester

¹H-NMR (CDCl₃): δ 7.96 (s, 1H), 7.88 (s, 2H), 7.76 (s, 1H), 7.55 (s, t, 2H), 5.86 (dd, J=9.13 Hz, 1H), 4.25 (m, 3H), 3.10 (m, 1H), 1.75 (m, 1H), 1.47 (m, 2H), 1.29 (t, 3H), 1.18 (t, 3H), 0.77 (t, 3H);

MS: 546.4 [M+H]+ found.

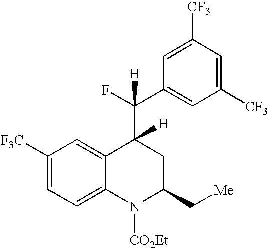

[(R, R, S)]-4-(3,5-bis-trifluoromethyl-phenyl)-fluoro-methyl]-2-ethyl-6-trifluoromethyl-3,4-dihydro-2H-quinoline-1-carboxylic acid ethyl ester

¹H-NMR (CDCl₃): δ 7.8 (s, 1H), 7.59 (d, 1H), 7.47 (d, 1H), 7.43 (s, 2H), 6.86 (s, 1H), 5.65 (dd, J=7.88 Hz, 1H), 4.60 (m, 1H), 4.28 (m, 2H), 3.21 (m, 1H), 2.65 (m, 3H), 1.74 (m, 1H), 1.41-1.62 (m, 2H), 1.31 (t, 3H), 0.86 (t, 3H);

MS: 546.3 [M+H]+ found

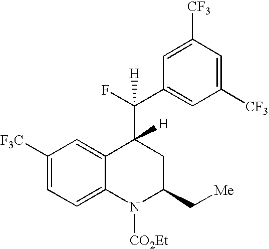

[(R, R, R)]-4-[(3,5-bis-trifluoromethyl-phenyl)-fluoro-methyl]-2-ethyl-6-trifluoromethyl-3,4-dihydro-2H-quinoline-1-carboxylic acid ethyl ester

¹H-NMR (CDCl₃): δ 7.88 (s, 1H), 7.63 (s, d, 3H), 7.52 (d, 1H), 7.41 (s, 1H), 5.58 (dd, J=7.47 Hz, 1H), 4.42 (m, 1H), 4.24 (m, 2H), 3.40 (m, 1H), 1.97 (m, 1H), 1.74 (m, 1H), 1.41 (m, 2H), 1.27 (t, 3H), 0.79 (t, 3H);

MS: 546.3 [M+H]+ found.

### EXAMPLES 471, 472, 473, AND 474

The following compounds were prepared from starting materials and procedures analogous to those described above, as shown in Scheme 2 whereby the addition of a suitable organometallic derivative such as magnesium or lithium derivative, prepared from a compound alkyl-Hal, wherein Hal represents a chlorine, bromine or iodine atom to XXV produced compounds shown in examples 471, 472, 473, and 474.

[(R, R), (S, S)]-4-[(3,5-Bis-trifluoromethyl-benzoyl)]-6,-trifluoromethyl -2-ethyl-3,4-dihydro-2H-quinoline-1-carboxylic acid ethyl ester (0.015 grams, 0.028 mmol) was placed in a small round bottomed flask containing a magnetic stir bar and dissolved in 0.50 mL of tetrathydrofuran. Methyl magnesium bromide solution (0.028 mL of 3.0 M in diethylether) was added to the reaction mixture at room temperature and stirred for 2 hours. The reaction mixture was then quenched with saturated ammonium chloride solution and extracted into ethyl acetate. The organic layer was washed with water, dried over magnesium sulafte, filtered and concentrated to provide the desired products as an crude oil. The alcohol diastereomers were separated by silica gel chromatography to provide [(R, R, R)],-4-[(3,5-bis-trifluoromethyl-phenyl)-1-hydroxyl-ethyl]-2-ethyl-6-trifluoromethyl-3,4-dihydro-2H-quinoline-1-carboxylic acid ethyl ester and [(R, R, S)]4-[(3,5-bis-trifluoromethyl-phenyl)-1-hydroxyl-ethyl]-2-ethyl-6-trifluoromethyl-3,4-dihydro-2H-quinoline-1-carboxylic acid ethyl ester.

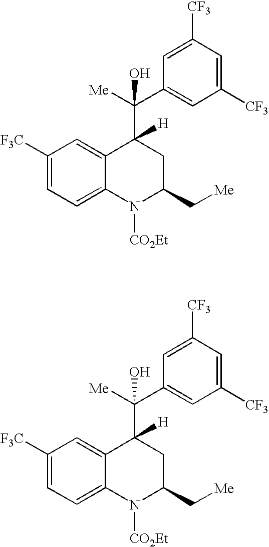

[(R, R, R)], (R, R, S)]-4-[(3,5-bis-trifluoromethyl-phenyl)-1-hydroxyl-ethyl]-2-ethyl-6-trifluoromethyl-3,4-dihydro-2H-quinoline-1-carboxylic acid ethyl ester

First eluting compound: ¹H-NMR (CDCl₃): δ 7.86 (s, 2H), 7.80 (s, 1H), 7.53 (dd, 2H), 7.34 (s, 1H), 4.3 (m, 2H), 4.18 (m, 1H), 3.18 (m, 1H), 1.95 (m, 1H), 1.80 (s, 1H), 1.5-1.63 (m, 4H), 1.24-1.45 (m, 5H), 0.70 (t, 3H);

MS: 588.3 [M+H]+ found.

Second eluting compound: ¹H-NMR (CDCl₃): δ 7.76 (m, 3H), 7.53 (d, 1H), 7.42 (d, 1H), 7.06 (s, 1H), 4.41 (m, 1H), 4.2 (q, 2H), 3.18 (m, 1H), 2.30 (m, 1H), 1.91 (br s, 1H), 1.62-1.74 (m, 4H), 1.24-1.43 (m, 5H), 0.77 (t, 3H);

MS: 588.3 [M+H]+ found.

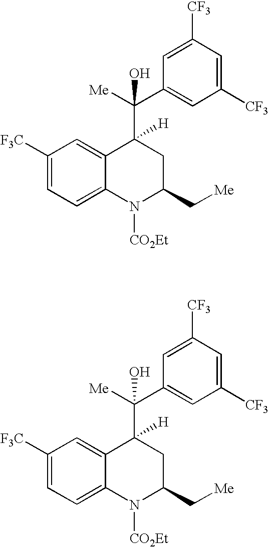

[(R, S, R)], (R, S, S)]-4-[(3,5-bis-trifluoromethyl-phenyl)-1-hydroxyl-ethyl]-2-ethyl-6-trifluoromethyl-3,4-dihydro-2H-quinoline-1-carboxylic acid ethyl ester

First eluting compound: ¹H-NMR (CDCl₃): δ 7.86 (s, 1H), 7.80 (s, 1H), 7.70 (s, 1H), 7.55 (m , 2H), 7.35 (s, 1H), 4.3 (m, 2H), 4.19 (m, 1H), 3.20 (m, 1H), 1.91 (m, 1H), 1.60 (s, 3H), 1.30 (t, 3H), 0.70 (t, 3H);

MS: 556.2 [M−H]⁻ found.

Second eluting compound: ¹H-NMR (CDCl₃): δ 8.0 (s, 2H), 7.80 (s, 1H), 7.50 (brs, 1H), 7.40 (dd, 2H), 4.4 (br m, 1H), 4.20 (m, 2H), 3.05 (d, 1H), 2.55 (br m, 1H), 1.74 (s, 3H), 1.63 (m, 1H), 1.43 (m, 2H), 1.30 (t, 3H), 0.90 (t, 3H);

MS: 558.3 [M+H]⁺ found.

### EXAMPLE 475 AND 476

The following compounds were prepared from starting materials and procedures analogous to those described in Scheme 1 above, particularly in Example 2.

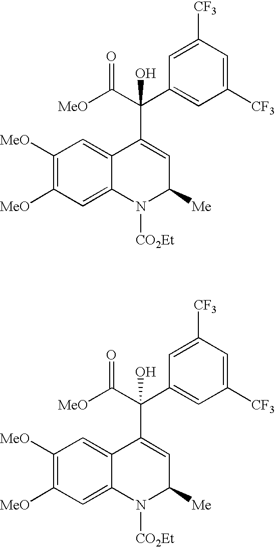

[(R, R)], (R, S)]-4-[(3,5-bis-trifluoromethyl-phenyl)-1-hydroxyl-methoxycarbonyl-methyl]-6,7-dimethoxy-2-methyl-2H-quinoline-1-carboxylic acid ethyl ester Diastereomer 1: ¹H-NMR (CDCl₃): δ 7.92 (s, 2H), 7.73 (s, 1H), 6.81 (s, 1H), 5.80 (d, 1H), 5.20 (br m, 1H), 4.40 (brs, 1H), 4.30 (m, 1H), 4.20 (br m, 1H), 3.84 (s, 3H), 3.80 (s, 3H), 3.50 (s, 3H), 1.34 (m, 4H), 1.09 (d, 3H);

MS: 578.5 [M+H]+ found.

Diastereomer 2: ¹H-NMR (CDCl₃): δ 8.18 (s, 2H), 7.87 (s, 1H), 6.67 (s, 1H), 5.65 (br s, 1H), 5.15 (br s, 1H), 4.31 (br m, 1H), 4.20 (br m, 1H), 3.86 (s, 3H), 3.80 (s, 3H), 3.58 (s, 3H), 1.33 (t, 3H), 1.09 (d, 3H);

MS: 578.6 [M+H]+ found.

### EXAMPLES 477 AND 478

The following compounds were prepared from starting materials and procedures analogous to those described in Scheme 1 above, particularly in Examples 7 and 8.

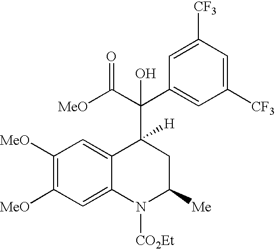

[(R, S, R), (R, S, S)]-4-[(3,5-bis-trifluoromethyl-phenyl)-hydroxyl-methoxycarbonyl-methyl]-6,7-dimethoxy-2-methyl-3,4-dihydro-2H-quinoline-1-carboxylic acid ethyl ester

First Eluting: ¹H NMR (CDCl₃): δ 8.18 (s, 2H), 7.88 (s, 1H), 7.03 (br s, 1H), 6.73 (s, 1H), 4.31 (m, 1H), 4.20 (m, 2H), 3.88 (s, 3H), 3.86 (s, 3H), 3.81 (s, 3H), 3.30 (d, 1H), 1.70 (m, 2H), 1.21-1.30 (t, m, 4H), 1.09 (d, 3H);

MS: 580.6 [M+H]+ found.

Second Eluting: ¹H NMR (CDCl₃): δ 8.25 (s, 2H), 7.84 (s, 1H), 6.92 (br s, 1H), 6.30 (s, 1H), 4.55 (m, 1H), 4.30 (m, 1H), 4.20 (m, 1H), 3.88 (s, 3H), 3.80 (s, 3H) 3.60 (d, 1H), 3.40 (s, 3H), 2.03 (m, 2H), 1.55 (m, 1H), 1.32 (t, 3H) 1.09 (d, 3H);

MS: 580.5 [M+H]+ found.

### EXAMPLES 479 AND 480

The following compounds were prepared from starting materials and procedures analogous to those described above, particularly in Examples 448, 449, 450 and 451.

[(R, R, R)], (R, R, S)]-4-[(3,5-bis-trifluoromethyl-phenyl)-methoxycarbonyl-methyl]-6,7-dimethoxy-2-methyl-3,4-dihydro-2H-quinoline-1-carboxylic acid ethyl ester

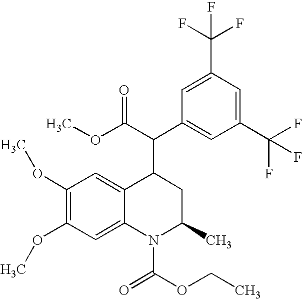

¹H NMR (CDCl₃): δ 7.97 (s, 2H), 7.83 (s, 1H), 7.0 (brs, 1H), 6.20 (s, 1H), 4.50 (m, 1H), 4.30 (m, 1H), 4.30 (m, 1H), 4.19 (m, 1H), 4.17 (d, 1H), 3.80 (s, 3H), 3.74 (s, 3H), 3.51 (s, 3H), 3.32 (m, 1H), 2.30 (m, 1H), 1.31 (t, 3H), 1.20 (d, 3H);

MS: 564.5 [M+H]+ found.

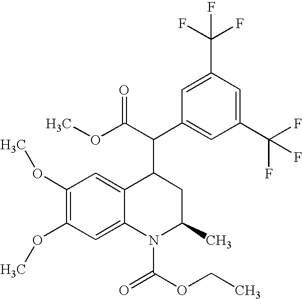

¹H NMR (CDCl₃): δ 7.87 (s, 3H), 7.03 (br s, 1H), 6.72 (s, 1H), 4.31 (m, 2H), 4.20 (m, 1H), 3.98 (d, 1H), 3.86 (s, 6H), 3.75 (s, 3H), 3.32 (t, 1H), 1.70 (m, 1H), 1.30 (t, 3H), 1.07 (d, 3H), 0.80 (m, 1H);

MS: 564.4 [M+H]+ found.

### EXAMPLE 481

The following compound was prepared from starting materials and procedures analogous to those described above, particularly in Examples 471, 472, 473 and 474.

[(R, R, R)], (R, R, S)]-4-[(3,5-bis-trifluoromethyl-phenyl)-1-hydroxyl-ethyl]-ethyl-6-trifluoromethyl-3,4-dihydro-2H-quinoline-1-carboxylic acid ethyl ester (0.079 grams, 0.142 mmol) was placed in a small round bottomed flask containing a magnetic stir bar and dissolved in 1.5 mL of anhydrous chloroform. To this reaction solution, 2,6-di-t-butyl-4-methylpyridine (0.117 grams, 0.568 mmol) and thionyl chloride (0.051 grams, 0.425 mmol) was added and allowed to stir at room temperature for four hours. The reaction mixture was quenched with water and extracted into methylene chloride. The organic layers was washed with 0.1 N HCl, dried over magnesium sulfate, filtered and concentrated to provide [(R, R, R)], (R, R, S)]-4-[1-(3,5-bis-trifluoromethyl-phenyl)-1-chloro-ethyl]-2-ethyl-6-trifluoromethyl-3,4-dihydro-2H-quinoline-1-carboxylic acid ethyl ester as an oil which was used without further purification. [(R, R, R)], (R, R, S)]4-[1-(3,5-bis-trifluoromethyl-phenyl)-1-chloro-ethyl]-2-ethyl-6-trifluoromethyl-3,4-dihydro-2H-quinoline-1-carboxylic acid ethyl ester (0.085 grams, 0.142 mmol) was placed in a small round bottomed flask containing a magnetic stir bar and dissolved in 0.48 mL THF. This solution was added glacial acetic acid (0.48 mmol), HCl (0.80 mmol), and zinc dust ((0.093 grams, 1.42 mmol). The reaction mixture was stirred at room temperature for three hours. The reaction mixture was quenched with water and extracted three times with ethyl acetate. The organic layer was washed with water, dried over magnesium sulfate, filtered and concentrated to provide [(R,R)-4-[1-(3,5-bis-trifluoromethyl-phenyl)-vinyl]-2-ethyl-6-trifluoromethyl-3,4-dihydro-2H-quinoline-1-carboxylic acid ethyl ester as an oil.

[(R,R)-4-[1-(3,5-bis-trifluoromethyl-phenyl)-vinyl]-2-ethyl-6-trifluoromethyl-3,4-dihydro-2H-quinoline-1-carboxylic acid ethyl ester (15 mg) was placed in a small round bottomed flask and dissolved in 10 mL of methanol. To this solution was added 10 mg of 10% Pd/C. The reaction mixture was hydrogenated at 45 psi for 12 hours. The reaction mixture was then filtered through Celite@ and washed with methanol. The filtrate was concentrated to an oil and purified by silica gel chromatography to provide [(R, R, R)], (R, R, S)]-4-[1-(3,5-bis-trifluoromethyl-phenyl)-ethyl]-6,7-trifluoromethyl-3,4-dihydro-2H-quinoline-1-carboxylic acid ethyl ester in 66% yield.

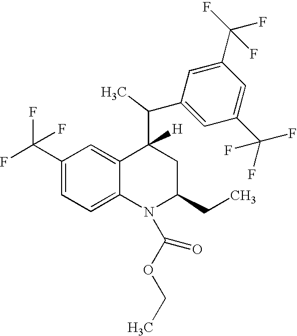

[(R, S, R)], (R, S, S)]-4-[1-(3,5-bis-trifluoromethyl-phenyl)-ethyl]-2-ethyl-6,7-trifluoromethyl-3,4-dihydro-2H-quinoline-1-carboxylic acid ethyl ester

¹H NMR (CDCl₃): δ 7.78 (s, 1H), 7.65 (s, 2H), 7.59 (dd, 2H), 7.41 (s, 1H), 4.30 (m, 1H), 4.20 (m, 2H), 2.95 (m, 2H), 1.80 (m, 1H), 1.58 (m, 2H), 1.40 (m, 1H), 1.30 (t, 3H), 1.20 (d, 3H), 0.72 (t, 3H);

MS: 542.3 [M+H]+ found.

### EXAMPLE 482

### (Stereo-Isomers of Examples 462, 463, 464 and 465)

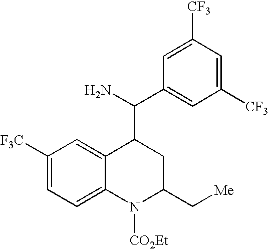

[(S, R, S)], 4-[Amino-(3,5-bis-trifluoromethyl-phenyl)-methyl]-2-ethyl-6-trifluoromethyl-3,4-dihydro-2H-quinoline-1-carboxylic acid ethyl ester;

[(S, S, S)], 4-[Amino-(3,5-bis-trifluoromethyl-phenyl)-methyl]-2-ethyl-6-trifluoromethyl-3,4-dihydro-2H-quinoline-1-carboxylic acid ethyl ester;

[(S, R, R)], 4-[Amino-(3,5-bis-trifluoromethyl-phenyl)-methyl]-2-ethyl-6-trifluoromethyl-3,4-dihydro-2H-quinoline-1-carboxylic acid ethyl ester;

[(S, S, R)], 4-[Amino-(3,5-bis-trifluoromethyl-phenyl)-methyl]-2-ethyl-6-trifluoromethyl-3,4-dihydro-2H-quinoline-1-carboxylic acid ethyl ester;

or a pharmaceutically acceptable salt of said compound.

The above stereo-isomers of examples 462, 463, 464 and 465 are prophetic and may be prepared in optically enriched form by resolution of the corresponding racemate indicated, or an intermediate in its synthesis, using methods analogous to those described herein.

### EXAMPLES 483 AND 484

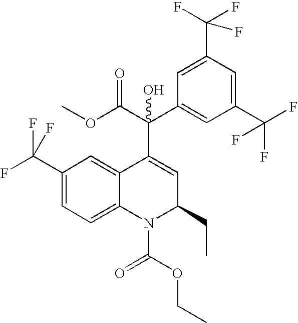

(R, R)-4-[(3,5-Bis-trifluoromethyl-phenyl)-hydroxyl-methoxycarbonyl-methyl]-2-ethyl-6-trifluoromethyl-2H-quinoline-1-carboxylic acid ethyl ester;

(R, S)-4-[(3,5-Bis-trifluoromethyl-phenyl)-hydroxyl-methoxycarbonyl-methyl]-2-ethyl-6-trifluoromethyl-2H-quinoline-1-carboxylic acid ethyl ester

The mixture of two diastereoisomers (Example 2, 1.0g) was chromatographed on silica (Flash 40M column, Biotage, Dyar Corp., Charlottesville, Va.) eluting with a methylene chloride-hexanes gradient from 2:3 to 4:1 to give the title compounds as white solids.

First Eluting Diastereoisomer (4a Stereochemistry not Determined):

MS: 597.9 [M−H]⁻ found

¹H-NMR (CDCl₃) δ 7.92 (s, 2H), 7.75 (s, 1H), 7.67 (s, 1H), 7.65 (d, J=8.30 Hz, 1H), 7.34 (dd, J=8.30, 1.66 Hz, 1H), 5.97 (d, J=6.64 Hz, 1H), 5.04 (m, 1H), 4.53 (s, 1H), 4.27 (m, 2H), 3.87 (s, 3H), 1.47 (m, 1H), 1.35 (m, 1H), 1.28 (t, J=7.47 Hz, 3H), 0.85 (d, J=6.64 Hz, 3H).

Second Eluting Diastereoisomer (4a Stereochemistry not Determined):

MS: 597.9 [M-H]-found

¹H-NMR (CDCl₃) δ 8.16 (s, 2H), 7.90 (s, 1H), 7.65 (d, J=8.30, 1H), 7.63 (s, 1H), 7.45 (dd, J=8.30, 1.66 Hz, 1H), 5.82 (d, J=6.42 Hz, 1H), 4.96 (m, 1H), 4.34 (s, 1H), 4.27 (m, 2H), 3.78 (s, 3H), 1.47 (m, 1H), 1.35 (m, 1H), 1.33 (t, J=7.47 Hz, 3H), 0.83 (d, J=7.47 Hz, 3H).

### EXAMPLE 485

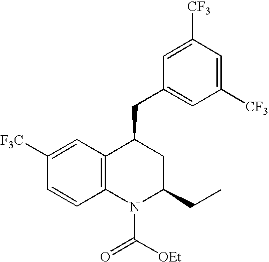

### (R, R)-4-(3,5-bis-trifluoromethyl-benzyl)-2-ethyl-6-trifluoromethyl-3, 4-dihydro-2H-quinoline-1-carboxylic Acid Ethyl Ester

[(R, R, R)]-4-[(3,5-bis-trifluoromethyl-phenyl)-methanesulfonyloxy-methyl]-2-ethyl-6-trifluoromethyl-3,4-dihydro-2H-quinoline-1-carboxylic acid ethyl ester (0.045 mmol, 0.0278 g) was placed in a 5 mL reaction flask. To this 1.3 mL of DMF followed by sodium borohydride (0.526 mmol, 0.019 g) was added. The reaction was heated to 85° C. for twelve hours. The reaction mixture was then diluted with ethyl actetate and washed with an aqueous brine solution. The organic layer was collected, dried over sodium sulfate, filtered and concentrated to dryness. The crude reaction mixture was purified on silica gel chromatography to provide 18.5 mg of the desired product (R, R)-4-(3,5-bis-trifluoromethyl-benzyl)-2-ethyl-6-trifluoromethyl-3, 4-dihydro-2H-quinoline-1-carboxylic acid ethyl ester in 78% yield.

¹H NMR (CDCl₃): 0.80 ppm (t, 3H), 1.10 (m, 1H), 1.29 (t, 3H), 1.41-1.62 (m, 2H & H₂O), 2.16 (m, 1H), 2.76-2.87 (m, 2H), 3.64 (d, 1H), 4.15-4.32 (m, 3H), 7.47 (s, 1H), 7.51-7.57 (m, 2H), 7.70 (s, 2H), 7.80 (s, 1H).

MS (ES+) m/z=528 (M+1).

### EXAMPLE 486

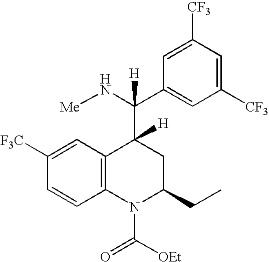

### (R, R, S) 4-[(3,5-bis-trifluoromethyl-phenyl)-methylaminomethyl]-2-ethyl-6-trifluoromethyl-3,4-dihydro-2H-quinoline-1-carboxylic Acid Ethyl Ester

(R, R, S) 4-[Amino-(3,5-bis-trifluoromethyl-phenyl)-methyl]-2-ethyl-6-trifluoromethyl-3,4-dihydro-2H-quinoline-1-carboxylic acid ethyl ester (0.193 g, 0.357 mmol) was placed in a sealed tube that contained 7 mL of ethyl formate. The tube was sealed and heated for twelve hours at 100° C. The reaction mixture was then concentrated and purified on silica gel chromatography to provide 0.165 g of the formamide in 81% yield. This product was then placed in a round bottomed flask equipped with a magnetic stir bar and dissolved in 7.3 mL of toluene. To this solution, 2.92 mL of borane methyl sulfide complex in toluene (2.0 M) was added. A reflux condenser was attached to the flask and the reaction was heated to 74° C. for twelve hours. The reaction mixture was then quenched with methanol and a few drops of HCl. This mixture was then heated for 1 hour. After cooling to room temperature, the mixture was quenched with aqueous NaHCO₃ and extracted 3 times with ethyl acetate. The organics were collected, dried over sodium sulfate and concentrated to dryness. The crude oil was purified on silica gel chromatography to provide (R, R, S) 4-[(3,5-bis-trifluoromethyl-phenyl)-methylaminomethyl]-2-ethyl-6-trifluoromethyl-3,4-dihydro-2H-quinoline-1-carboxylic acid ethyl ester in 41% yield.

¹H NMR (CDCl₃): 0.81 ppm (t, 3H), 1.33 (t, 3H), 1.36-1.52 (m, 3H), 2.25 (s, 3H), 2.52 (m, 1H), 2.97 (m, 1H), 3.99 (d, 1H), 4.28 (m, 2H), 4.52 (m, 1H), 7.04 (s, 1H), 7.41 (m, 1H), 7.59-7.61 (m, 3H), 7.72 (s, 1H).

MS (ES+) m/z=557 (M+1).

### EXAMPLES 487-500

These compounds were prepared from appropriate starting materials procedures analogous to those described in Examples 9, 10, 433, 434, 438, 443, 444, 452, 454, 457 and described generally in Scheme 2. The appropriately substituted bromo intermediates designated as Formula XVII in Scheme 2, were prepared using procedures described by Matsugi in Tetrahedron Lett 2000, 41, 8523 and Hardy in U.S. Pat. No. 6,288,075.

### EXAMPLE 487

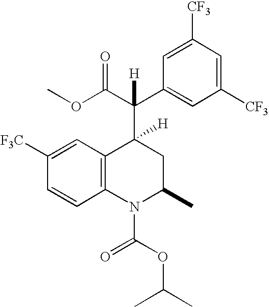

### (RS, RS, SR)-4-[(3,5-Bis-trifluoromethyl-phenyl)-methoxycarbonyl-methyl]-6-fluoro-2-methyl-3,4-dihydro-2H-quinoline-1-carboxylic Acid Isopropyl Ester

GCMS (EI): 535 (M+)

¹H-NMR (CDCl₃): δ 0.87 (m, 1H), 1.04 (d, 3H), 1.25 (m, 3H), 1.32 (d, 3H), 1.69 (m, 1H), 3.33 (m, 1H), 3.76 (s, 3H), 3.92 (d, 1H), 4.29 (m, 1H), 5.03 (m, 1H), 6.81 (dd, 1H), 6.87 (m, 1H), 7.38 (bs, 1H), 7.85 (s, 2H), 7.87 (s, 1H).

### EXAMPLE 488

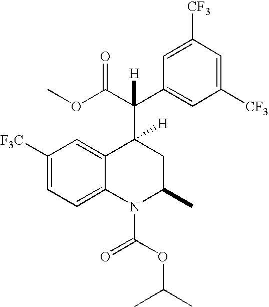

### (R, R, S)-4-[(3,5-Bis-trifluoromethyl-phenyl)-methoxycarbonyl-methyl]-6-fluoro-2-methyl-3,4-dihydro-2H-quinoline-1-carboxylic Acid Isopropyl Ester

GCMS (EI): 535 (M+)

¹H-NMR (CDCl₃): δ 0.83 (m, 1H), 1.04 (d, 3H), 1.26 (d, 3H), 1.32 (d, 3H), 1.72 (m, 1H), 3.33 (m, 1H), 3.77 (s, 3H), 3.91 (d, 1H), 4.28 (m, 1H), 5.03 (m, 1H), 6.82 (dd, 1H), 6.95 (m, 1H), 7.38 (bs, 1H), 7.84 (s, 2H), 7.87 (s, 1H).

### EXAMPLE 489

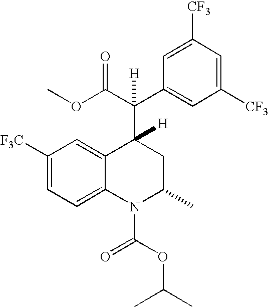

### (S, S, R)-4-[(3,5-Bis-trifluoromethyl-phenyl)-methoxycarbonyl-methyl]-6-fluoro-2-methyl-3,4-dihydro-2H-quinoline-1-carboxylic Acid Isopropyl Ester

GCMS (EI): 535 (M+)

¹H-NMR (CDCl₃): δ 0.83 (m, 1H), 1.04 (d, 3H), 1.26 (d, 3H), 1.32 (d, 3H), 1.69 (m, 1H), 3.33 (m, 1H), 3.77 (s, 3H), 3.91 (d, 1H), 4.28 (m, 1H), 5.03 (m, 1H), 6.82 (dd, 1H), 6.95 (m, 1H), 7.38 (bs, 1H), 7.84 (s, 2H), 7.87 (s, 1H).

### EXAMPLE 490

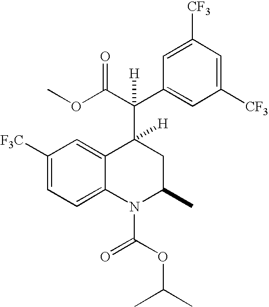

### (RS, RS, RS)-4-[(3,5-Bis-trifluoromethyl-phenyl)-methoxycarbonyl-methyl]-6-fluoro-2-methyl-3,4-dihydro-2H-quinoline-1-carboxylic Acid Isopropyl Ester

GCMS (EI): 535 (M+)

¹H-NMR (CDCl₃): δ 0.87 (m, 1H), 1.17 (d, 3H), 1.26 (m, 3H), 1.32 (d, 3H), 2.31 (m, 1H), 3.28 (m, 1H), 3.74 (s, 3H), 4.17 (d, 1H), 4.46 (m, 1H), 5.04 (m, 1H), 6.44 (dd, 1H), 6.87 (m, 1H), 7.38 (m, 1H), 7.84 (s, 1H), 7.89 (s, 2H).

### EXAMPLE 491

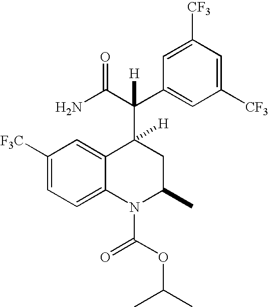

### (R, R, S)-4-[(3,5-Bis-trifluoromethyl-phenyl)-carbamoyl-methyl]-6-fluoro-2-methyl-3,4-dihydro-2H-quinoline-1-carboxylic Acid Isopropyl Ester

GCMS (EI): 520 (M+)

¹H-NMR (CDCl₃): δ 0.85 (m, 1H), 1.05 (d, 3H), 1.25 (d, 3H), 1.30 (d, 3H), 1.64 (m, 1H), 3.38 (m, 1H), 3.71 (d, 1H), 4.26 (m, 1H), 5.01 (m, 1H), 5.53 (bs, 1H), 5.78 (bs, 1H), 6.93 (m, 2H), 7.37 (m, 1H), 7.85 (s, 2H), 7.87 (s, 1H).

### EXAMPLE 492

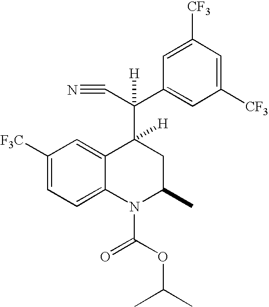

### (RS, RS, RS)-4-[(3,5-Bis-trifluoromethyl-phenyl)-cyano-methyl]-6-fluoro-2-methyl-3,4-dihydro-2H-quinoline-1-carboxylic Acid Isopropyl Ester

LCMS (ESI+): 503 (MH+)

¹H-NMR (CDCl₃): δ 1.19 (d, 3H), 1.24 (d, 3H), 1.28 (d, 3H), 1.45 (m, 1H), 2.18 (m, 1H), 2.87 (m, 1H), 4.31 (m, 1H), 4.77 (d, 1H), 4.99 (m, 1H), 7.04 (bs, 1H), 7.06 (bs, 1H), 7.45 (m, 1H), 7.90 (s, 2H), 7.96 (s, 1H).

### EXAMPLE 493

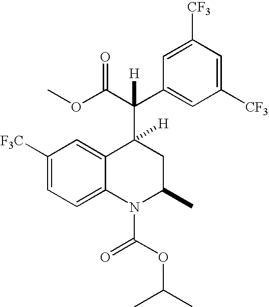

### (RS, RS, SR)-4-[(3,5-Bis-trifluoromethyl-phenyl)-methoxycarbonyl-methyl]-2-methyl-6-trifluoromethyl-3,4-dihydro-2H-quinoline-1-carboxylic Acid Isopropyl Ester

GCMS (EI): 585 (M+)

¹H-NMR (CDCl₃): δ 0.91 (m, 1H), 1.09 (d, 3H), 1.29 (d, 3H), 1.34 (d, 3H), 1.75 (m, 1H), 3.38 (t, 1H), 3.77 (s, 3H), 4.02 (d, 1H), 4.31 (m, 1H), 5.06 (m, 1H), 7.38 (s, 1H), 7.55 (m, 2H), 7.88 (bs, 3H).

### EXAMPLE 494

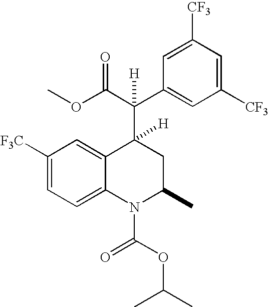

### (RS, RS, RS)-4-[(3,5-Bis-trifluoromethyl-phenyl)-methoxycarbonyl-methyl]-2-methyl-6-trifluoromethyl-3,4-dihydro-2H-quinoline-1-carboxylic Acid Isopropyl Ester

LCMS (ESI+): 585 (M+)

¹H-NMR (CDCl₃): δ 1.22 (d, 3H), 1.28 (d, 3H), 1.33 (d, 3H), 1.39 (m, 1H), 2.36 (m, 1H), 3.27 (m, 1H), 3.76 (s, 3H), 4.27 (d, 1H), 4.46 (m, 1H), 5.06 (m, 1H), 7.08 (s, 1H), 7.43 (d, 1H), 7.57 (d, 1H), 7.85 (s, 1H), 7.93 (s, 2H).

### EXAMPLE 495

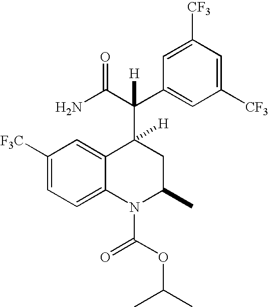

### (RS, RS, SR)-4-[(3,5-Bis-trifluoromethyl-phenyl)-carbamoyl-methyl]-2-methyl-6-trifluoromethyl-3,4-dihydro-2H-quinoline-1-carboxylic Acid Isopropyl Ester

LCMS (ESI+): 571 (MH+)

¹H-NMR (CDCl₃): δ 0.91 (m, 1H), 1.09 (d, 3H), 1.27 (d, 3H), 1.33 (d, 3H), 1.68 (m, 1H), 3.46 (t, 1H), 3.81 (d, 1H), 4.28 (m, 1H), 5.04 (m, 1H), 5.54 (bs, 1H), 5.82 (bs, 1H), 7.46 (s, 1H), 7.50 (d, 1H), 7.56 (d, 1H), 7.88 (s, 3H).

### EXAMPLE 496

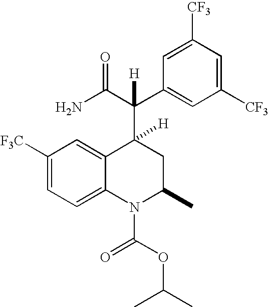

### (R, R, S)-4-[(3,5-Bis-trifluoromethyl-phenyl)-carbamoyl-methyl]-2-methyl-6-trifluoromethyl-3,4-dihydro-2H-quinoline-1-carboxylic Acid Isopropyl Ester

LCMS (ESI+): 571 (MH+)

¹H-NMR (CDCl₃): δ 0.87 (m, 1H), 1.09 (d, 3H), 1.26 (d, 3H), 1.33 (d, 3H), 1.69 (m, 1H), 3.45 (t, 1H), 3.83 (d, 1H), 4.27 (m, 1H), 5.03 (m, 1H), 5.65 (bs, 1H), 5.99 (bs, 1H), 7.46 (s, 1H), 7.49 (d, 1H), 7.55 (d, 1H), 7.88 (s, 3H).

### EXAMPLE 497

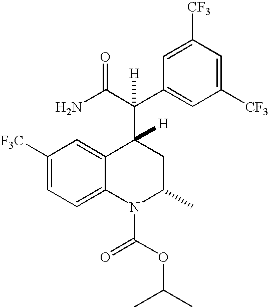

### (S, S, R)-4-[(3,5-Bis-trifluoromethyl-phenyl)-carbamoyl-methyl]-2-methyl-6-trifluoromethyl-3,4-dihydro-2H-quinoline-1-carboxylic Acid Isopropyl Ester

LCMS (ESI+): 571 (MH+)

¹H-NMR (CDCl₃): δ 0.87 (m, 1H), 1.08 (d, 3H), 1.26 (d, 3H), 1.32 (d, 3H), 1.68 (m, 1H), 3.44 (t, 1H), 3.84 (d, 1H), 4.26 (m, 1H), 5.02 (m, 1H), 5.69 (bs, 1H), 6.01 (bs, 1H), 7.47 (s, 1H), 7.49 (d, 1H), 7.54 (d, 1H), 7.87 (s, 3H).

### EXAMPLE 498

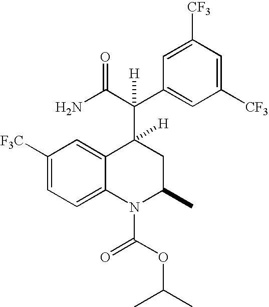

### (RS, RS, RS)-4-[(3,5-Bis-trifluoromethyl-phenyl)-carbamoyl-methyl]-2-methyl-6-trifluoromethyl-3,4-dihydro-2H-quinoline-1-carboxylic Acid Isopropyl Ester

LCMS (ESI+): 571 (MH+)

¹H-NMR (CDCl₃): δ 0.87 (m, 1H), 1.18 (d, 3H), 1.24 (d, 3H), 1.28 (d, 3H), 2.54 (m, 1H), 3.37 (t, 1H), 4.00 (d, 1H), 4.48 (m, 1H), 5.04 (m, 1H), 6.02 (bs, 1H), 6.33 (bs, 1H), 7.00 (s, 1H), 7.36 (d, 1H), 7.55 (d, 1H), 7.81 (s, 1H), 7.98 (s, 2H).

### EXAMPLE 499

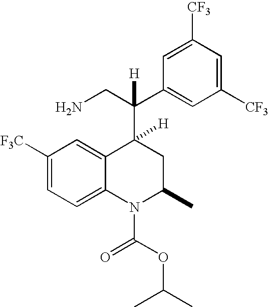

### (RS, RS, SR)-4-[2-Amino-1-(3,5-bis-trifluoromethyl-phenyl)-ethyl]-2-methyl-6-trifluoromethyl-3,4-dihydro-2H-quinoline-1-carboxylic Acid Isopropyl Ester

LCMS (ESI+): 557 (MH+)

¹H-NMR (CDCl₃): δ 0.80 (m, 1H), 1.04 (d, 3H), 1.28 (d, 3H), 1.34 (d, 3H), 1.76 (m, 1H), 2.75 (t, 1H), 3.11 (m, 1H), 3.35 (m, 1H), 3.46 (m, 1H), 4.26 (m, 1H), 5.06 (m, 1H), 7.54 (m, 3H), 7.77 (s, 2H), 7.84 (s, 1H).

### EXAMPLE 500

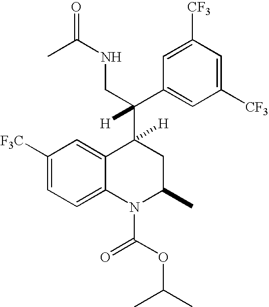

### (RS, RS, SR)-4-[2-Acetylamino-1-(3,5-bis-trifluoromethyl-phenyl)-ethyl]-2-methyl-6-trifluoromethyl-3,4-dihydro-2H-quinoline-1-carboxylic Acid Isopropyl Ester

LCMS (ESI+): 599 (MH+)

¹H-NMR (CDCl₃): δ 0.78 (m, 1H), 1.04 (d, 3H), 1.28 (d, 3H), 1.34 (d, 3H), 1.76 (m, 1H), 1.93 (s, 3H), 2.68 (s, 1H), 3.01 (m, 1H), 3.72 (m, 1H), 4.26 (m, 1H), 4.37 (m, 1H), 5.06 (m, 1H), 5.40 (m, 1H), 7.53 (s, 2H), 7.70 (s, 2H), 7.85 (s, 1H), 8.41 (s, 1H).

### EXAMPLES 501 AND 502

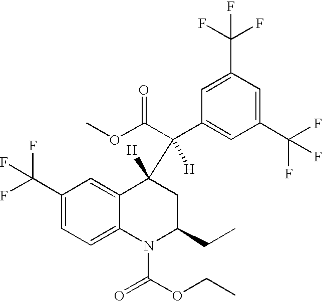

### (R, S, R)-4-[(3,5-Bis-trifluoromethyl-phenyl)-methoxycarbonyl-methyl]-2-ethyl-6-trifluoromethyl-3,4-dihydro-2H-quinoline-1-carboxylic Acid Ethyl Ester

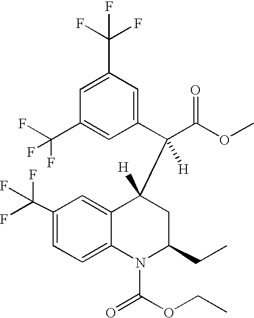

### (R, S, S)-4-[(3,5-Bis-trifluoromethyl-phenyl)-methoxycarbonyl-methyl]-2-ethyl-6-trifluoromethyl-3,4-dihydro-2H-quinoline-1-carboxylic Acid Ethyl Ester

To a solution of 3,5-bis(trifluoromethylphenyl)acetic acid methyl ester (1.41 g, 4.93 mmol) in anhydrous N,N-dimethylformamide (3 mL) was added sodium hydride (60% mineral oil dispersion, 6.55 mmol, 262 mg) and the mixture was stirred at room temperature for 60 min. A solution of (R)-4-chloro-2-ethyl-6-trifluoromethyl-3,4-dihydro-2H-quinoline-1-carboxylic acid ethyl ester (1.10 g, 3.28 mmol, mixture of isomers, Preparation 14) in anhydrous N,N-dimethylformamide (1.5 mL) was added and the mixture was stirred at room temperature for 72 hr. Water (20 mL) was added and the mixture was extracted with diethyl ether (3×50 mL) and the organic extract was dried over anhydrous sodium sulfate and the solvent was removed under vacuum to give the crude product as a yellow oil (˜2 g). Purification was achieved using radial chromatography (Chromatron model 7924T, Harrison Research, Palo Alto, Calif.) with a 4 mm silica gel rotor eluting with hexanes/ethyl acetate 9:1 to give the title compounds:

(R, S, R)-4-[(3,5-Bis-trifluoromethyl-phenyl)-methoxycarbonyl-methyl]-2-ethyl-6-trifluoromethyl-3,4-dihydro-2H-quinoline-1-carboxylic acid ethyl ester (first eluting diastereoisomer)

MS: 586.0 [M+H]⁺ found

¹H-NMR (CDCl₃) δ 7.90 (s, 2H), 7.87 (s, 1H), 7.59 (d, J=8.30 Hz, 1H), 7.62 (d, J=8.30 Hz, 1H), 7.61 (s, 1H), 4.39-4.27 (m, 2H), 4.27-4.18 (m, 1H), 3.78 (d, J=11.61 Hz, 1H), 3.59 (m, 1H), 3.48 (s, 3H), 1.76 (ddd, J=14.10, 8.30, 3.30 Hz, 1H), 1.61-1.55 (m, 1H), 1.57-1.50 (m, 1H), 1.48-1.40 (m, 1H), 1.35 (t, J=7.47 Hz, 3H), 0.73 (t, J=7.47 Hz, 3H).

(R, S, S)-4-[(3,5-Bis-trifluoromethyl-phenyl)-methoxycarbonyl-methyl]-2-ethyl-6-trifluoromethyl-3,4-dihydro-2H-quinoline-1-carboxylic acid ethyl ester (536 mg, second eluting diastereoisomer)

MS: 586.0 [M+H]⁺ found

¹H-NMR (CDCl₃) δ 7.66 (s, 1H), 7.42 (d, J=8.30 Hz, 1H), 7.40 (s, 2H), 7.33 (dd, J=8.30, 1.66 Hz, 1H), 6.47 (d, J=1.66 Hz, 1H), 4.55-4.47 (m, 1H), 4.34 (m, 1H), 4.32 (m, 1H), 3.83 (d, J=11.61 Hz, 1H), 3.80 (s, 3H), 3.43 (ddd, J=11.61, 4.98, 2.49 Hz, 1H), 2.44 (ddd, J=14.11, 8.30, 2.49 Hz, 1H), 1.81 (ddd, J=14.10, 8.30, 4.98 Hz, 1H), 1.67 (m, 1H), 1.51 (m, 1H), 1.33 (t, J=7.47 Hz, 3H), 0.85 (t, J=7.47 Hz, 3H).

### EXAMPLES 503 AND 504

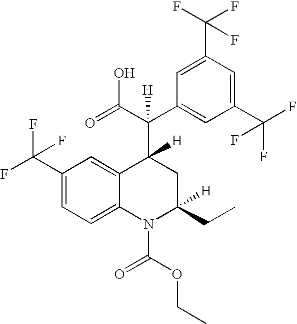

### (R, S, R)-4-[(3,5-Bis-trifluoromethyl-phenyl)-carboxy-methyl]-2-ethyl-6-trifluoromethyl-3,4-dihydro-2H-quinoline-1-carboxylic Acid Ethyl Ester

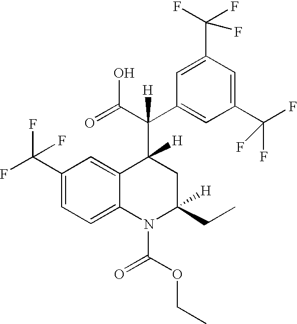

### (R, S, S)-4-[(3,5-Bis-trifluoromethyl-phenyl)-carboxy-methyl]-2-ethyl-6-trifluoromethyl-3,4-dihydro-2H-quinoline-1-carboxylic Acid Ethyl Ester

A mixture of (R, S, S)-4-[(3,5-bis-trifluoromethyl-phenyl)-methoxycarbonyl-5 methyl]-2-ethyl-6-trifluoromethyl-3,4-dihydro-2H-quinoline-1-carboxylic acid ethyl ester (Example 502, 100 mg, 0.171 mmol), aqueous sodium hydroxide (2N, 0.512 mL, 1.024 mmol) and anhydrous tetrahydrofuran (2 mL) was stirred at room temperature for 2 days before adding 2N hydrochloric acid (1.5 mL). The mixture was diluted with acetonitrile and evaporated to dryness under vacuum to give the crude product as an oil (128 mg). Purification was achieved using radial chromatography (Chromatron model 7924T, Harrison Research, Palo Alto, Calif.) with a 4 mm silica gel rotor eluting with hexanes/ethyl acetate 55:45 to give the title compounds:

First eluting: (R, S, R)-4-[(3,5-Bis-trifluoromethyl-phenyl)-carboxy-methyl]-2-ethyl-6-trifluoromethyl-3,4-dihydro-2H-quinoline-1-carboxylic acid ethyl ester (14 mg);

¹H-NMR (CDCl₃) δ 7.88 (s, 1H), 7.88 (s, 2H), 7.55 (m, 1H), 7.51 (m, 1H), 7.49 (m, 1H), 4.32 (m, 1H), 4.27 (m, 1H), 4.21 (m, 1H), 3.80 (d, J=10.79 Hz, 1H), 3.57 (m, 1H), 1.78 (m, 1H), 1.55 (m, 2H), 1.43 (m, 1H), 1.32 (t, J=7.47 Hz, 3H), 0.71 (t, J=7.47 Hz, 3H).

Second eluting: (R, S, S)-4-[(3,5-Bis-trifluoromethyl-phenyl)-carboxy-methyl]-2-ethyl-6-trifluoromethyl-3,4-dihydro-2H-quinoline-1-carboxylic acid ethyl ester (32 mg)

¹H-NMR (CDCl₃) δ 7.67 (s, 1H), 7.45 (s, 2H), 7.41 (m, 1H), 7.34 (m, 1H), 6.51 (brs, 1H), 4.55 (m, 1H), 4.32 (m, 2H), 3.85 (d, J=10.79 Hz, 1H), 3.43 (m, 1H), 2.55 (m, 1H), 1.81 (m, 1H), 1.63 (m, 1H), 1.60 (m, 1H), 1.31 (t, J=7.47 Hz, 3H), 0.84 (t, J=7.47 Hz, 3H).

### EXAMPLES 505 AND 506

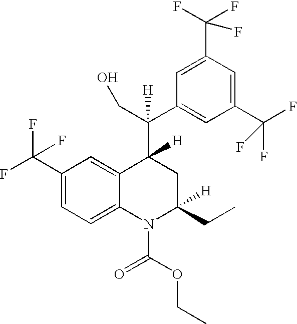

### (R, S, R)-4-[1-(3,5-Bis-trifluoromethyl-phenyl)-2-hydroxy-ethyl]-2-ethyl-6-trifluoromethyl-3,4-dihydro-2H-quinoline-1-carboxylic Acid Ethyl Ester

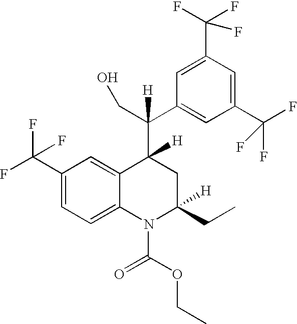

### (R, S, S)-4-[1-(3,5-Bis-trifluoromethyl-phenyl)-2-hydroxy-ethyl]-2-ethyl-6-trifluoromethyl-3,4-dihydro-2H-quinoline-1-carboxylic Acid Ethyl Ester

To a solution of (R, S, R)- and (R, S, S)-4-[(3,5-bis-trifluoromethyl-phenyl)-carboxy-methyl]-2-ethyl-6-trifluoromethyl-3,4-dihydro-2H-quinoline-1-carboxylic acid ethyl ester (Examples 503 and 504 as prepared above but isomers not separated, 143 mg, 0.25 mmol) in tetrahydrofuran (3 mL) under nitrogen was added borane-dimethylsulfide complex (2M in tetrahydrofuran, 0.25 mL, 0.5 mmol). After 24 hr the mixture was diluted with methanol (1 mL) and evaporated to dryness under vacuum. To the residue was added 2N hydrochloric acid (3 mL), the mixture was stirred for 10 min and then extracted with diethyl ether (3×15 mL). The organic layer was dried over anhydrous sodium sulfate and the solvent was evaporated under vacuum to give the crude product as an oil (175 mg). Purification was achieved using radial chromatography (Chromatron model 7924T, Harrison Research, Palo Alto, Calif.) with a 1 mm silica gel rotor eluting with a hexanes/ethyl acetate gradient from 4:1 to 7:3 to give the title compounds:

First eluting: (R, S, R)-4-[1-(3,5-Bis-trifluoromethyl-phenyl)-2-hydroxy-ethyl]-2-ethyl-6-trifluoromethyl-3,4-dihydro-2H-quinoline-1-carboxylic acid ethyl ester (9 mg)

MS: 558.3 [M+H]⁺ found.

¹H-NMR (CDCl₃) δ 7.83 (s, 1H), 7.79 (s, 2H), 7.60 (m, 1H), 7.53 (brs, 1H), 7.52 (m, 1H), 4.35 (m, 1H), 4.24 (m, 1H), 4.16 (m, 1H), 3.71 (m, 2H), 3.34 (m, 1H), 2.99 (m, 1H), 1.78 (m, 1H), 1.57 (m, 2H), 1.57 (m, 1H), 1.45 (m, 1H), 1.33 (t, J=7.47 Hz, 3H), 0.71 (t, J=7.47 Hz, 3H).

Second eluting: (R, S, S)-4-[1-(3,5-Bis-trifluoromethyl-phenyl)-2-hydroxy-ethyl]-2-ethyl-6-trifluoromethyl-3,4-dihydro-2H-quinoline-1-carboxylic acid ethyl ester (30 mg)

MS: 558.5 [M+H]⁺ found.

¹H-NMR (CDCl₃) δ 7.63 (s, 1H), 7.40 (m, 1H), 7.39 (s, 2H), 7.32 (m, 1H), 6.62 (brs, 1H) 4.48 (m, 1H), 4.25 (m, 2H), 4.15 (m, 2H), 3.13 (m, 2H), 2.60 (m, 1H), 1.69 (m, 3H), 1.50 (m, 1H), 1.29 (t, J=7.47 Hz, 3H), 0.86 (t, J=7.47 Hz, 3H).

### EXAMPLE 507

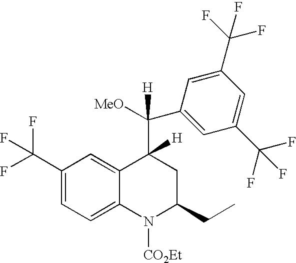

### (R, R, S)-4-[(3,5-Bis-trifluoromethyl-phenyl)-methoxy-methyl]-2-ethyl-6-trifluoromethyl-3,4-dihydro-2H-quinoline-1-carboxylic Acid Ethyl Ester

[(R, R, S)]-4-[(3,5-Bis-trifluoromethyl-phenyl)-hydroxy-methyl]-2-ethyl-6-trifluoromethyl-3,4-dihydro-2H-quinoline-1-carboxylic acid ethyl ester (0.030 grams, 0.055 mmol, 1 eq) was placed in a round bottomed flask equipped with a magnetic stir bar. DMSO (1.0 mL) was added followed by the addition of (0.012 grams 0.221 mmol, 4 eq) potassium hydroxide and (0.016 grams, 0.110 mmol, 2 eq) methyl iodide at room temperature. After 1.5 hour, the reaction mixture was quenched with 1N HCl, extracted 3 times with ethyl acetate and dried over sodium sulfate. The crude material was purified on silica gel chromatography to provide the title compound.

LCMS (ESI+): 551 (MH+).

¹H NMR (CDCl₃): δ?0.95 (t, 3H), 1.31 (t, 3H), 1.41 (m, 1H), 1.60 (m, 1H), 2.6 (m, 1H), 2.90 (m, 1H), 3.20 (s, 3H), 4.33 (m, 2H), 4.45 (m, 1H), 6.60 (s, 1H), 7.2 (s, 2H), 7.30 (d, 1H), 7.5 (d, 1H) 7.70 (s, 1H).

Throughout this application, various publications are referenced. The disclosures of these publications in their entireties are hereby incorporated by reference into this application for all purposes.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A compound according to Formula I 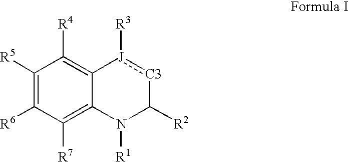
Wherein
C3 is carbon;
J is nitrogen or carbon, wherein if J is carbon, then the bond between C3 and J is a single or double bond and if J is nitrogen, then the bond between C3 and J is a single bond;
R¹ is Y, W—X or W—Y¹; wherein W is a carbonyl, thiocarbonyl, sulfinyl or sulfonyl; X is —O—Y, —S—Y, —N(H)—Y or —N—(Y)₂; Y for each occurrence is independently Z or a fully saturated, partially unsaturated or fully unsaturated one to ten membered straight or branched carbon chain wherein each carbon, other than the connecting carbon, may optionally be replaced with one or two heteroatoms selected independently from oxygen, sulfur and nitrogen and said carbon is optionally mono-, di- or tri-substituted independently with halo, said carbon is optionally mono-substituted with hydroxy, said carbon is optionally mono-substituted with oxo, said sulfur is optionally mono- or di-substituted with oxo, said nitrogen is optionally mono-, or di-substituted with oxo, and said carbon chain is optionally mono-substituted with Z; and Y¹ for each occurrence is independently Z or a fully saturated, partially unsaturated or fully unsaturated one to ten membered straight or branched carbon chain wherein each carbon, other than the connecting carbon, may optionally be replaced with one or two heteroatoms selected independently from oxygen, sulfur and nitrogen and said carbon is optionally mono-, di- or tri-substituted independently with halo, said carbon is optionally mono-substituted with hydroxy, said carbon is optionally mono-substituted with oxo, said sulfur is optionally mono- or di-substituted with oxo, said nitrogen is optionally mono-, or di-substituted with oxo, and said carbon chain is optionally mono-substituted with Z; wherein Z is a partially saturated, fully saturated or fully unsaturated three to eight membered ring optionally having one to four heteroatoms selected independently from oxygen, sulfur and nitrogen, or a bicyclic ring consisting of two fused partially saturated, fully saturated or fully unsaturated three to six membered rings, taken independently, optionally having one to four heteroatoms selected independently from nitrogen, sulfur and oxygen; and said Z substituent is optionally mono-, di- or tri-substituted independently with halo, (C₂-C₆)alkenyl, (C₁-C₆) alkyl, hydroxy, (C₁-C₆)alkoxy, (C₁-C₄)alkylthio, amino, nitro, cyano, oxo, carboxy, (C₁-C₆)alkyloxycarbonyl, mono-N— or di-N,N—(C₁-C₆)alkylamino wherein said (C₁-C₆)alkyl substituent is optionally mono-, di- or tri-substituted independently with halo, hydroxy, (C₁-C₆)alkoxy, (C₁-C₄)alkylthio, amino, nitro, cyano, oxo, carboxy, (C₁-C₆)alkyloxycarbonyl, mono-N— or di-N,N—(C₁-C₆)alkylamino, said (C₁-C₆)alkyl or (C₁-C₆)alkoxy substituent is also optionally substituted with from one to nine fluorines;
R² is a partially saturated, fully saturated or fully unsaturated one to six membered straight or branched carbon chain wherein each carbon, other than the connecting carbon, may optionally be replaced with one or two heteroatoms selected independently from oxygen and sulfur, and said carbon is optionally mono-, di- or tri-substituted independently with halo, said carbon chain is optionally mono-substituted with oxo, said carbon is optionally mono-substituted with hydroxy, said sulfur is optionally mono- or di-substituted with oxo,; or said R² is a partially saturated, fully saturated or fully unsaturated three to seven membered ring optionally having one to two heteroatoms selected independently from oxygen and sulfur, wherein said R² ring is optionally attached through (C₁-C₄)alkyl; wherein said R² ring is optionally mono-, di- or tri-substituted independently with halo, (C₂-C₆)alkenyl, (C₁-C₆) alkyl, hydroxy, (C₁-C₆)alkoxy, (C₁-C₄)alkylthio, amino, nitro, cyano, oxo, carboxy, (C₁-C₆)alkyloxycarbonyl, mono-N— or di-N,N—(C₁-C₆)alkylamino wherein said (C₁-C₆)alkyl substituent is optionally mono-, di- or tri-substituted independently with halo, hydroxy, (C₁-C₆)alkoxy, (C₁-C₄)alkylthio, oxo or (C₁-C₆)alkyloxycarbonyl;
R³ is a fully saturated, partially unsaturated or fully unsaturated one to six membered straight or branched carbon chain having C4a, wherein C4a is a carbon atom that connects to J, wherein each carbon in the carbon chain may optionally be replaced with one heteroatom selected from oxygen, sulfur and nitrogen, and said carbon is optionally mono-, di- or tri-substituted with halo, said carbon is optionally mono-substituted with hydroxy, said carbon is optionally mono-substituted with oxo or nitrogen, said sulfur is optionally mono- or di-substituted with oxo, said nitrogen is optionally mono- or di-substituted with hydrogen or oxo, and said carbon chain is mono, di-, or tri-substituted with V at C4a or the R³ carbon adjacent to C4a; provided that in R³, when J is carbon, it is other than C4a that is optionally replaced with one heteroatom; and provided that in R³, when J is nitrogen, it is other than C4a that is optionally replaced with a heteroatom and it is other than C4a that is optionally mono-substituted with hydroxy or nitrogen; wherein V is a partially saturated, fully saturated or fully unsaturated three to eight membered ring optionally having one to four heteroatoms selected independently from oxygen, sulfur and nitrogen such that V is not imidazolyl or a fully saturated heterocyclic nitrogen-containing ring wherein nitrogen of the ring is connected to the R³ group; a bicyclic ring consisting of two fused partially saturated, fully saturated or fully unsaturated three to six membered rings, taken independently, optionally having one to four heteroatoms selected independently from nitrogen, sulfur and oxygen; or a tricyclic ring consisting of three fused partially saturated, fully saturated or fully unsaturated three to six membered rings, taken independently, optionally having one to four heteroatoms selected independently from nitrogen, sulfur and oxygen; and said V substituent is optionally mono-, di-, tri-, tetra- or penta-substituted independently with V¹, (C₁-C₆)alkyl-V¹, C(O)—V¹, O—(C₀-C₆)alkyl-V¹, (C₁-C₆)alkyl-O—V¹, C(O)-mono-N— or di-N,N—(C₁-C₆)alkyl-V¹, halo, (C₁-C₆)alkyl, (C₂-C₆)alkenyl, hydroxy, (C₁-C₆)alkoxy, (C₁-C₄)alkylthio, (C₁-C₄)alkylsulfinyl, (C₁-C₄)alkylsulfonyl, mono-N— or di-N,N—(C₁-C₆)alkylsulfonyl, amino, nitro, cyano, oxo, carboxamoyl, mono-N— or di-N,N—(C₁-C₆) alkylcarboxamoyl, carboxy, (C₁-C₆)alkyloxycarbonyl, mono-N— or di-N,N—(C₁-C₆)alkylamino, wherein said (C₁-C₆)alkyl or (C₂-C₆)alkenyl substituent is optionally mono-, di- or tri-substituted independently with hydroxy, (C₁-C₆)alkoxy, (C₁-C₄)alkylthio, amino, nitro, cyano, oxo, carboxy, (C₁-C₆)alkyloxycarbonyl, mono-N— or di-N,N—(C₁-C₆)alkylamino, wherein each (C₁-C₆)alkyl, (C₁-C₆)alkoxy, (C₁-C₄)alkylthio, (C₁-C₄)alkylsulfonyl or (C₂-C₆)alkenyl substituents are also optionally substituted with from one to nine fluorines; wherein V¹ is a partially saturated, fully saturated or fully unsaturated three to six membered ring optionally having one to two heteroatoms selected independently from oxygen, sulfur and nitrogen, or a bicyclic ring consisting of two fused partially saturated, fully saturated or fully unsaturated three to six membered rings, taken independently, optionally having one to four heteroatoms selected independently from nitrogen, sulfur and oxygen; and said V¹ substituent is optionally mono-, di-, tri-, tetra- or penta-substituted independently with halo, (C₁-C₆)alkyl, (C₁-C₆)alkoxy, hydroxy, oxo, amino, nitro, cyano, (C₁-C₆)alkyloxycarbonyl, mono-N— or di-N,N—(C₁-C₆)alkylamino wherein said (C₁-C₆)alkyl substituent is optionally mono-substituted with oxo, said (C₁-C₆)alkyl or (C₁-C₆)alkoxy substituent is also optionally substituted with from one to nine fluorines; and
each of R⁴, R⁵, R⁶ and R⁷ are independently hydrogen, a bond, nitro or halo wherein said bond is substituted with T or a partially saturated, fully saturated or fully unsaturated (C₁-C₁₂) straight or branched carbon chain wherein each carbon may optionally be replaced with one or two heteroatoms per carbon chain wherein the heteroatoms are selected independently from oxygen, sulfur and nitrogen, wherein said carbon is optionally mono-, di- or tri-substituted independently with halo, said carbon is optionally mono-substituted with hydroxy, said carbon is optionally mono-substituted with oxo or nitrogen, said sulfur is optionally mono- or di-substituted with oxo, said nitrogen is optionally mono- or di-substituted with hydrogen or oxo, and said carbon chain is optionally mono-substituted with T; wherein T is a partially saturated, fully saturated or fully unsaturated three to twelve membered ring optionally having one to four heteroatoms selected independently from oxygen, sulfur and nitrogen, or a bicyclic ring consisting of two fused partially saturated, fully saturated or fully unsaturated three to six membered rings, taken independently, optionally having one to four heteroatoms selected independently from nitrogen, sulfur and oxygen; and said T substituent is optionally mono-, di- or tri-substituted independently with halo, (C₁-C₆)alkyl, (C₂-C₆)alkenyl, hydroxy, (C₁-C₆)alkoxy, (C₁-C₄)alkylthio, amino, nitro, cyano, oxo, carboxy, (C₁-C₆)alkyloxycarbonyl, mono-N— or di-N,N—(C₁-C₆)alkylamino wherein said (C₁-C₆)alkyl substituent is optionally mono-, di- or tri-substituted independently with hydroxy, (C₁-C₆)alkoxy, (C₁-C₄)alkylthio, amino, nitro, cyano, oxo, carboxy, (C₁-C₆)alkyloxycarbonyl, mono-N— or di-N,N—(C₁-C₆)alkylamino, said (C₁-C₆)alkyl or (C₁-C₆)alkoxy substituent also optionally has from one to nine fluorines;
R⁴ and R ⁵, R⁵ and R⁶, and/or R⁵ and R⁷ may optionally be taken together and can form at least one ring that is a partially saturated or fully unsaturated four to eight membered ring optionally having one to three heteroatoms independently selected from nitrogen, sulfur and oxygen; wherein each ring formed by R⁴ and R⁵, or R⁵ and R⁶, and/or R⁵ and R⁷ is optionally mono-, di- or tri-substituted independently with halo, (C₁-C₆)alkyl, (C₁-C₄)alkylsulfonyl, (C₂-C₆)alkenyl, hydroxy, (C₁-C₆)alkoxy, (C₁-C₄)alkylthio, amino, nitro, cyano, oxo, carboxy, (C₁-C₆)alkyloxycarbonyl, mono-N— or di-N,N—(C₁-C₆)alkylamino wherein said (C₁-C₆)alkyl substituent is optionally mono-, di- or tri-substituted independently with hydroxy, (C₁-C₆)alkoxy, (C₁-C₄)alkylthio, amino, nitro, cyano, oxo, carboxy, (C₁-C₆)alkyloxycarbonyl, mono-N— or di-N,N—(C₁-C₆)alkylamino, said (C₁-C₆)alkyl substituent also optionally has from one to nine fluorines;
or a pharmaceutically acceptable salt or prodrug thereof;
with the following provisos:
a) when there is a single bond between C3 and J, and R³ is a fully saturated one to six membered straight or branched carbon chain substituted on C4a with V then R¹ is other than C(O)—(C₁-C₄)alkyl optionally mono-, di- or tri-substituted with halo and R¹ is other than C(O)-monocyclicaromatic ring; or
b) when there is a single bond between C3 and J, and R is —C(O)—O—V, and R² is phenyl then R¹ is other than (C₁-C₄)alkyl; and
c) when there is a double bond between C3 and J, and R² is methyl then R³ is other than —CH₂—O—V, —CH₂—V or —CH₂—CH₂—V.

2. A compound of claim 1 wherein
J is carbon;
R¹ is W—X;
W is carbonyl;
X is —O—Y;
Y for each occurrence is independently (C₁-C₆)alkyl, said (C₁-C₆)alkyl optionally having one to nine fluorines or said (C₁-C₆)alkyl optionally mono-substituted with Z;
wherein Z is a partially saturated, fully saturated or fully unsaturated three to six membered ring optionally having one to two heteroatoms selected independently from oxygen, sulfur and nitrogen;
wherein said Z substituent is optionally mono-, di- or tri-substituted independently with halo, (C₁-C₄)alkyl, (C₁-C₄)alkoxy, (C₁-C₄)alkylthio, nitro, cyano, oxo, or (C₁-C₄)alkyloxycarbonyl, said (C₁-C₄)alkyl or (C₁-C₄)alkoxy is optionally substituted with from one to nine fluorines;
R² is beta and is a partially saturated, fully saturated or fully unsaturated (C₁-C₄) straight or branched carbon chain wherein one carbon, other than the connecting carbon, may optionally be replaced with oxygen or sulfur and wherein said carbon is optionally mono-, di- or tri-substituted independently with halo, said carbon chain is optionally mono-substituted with oxo or hydroxy, said sulfur is optionally mono- or di-substituted with oxo,; or said R² is a partially saturated, fully saturated or fully unsaturated three to five membered ring optionally having one heteroatom selected independently from oxygen and sulfur;
wherein said R² ring is optionally mono-, di- or tri-substituted independently with halo, hydroxy, (C₁-C₆)alkoxy, amino, nitro, (C₁-C₄)alkyloxycarbonyl or carboxy;
wherein R³ is a fully saturated, partially unsaturated or fully unsaturated one to six membered straight or branched carbon chain wherein each carbon, other than C4a, may optionally be replaced with one heteroatom selected from oxygen, sulfur and nitrogen, and said carbon is optionally mono-, di- or tri-substituted independently with halo, said carbon is optionally mono-substituted with hydroxy, said carbon is optionally mono-substituted with cyano, said carbon is optionally mono-substituted with oxo or nitrogen, said sulfur is optionally mono-or di-substituted with oxo, said nitrogen is optionally mono- or di-substituted with hydrogen or oxo, and said carbon chain is optionally mono, di-, or tri-substituted with V at C4a or at the R³ carbon adjacent to C4a; V is a three, four, five or six membered partially saturated, fully saturated or fully unsaturated ring optionally having one to three heteroatoms selected independently from oxygen, sulfur and nitrogen such that V is not imidazolyl or a fully saturated heterocyclic nitrogen-containing ring wherein nitrogen of the ring is connected to the R³ group;
wherein said V ring is optionally mono-, di-, tri-, tetra- or penta-substituted independently with halo, (C₁-C₆)alkyl, hydroxy, (C₁-C₆)alkoxy, (C₁-C₆)alkoxycarbonyl, nitro, cyano or oxo, wherein said (C₁-C₆)alkyl or (C₁-C₆)alkoxy substituent optionally has from one to nine fluorines;
R⁴ is hydrogen;
R⁵ and R⁶ are each independently hydrogen, halo, T, (C₁-C₆)alkoxy or (C₁-C₆)alkyl, said (C₁-C₆)alkoxy or (C₁-C₆)alkyl substituent optionally having from one to nine fluorines or said (C₁-C₆)alkoxy or (C₁-C₆)alkyl substituent optionally mono-substituted with T;
wherein T is a partially saturated, fully saturated or fully unsaturated five to six membered ring optionally having one to two heteroatoms selected independently from oxygen, sulfur and nitrogen;
wherein said T substituent is optionally mono-, di- or tri-substituted independently with halo, (C₁-C₆)alkyl, hydroxy, (C₁-C₆)alkoxy, (C₁-C₄)alkylthio, amino, oxo, carboxy, (C₁-C₆)alkyloxycarbonyl, mono-N— or di-N,N—(C₁-C₆)alkylamino wherein said (C₁-C₆)alkyl or (C₁-C₆)alkoxy substituent optionally has from one to nine fluorines;
R⁷ is hydrogen;
or a pharmaceutically acceptable salt thereof.

3. A compound of claim 2 wherein
Y is (C₁-C₄)alkyl, wherein said (C₁-C₄)alkyl substituent optionally has one to nine fluorines;
R² is (C₁-C₄)alkyl, cyclopropyl or cyclobutyl;
R³ is —((C₁-C₄)alkyl)(NH₂)(V), —((C₁-C₃)alkyl)(NH(C₁-C₂)alkyl))(V), —((C₁-C₄)alkyl)(OH)(V), —((C₁-C₄)alkyl)(F)(V), —((C₁-C₂)alkyl)(O—C(O)(C₁-C₂)alkyl)(V), —C(O)—V, —C(OH)(C(O)O(C₁-C₃)alkyl)(V), —CF₂(V), —((C₁-C₂)alkyl)(NHC(O)(C₁-C₂)alkyl)(V), —CH₂(V), —((C₁-C₂)alkyl)(C(O)O(C₁-C₂)alkyl)(V), —((C₁-C₄)alkyl)(C(O)NH₂)(V), —((C₁-C₄)alkyl)(CN)(V), or —((C₁-C₃)alkyl)((C₁-C₃)alkoxy)(V),
V is phenyl optionally mono-, di- or tri-substituted independently with halo, (C₁-C₆)alkyl, hydroxy, (C₁-C₆)alkoxy, nitro, cyano or oxo wherein said (C₁-C₆)alkyl or (C₁-C₆)alkoxy substituent optionally has from one to nine fluorines;
R⁵ and R⁶ are each independently hydrogen, halo, (C₁-C₃)alkoxy or (C₁-C₆)alkyl, said (C₁-C₃)alkoxy optionally having from one to seven halo, said (C₁-C₆)alkyl optionally having from one to nine halo;
or a pharmaceutically acceptable salt thereof.

4. A compound of claim 3 wherein
Y is methyl, ethyl, 1-propyl, 2-propyl or tert-butyl;
R² is methyl, ethyl, 2-propyl, cyclopropyl or cyclobutyl;
R³ is —C(O)—V, —C(OH)(C(O)OCH₃)(V), —CH(F)(V), —CF₂(V), —CH(OCH₃)(V), —CH(C(O)OCH₃)(V), —CH(CN)(V), —CH(OH)(V), —CH₂(V), —CH(NH₂)(V), —CH(NH(CH₃))(V), —CH(C(O)NH₂)(V), —CH(CH₂OH)V, —CH(CH₂OCH₃)V, —CH(CH₂OC(O)CH₃)V, —CH(CH₂F)V, or —CH(CH₂NH₂)V; and
V is phenyl optionally mono-, di- or tri-substituted independently with halo, nitro, or (C₁-C₂)alkyl, wherein said (C₁-C₂)alkyl optionally has from one to five fluorines;
R⁵ and R⁶ are each independently hydrogen, methyl, methoxy or chloro; said methoxy optionally having from one to three fluorines, said methyl optionally having from one to three fluorines;
or a pharmaceutically acceptable salt thereof.

5. A compound of claim 4 wherein
Y is ethyl;
R² is ethyl or methyl;
R³ is (3,5-bis-(trifluoromethyl)-phenyl)-hydroxy-methoxycarbonyl-methyl; (3,5-bis-trifluoromethyl-phenyl)-methoxycarbonyl-methyl; (3,5-bis-trifluoromethyl-phenyl)-cyano-methyl, 3,5-bis-trifluoromethyl-benzoyl; (3,5-bis-trifluoromethyl-phenyl)-hydroxy-methyl; (3,5-bis-trifluoromethyl-phenyl)-fluoro-methyl; (3,5-bis-trifluoromethyl-phenyl)-difluoro-methyl; (3,5-bis-(trifluoromethyl)-benzyl); (3,5-bis-trifluoromethyl-phenylcarbamoyl)-methyl; amino-(3,5-bis-(trifluoromethyl)-phenyl)-methyl; (3,5-bis-(trifluoromethyl)-phenyl)-methylamine-methyl; 1-(3,5-bis-(trifluoromethyl)-phenyl)-2-amino-ethyl; 1-(3,5-bis-(trifluoromethyl)-phenyl)-2-fluoro-ethyl; 1-(3,5-bis-(trifluoromethyl)-phenyl)-2-methoxy-ethyl; 1-(3,5-bis-(trifluoromethyl)-phenyl)-2-hydroxy-ethyl; or 2-acetoxy-1-(3,5-bis-(trifluoromethyl)-phenyl)-ethyl;
R⁵ is methoxy or trifluoromethyl; and
R⁶ is hydrogen or methoxy;
or a pharmaceutically acceptable salt thereof.

6. A compound according to claim 5 wherein the bond between C3 and J is a single bond.

7. A compound according to claim 5 wherein the bond between C3 and J is a double bond.

8. A compound selected from the group consisting of:
(R, R, S)-4-[Amino-(3,5-bis-trifluoromethyl-phenyl)-methyl]-2-ethyl-6-trifluoromethyl-3,4-dihydro-2H-quinoline-1-carboxylic acid ethyl ester;
(R, S, S)-4-[Amino-(3,5-bis-trifluoromethyl-phenyl)-methyl]-2-ethyl-6-trifluoromethyl-3,4-dihydro-2H-quinoline-1-carboxylic acid ethyl ester;
(R, R)-4-(3,5-bis-trifluoromethyl-benzyl)-2-ethyl-6-trifluoromethyl-3,4-dihydro-2H-quinoline-1-carboxylic acid ethyl ester;
(R, R, S)-4-[(3,5-bis-trifluoromethyl-phenyl)-methylaminomethyl]-2-ethyl-6-trifluoromethyl-3,4-dihydro-2H-quinoline-1-carboxylic acid ethyl ester;
(R, S, S)-4-[(3,5-bis-trifluoromethyl-phenyl)-methylaminomethyl]-2-ethyl-6-trifluoromethyl-3,4-dihydro-2H-quinoline-1-carboxylic acid ethyl ester;
(R, R)-4-[(3,5-bis-trifluoromethyl-phenyl)-hydroxyl-methoxycarbonyl-methyl]-6,7-dimethoxy-2-methyl-2H-quinoline-1-carboxylic acid ethyl ester;
(R, S)-4-[(3,5-bis-trifluoromethyl-phenyl)-hydroxyl-methoxycarbonyl-methyl]-6,7-dimethoxy-2-methyl-2H-quinoline-1-carboxylic acid ethyl ester;
(R, S, R)-4-[(3,5-bis-trifluoromethyl-phenyl)-hydroxyl-methoxycarbonyl-methyl]-6,7-dimethoxy-2-methyl-3,4-dihydro-2H-quinoline-1-carboxylic acid ethyl ester;
(R, S, S)-4-[(3,5-bis-trifluoromethyl-phenyl)-hydroxyl-methoxycarbonyl-methyl]-6,7-dimethoxy-2-methyl-3,4-dihydro-2H-quinoline-1-carboxylic acid ethyl ester;
(R, R, S)-4-[(3,5-bis-trifluoromethyl-phenyl)-hydroxyl-methoxycarbonyl-methyl]-6,7-dimethoxy-2-methyl-3,4-dihydro-2H-quinoline-1-carboxylic acid ethyl ester;
(R, R, R)-4-[(3,5-bis-trifluoromethyl-phenyl)-hydroxyl-methoxycarbonyl-methyl]-6,7-dimethoxy-2-methyl-3,4-dihydro-2H-quinoline-1-carboxylic acid ethyl ester;
(R, S, R)-4-[(3,5-Bis-trifluoromethyl-phenyl)-hydroxy-methoxycarbonyl-methyl]-2-ethyl-6-trifluoromethyl-3,4-dihydro-2H-quinoline-1-carboxylic acid ethyl ester;
(R, S, S)-4-[(3,5-Bis-trifluoromethyl-phenyl)-hydroxy-methoxycarbonyl-methyl]-2-ethyl-6-trifluoromethyl-3,4-dihydro-2H-quinoline-1-carboxylic acid ethyl ester;
(R, R, S)-4-[(3,5-Bis-trifluoromethyl-phenyl)-hydroxy-methoxycarbonyl-methyl]-2-ethyl-6-trifluoromethyl-3,4-dihydro-2H-quinoline-1-carboxylic acid ethyl ester;
(R, R, R)-4-[(3,5-Bis-trifluoromethyl-phenyl)-hydroxy-methoxycarbonyl-methyl]-2-ethyl-6-trifluoromethyl-3,4-dihydro-2H-quinoline-1-carboxylic acid ethyl ester;
(R, R, S)-4-[(3,5-Bis-trifluoromethyl-phenyl)-methoxycarbonyl-methyl]-6,7-dimethoxy-2-methyl-3,4-dihydro-2H-quinoline-1-carboxylic acid ethyl ester;
(R, R, S)-4-[(3,5-Bis-trifluoromethyl-phenyl)-methoxy-methyl]-2-ethyl-6-trifluoromethyl-3,4-dihydro-2H-quinoline-1-carboxylic acid ethyl ester;
(R, R)-4-[(3,5-Bis-trifluoromethyl-phenyl)-hydroxyl-methoxycarbonyl-methyl]-2-ethyl-6-trifluoromethyl-2H-quinoline-1-carboxylic acid ethyl ester;
(R, S)-4-[(3,5-Bis-trifluoromethyl-phenyl)-hydroxyl-methoxycarbonyl-methyl]-2-ethyl-6-trifluoromethyl-2H-quinoline-1-carboxylic acid ethyl ester;
(R, R, S)-4-[1-(3,5-Bis-trifluoromethyl-phenyl)-2-hydroxy-ethyl]-2-ethyl-6-trifluoromethyl-3,4-dihydro-2H-quinoline-1-carboxylic acid ethyl ester;
(R, S, S)-4-[1-(3,5-Bis-trifluoromethyl-phenyl)-2-hydroxy-ethyl]-2-ethyl-6-trifluoromethyl-3,4-dihydro-2H-quinoline-1-carboxylic acid ethyl ester;
(R, R, S)-4-[2-Acetoxy-1-(3,5-bis-trifluoromethyl-phenyl)-ethyl]-2-ethyl-6-trifluoromethyl-3,4-dihydro-2H-quinoline-1-carboxylic acid ethyl ester;
(R, S, S)-4-[2-Acetoxy-1-(3,5-bis-trifluoromethyl-phenyl)-ethyl]-2-ethyl-6-trifluoromethyl-3,4-dihydro-2H-quinoline-1-carboxylic acid ethyl ester;
(R, R, S)-4-[l-(3,5-Bis-trifluoromethyl-phenyl)-2-methoxy-ethyl]-2-ethyl-6-trifluoromethyl-3,4-dihydro-2H-quinoline-1-carboxylic acid ethyl ester;
(R, S, S)-4-[1-(3,5-Bis-trifluoromethyl-phenyl)-2-methoxy-ethyl]-2-ethyl-6-trifluoromethyl-3,4-dihydro-2H-quinoline-1-carboxylic acid ethyl ester;
(R, R, S)-4-[1-(3,5-Bis-trifluoromethyl-phenyl)-2-fluoro-ethyl]-2-ethyl-6-trifluoromethyl-3,4-dihydro-2H-quinoline-1-carboxylic acid ethyl ester;
(R, S, S)-4-[1-(3,5-Bis-trifluoromethyl-phenyl)-2-fluoro-ethyl]-2-ethyl-6-trifluoromethyl-3,4-dihydro-2H-quinoline-1-carboxylic acid ethyl ester;
(R, R, S)-4-[1-(3,5-Bis-trifluoromethyl-phenyl)-2-amino-ethyl]-2-ethyl-6-trifluoromethyl-3,4-dihydro-2H-quinoline-1-carboxylic acid ethyl ester;
(R, S, S)-4-[1-(3,5-Bis-trifluoromethyl-phenyl)-2-amino-ethyl]-2-ethyl-6-trifluoromethyl-3,4-dihydro-2H-quinoline-1-carboxylic acid ethyl ester;
(R,R)-4-[(3,5-Bis-trifluoromethyl-phenyl)-methoxycarbonyl-methyl]-2-ethyl-6-trifluoromethyl-2H-quinoline-1-carboxylic acid ethyl ester;
(R,S)-4-[(3,5-Bis-trifluoromethyl-phenyl)-methoxycarbonyl-methyl]-2-ethyl-6-trifluoromethyl-2H-quinoline-1-carboxylic acid ethyl ester;
(R,S,S)-4-[(3,5-Bis-trifluoromethyl-phenyl)-methoxycarbonyl-methyl]-2-ethyl-6-trifluoromethyl-3,4-dihydro-2H-quinoline-1-carboxylic acid ethyl ester;
(R,R,S)-4-[(3,5-Bis-trifluoromethyl-phenyl)-methoxycarbonyl-methyl]-2-ethyl-6-trifluoromethyl-3,4-dihydro-2H-quinoline-1-carboxylic acid ethyl ester;
(R,S,S)-4-[(3,5-Bis-trifluoromethyl-phenyl)-cyano-methyl]-2-ethyl-6-trifluoromethyl-3,4-dihydro-2H-quinoline-1-carboxylic acid ethyl ester;
(R,S,R)-4-[(3,5-Bis-trifluoromethyl-phenyl)-cyano-methyl]-2-ethyl-6-trifluoromethyl-3,4-dihydro-2H-quinoline-1-carboxylic acid ethyl ester;
(R,R,S)-4-[(3,5-Bis-trifluoromethyl-phenyl)-cyano-methyl]-2-ethyl-6-trifluoromethyl-3,4-dihydro-2H-quinoline-1-carboxylic acid ethyl ester;
(R,R,R)-4-[(3,5-Bis-trifluoromethyl-phenyl)-cyano-methyl]-2-ethyl-6-trifluoromethyl-3,4-dihydro-2H-quinoline-1-carboxylic acid ethyl ester;
(R,S)-4-[(3,5-Bis-trifluoromethyl-phenyl)-cyano-methyl]-2-ethyl-6-trifluoromethyl-2H-quinoline-1-carboxylic acid ethyl ester;
(R,R)-4-[(3,5-Bis-trifluoromethyl-phenyl)-cyano-methyl]-2-ethyl-6-trifluoromethyl-2H-quinoline-1-carboxylic acid ethyl ester;
(R,S)-4-(3,5-Bis-trifluoromethyl-benzoyl)-6,7-dimethoxy-2-methyl-3,4-dihydro-2H-quinoline-1-carboxylic acid ethyl ester;
(R,R)-4-[(3,5-Bis-trifluoromethyl-benzoyl)-6,7-dimethoxy-2-methyl-3,4-dihydro-2H-quinoline-1-carboxylic acid ethyl ester;
(R,R,R)-4-[(3,5-Bis-trifluoromethyl-phenyl)-fluoro-methyl]-6,7-dimethoxy-2-methyl-3,4-dihydro-2H-quinoline-1-carboxylic acid ethyl ester;
(R,R,S)-4-[(3,5-Bis-trifluoromethyl-phenyl)-fluoro-methyl]-6,7-dimethoxy-2-methyl-3,4-dihydro-2H-quinoline-1-carboxylic acid ethyl ester;
(R,S,R)-4-[(3,5-Bis-trifluoromethyl-phenyl)-fluoro-methyl]-6,7-dimethoxy-2-methyl-3,4-dihydro-2H-quinoline-1-carboxylic acid ethyl ester;
(R,S,S)-4-[(3,5-Bis-trifluoromethyl-phenyl)-fluoro-methyl]-6,7-dimethoxy-2-methyl-3,4-dihydro-2H-quinoline-1-carboxylic acid ethyl ester;
(R,R)-4-(3,5-bis-trifluoromethyl-benzoyl)-2-ethyl-6-trifluoromethyl-3,4-dihydro-2H-quinoline-1-carboxylic acid ethyl ester;
(R,S)-4-(3,5-bis-trifluoromethyl-benzoyl)-2-ethyl-6-trifluoromethyl-3,4-dihydro-2H-quinoline-1-carboxylic acid ethyl ester;
(R,R,R)-4-[(3,5-Bis-trifluoromethyl-phenyl)-hydroxy-methyl]-6,7-dimethoxy-2-methyl-3,4-dihydro-2H-quinoline-1-carboxylic acid ethyl ester;
(R,R,S)-4-[(3,5-Bis-trifluoromethyl-phenyl)-hydroxy-methyl]-6,7-dimethoxy-2-methyl-3,4-dihydro-2H-quinoline-1-carboxylic acid ethyl ester;
(R,S,R)-4-[(3,5-Bis-trifluoromethyl-phenyl)-hydroxy-methyl]-6,7-dimethoxy-2-methyl-3,4-dihydro-2H-quinoline-1-carboxylic acid ethyl ester;
(R,S,S)-4-[(3,5-Bis-trifluoromethyl-phenyl)-hydroxy-methyl]-6,7-dimethoxy-2-methyl-3,4-dihydro-2H-quinoline-1-carboxylic acid ethyl ester;
(R,S,S)-4-[(3,5-Bis-trifluoromethyl-phenyl)-hydroxy-methyl]-2-ethyl-6-trifluoromethyl-3,4-dihydro-2H-quinoline-1-carboxylic acid ethyl ester;
(R,S,R)-4-[(3,5-Bis-trifluoromethyl-phenyl)-hydroxy-methyl]-2-ethyl-6-trifluoromethyl-3,4-dihydro-2H-quinoline-1-carboxylic acid ethyl ester;
(R,R,S)-4-[(3,5-Bis-trifluoromethyl-phenyl)-hydroxy-methyl]-2-ethyl-6-trifluoromethyl-3,4-dihydro-2H-quinoline-1-carboxylic acid ethyl ester; and
(R,R,R)-4-[(3,5-Bis-trifluoromethyl-phenyl)-hydroxy-methyl]-2-ethyl-6-trifluoromethyl-3,4-dihydro-2H-quinoline-1-carboxylic acid ethyl ester;
or a pharmaceutically acceptable salt or prodrug thereof.

9. A compound of claim 1 wherein
J is nitrogen;
the bond between C3 and J is a single bond;
R¹ is W—X;
W is carbonyl;
X is —O—Y;
Y for each occurrence is independently (C₁-C₆)alkyl, said (C₁-C₆)alkyl optionally having one to nine fluorines or said (C₁-C₆)alkyl optionally mono-substituted with Z;
wherein Z is a partially saturated, fully saturated or fully unsaturated three to six membered ring optionally having one to two heteroatoms selected independently from oxygen, sulfur and nitrogen;
wherein said Z substituent is optionally mono-, di- or tri-substituted independently with halo, (C₁-C₄)alkyl, (C₁-C₄)alkoxy, (C₁-C₄)alkylthio, nitro, cyano, oxo, or (C₁-C₄)alkyloxycarbonyl, said (C₁-C₄)alkyl or (C₁-C₄)alkoxy optionally substituted with from one to nine fluorines;
R²is a partially saturated, fully saturated or fully unsaturated (C₁-C₄) straight or branched carbon chain wherein one carbon, other than the connecting carbon, may optionally be replaced with oxygen or sulfur and wherein said carbon is optionally mono-, di- or tri-substituted independently with halo, said carbon chain is optionally mono-substituted with oxo, said carbon is optionally monosubstituted with hydroxy, said sulfur is optionally mono- or di-substituted with oxo,; or said R² is a partially saturated, fully saturated or fully unsaturated three to five membered ring optionally having one heteroatom selected independently from oxygen and sulfur; wherein said R² ring is optionally mono-, di- or tri-substituted independently with halo, hydroxy, (C₁-C₆)alkoxy, amino, nitro, (C₁-C₄)alkyloxycarbonyl or carboxy;
wherein R³ is a fully saturated, partially unsaturated or fully unsaturated one to six membered straight or branched carbon chain wherein each carbon, other than C4a or the R³ carbon adjacent to C4a, may optionally be replaced with one heteroatom selected from oxygen, sulfur and nitrogen, and said carbon is optionally mono-, di- or tri-substituted independently with halo, said carbon, other than C4a, is optionally mono-substituted with hydroxy, said carbon is optionally mono-substituted with cyano, said carbon is optionally mono-substituted with oxo or nitrogen, said sulfur is optionally mono- or di-substituted with oxo, said nitrogen is optionally mono- or di-substituted with hydrogen or oxo, and said carbon chain is optionally mono, di-, or tri-substituted with V at C4a or at the the R³ carbon adjacent to C4a;
V is a five or six membered partially saturated, fully saturated or fully unsaturated ring optionally having one to three heteroatoms selected independently from oxygen, sulfur and nitrogen such that V is not imidazolyl or a fully saturated heterocyclic nitrogen-containing ring wherein nitrogen of the ring is connected to the R³ group; wherein said V ring is optionally mono-, di-, tri-, tetra- or penta-substituted independently with halo, (C₁-C₆)alkyl, hydroxy, (C₁-C₆)alkoxy, (C₁-C₆)alkoxycarbonyl, nitro, cyano or oxo, wherein said (C₁-C₆)alkyl or (C₁-C₆)alkoxy substituent optionally has from one to nine fluorines;
R⁴ is hydrogen;
R⁵ and R⁶ are each independently hydrogen, halo, T, (C₁-C₆)alkoxy or (C₁-C₆)alkyl, said (C₁-C₆)alkoxy or (C₁-C₆)alkyl substituent optionally having from one to nine fluorines or said (C₁-C₆)alkoxy or (C₁-C₆)alkyl substituent optionally mono-substituted with T;
wherein T is a partially saturated, fully saturated or fully unsaturated five to six membered ring optionally having one to two heteroatoms selected independently from oxygen, sulfur and nitrogen;
wherein said T substituent is optionally mono-, di- or tri-substituted independently with halo, (C₁-C₆)alkyl, hydroxy, (C₁-C₆)alkoxy, (C₁-C₄)alkylthio, amino, oxo, carboxy, (C₁-C₆)alkyloxycarbonyl, mono-N— or di-N,N—(C₁-C₆)alkylamino wherein said (C₁-C₆)alkyl or (C₁-C₆)alkoxy substituent optionally has from one to nine fluorines;
R⁷ is hydrogen;
or a pharmaceutically acceptable salt thereof.

10. A compound of claim 9 wherein
Y is (C₁-C₄)alkyl, wherein said (C₁-C₄)alkyl substituent optionally has one to nine fluorines;
R² is (C₁-C₄)alkyl, cyclopropyl or cyclobutyl;
R³ is —C(O)—V, —CH(C(O)O(C₁-C₃)alkyl)(V), or —CH(CN)(V);
V is phenyl optionally mono-, di- or tri-substituted independently with halo, (C₁-C₆)alkyl, hydroxy, (C₁-C₆)alkoxy, nitro, cyano or oxo wherein said (C₁-C₆)alkyl substituent optionally has from one to nine fluorines;
R⁵ and R⁶ are each independently hydrogen, (C₁-C₃)alkoxy or (C₁-C₆)alkyl, said (C₁-C₃)alkoxy optionally having from one to nine fluorines, said (C₁-C₆)alkyl optionally having from one to seven fluorines;
or a pharmaceutically acceptable salt thereof.

11. A compound of claim 10 wherein
Y is methyl, ethyl, 1-propyl, 2-propyl or tert-butyl;
R² is methyl, ethyl, 2-propyl, cyclopropyl or cyclobutyl;
R³ is 3,5-bis-trifluoromethyl-benzoyl, (3,5-bis-trifluoromethyl-phenyl)-cyano-methyl, or (3,5-bis-trifluoromethyl-phenyl)-methoxycarbonyl-methyl;
R⁵ is methyl or trifluoromethyl;
R⁶ is hydrogen or methyl.

12. A compound selected from the group consisting of:
(R)-4-(3,5-Bis-trifluoromethyl-benzoyl)-2-ethyl-6,7-dimethyl-3,4-dihydro-2H-quinoxaline-1-carboxylic acid ethyl ester;
(R,R)-4-[(3,5-Bis-trifluoromethyl-phenyl)-cyano-methyl]-2-ethyl-6,7-dimethyl-3,4-dihydro-2H-quinoxaline-1-carboxylic acid ethyl ester;
(R,S)-4-[(3,5-Bis-trifluoromethyl-phenyl)-cyano-methyl]-2-ethyl-6,7-dimethyl-3,4-dihydro-2H-quinoxaline-1-carboxylic acid ethyl ester;
(R,R)-4-[(3,5-Bis-trifluoromethyl-phenyl)-methoxycarbonyl-methyl]-2-ethyl-6,7-dimethyl-3,4-dihydro-2H-quinoxaline-1-carboxylic acid ethyl ester;
(R,S)-4-[(3,5-Bis-trifluoromethyl-phenyl)-methoxycarbonyl-methyl]-2-ethyl-6,7-dimethyl-3,4-dihydro-2H-quinoxaline-1-carboxylic acid ethyl ester;
(R,R)-4-[(3,5-Bis-trifluoromethyl-phenyl)-methoxycarbonyl-methyl]-2-ethyl-6,7-dimethyl-3,4-dihydro-2H-quinoxaline-1-carboxylic acid methyl ester;
(R,S)-4-[(3,5-Bis-trifluoromethyl-phenyl)-methoxycarbonyl-methyl]-2-ethyl-6,7-dimethyl-3,4-dihydro-2H-quinoxaline-1-carboxylic acid methyl ester;
(R,R)-4-[(3,5-Bis-trifluoromethyl-phenyl)-methoxycarbonyl-methyl]-2-ethyl-6,7-dimethyl-3,4-dihydro-2H-quinoxaline-1-carboxylic acid isopropyl ester;
(R,S)-4-[(3,5-Bis-trifluoromethyl-phenyl)-methoxycarbonyl-methyl]-2-ethyl-6 ,7-dimethyl-3,4-dihydro-2H-quinoxaline-1-carboxylic acid isopropyl ester;
(R,R)-4-[(3,5-Bis-trifluoromethyl-phenyl)-methoxycarbonyl-methyl]-2-ethyl-6-trifluoromethyl-3,4-dihydro-2H-quinoxaline-1-carboxylic acid ethyl ester; and
(R,S)-4-[(3,5-Bis-trifluoromethyl-phenyl)-methoxycarbony-methyl]-2-ethyl-6-trifluoromethyl-3,4-dihydro-2H-quinoxaline-1-carboxylic acid ethyl ester;
or a pharmaceutically acceptable salt or prodrug thereof.

13. A compound according to Formula II 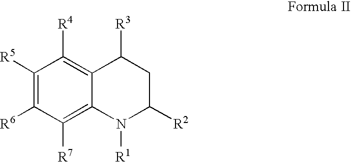
wherein
R¹ is W—X;
W is carbonyl;
X is —O—Y;
Y for each occurrence is independently (C₁-C₆)alkyl, said (C₁-C₆)alkyl optionally having one to nine fluorines or said (C₁-C₆)alkyl optionally mono-substituted with Z;
wherein Z is a partially saturated, fully saturated or fully unsaturated three to six membered ring optionally having one to two heteroatoms selected independently from oxygen, sulfur and nitrogen;
wherein said Z substituent is optionally mono-, di- or tri-substituted independently with halo, (C₁-C₄)alkyl, (C₁-C₄)alkoxy, (C₁-C₄)alkylthio, nitro, cyano, oxo, or (C₁-C₄)alkyloxycarbonyl, said (C₁-C₄)alkyl optionally substituted with from one to nine fluorines;
R² is a partially saturated, fully saturated or fully unsaturated (C₁-C₄) straight or branched carbon chain wherein each carbon, other than the connecting carbon, may optionally be replaced with one heteroatom selected independently from oxygen and sulfur wherein said carbon is optionally mono-, di- or tri-substituted independently with halo, said carbon chain is optionally mono-substituted with oxo, said carbon is optionally mono-substituted with hydroxy, said sulfur is optionally mono- or di-substituted with oxo,; or said R² is a partially saturated, fully saturated or fully unsaturated three to five membered ring optionally having one heteroatom selected independently from oxygen and sulfur;
wherein said R² ring is optionally mono-, di- or tri-substituted independently with halo or (C₁-C₆)alkoxy;
wherein R³ is —CH₂NR⁸R⁹ or —C(O)NR⁸R⁹;
wherein R⁸ and R⁹ are independently hydrogen or a (C₁-C₂) straight carbon chain wherein at least one of R⁸ and R⁹ are not hydrogen and wherein said carbon is optionally mono-, di- or tri-substituted independently with halo, said carbon, other than the connecting carbon, is optionally mono-substituted with hydroxy, said carbon is optionally mono-substituted with oxo, and said carbon chain is optionally mono, di-, or tri-substituted with V³, wherein either R⁸ or R⁹ is substituted with V³, or both R⁸ and R⁹ is substituted with V^{3,};
wherein V³ is a partially saturated, fully saturated or fully unsaturated three to six membered ring optionally having one to two heteroatoms selected independently from oxygen, sulfur and nitrogen, or a bicyclic ring consisting of two fused partially saturated, fully saturated or fully unsaturated three to six membered rings, taken independently, optionally having one to four heteroatoms selected independently from nitrogen, sulfur and oxygen;
wherein said V³ substituent is optionally mono-, di-, tri-, tetra- or penta-substituted independently with V⁴, (C₁-C₆)alkyl-V⁴, C(O)—V⁴, O—(C₀-C₆)alkyl-V⁴, (C₁-C₆)alkyl-O—V⁴, halo, (C₁-C₆)alkyl, (C₁-C₆)alkoxy, hydroxy, oxo, amino, nitro, cyano, (C₁-C₄)alkylthio, (C₁-C₄)alkylsulfinyl, (C₁-C₄)alkylsulfonyl, mono-N— or di-N,N—(C₁-C₆)alkylsulfonyl, (C₁-C₆)alkyloxycarbonyl, mono-N— or di-N,N—(C₁-C₆)alkylamino, wherein said (C₁-C₆)alkyl substituent is optionally mono-substituted with oxo, wherein said (C₁-C₆)alkyl substituent is optionally mono-substituted with hydroxy, said (C₁-C₆)alkyl or (C₁-C₆)alkoxy substituents are also optionally substituted with from one to nine fluorines;
wherein V⁴ is a partially saturated, fully saturated or fully unsaturated three to six membered ring optionally having one to two heteroatoms selected independently from oxygen, sulfur and nitrogen, or a bicyclic ring consisting of two fused partially saturated, fully saturated or fully unsaturated three to six membered rings, taken independently, optionally having one to four heteroatoms selected independently from nitrogen, sulfur and oxygen;
wherein said V⁴ substituent is optionally mono-, di-, tri-, or tetra-substituted independently with halo, (C₁-C₆)alkyl, (C₁-C₆)alkoxy, hydroxy, oxo, amino, nitro, cyano, (C₁-C₆)alkyloxycarbonyl, mono-N— or di-N,N—(C₁-C₆)alkylamino wherein said (C₁-C₆)alkyl substituent is optionally mono-substituted with oxo, said (C₁-C₆)alkyl or (C₁-C₆)alkoxy substituent is also optionally substituted with from one to nine fluorines;
or wherein R⁸and R⁹ are taken together to form a partially saturated, fully saturated or fully unsaturated three to eight membered ring optionally having one to four heteroatoms selected independently from oxygen, sulfur, and nitrogen or a bicyclic ring consisting of two fused partially saturated, fully saturated or fully unsaturated three to six membered rings, selected independently from nitrogen, sulfur and oxygen;
R⁴ is hydrogen;
R⁵ and R⁶ are each independently hydrogen, halo, T, (C₁-C₆)alkoxy or (C₁-C₆)alkyl, said (C₁-C₆)alkoxy or (C₁-C₆)alkyl substituent optionally having from one to nine fluorines or said (C₁-C₆)alkoxy or (C₁-C₆)alkyl substituent optionally mono-substituted with T;
wherein T is a partially saturated, fully saturated or fully unsaturated five to six membered ring optionally having one to two heteroatoms selected independently from oxygen, sulfur and nitrogen;
wherein said T substituent is optionally mono-, di- or tri-substituted independently with halo, (C₁-C₆)alkyl, hydroxy, (C₁-C₆)alkoxy, (C₁-C₄)alkylthio, amino, oxo, carboxy, (C₁-C₆)alkyloxycarbonyl, mono-N— or di-N,N—(C₁-C₆)alkylamino wherein said (C₁-C₆)alkyl substituent optionally has from one to nine fluorines;
R⁷ is hydrogen;
or a pharmaceutically acceptable salt or prodrug thereof.

14. A method for treating atherosclerosis, coronary artery disease, coronary heart disease, coronary vascular disease, peripheral vascular disease, dyslipidemia, hyperbetalipoproteinemia, hypoalphalipoproteinemia, hypercholesterolemia, hypertriglyceridemia, familial-hypercholesterolemia or myocardial infarction in a mammal by administering to a mammal in need of such treatment an atherosclerosis, coronary artery disease, coronary heart disease, coronary vascular disease, peripheral vascular disease, dyslipidemia, hyperbetalipoproteinemia, hypoalphalipoproteinemia, hypercholesterolemia, hypertriglyceridemia, familial-hypercholesterolemia or myocardial infarction treating amount of a compound of claim 1, 8, 12 or 13, a prodrug thereof, or a pharmaceutically acceptable salt of said compound or of said prodrug.

15. A method as recited in claim 14 wherein atherosclerosis is treated.

16. A method as recited in claim 14 wherein peripheral vascular disease is treated.

17. A method as recited in claim 14 wherein dyslipidemia is treated.

18. A method as recited in claim 14 wherein hyperbetalipoproteinemia is treated.

19. A method as recited in claim 14 wherein hypoalphalipoproteinemia is treated.

20. A method as recited in claim 14 wherein familial-hypercholesterolemia is treated.

21. A method as recited in claim 14 wherein coronary artery disease is treated.

22. A method as recited in claim 14 wherein myocardial infarction is treated.

23. A pharmaceutical composition which comprises a therapeutically effective amount of a compound of claim 1, 8, 12, or 13, a prodrug thereof, or a pharmaceutically acceptable salt of said compound or of said prodrug and a pharmaceutically acceptable vehicle, diluent or carrier.

24. A pharmaceutical composition for the treatment of atherosclerosis, coronary artery disease, coronary heart disease, coronary vascular disease, peripheral vascular disease, dyslipidemia, hyperbetalipoproteinemia, hypoalphalipoproteinemia, hypercholesterolemia, hypertriglyceridemia, familial-hypercholesterolemia or myocardial infarction in a mammal which comprises a therapeutically effective amount of a compound of claim 1, 8, 12, or 13, a prodrug thereof, or a pharmaceutically acceptable salt of said compound or of said prodrug and a pharmaceutically acceptable vehicle, diluent or carrier.

25. A pharmaceutical composition for the treatment of atherosclerosis in a mammal which comprises an atherosclerosis treating amount of a compound of claim 1, 8, 12, or 13, a prodrug thereof, or a pharmaceutically acceptable salt of said compound or of said prodrug and a pharmaceutically acceptable vehicle, diluent or carrier.

26. A pharmaceutical combination composition comprising: a therapeutically effective amount of a composition comprising
a first compound, said first compound being a compound of claim 1, 8, 12, or 13, a prodrug thereof, or a pharmaceutically acceptable salt of said compound or of said prodrug;
a second compound, said second compound being an HMG CoA reductase inhibitor, an MTP/Apo B secretion inhibitor, a PPAR modulator, a bile acid reuptake inhibitor, a cholesterol absorption inhibitor, a cholesterol synthesis inhibitor, a fibrate, niacin, slow-release niacin, a combination of niacin and lovastatin, an ion-exchange resin, an antioxidant, an ACAT inhibitor or a bile acid sequestrant; and
a pharmaceutical vehicle, diluent or carrier.

27. A pharmaceutical combination composition as recited in claim 26 wherein the second compound is an HMG-CoA reductase inhibitor or a PPAR modulator.

28. A pharmaceutical combination composition as recited in claim 27 wherein the second compound is lovastatin, simvastatin, pravastatin, fluvastatin, atorvastatin, rivastatin, rosuvastatin or pitavastatin.

29. A pharmaceutical combination composition as recited in claim 28 further comprising a cholesterol absorption inhibitor.

30. A pharmaceutical combination composition as recited in claim 29 wherein the cholesterol absorption inhibitor is ezetimibe.

31. A method for treating atherosclerosis in a mammal comprising administering to a mammal in need of treatment thereof;
a first compound, said first compound being a compound of claim 1, 8, 12, or 13, a prodrug thereof, or a pharmaceutically acceptable salt of said compound or of said prodrug; and
a second compound, said second compound being an HMG CoA reductase inhibitor, a PPAR modulator, a cholesterol absorption inhibitor, a cholesterol synthesis inhibitor, a fibrate, niacin, slow-release niacin, a combination of niacin and lovastatin, an ion-exchange resin, an antioxidant, an ACAT inhibitor or a bile acid sequestrant
wherein the amounts of first and second compounds result in a therapeutic effect.

32. A method for treating atherosclerosis as recited in claim 31 wherein the second compound is an HMG-CoA reductase inhibitor or a PPAR modulator.

33. A method for treating atherosclerosis as recited in claim 32 wherein the second compound is lovastatin, simvastatin, pravastatin, fluvastatin, atorvastatin, rivastatin, rosuvastatin or pitavastatin.

34. A method for treating atherosclerosis as recited in claim 33 further comprising administering a cholesterol absorption inhibitor.

35. A method for treating atherosclerosis as recited in claim 34 wherein the cholesterol absorption inhibitor is ezetimibe.

36. A kit for achieving a therapeutic effect in a mammal comprising packaged in association a first therapeutic agent comprising a therapeutically effective amount of a compound of claim 1, 8, 12, or 13, a prodrug thereof, or a pharmaceutically acceptable salt of said compound or of said prodrug and a pharmaceutically acceptable carrier, a second therapeutic agent comprising a therapeutically effective amount of an HMG CoA reductase inhibitor, a PPAR modulator, a cholesterol absorption inhibitor, a cholesterol synthesis inhibitor, a fibrate, niacin, slow-release niacin, a combination of niacin and lovastatin, an ion-exchange resin, an antioxidant, an ACAT inhibitor or a bile acid sequestrant and a pharmaceutically acceptable carrier and directions for administration of said first and second agents to achieve the therapeutic effect.

37. A kit as recited in claim 36 wherein said second therapeutic agent comprises an HMG-CoA reductase inhibitor or a PPAR modulator.

38. A kit as recited in claim 37 wherein said second therapeutic agent comprises lovastatin, simvastatin, pravastatin, fluvastatin, atorvastatin, rivastatin, rosuvastatin or pitavastatin.

39. A kit as recited in claim 38 further comprising a cholesterol absorption inhibitor.

40. A kit as recited in claim 39 wherein the cholesterol absorption inhibitor is ezetimibe.

41. A compound of Formula III 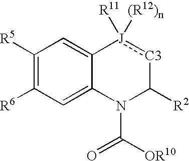
wherein
C3 is carbon;
J is carbon, wherein the bond between C3 and J is a single or double bond;
n is zero if the bond between C3 and J is a double bond or one if the bond between C3 and J is a single bond;
R² is (C₁-C₄)alkyl, cyclopropyl or cyclobutyl;
R⁵ is CF₃;
R⁶ is hydrogen;
R¹⁰ is a fully saturated (C₁-C₄) straight or branched carbon chain;
R¹¹ is halo, hydroxy, —C(O)(O(C₁-C₄)alkyl), —C(O)C(O)(O(C₁-C₄)alkyl), —C(O)NH(O(C₁-C₄)alkyl), or —C(O)N((C₁-C₄)alkyl)(O(C₁-C₄)alkyl);
R¹² is hydrogen or halo, wherein R¹¹ is not halo when R¹² is halo;
or R¹¹ and R¹² are taken together to form oxo or N₂;
or a pharmaceutically acceptable salt or prodrug thereof.

42. A compound according to claim 41, wherein the compound is:
2-Ethyl-4-iodo-6-trifluoromethyl-2H-quinoline-1-carboxylic acid ethyl ester;
2-Ethyl-4-iodo-6-trifluoromethyl-2H-quinoline-1-carboxylic acid ethyl ester;
4-Chloro-2-ethyl-6-trifluoromethyl-3,4-dihydro-2H-quinoline-1-carboxylic acid ethyl ester;
4-Bromo-2-ethyl-6-trifluoromethyl-3,4-dihydro-2H-quinoline-1-carboxylic acid ethyl ester;
4-Diazo-2-ethyl-6-trifluoromethyl-3,4-dihydro-2H-quinoline-1-carboxylic acid ethyl ester;
4-Chloro-2-ethyl-6-trifluoromethyl-3,4-dihydro-2H-quinoline-1,4-dicarboxylic acid 1-ethyl ester 4-methyl ester;
2-Ethyl-6-trifluoromethyl-3,4-dihydro-2H-quinoline-1,4-dicarboxylic acid-1-ethyl ester-4-methyl ester;
2-Ethyl-4-(methoxy-methyl-carbamoyl)-6-trifluoromethyll-3,4-dihydro-2H-quinoline-1-carboxylic acid ethyl ester;
2-Ethyl-6-trifluoromethyl-2H-quinoline-1,4-dicarboxylic acid-1-ethyl ester-4-methyl ester;
4-Chloro-2-ethyl-4-methoxycarboncarbonyl-6-trifluoromethyl-3,4-dihydro-2H-quinoline-1-carboxylic acid ethyl ester;
2-Ethyl-4-methoxycarboncarbonyl-6-trifluoromethyl-2H-quinoline-1-carboxylic acid-ethyl ester;
4-Diazo-6,7-dimethoxy-2-methyl-3,4-dihydro-2H-quinoline-1-carboxylic acid ethyl ester;
6,7-Dimethoxy-4-methoxycarboncarbonyl-2-methyl-2H-quinoline-1-carboxylic acid-ethyl ester;
6,7-Dimethoxy-2-methyl-3,4-dihydro-2H-quinoline-1,4-dicarboxylic acid-1-ethyl ester-4-methyl ester;
6,7-Dimethoxy-4-(methoxy-methyl-carbamoyl)-2-methyl-3,4-dihydro-2H-quinoline-1-carboxylic acid ethyl ester;
2-Ethyl-4-hydroxy-6-trifluoromethyl-3,4-dihydro-2H-quinoline-1-carboxylic acid ethyl ester;
2-Ethyl-4-oxo-6-trifluoromethyl-3,4-dihydro-2H-quinoline-1-carboxylic acid ethyl ester;
2-Ethyl-4-hydroxy-6-trifluoromethyl-3,4-dihydro-2H-quinoline-1-carboxylic acid methyl ester;
2-Ethyl-4-oxo-6-trifluoromethyl-3,4-dihydro-2H-quinoline-1-carboxylic acid methyl ester;
2-Ethyl-4-hydroxy-6-trifluoromethyl-3,4-dihydro-2H-quinoline-1-carboxylic acid 1-propyl ester;
2-Ethyl-4-oxo-6-trifluoromethyl-3,4-dihydro-2H-quinoline-1-carboxylic acid 1-propyl ester;
2-Ethyl-4-hydroxy-6-trifluoromethyl-3,4-dihydro-2H-quinoline-1-carboxylic acid 2-propyl ester;
2-Ethyl-4-oxo-6-trifluoromethyl-3,4-dihydro-2H-quinoline-1-carboxylic acid 2-propyl ester;
2-Ethyl-4-hydroxy-6-trifluoromethyl-3,4-dihydro-2H-quinoline-1-carboxylic acid tert-butyl ester;
2-Ethyl-4-oxo-6-trifluoromethyl-3,4-dihydro-2H-quinoline-1-carboxylic acid tert-butyl ester; or
2-Ethyl-6-trifluoromethyl-3,4-dihydro-2H-quinoline-1,4-dicarboxylic acid-1-ethyl ester-4-methyl ester;
or a pharmaceutically acceptable salt or prodrug thereof.
